(19) United States
(12) Patent Application Publication
(10) Pub. No.: US 2025/0163468 A1
(43) Pub. Date: May 22, 2025

(54) COVALENTLY SURFACE MODIFIED ADENO-ASSOCIATED VIRUS PRODUCTION BY IN VITRO CONJUGATION, AND PURIFICATION OF COVALENTLY SURFACE MODIFIED ADENO-ASSOCIATED VIRUS

(71) Applicant: Regeneron Pharmaceuticals, Inc., Tarrytown, NY (US)

(72) Inventors: Garima Thakur, White Plains, NY (US); Zhe Zhang, Scarsdale, NY (US); Sheldon Robert Mink, Hawthorne, NY (US); Andrew David Tustian, Millwood, NY (US)

(21) Appl. No.: 18/955,344

(22) Filed: Nov. 21, 2024

Related U.S. Application Data
(60) Provisional application No. 63/713,916, filed on Oct. 30, 2024.
(60) Provisional application No. 63/691,544, filed on Sep. 6, 2024.
(60) Provisional application No. 63/654,478, filed on May 31, 2024.
(60) Provisional application No. 63/620,682, filed on Jan. 12, 2024.
(60) Provisional application No. 63/620,055, filed on Jan. 11, 2024.
(60) Provisional application No. 63/601,611, filed on Nov. 21, 2023.

(51) Int. Cl. C12N 15/86; C12N 7/00

(52) U.S. Cl. CPC C12N 15/86; C12N 7/00; C12N 2750/14122; C12N 2750/14143; C12N 2750/14145

(57) ABSTRACT

The present inventions provide covalently surface modified adeno-associated viruses can comprise genes of interest (GOIs) and advantageously can be targeted to certain cell and tissue types for preventative and therapeutic purposes. The covalently surface modified adeno-associated viruses also can be mutated to detarget certain tissues and organs, such as the liver. The present inventions further provide systems and methods for engineering adeno-associated virus (AAV) to create covalently surface modified adeno-associated viruses, and methods of purifying such covalently surface modified adeno-associated viruses. The inventions further include covalently surface modified adeno-associated viruses and preparations and products comprising such covalently surface modified adeno-associated viruses.

This application claims priority to U.S. Application Ser. No. 63/713,916, filed Oct. 30, 2024; U.S. Application Ser. No. 63/691,544, filed Sep. 6, 2024; U.S. Application Ser. No. 63/654,478, filed May 31, 2024; U.S. Application Ser. No. 63/620,682, filed Jan. 12, 2024; U.S. Application Ser. No. 63/620,055, filed Jan. 11, 2024; and U.S. Application Ser. No. 63/601,611, filed Nov. 21, 2023. These applications are incorporated by reference in their entirety.

### FIELD OF THE INVENTIONS

The present inventions provide systems and methods for engineering adeno-associated virus (AAV) to create covalently surface modified adeno-associated virus species using in vitro conjugation, and methods of purifying such covalently surface modified adeno-associated viruses from unconjugated retargeting molecule species.

### REFERENCE TO ELECTRONIC SEQUENCE LISTING

This application contains a Sequence Listing which has been submitted electronically in .XML format and is hereby incorporated by reference in its entirety. The .XML copy, created on Nov. 8, 2024, is named “11547.xml” and is 334,915 bytes in size. The sequence listing contained in this .XML file is part of the specification and is hereby incorporated by reference herein in its entirety.

### BACKGROUND OF THE INVENTIONS

Adeno-associated virus (AAV) is a non-enveloped, single-stranded DNA virus and is used as a gene delivery vector for both research and therapeutics. Weitzman and Linden, Adeno-Associated Virus Biology (chapter 1), Meth. Molec. Biol. 807: 1-23 (2011). There are numerous AAV serotypes and variants thereof. AAV serotypes include, for example, AAV1, 2, 3, 4, 5, 6, 7, 8, 9, 10, 11, 12, 13, rh74, as well as variants thereof. AAV serotypes share common properties, structure, and genomic sequence and organization. See, for example, Issa et al, Cells 12: 285 (2023); Goedeker et al., Ther. Adv. Neurol. Disord. 16: 1-7 (2023).

Gene transfer vectors based on AAV have demonstrated promise for human gene therapy based on their safety profile and potential to achieve long-term efficacy in animal models. Wang et al., Nature, 18: 358-78 (2019). A major challenge for advancing AAV-based therapies into clinical development is the difficulty and cost of producing sufficient quantities of AAV through transient methodologies. Additionally, recombinant AAVs containing genes of interest (GOIs), but lacking surface modifications, are currently being used in preventative and therapeutic capacities, such as in vaccines and in gene therapy. However, such AAVs have limited tissue specificity and a propensity to accumulate in the liver.

The wild type AAV genome includes a capsid gene referred to as “Cap” or “cap”. Cap in nature is translated to produce, via alternative start codons and transcript splicing, three size-variant structural proteins referred to as VP1 (about 90 kDa), VP2 (about 72 kDa) and VP3 (about 60 kDa). An AAV capsid contains 60 subunits total of the VP proteins. A ratio of 1:1:10 is considered the most typical ratio for VP1:VP2:VP3, with a stoichiometry of 5 VP1 subunits:5 VP2 subunits:50 VP3 subunits. However, there can be variation. Wömner et aL., Nature Communications 12:1642 (2021).

Recombinant AAV (rAAV) has been produced in HEK 293, HeLa BHK, human amniotic (for example, epithelial cells such as HAEpiC), CHO and Sf9 lines, for example. First generation rAAV was comprised of a GOI replacing the AAV Cap and Rep genes. The GOI would be flanked by AAV inverted terminal repeats (ITRs) so that the GOI could be packaged within an AAV capsid.

Covalently surface modified AAVs have been produced. See WO 2019/006046. However, there exists a need to address by-products, such as excess unconjugated retargeting molecule species, that will exist freely as contaminants. The present invention provides production and purification solutions.

### SUMMARY OF THE INVENTIONS

Many potential gene therapy applications would benefit from cell/tissue-specific retargeting and detargeting that is beyond current AAV vector control capabilities. The current inventions advantageously employ covalently surface modified AAV for retargeting. Additionally, according to the inventions, mutations optionally can be introduced into the AAV Cap proteins to detarget tissues and organs, such as the liver.

The inventions provide methods of producing covalently surface modified adeno-associated virus (AAV) using in vitro conjugation, wherein the method comprises the steps of (1) combining recombinant AAV with retargeting molecules in a ratio selected to achieve a desired level of conjugation; and (II) incubating the AAV and retargeting molecules for a period of time and under conditions to achieve the desired level of conjugation. The recombinant AAV can obtained by (A) transfecting a cell with: (1) a plasmid comprising a gene of interest flanked by AAV inverted terminal repeats; (2) a plasmid comprising an AAV rep gene and an AAV cap gene; (3) a plasmid comprising AAV rep and cap genes and a polynucleotide sequence encoding a first member of a specific binding pair; and (4) a plasmid comprising adenovirus helper genes E4 and E2, and VA RNA; (B) culturing the transfected cell from step (A) to allow expression of plasmids (1) to (4) and assembly of proteins formed by the expression of plasmids (1) to (4); and (C) harvesting the recombinant AAV. The cell can be a mammalian cell, such as a human cell. Other viruses can be the source of helper polynucleotide sequences encoding helper products, such as herpes simplex virus (UL5, UL8, UL9, UL29, UL30, UL42 and UL52 polynucleotides), human papilloma virus (E1 or E1 carboxyl domain polynucleotides), bocavirus or baculovoirus.

The inventions also provide methods of producing covalently surface modified adeno-associated virus using in vitro conjugation, wherein the method comprises the steps of (1) combining recombinant AAV (rAAV) with retargeting molecules in a ratio selected to achieve a desired level of conjugation; (II) incubating the AAV and retargeting molecules for a period of time and under conditions to achieve the desired level of conjugation. The rAAV is obtained by (A) transfecting a cell with: (1) a plasmid comprising a gene of interest flanked by adeno-associated virus (AAV) inverted terminal repeats; (2) a plasmid comprising an AAV rep gene and an AAV cap gene; (3) a plasmid comprising AAV rep and cap genes and a polynucleotide sequence encoding a first member of a specific binding pair; and (4) a plasmid comprising one or more helper polynucleotide sequences; (B) culturing the transfected cell from step (A) to allow expression of plasmids (1) to (4) and assembly of proteins formed by the expression of plasmids (1) to (4); and (C) harvesting the recombinant AAV. The cell can be a mammalian cell, such as a human cell. The recombinant AAV can comprise a gene of interest. The recombinant AAV can comprise a recombinant capsid protein. The recombinant capsid protein can comprise an amino acid sequence of a first member of a specific binding pair, such as SpyTag. The retargeting molecule can be bound to a second cognate member of a specific binding pair, such as SpyCatcher or fragments/derivatives thereof. The retargeting molecule can be an Fc-containing protein, such as a monoclonal antibody, or an Fc-Fusion protein, or a monoclonal antibody fragment, or any molecule containing an antigen-binding region.

According to the methods of the inventions, the ratio of AAV with FM (First Member) to retargeting molecule with SCM (Second Cognate Member) is 1:20 or a greater ratio difference, for example about 1:30 to about 1:300, about 1:300 to about 1:1000 or about 1:30 to about 1:1000. The populations of FMs and SCMs can be homogenous or heterogeneous as long as the selected FMs can come in contact with appropriate SCMs so that binding can occur.

Preferred levels of molar ratios of AAV-FM to RM-SCM as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

The ratio of the plasmids comprising AAV rep and cap genes and a polynucleotide sequence encoding a first member of a specific binding pair to total plasmids comprising AAV rep and cap genes (termed “mosaicisms”) can include the following ratios and those thereabout or therebetween: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9 or 1/60.

Preferred mosaicisms are: 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13. More preferred mosaicisms are: 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12. Still more preferred mosaicisms are 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11. Ranges of mosaicisms can be 1/1 to 1/60. Preferred ranges of mosaicisms are 1/4 to 1/13; 1/5 to 1/12; 1/6 to 1/11, 1/7 to 1/10.5 or 1/7.5 to 1/10.

The incubating step can about 4 to 72 hours long. The (I) combining step can further comprise an additive, such as isopropyl alcohol (IPA), preferably at a concentration above 5%. IPA concentrations can be about 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25% or more. Preferably, the concentration can be about 10% to 25%, more preferably 10% to 20%.

The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 90%, 70% to 90%, 75% to 90%, 75% to 85%, 75% to 80%, or 80% to 85%, and any ranges made by combining these ranges, such as 65% to 85%, for example. More preferably, the range is 65% to 95%.

The helper polynucleotide sequences can encode adenovirus E4, adenovirus E2, and VA RNA, or be from another virus, such as herpes simplex virus (UL5, UL8, UL9, UL29, UL30, UL42 and UL52 polynucleotides), human papilloma virus (E1 or E1 carboxyl domain polynucleotides), bocavirus or baculovoirus.

The inventions also provide methods of separating a covalently surface modified adeno-associated virus (AAV) species from unconjugated retargeting molecule species, wherein the method comprises the steps of (a) providing a sample comprising the covalently surface modified adeno-associated virus species produced using in vitro conjugation; (b) subjecting the covalently surface modified adeno-associated virus to anion exchange media using a buffering agent and a salt to separate the covalently surface modified AAV species from retargeting molecule species to provide at least one fraction that is enriched in covalently surface modified AAV species; and (c) harvesting the enriched fraction comprising covalently surface modified AAV species with a defined level of covalent surface modifications. The buffering agent can be selected from the group consisting of Bis-Tris-Propane, Bis-Tris, Tris, Glycine, Bicine, Tricine, Acetate, Borate, Citrate, Carbonate, Phosphate, Formate, Sulfate, Succinic acid, Sulfonic acid and variants thereof (for example, MES, PIPES, HEPES, CHES, CAPS, MMS, PBMS), Diethanolamine, and Imidazole, and the salt is selected from the group consisting of NaCl, KCl, MgCl₂, CaCl₂), NH₄Cl, Na₂SO₄, CaSO4, K₂SO₄, MgSO₄, (NH₄)₂SO₄, sodium citrate, and tetramethylammonium chloride (TMAC). The anion exchange media is selected from the group consisting of CIM QA, CIM DEAE, PRIMA T, Capto Q, Capto Q ImpRes, CAPTO DEAE, POROS HQ, POROS XQ, POROS PI, POROS D, Fractogel EMD TMAE, Fractogel EMD DEAE, Nuvia Q, Nuvia HP-Q, Sartobind STIC PA, Sartobind Q, Natrix Q. Enrichment can result in greater than 75%, 80%, 85%, 90%, 95%, 96%, 97%, 98%, 99% or more of unconjugated retargeting molecule species being separated from the covalently surface modified AAV species. Enrichment can result in reducing the presence of unconjugated retargeting molecule species to undetectable levels.

The inventions further provide methods of separating a covalently surface modified adeno-associated virus (AAV) species from unconjugated retargeting molecule species, wherein the method comprises the steps of (a) providing a sample comprising the covalently surface modified adeno-associated virus species produced using an in vitro conjugation; (b) subjecting the covalently surface modified adeno-associated virus to cation exchange media using a buffering agent and a salt to separate the covalently surface modified AAV species from unconjugated retargeting molecule species to provide at least one fraction that is enriched in covalently surface modified AAV species; and (c) harvesting the enriched fraction comprising covalently surface modified AAV species. The cation exchange media can be selected from the group consisting of CIM SO3, Capto S, Capto S ImpRes, Capto S ImpAct, Capto SP, POROS HS, POROS XS, CM Sepharose, Nuvia S, Nuvia HR-Sartobind S, Natrix CH. Enrichment can result in greater than 75%, 80%, 85%, 90%, 95%, 96%, 97%, 98%, 99% or more of unconjugated retargeting molecule species being separated from the covalently surface modified AAV species. Enrichment can result in reducing the presence of unconjugated retargeting molecule species to undetectable levels.

The inventions further provide methods of separating a covalently surface modified adeno-associated virus (AAV) species from unconjugated retargeting molecule species, wherein the method comprises the steps of (a) providing a sample comprising the covalently surface modified adeno-associated virus species produced using an in vitro conjugation; (b) subjecting the covalently surface modified adeno-associated virus to multimodal ion exchange using a buffering agent and a salt and an inorganic salt for loading and a low salt and low pH buffer for elution in order to separate the covalently surface modified AAV species from unconjugated retargeting molecule species to provide a fraction that is enriched in covalently surface modified AAV species; and (c) harvesting at least one enriched fraction comprising covalently surface modified AAV species. Enrichment can result in greater than 50%, 55%, 60%, 70%, 75%, 80%, 85%, 90%, 95%, 96%, 97%, 98%, 99% or more of unconjugated retargeting molecule species being separated from the covalently surface modified AAV species. Enrichment can result in reducing the presence of unconjugated retargeting molecule species to undetectable levels.

The inventions further provide methods of separating a covalently surface modified adeno-associated virus (AAV) species from unconjugated retargeting molecule species, wherein the method comprises the steps of (a) providing a sample comprising the covalently surface modified adeno-associated virus species produced using an in vitro conjugation; (b) subjecting the covalently surface modified adeno-associated virus to multimodal binding and size separation bead chromatography using a multimodal buffer comprising in order to separate the covalently surface modified AAV species from unconjugated retargeting molecule species to provide a fraction that is enriched in covalently surface modified AAV species; and (c) harvesting the enriched fraction comprising covalently surface modified AAV species. The multimodal binding and size separation bead chromatography can be functionalized with an octyl amine that is hydrophobic and positively charged. The beads can comprise a matrix that provides size exclusion, and can be Capto™ Core 400 or Capto™ Core 700. Enrichment can result in greater than 50%, 55%, 60%, 70%, 75%, 80%, 85%, 90%, 95%, 96%, 97%, 98%, 99% or more of unconjugated retargeting molecule species being separated from the covalently surface modified AAV species. Enrichment can result in reducing the presence of unconjugated retargeting molecule species to undetectable levels.

The inventions further provide methods of purifying the covalently surface modified AAV of the inventions described herein using approaches including improved depth filtration, improved tangential flow filtration (such as single-pass tangential flow filtration), improved affinity capture and improved ionic exchange chromatography.

The inventions further comprise covalently surface modified adeno-associated viruses (AAV) produced and/or purified by the methods.

The inventions also provide AAV preparations produced by any of the above the methods, and drug products made from the AAV preparations.

The inventions are amendable to use with all AAV serotypes and variants, including but not limited to AAV1, AAV2, AAV2quad(Y-F), AAV3, AAV4, AAV5, AAV6, AAV7, AAV8, AAV9, AAV10, AAV11, AAV12, AAV13, AAV rh10, AAV rh39, AAV rh43, and AAV rh74.

Covalently surface modified adeno-associated viruses (AAV) made according to an example selected from the group consisting of Example 15, Example 16, Example 17, Example 18, Example 19, Example 20, Example 21, Example 22, Example 23, Example 24, Example 25, Example 26, Example 27, Example 28, Example 29, Example 30 and Example 31 also are provided, and can be produced using cells comprising at least one sequence set forth in an example selected from the group consisting of examples selected from the group consisting of Example 32, Example 33, Example 34, Example 35, Example 36, Example 37, Example 38 and Example 39.

AAV preparations comprising the covalently surface modified adeno-associated viruses and biologic drug products comprising the covalently surface modified adeno-associated virus also are provided.

### BRIEF DESCRIPTION OF THE FIGURES

The Figures (Fig. or Figs.) below depict schematic representations of aspects of the inventions and data from the inventions.

FIG. 1A schematically depicts the species heterogeneity involved in in-vitro conjugation reactions between AAV9 comprising a SpyTag peptide (rAAV9-SpyT) and SpyCatcher fused to a monoclonal antibody (SpyC-mAb species). The top row depicts the heterogeneity at the level of the AAV9-SpyT capsid, depicting the “average” AAV species with 6 SpyTags, as well as AAV species with lower (3) and higher number (10) of SpyTags expressed on the capsid surface. SpyTag is an example of a first member (FM). The middle and bottom rows respectively depict incomplete and optimum conjugation, also referred to as ‘complete conjugation’.

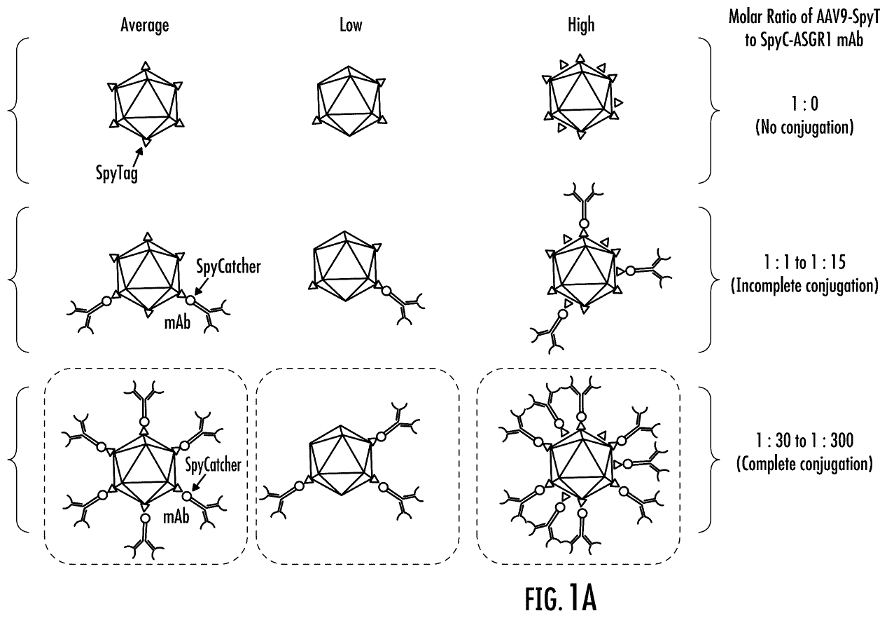

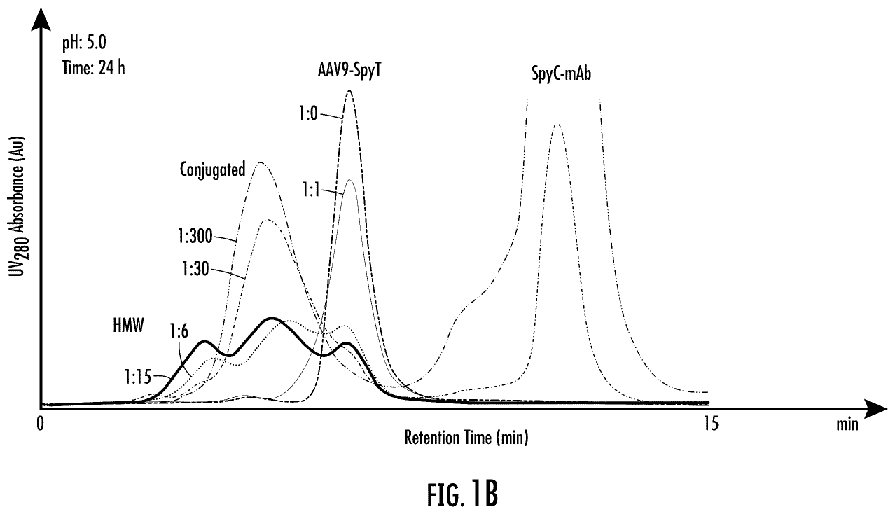

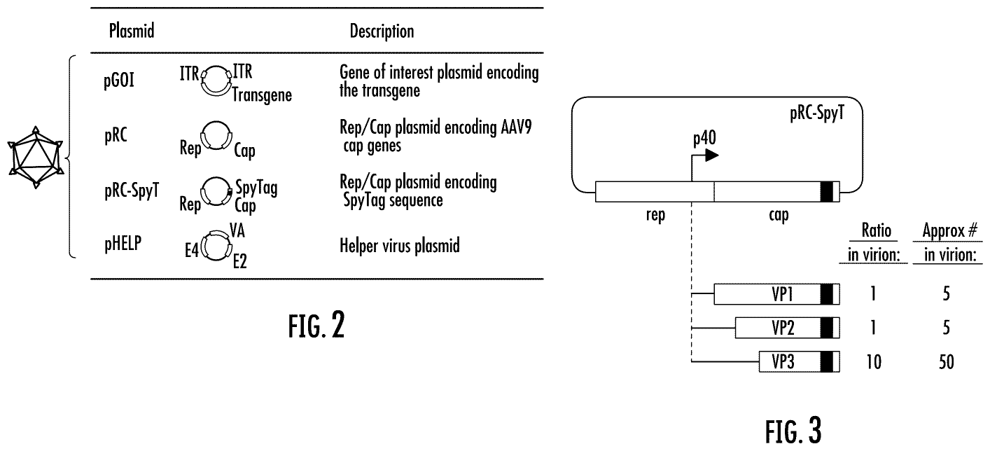

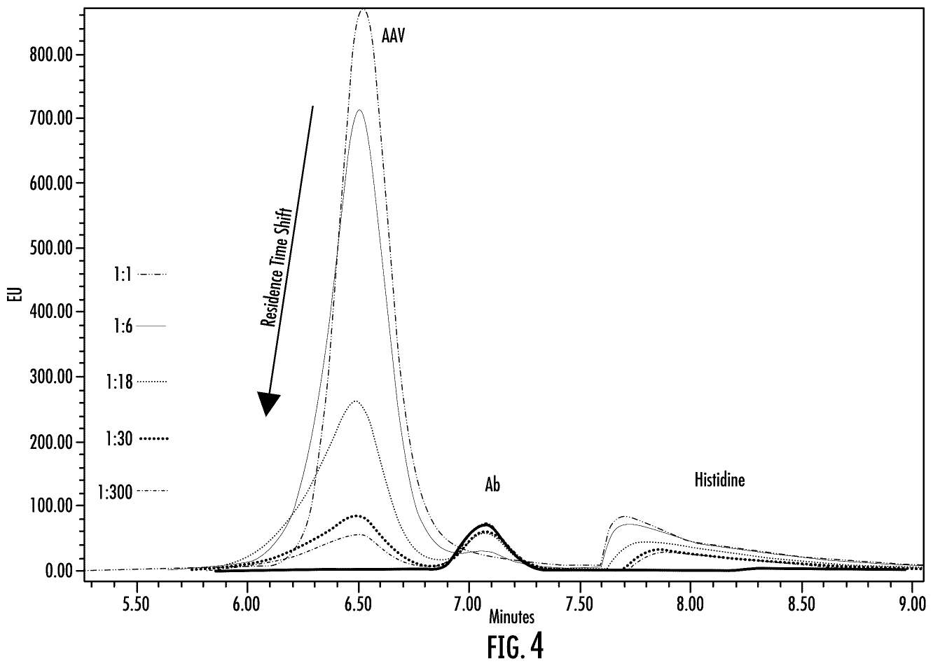

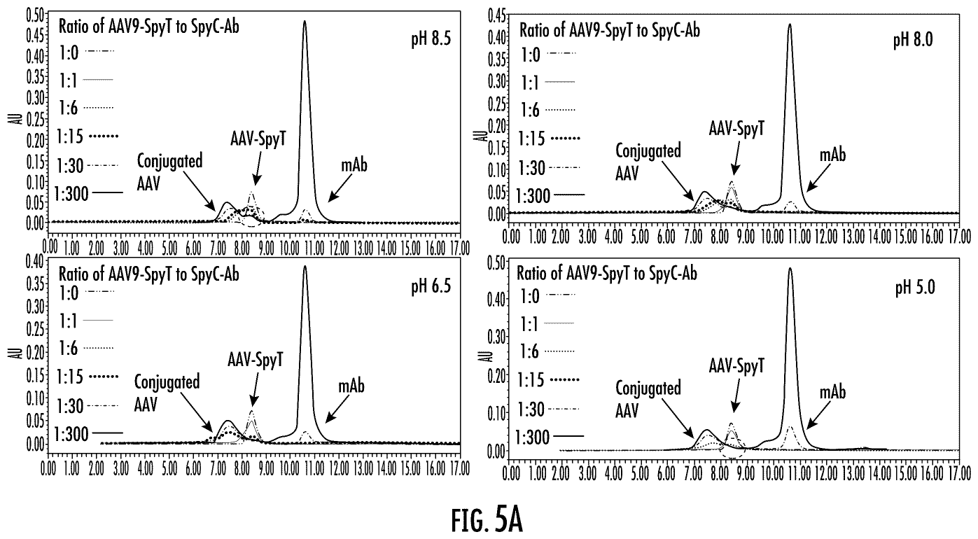

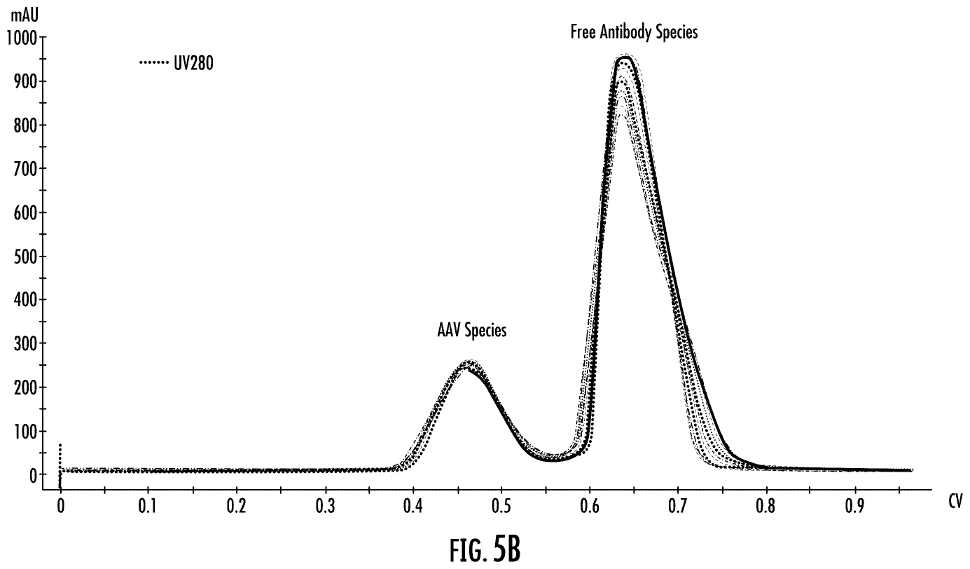

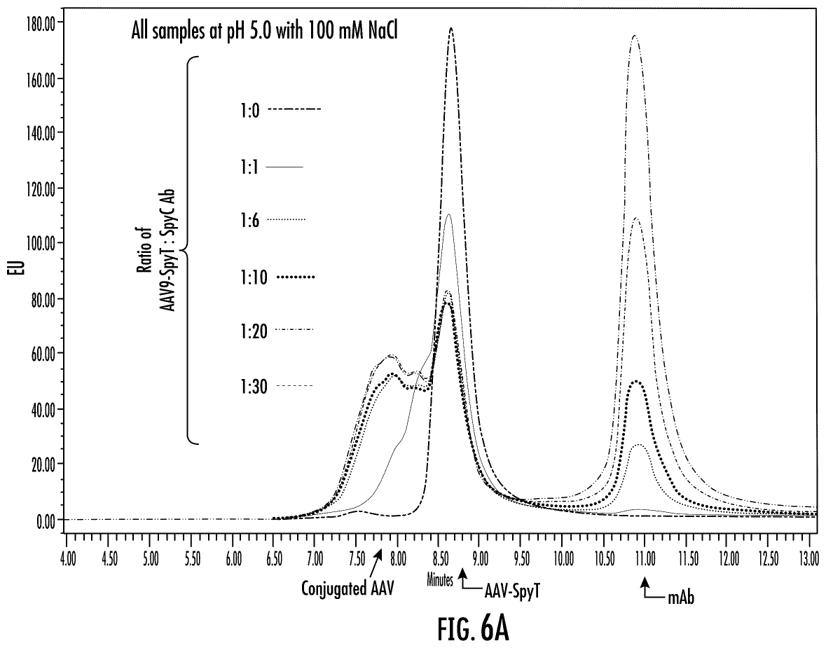

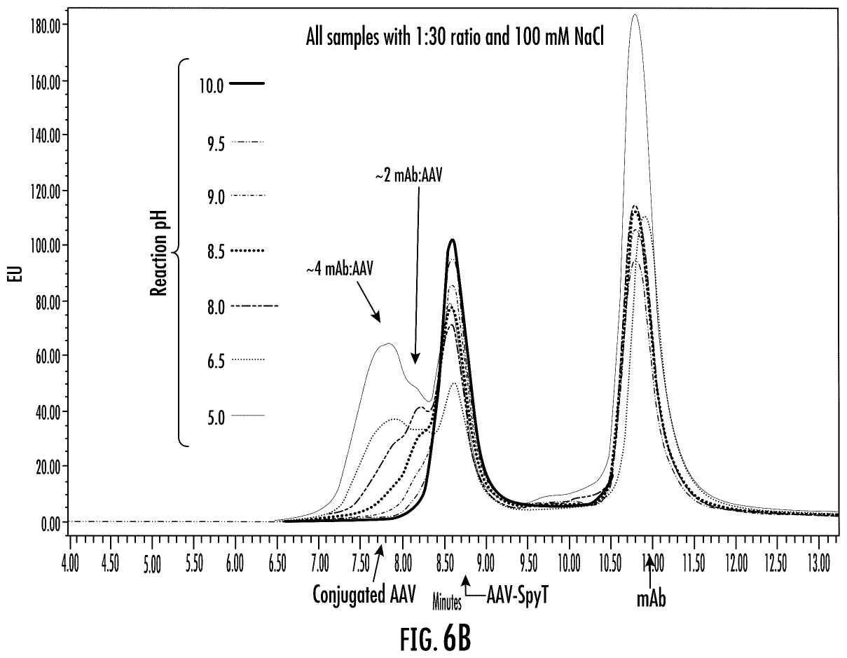

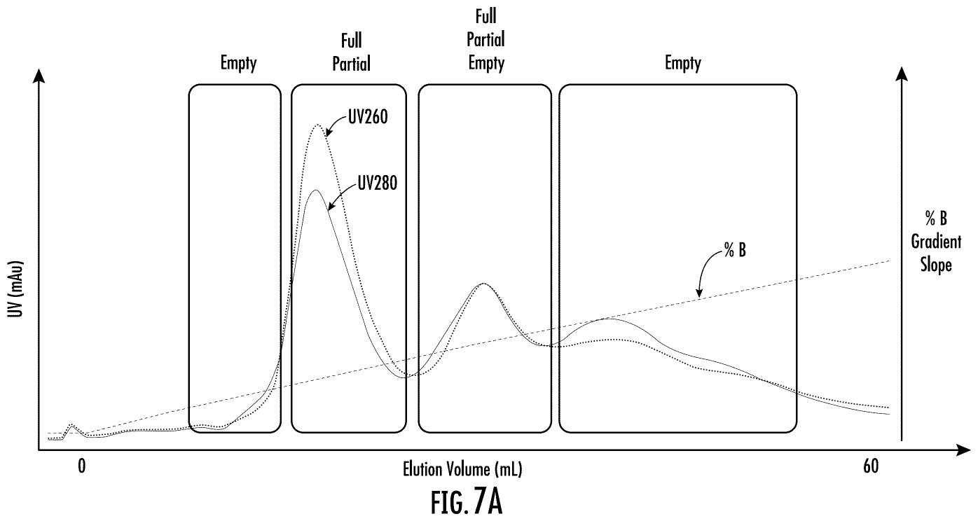

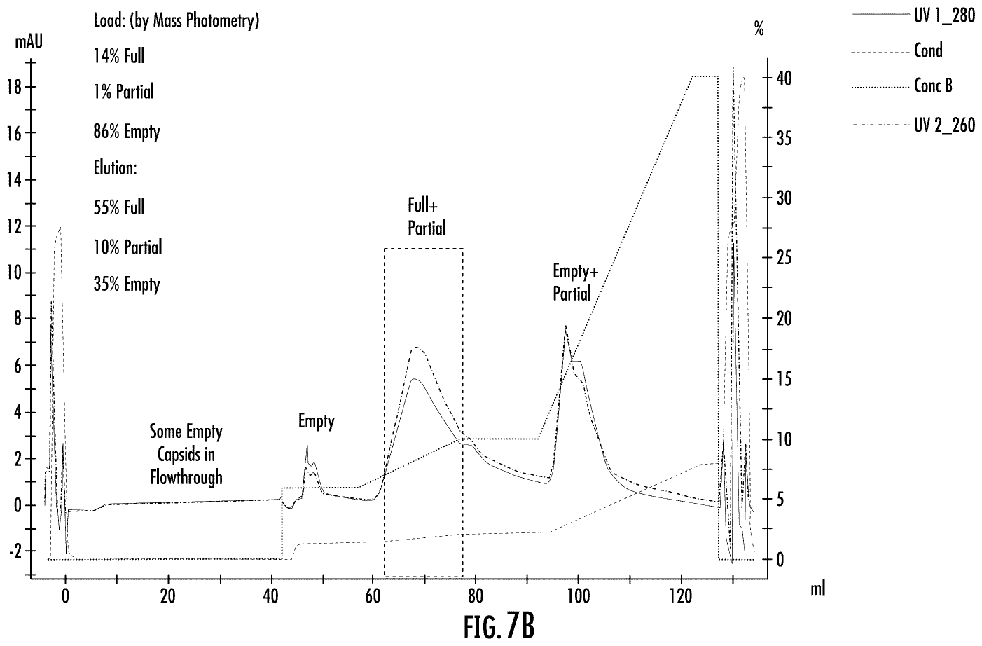

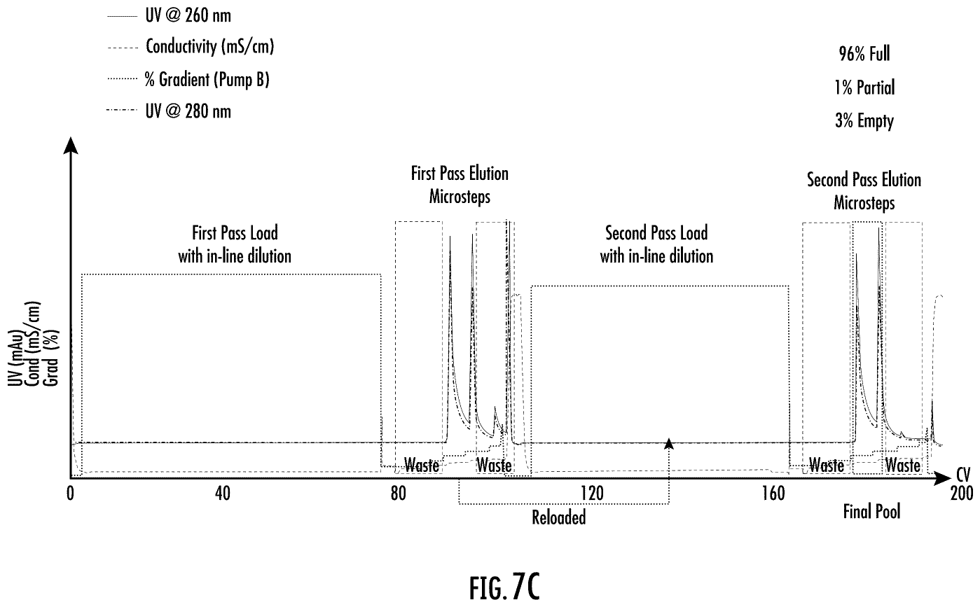

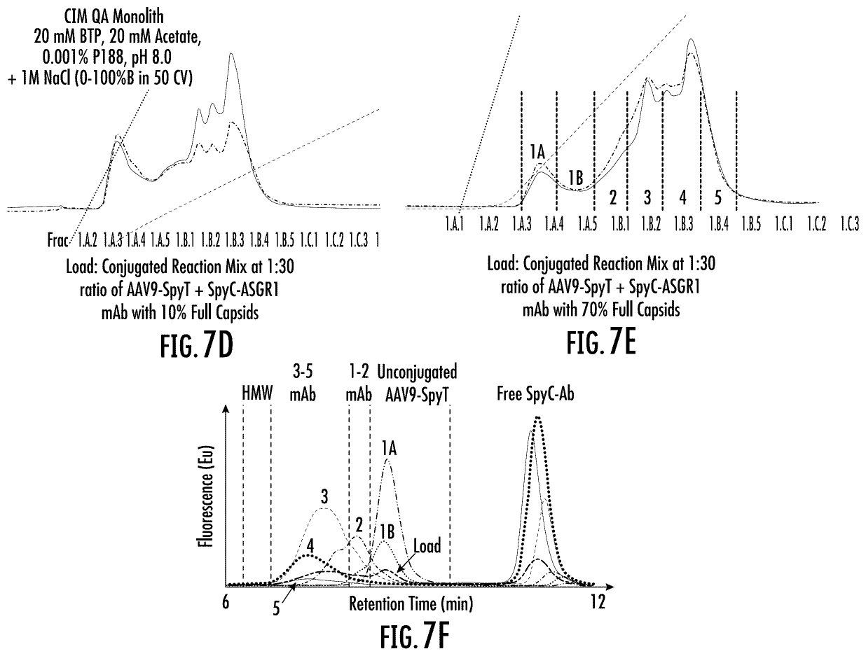

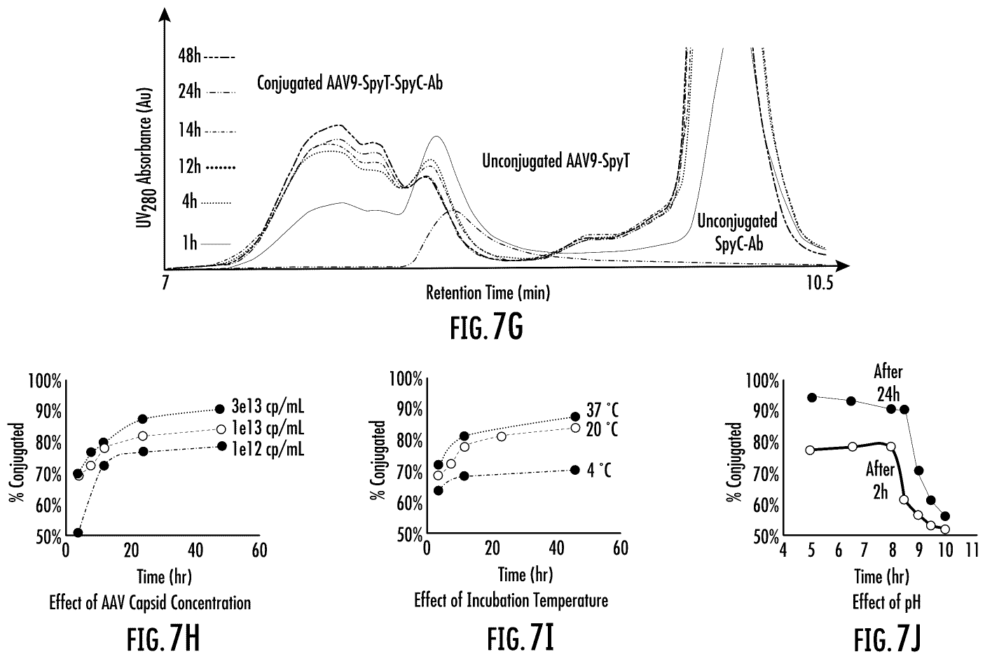

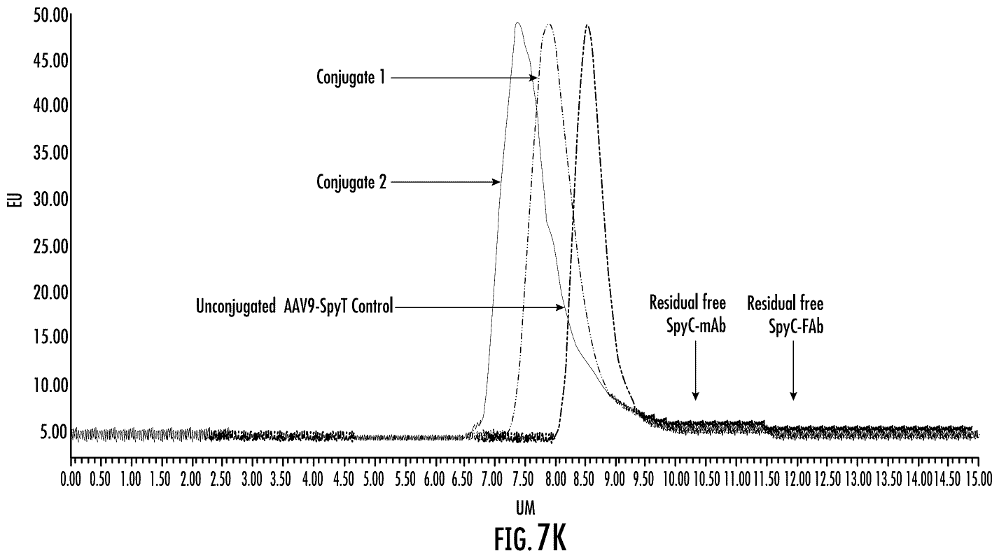

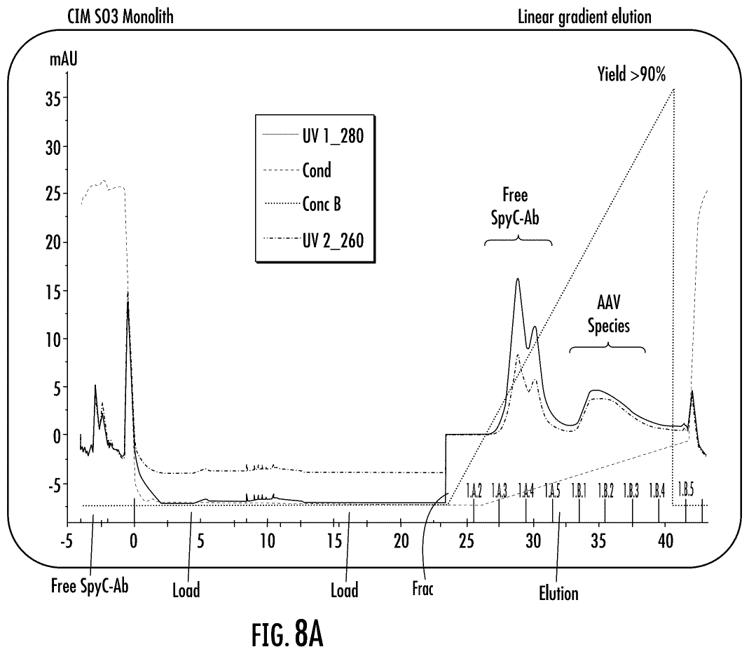

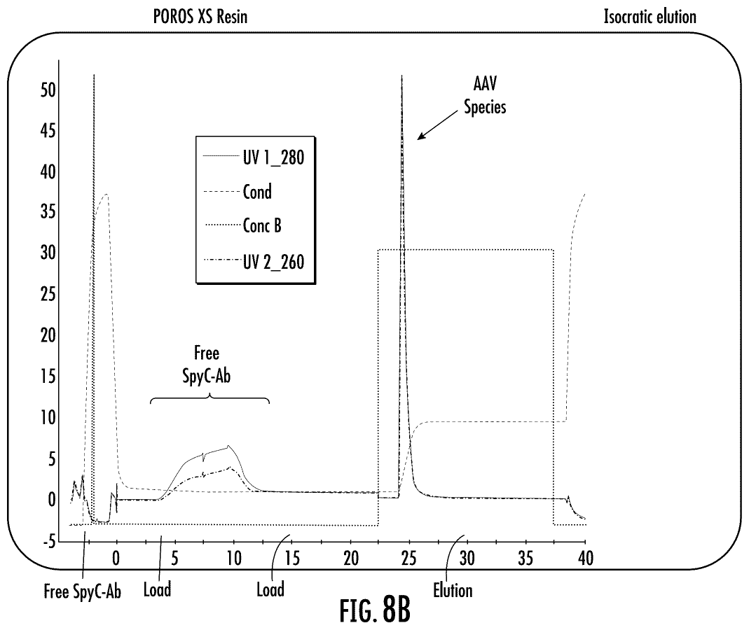

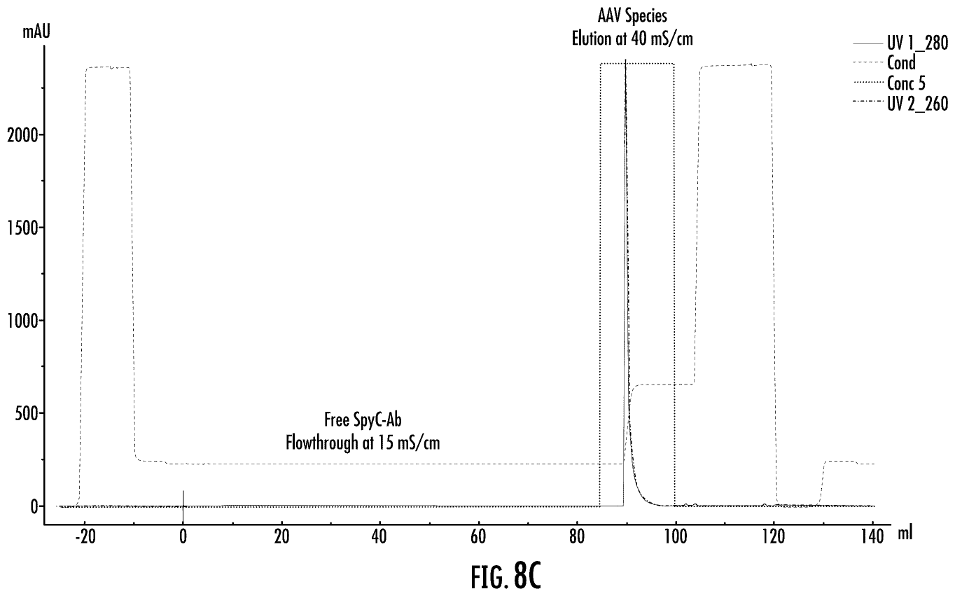

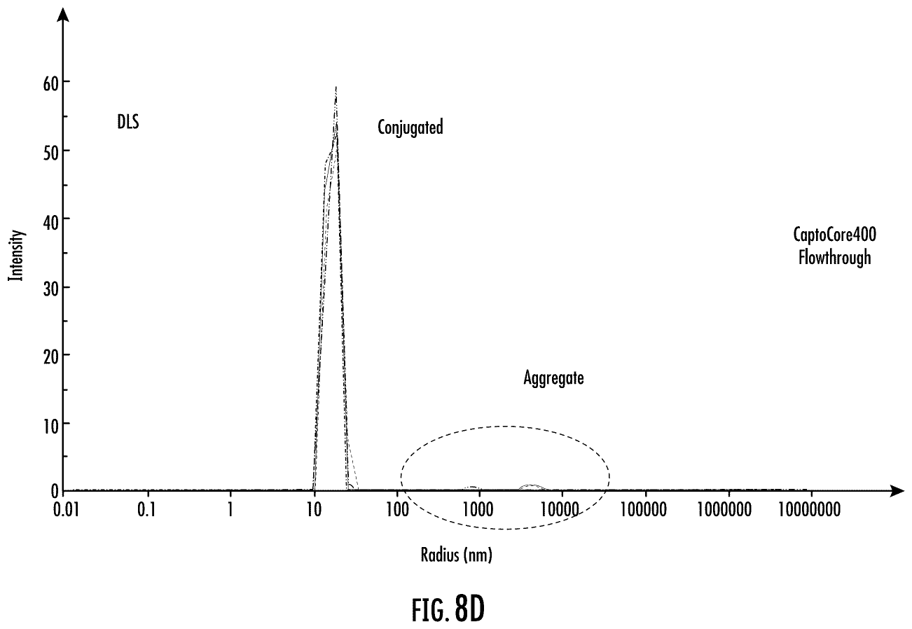

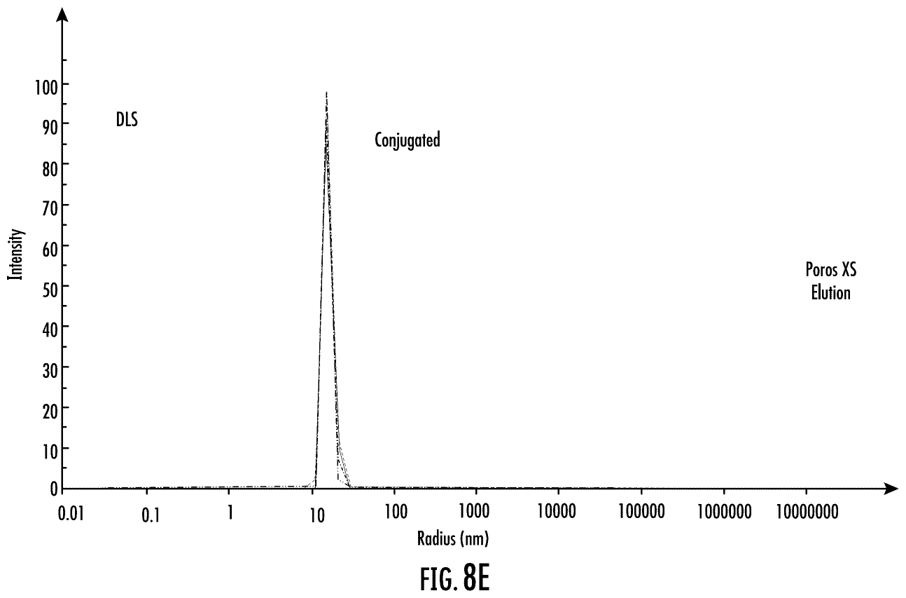

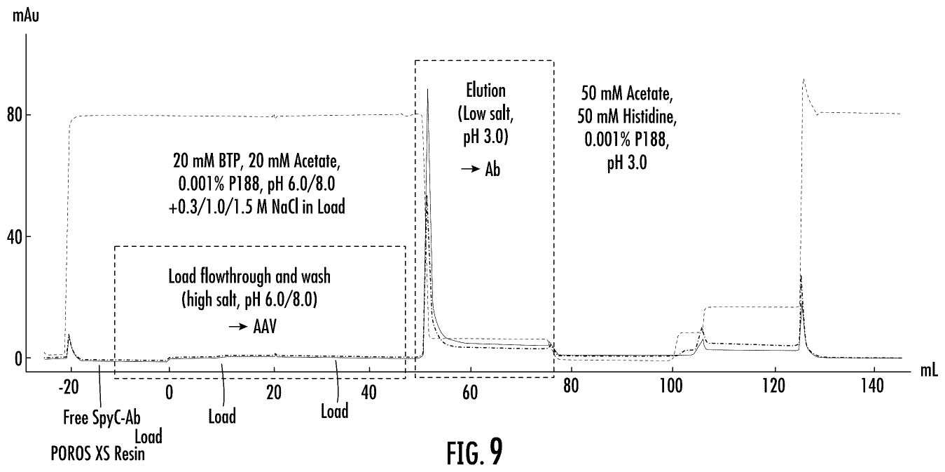

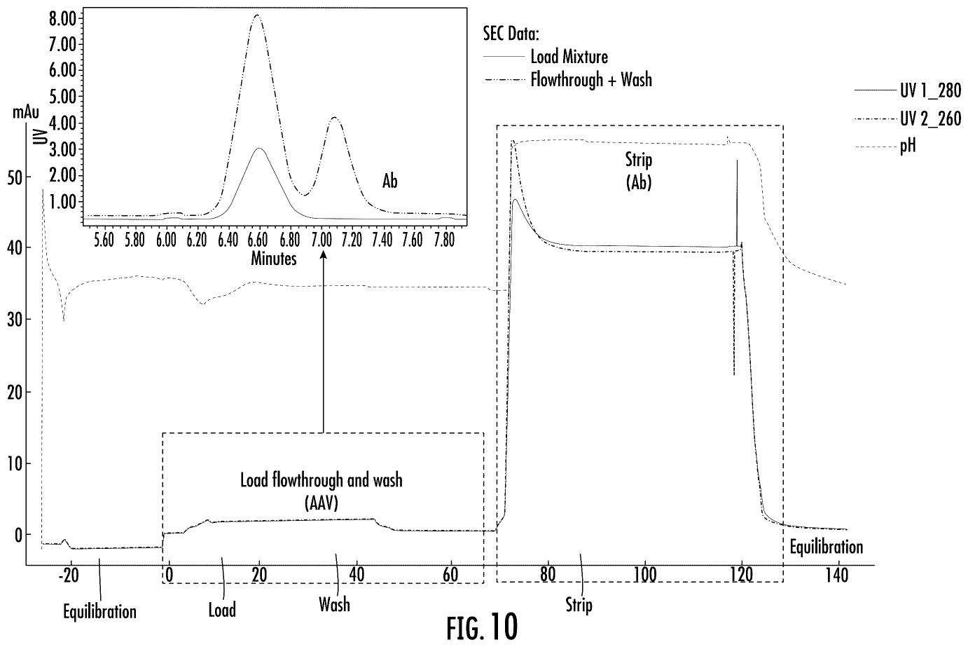

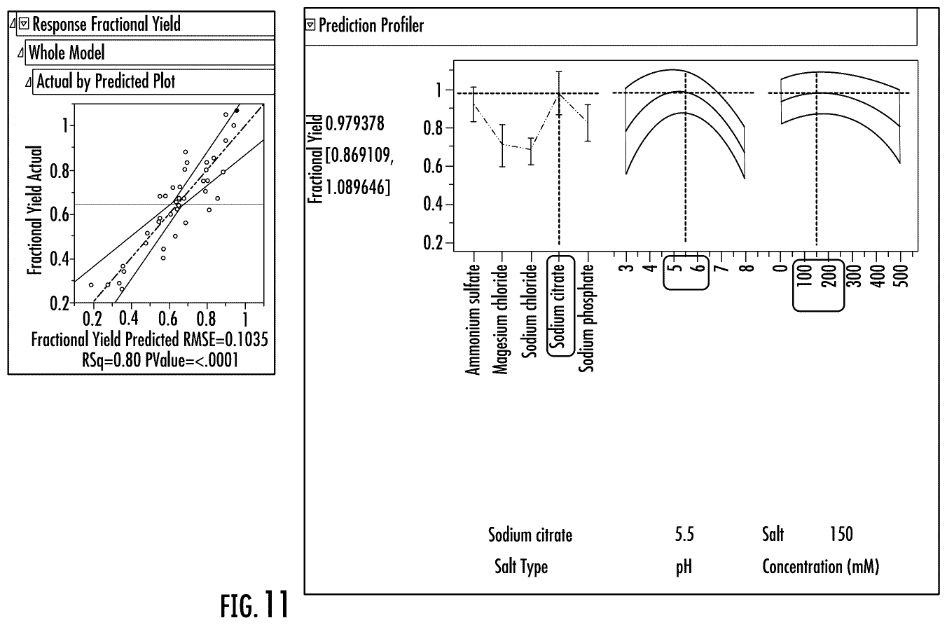

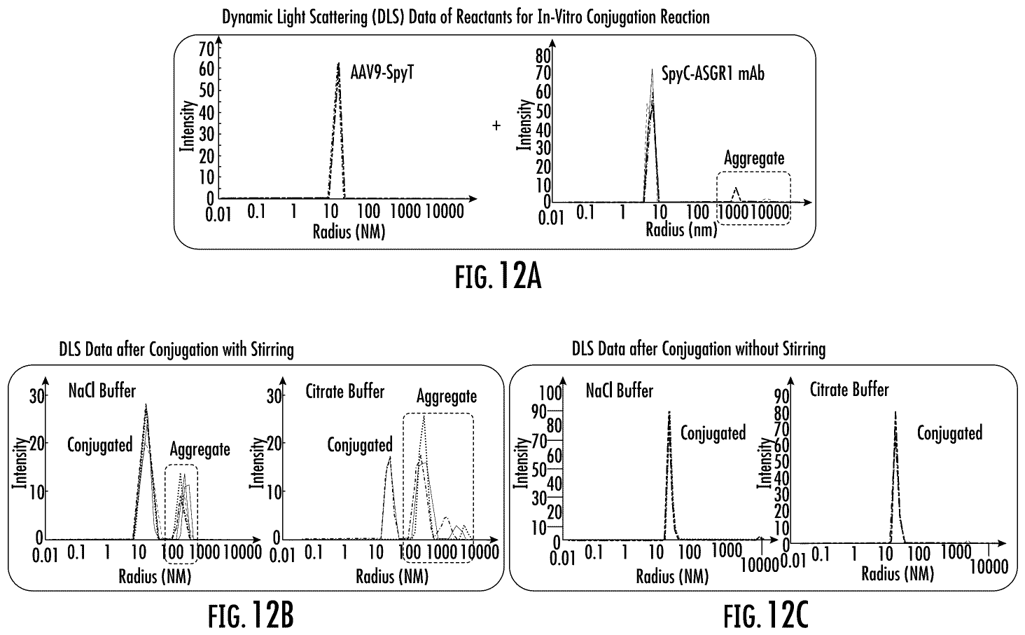

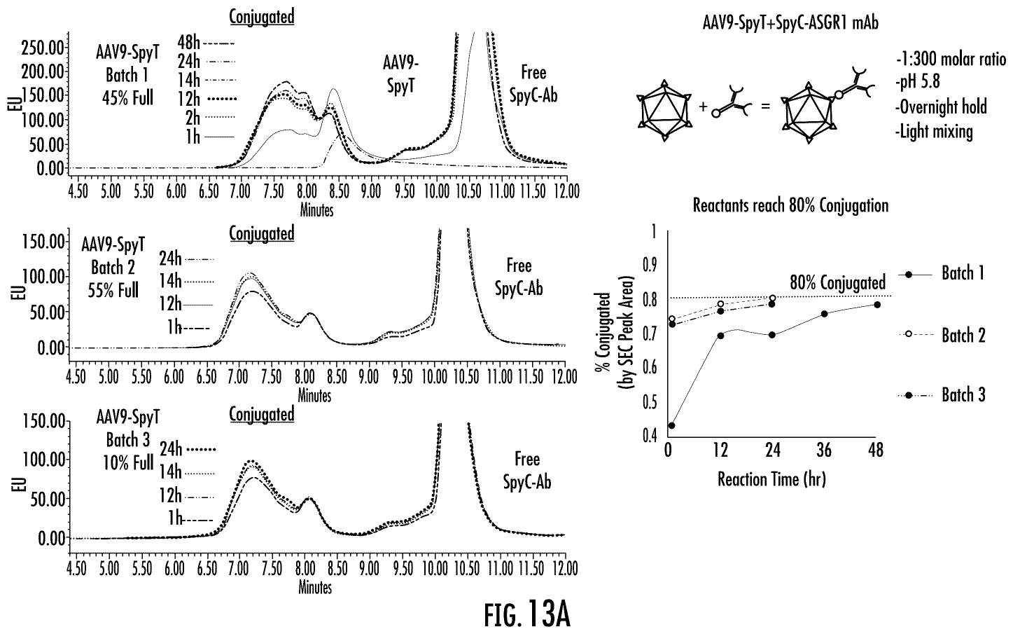

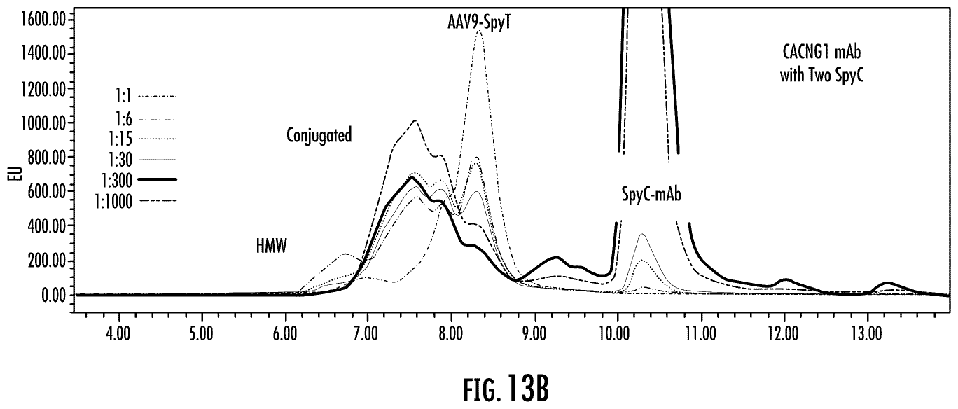

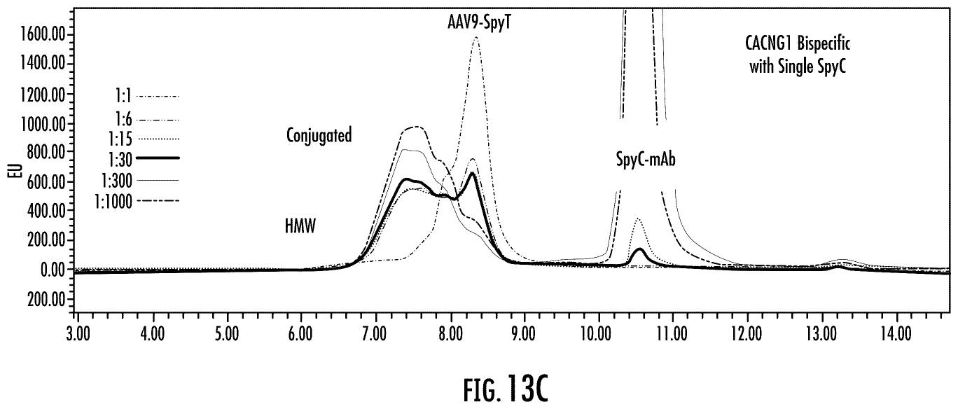

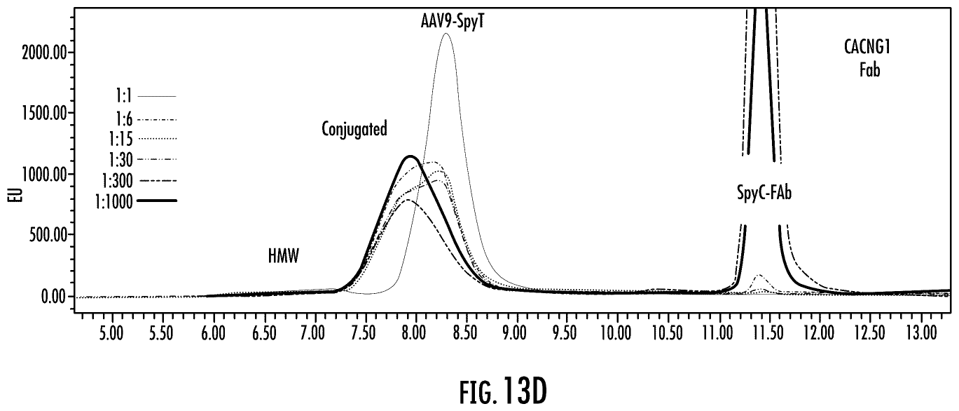

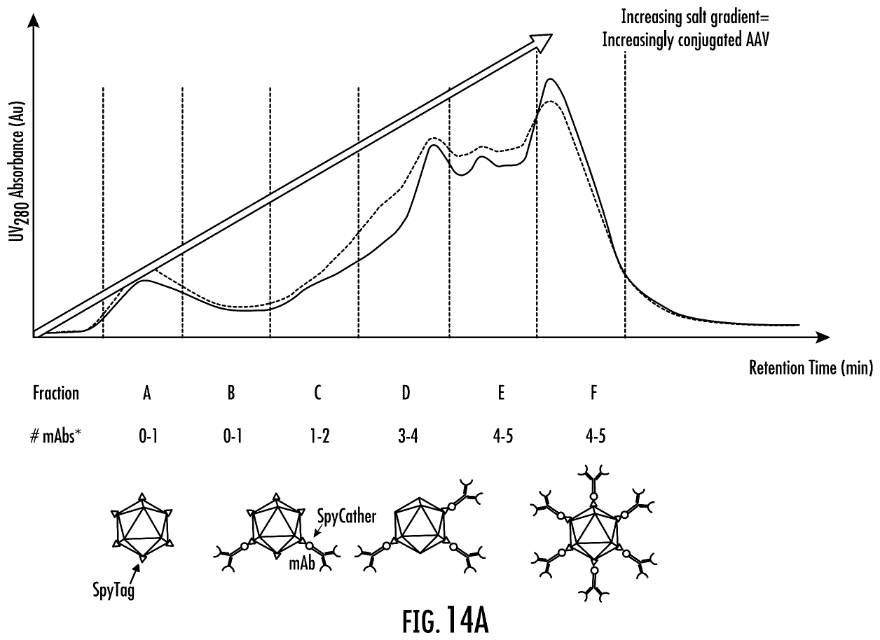

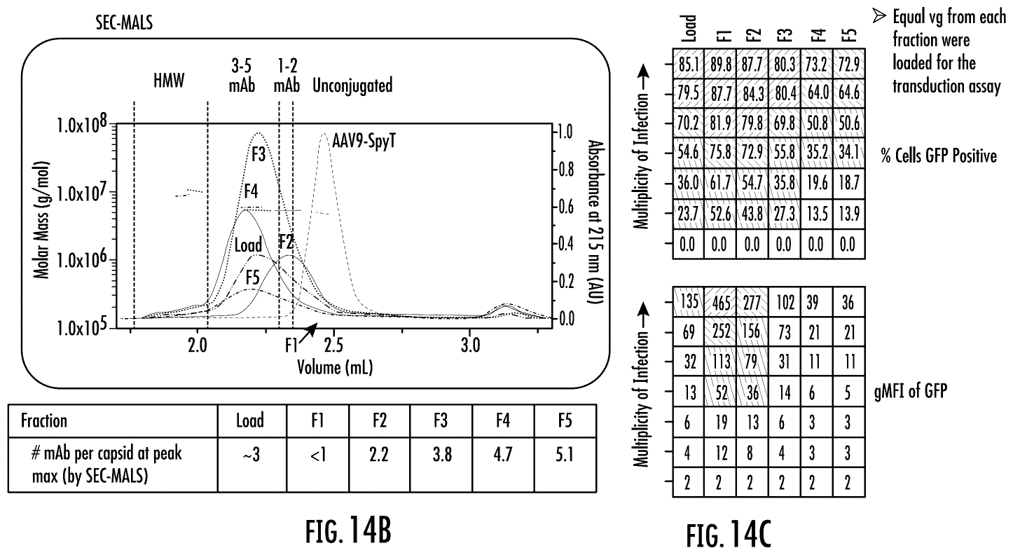

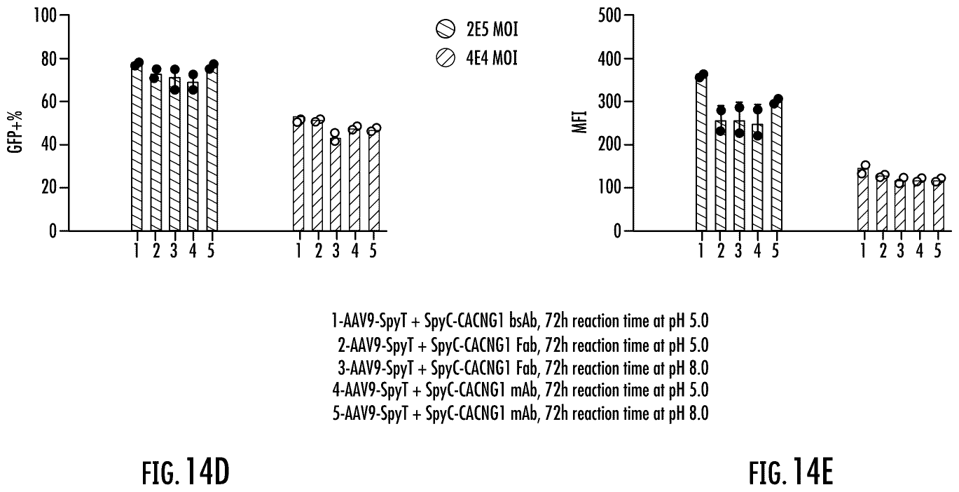

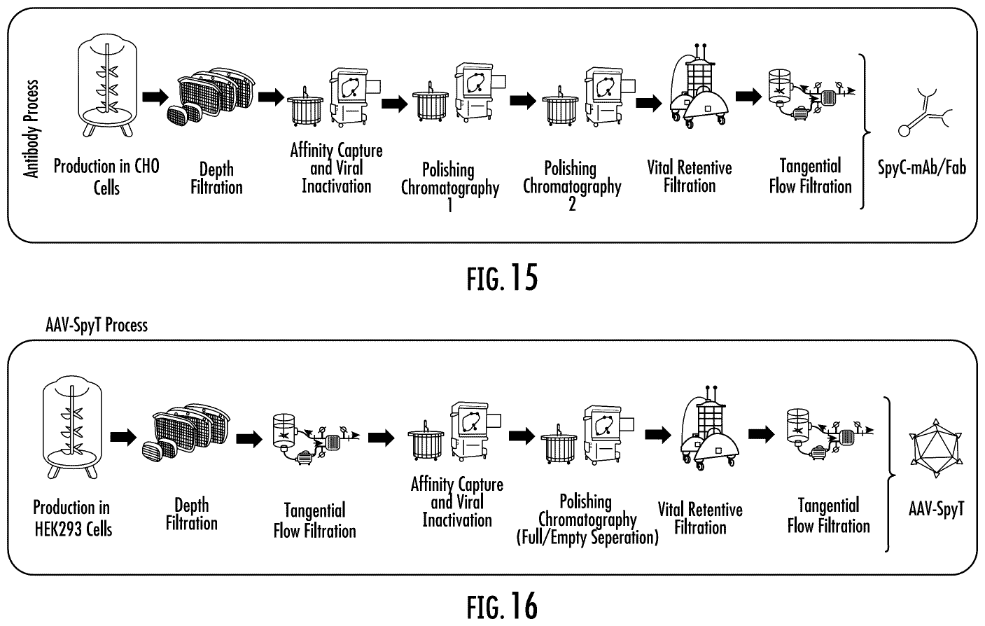

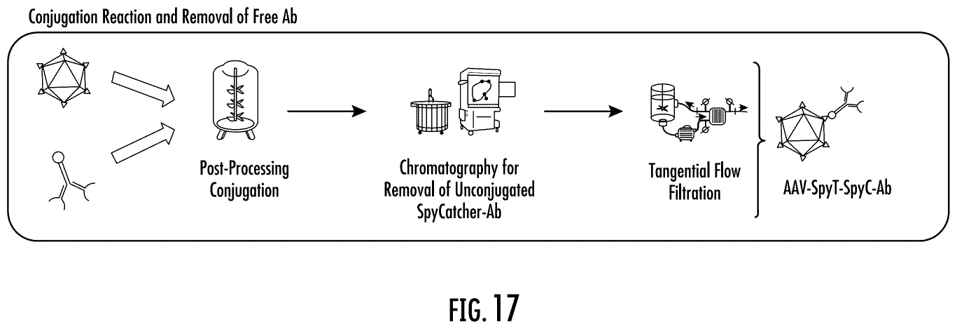

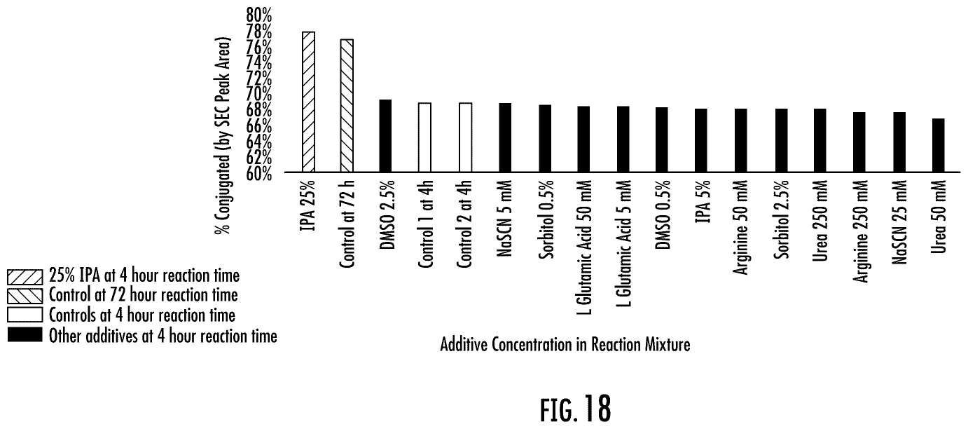

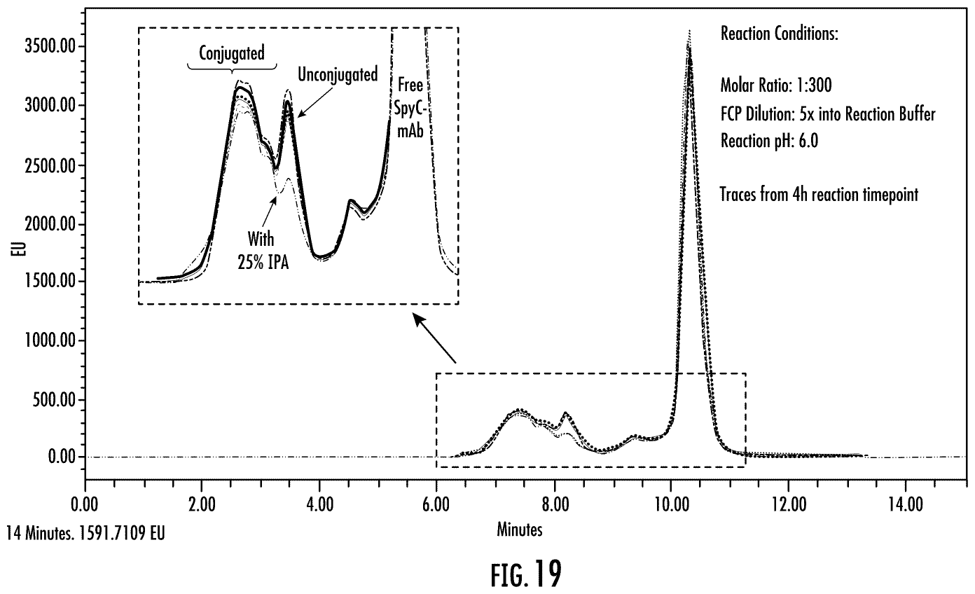

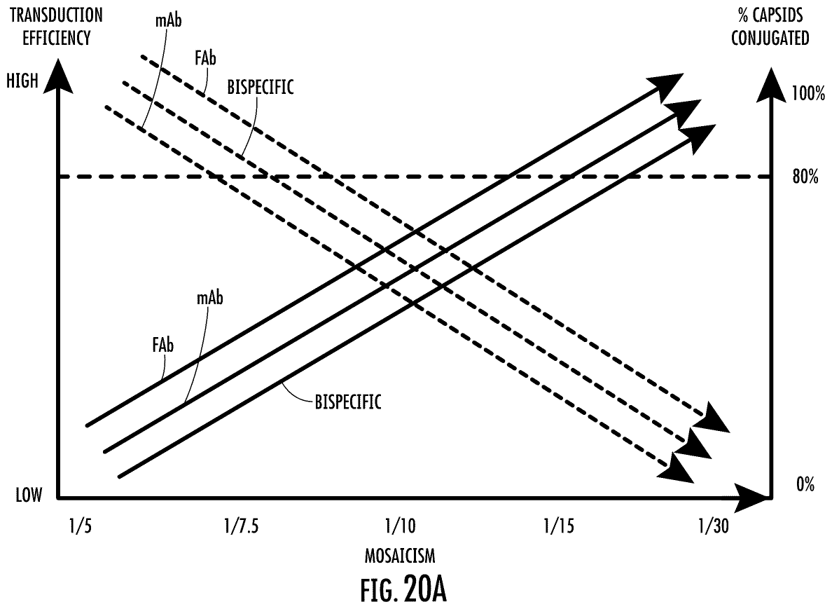

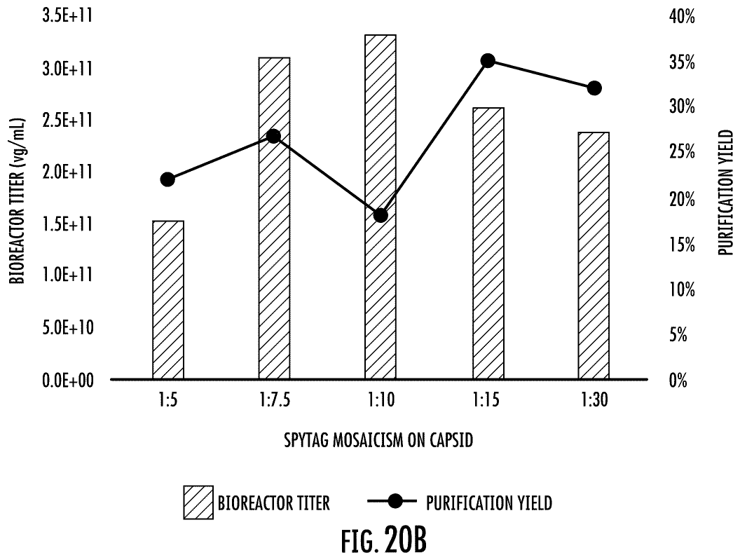

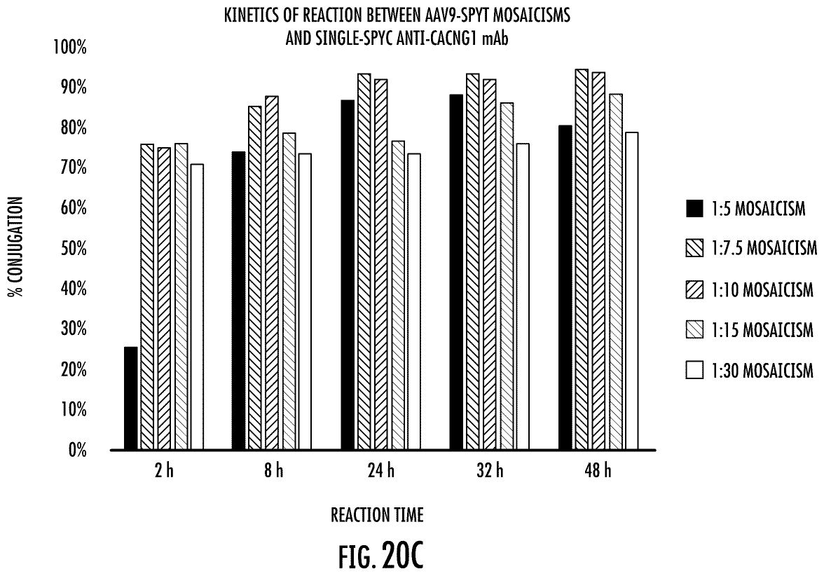

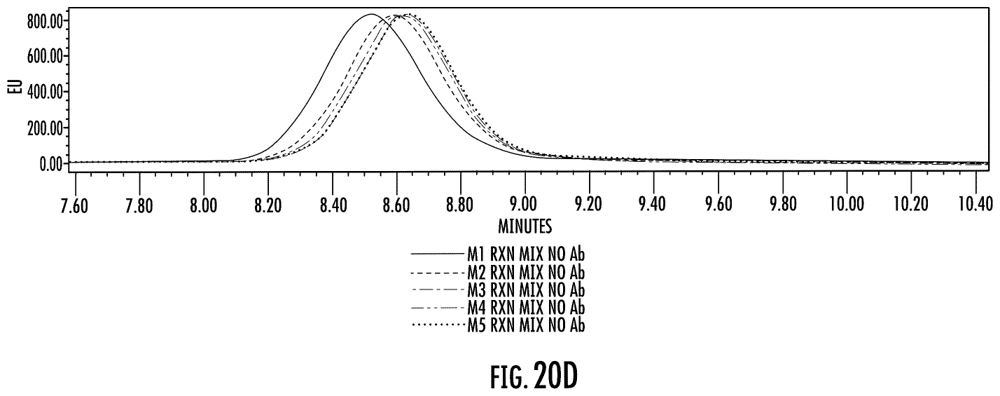

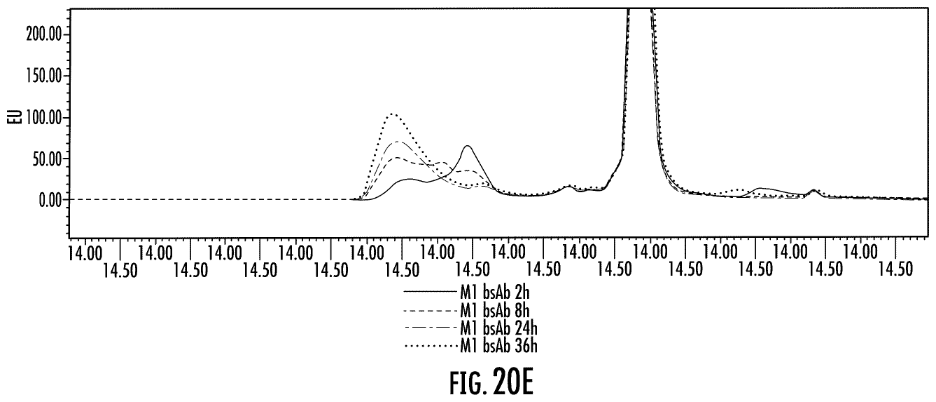

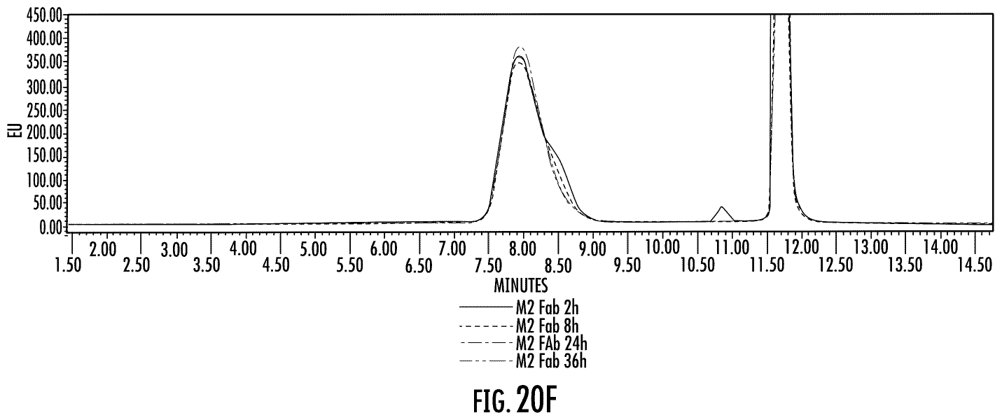

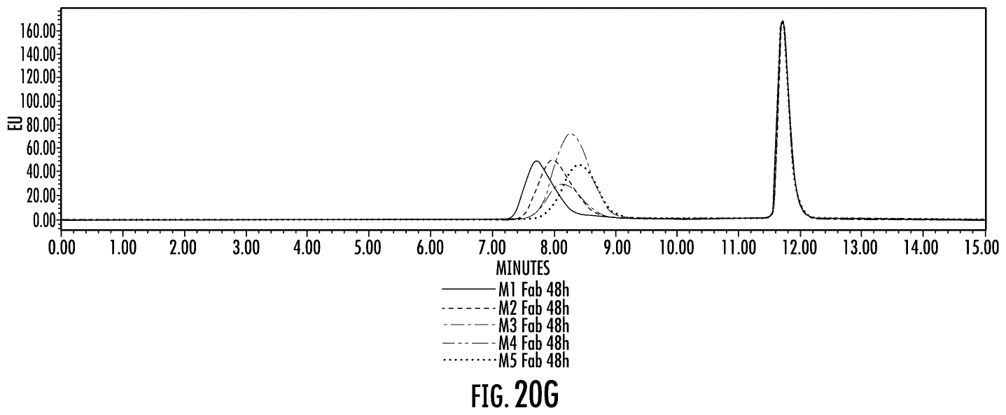

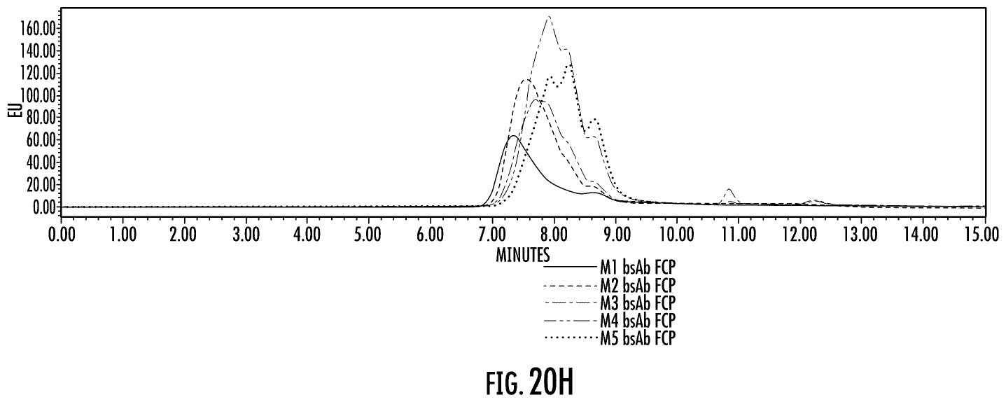

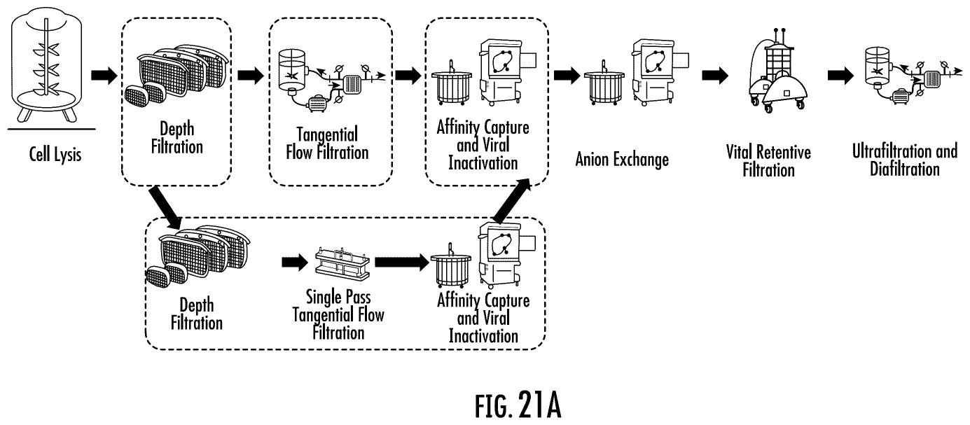

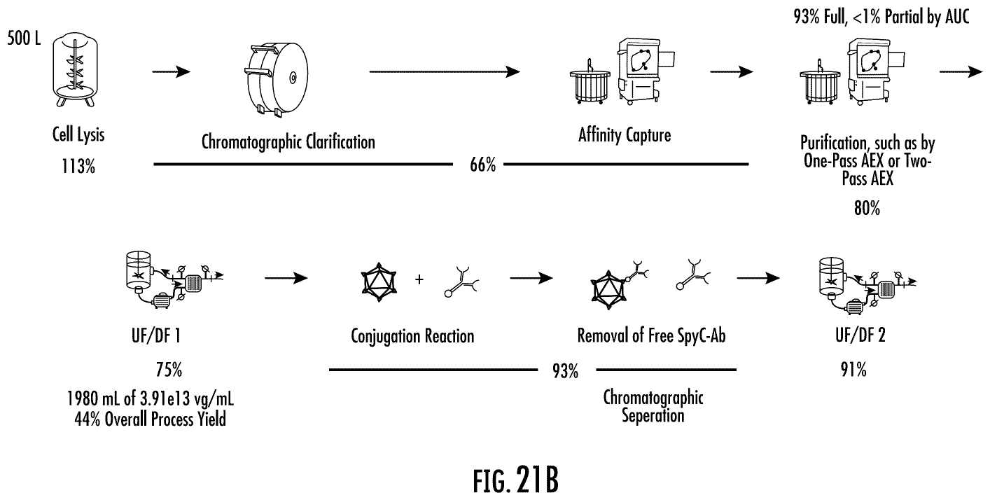

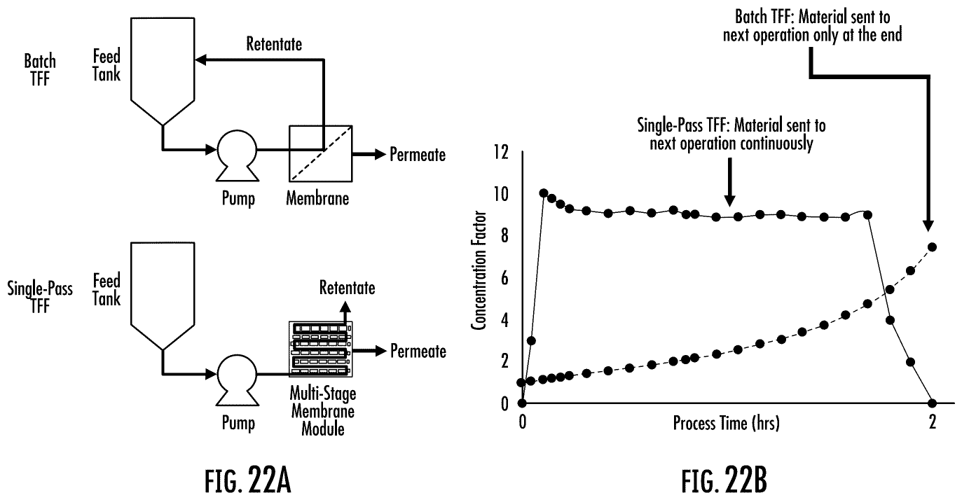

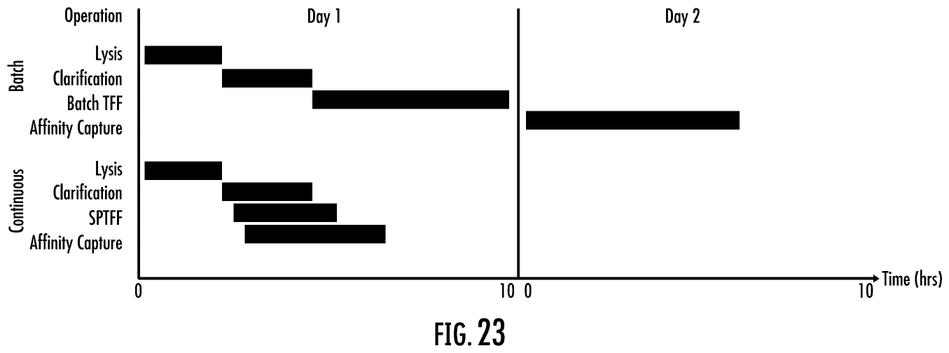

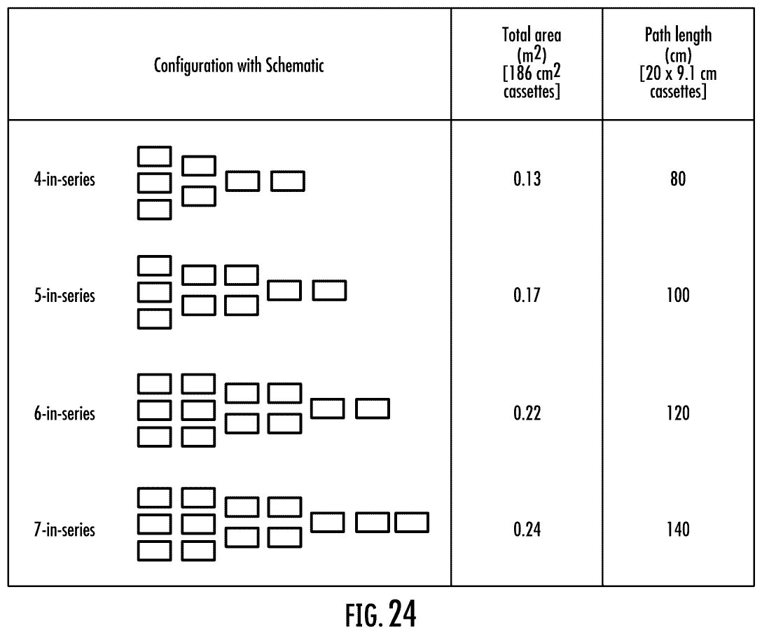

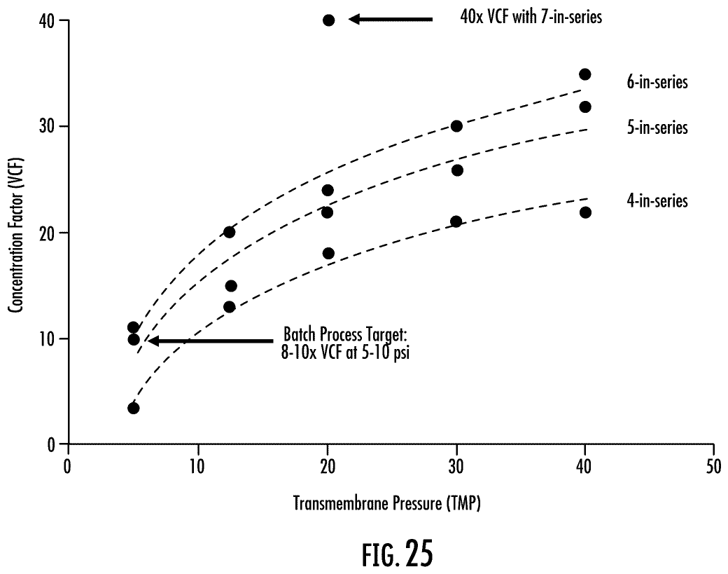

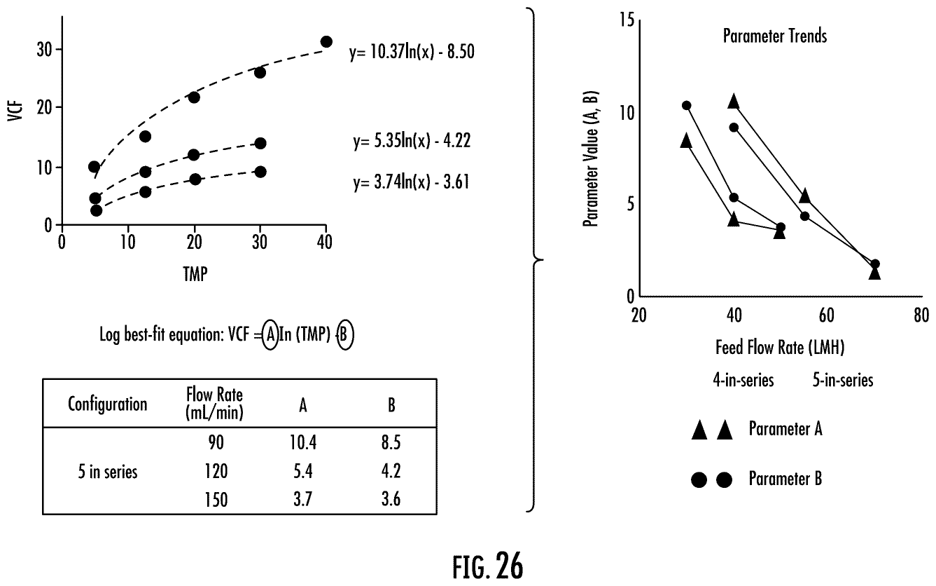

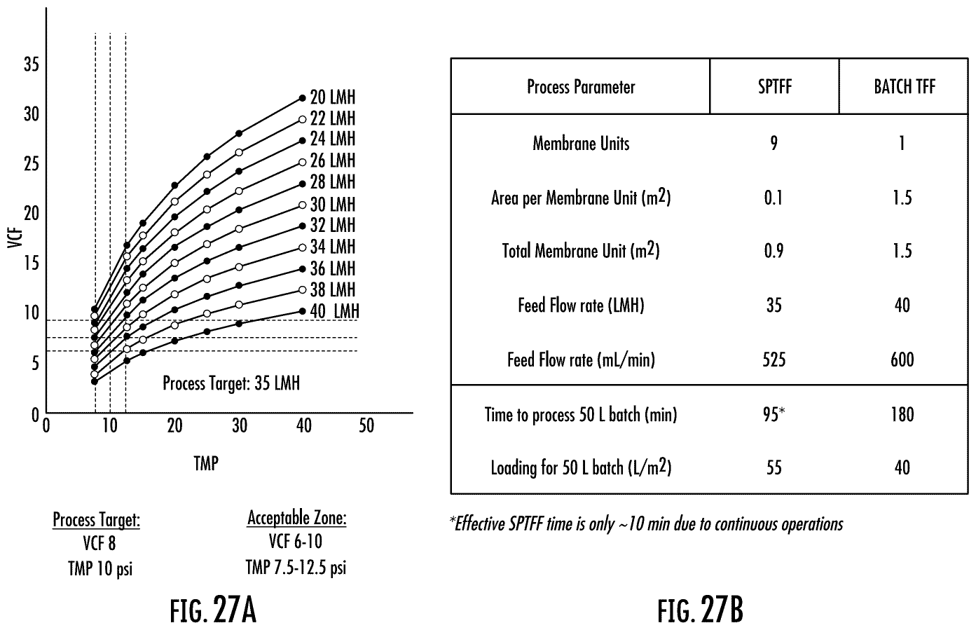

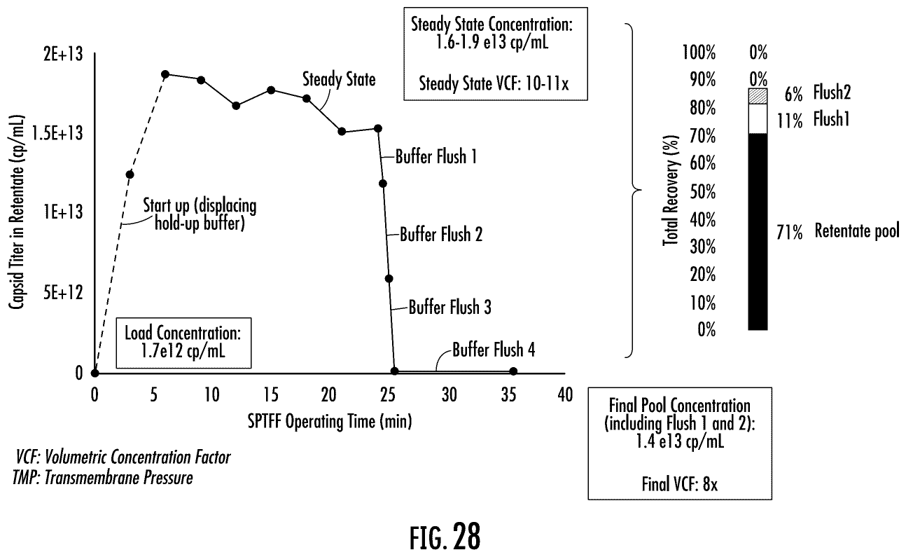

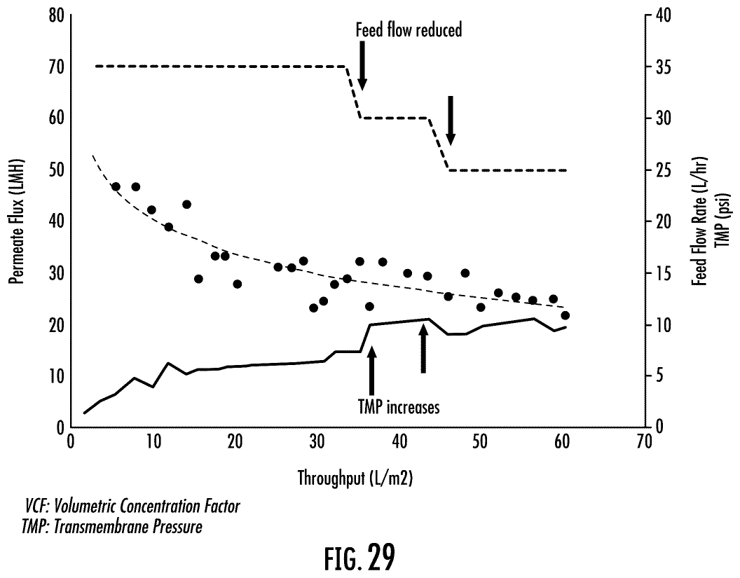

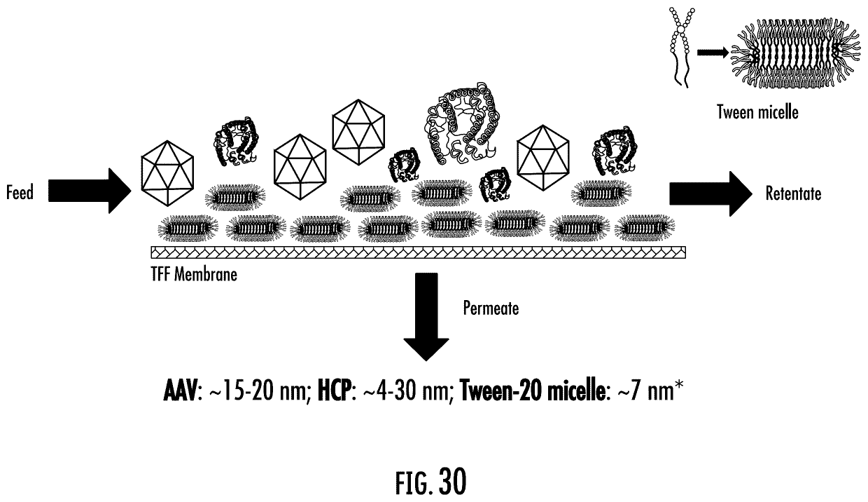

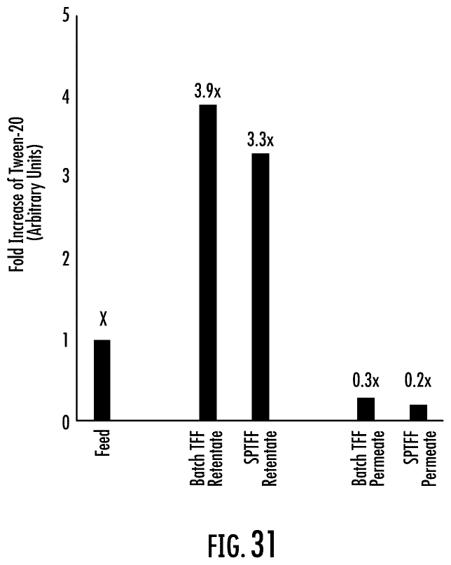

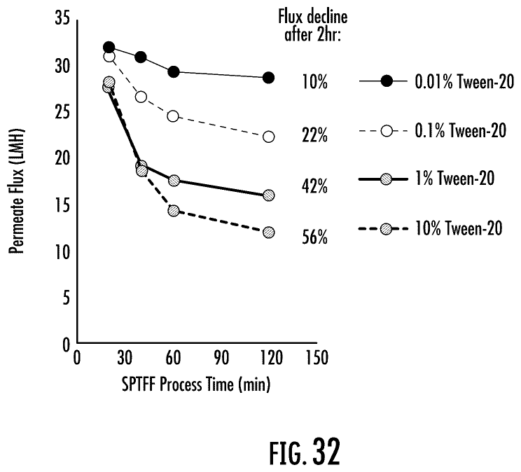

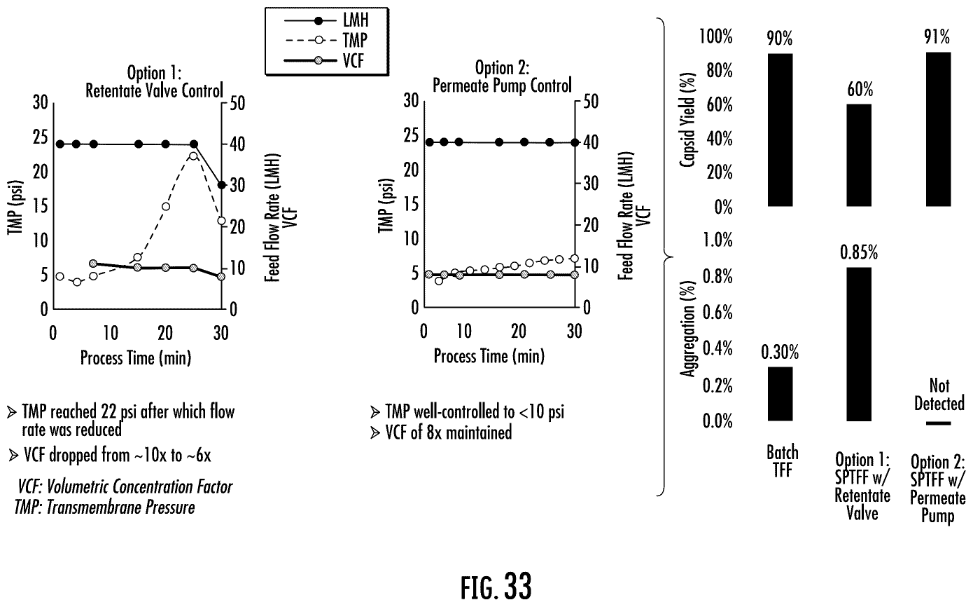

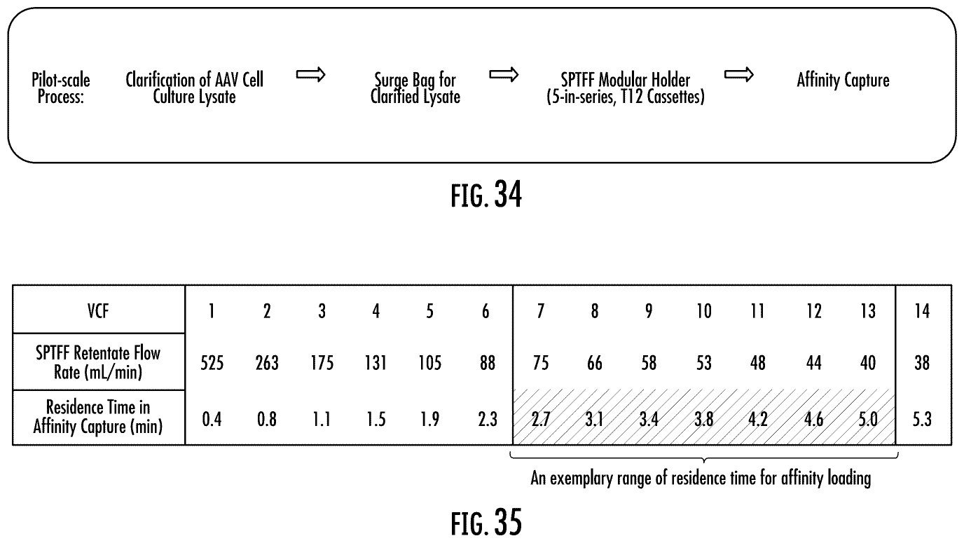

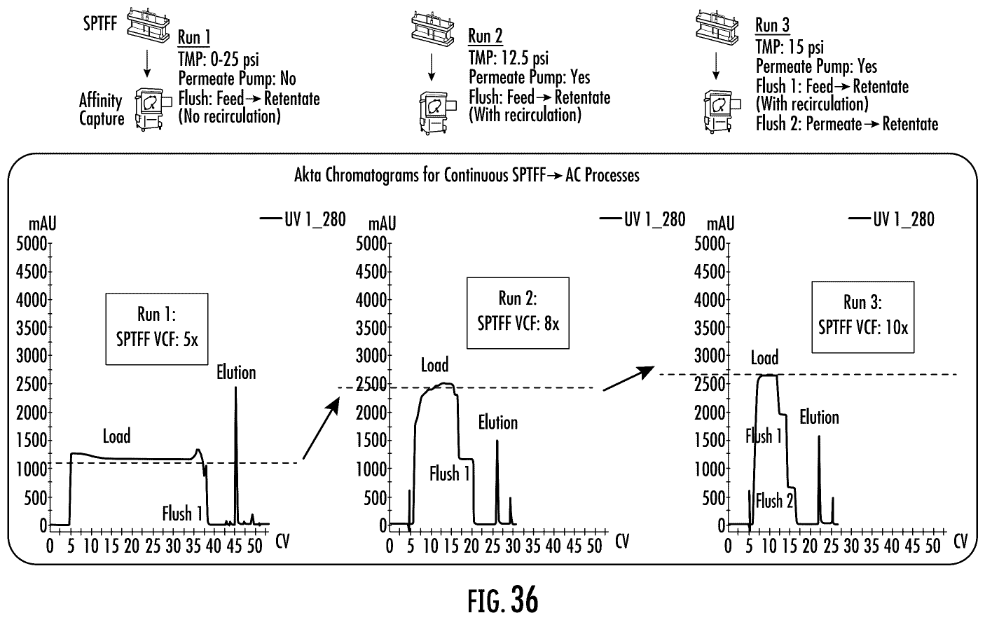

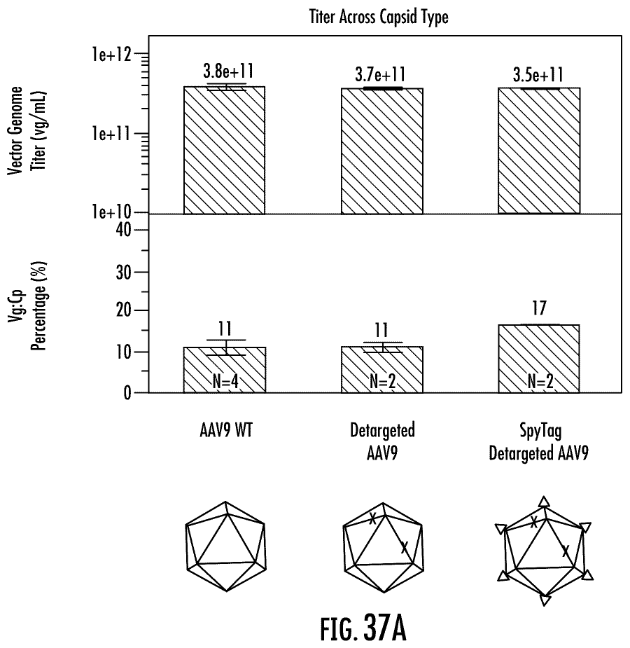

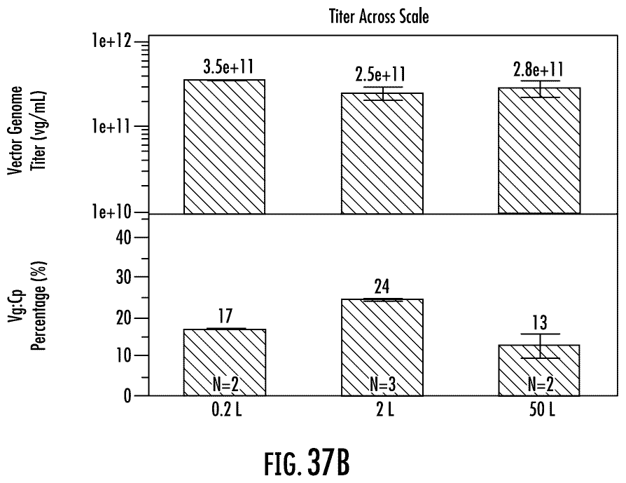

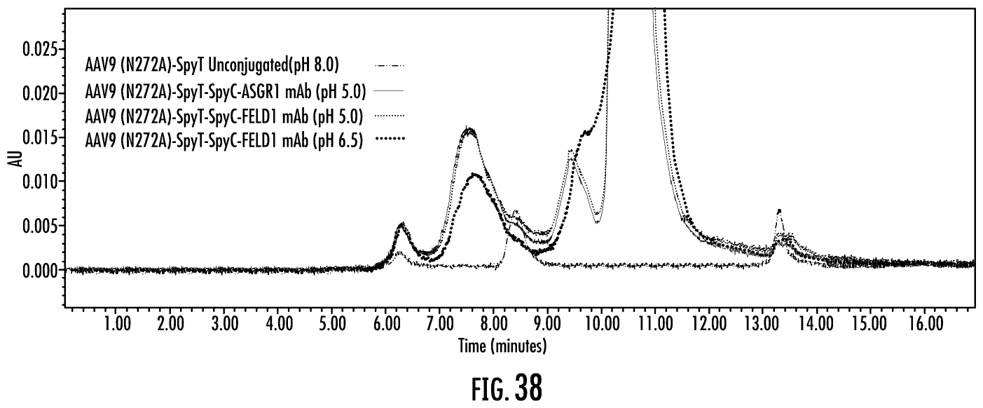

Optimum (often referred to as ‘complete’ since it is based on the desired purpose) conjugation occurs when a substantial portion, preferably a majority, of accessible first members of a specific binding pair are bound to second cognate members of the specific binding pair, which is the desired outcome. For example, FIG. 1A (bottom row) schematically depicts instances of all and nearly all accessible SpyTags bound by SpyCatcher on covalently surface modified AAVs. First member (for example, SpyTag) accessibility in the capsid can have an effect, however. Thus, optimal (complete) conjugation is the point where no unacceptable increase of conjugated species is observed (for example, no unacceptable shift of peaks from unconjugated to conjugated species on analytical size-exclusion chromatography as shown in, for instance, FIG. 7G).

FIG. 1B is a graph that depicts retention time (minutes) of high molecular weight (HMV) variants comprising conjugated AAV9-SpyT-SpyC-Ab species, conjugated AAV9-SpyT-SpyC-Ab, unconjugated AAV9-SpyT and unconjugated SpyC-mAb. Ratios of AAV9-SpyT to SpyC-mAb of 1:30 and 1:300 provided the best results.

FIG. 2 schematically depicts and describes quad transfection systems and methods for producing covalently surface modified AAV in a eukaryotic cell, such as HEK 293, for example HEK293F. As described in this figure, the quad transfection system utilizes four plasmids (schematically depicted) to produce AAV comprising SpyTag sequences in the capsids. The plasmids are as follows:
(i) pGOI—a plasmid comprising a GOI (for example, a transgene) flanked by two AAV inverted terminal repeats (ITRs), and optionally a selection marker gene, such as an antibiotic resistance gene (for example, ampicillin resistance);
(ii) pRC—a plasmid comprising AAV (here AAV9) rep and cap genes, and optionally a selection marker gene, such as an antibiotic resistance gene (for example, ampicillin resistance);
(iii) pRC-SpyT—a plasmid as above in (ii) and further comprising a polynucleotide sequence encoding a 13 amino acid Spy Tag peptide (a first member of a specific binding pair), and optionally a selection marker gene, such as an antibiotic resistance gene (for example, ampicillin resistance). This is an exemplary plasmid pRC-first member (pRC-FM); and
(iv) pHELP—a plasmid comprising adenovirus helper genes E4 and E2, and VA RNA (exemplary helper genes, and a optionally selection marker gene, such as an antibiotic resistance gene (for example, ampicillin resistance).

FIG. 3 schematically depicts at the top a pRC-SpyTag plasmid comprising the rep and cap genes and the p40 promoter, and at the bottom schematically shows the insertion of the SpyTag 13 amino acid peptide sequence to form a fusion protein comprising SpyTag and Cap peptide sequences, thereby resulting in mutant VP1, VP2 and VP3 proteins fused to the SpyTag peptide sequence. The AAV still possesses the 5:5:50 stoichiometry of the VP1, 2 and 3 proteins.

FIG. 4 depicts size exclusion chromatograms (SEC) of in vitro conjugation reaction mixtures of rAAV9-FM to antibody-SCM in ratios of 1:1, 1:6, 1:18, 1:30 and 1:300 at pH 8.0 and using rAAV concentration of 5×10¹⁰ to 1×10¹² capsids (cp) per milliliter on a size exclusion chromatography column with pore size of 1000 Å. The shift in retention time of the AAV peak is highlighted with an arrow showing shifting of the peak maximum to the left indicating conjugation reaction completion. The x-axis is minutes for elution and the y-axis is emission units (EU) for fluorescence.

FIG. 5A depicts analytical size exclusion chromatograms of in-vitro conjugation reaction mixtures of rAAV9-FM to antibody-SCM in ratios of 1:0, 1:1, 1:6, 1:15, 1:30 and 1:300 at pH 5.0, 6.5, 8.0 and 8.5 and using rAAV concentration of 2.3×10¹³ cp/mL on a size exclusion chromatography column with pore size of 450 Å. Reaction conditions included brief mixing followed by overnight incubation, resulting in maximum conjugation as shown by the almost complete disappearance of the AAV-SpyT peak and formation of a new conjugated AAV peak at ratios of 1:30 and 1:300. FIG. 5B is a preparative scale SEC on a 100 ml SEPAX SRT 500A column to remove unconjugated antibody. UV280 shows the antibody-AAV separation. The multiple overlaid curves are different injections on the same column, as about 30 injections were required to process all of the material.

FIGS. 6A and 6B depict size exclusion chromatograms of in-vitro conjugation reaction mixtures of rAAV9 to antibody in ratios of 1:0, 1:1, 1:6, 1:10, 1:20, and 1:30 at pH 5.0 (FIG. 6A), and at a 1:30 rAAV9 to antibody ratio and pH values of 5.0, 6.5, 8.0, 8.5, 9.0, 9.5, 10.0 (FIG. 6B). Recombinant AAV concentration was 2.3×10¹³ cp/mL and separated on a 450 Å size exclusion chromatography column. Reaction conditions included brief mixing followed by 1 hour incubation prior to freezing, resulting in incomplete conjugation shown by the presence of AAV-SpyT peak at all molar ratios.

FIGS. 7A to 7K are as follows: FIG. 7A depicts exemplary elution profiles for AAV9 and AAV9-SpyT for empty, partially filled and full capsids, and serves as a model for other AAVs. There will exist empty and partially-filled capsid populations can have isoelectric points (pI) that are above full capsids and other empty and partially-filled capsid populations will have isoelectric points that are below full capsids. FIG. 7B is a graph depicting separation empty, partially filled and full AAV9-SpyT capsids using a hybrid gradient combining linear and step gradients. The column was a CIM QA monolith loaded with 5×10¹³ to 5×10¹⁴ capsids (cp/ml). The buffer was 20 mM Bis-Tris-Propane, 0.001% P188, pH 9.6 and 1 M NaCl and the process resulted in enrichment of full capsids from 14% to 55%. This hybrid gradient enabled improved resolution of the full capsids (central peak) from the empty capsid populations on either side. However, the hybrid gradient is operationally challenging at scale and also does not result in full capsids above the required threshold of 70%.

FIG. 7C depicts data from a two-pass anion exchange chromatogram for a 500 L purification of AAV-SpyTag with first pass and second pass microsteps. This process achieved a recovery of 96% full capsids. Partially-filled capsids were only 1% of total and empty capsids were only 3% of total.

FIGS. 7D to 7F depict data from an experiment using anion exchange to separate a covalently surface modified adeno-associated virus species from unconjugated antibody species. A CIM QA 1 mL monolith was used as the anion exchange column, and the column was loaded with an in-vitro conjugation reaction mixture of AAV9-SpyT and SpyC-ASGR1 mAb mixed in a 1:30 molar ratio. In FIG. 7D, the AAV9-SpyT capsids were 10% full, whereas in FIG. 7E the AAV9-SpyT capsids were 70% full. FIG. 7F depicts the size exclusion chromatograms combined with multi-angle light scattering (MALS) data of six elution fractions collected from FIG. 7E, showing the increasing conjugation level, and increasing mass distribution of the later eluting fractions. FIG. 7G is a graph of the retention time of conjugated AAV9 using SpyTag-SpyCatcher with an antibody, unconjugated AAV-SpyTag and unconjugated SpyCatcher-antibody. FIG. 7H is a graph depicting the effect of AAV capsid concentration on percent conjugation. FIG. 7I is a graph depicting the effect of incubation temperature on percent conjugation, and FIG. 7J is a graph depicting the effect of pH on percent conjugation. FIG. 7K depicts data for conjugation of AAV-SpyT conjugated to Spy C-Fab (Construct 1) and AAV-SpyT conjugated to Spy C-mAb (Construct 2). Both AAVs have the W503A detargeting mutation and HA-MTM1 as a transgene.

FIGS. 8A to 8E depict data from experiments using cation exchange to separate a covalently surface modified adeno-associated virus species from unconjugated antibody species. In FIG. 8A, a CIM SO3 1 mL monolith was used as the cation exchange column, and the column was loaded with an in-vitro conjugation reaction mixture of AAV9-SpyT and SpyC-ASGR1 mAb mixed in a 1:30 molar ratio. FIG. 8A depicts a linear gradient elution and highlights the peaks corresponding to the SpyC-Ab and the conjugated AAV9-SpyT-SpyC-Ab species.

FIG. 8B depicts a step elution in which the in-vitro conjugation reaction mixture was adjusted with sodium chloride solution to a conductivity of 15 mS/cm and the mixture was loaded onto a Poros XS cation exchange column, leading to all SpyC-Ab species not binding onto the column and being removed in the flow-through, while all AAV species bound and were removed later in an isocratic elution at 40 mS/cm conductivity. FIG. 8C depicts data showing that Poros XS cation exchange can separate free SpyC-Ab species following the conjugation reaction, shown for a separation of AAV9-SpyT containing CAGG-eGFP transgene conjugated to a SpyC-ASGR1 mAb. FIGS. 8D and 8E compare CaptoCore400 flow through (size exclusion and anion exchange) (FIG. 8D) to Poros XS elution (cation exchange) (FIG. 8E).

FIG. 9 depicts an experiment using multimodal anion exchange to separate a covalently surface modified adeno-associated virus species from unconjugated antibody species.

FIG. 10 depicts data from an experiment using multimodal binding and size separation bead chromatography exchange on CaptoCore400 resin to separate a covalently surface modified adeno-associated virus species from unconjugated antibody species. The relative size of species was about 8 nm for the SpyC-Ab, about 15 nm for the AAV9-SpyT, and about 17 nm for the conjugated AAV9-SpyT-SpyC-Ab. The figure includes a size exclusion chromatogram showing that the load material included two peaks (AAV and SpyC-Ab), but the flow through included only one peak (only AAV species), showing removal of free SpyC-Ab species using this technique.

FIG. 11 depicts a multivariate model from experiments using different salt and pH conditions to induce a change in the size and surface characteristics of AAVs, leading to improved yields in the flow through of CaptoCore400 chromatography. The model is a yield profiler and predicts an optimum condition of adjusting the load material with sodium citrate to a final concentration of 150 mM at pH 5.5, resulting in a predicted yield of 100% and an experimental yield of 107% as measured by ddPCR quantification.

FIGS. 12A-12C depict dynamic light scattering (DLS) data from AAV9-SpyT, SpyC-ASGR1 mAb (FIG. 12A), and in-vitro conjugation reactions between these two species under stirred and unstirred conditions in sodium chloride and sodium citrate based buffers. The DLS data shows the formation of large aggregates in the conjugation reaction under stirred conditions (FIG. 12B), but no such aggregates in the unstirred condition (FIG. 12C) (incubation only).

FIG. 13A shows size exclusion chromatography data tracking the rate of reaction between AAV9-SpyT and SpyC-ASGR1 mAb for three different batches of AAV9-SpyT with different % full capsids. The percentage of conjugated species is measured by relative peak area between the AAV9-SpyT peak at about 8.3 min RT and the conjugated species peaks at 6.5 to 8.0 RT on the size exclusion chromatograms, and tracked in the chart on the right. All batches reached 80% conjugation, but Batch 1 took 48 hours to reach an example of an optimal conjugation level (80%) while Batches 2 and 3 took 24 hours.

FIG. 13B depicts conjugation data with AAV9-SpyT and SpyC-mAb (anti-CACNG1). The mAb has two SpyC. FIG. 13C depicts conjugation data with AAV9-SpyT and SpyC-bispecific Ab (anti-CACNG1). This bispecific mAb has one SpyC. FIG. 13D depicts conjugation data with AAV9-SpyT and SpyC-Fab (anti-CACNG1). The Fab has one SpyC. Molar ratios of AAV:Ab (antibody with 2-SpyC, bispecific antibody with one SpyC, or Fab with one SpyC) ratios of 1:1, 1:6, 1:15, 1:30, 1:300 and 1:1000 were tested.

FIG. 14A is a graph depicting how anion exchange chromatography separates unconjugated AAV from increasingly conjugated species, which are schematically depicted underneath the graph. FIG. 14B depicts SEC-MALS data of samples with different levels of conjugation. The samples were obtained from an anion exchange chromatography method similar to the one described in FIGS. 7D to 7F that can be used to separate populations of differently conjugated species. The chart underneath the graph summarizes the SEC-MALS analysis of the samples which is used to estimate the number of mAbs conjugated on average in each sample, ranging from less than 1 mAb in the first fraction to more than 5 mAbs in the last fraction. FIG. 14C shows the results of a transduction assay where these differently conjugated species were compared for transduction ability using cells expressing ASGR1 receptor. It was observed that lower conjugated species resulted in higher transduction efficiency than the combined pool (Load) as well as heavier conjugated species, suggesting a negative impact of higher conjugation levels on transduction ability, despite potential advantages in targeting ability. FIG. 14D and FIG. 14E collectively concern transduction efficiency at different pHs and percent conjugation. FIG. 14D depicts green fluorescent protein (GFP) percentage of bispecific antibody (bsAb or bsmab) with one SpyC, a monospecific monoclonal antibody (mAb) with two SpyCs, or an antigen-binding antibody fragment (Fab) with one SpyC, at reaction pH of 5 or 8. FIG. 14E depicts mean fluorescence intensities (MFI) for the bsAb, mAb and Fab at pH 5 or 8.

FIG. 15 schematically depicts the production of an antibody or Fab fused to SpyCatcher.

FIG. 16 schematically depicts the production of AAV comprising SpyTag on the capsid surface using cells transfected with the four plasmids of the quad transfection system and method. See FIG. 2.

FIG. 17 schematically depicts the conjugation of AAV-SpyT to an antibody fused to SpyCatcher and then enrichment using chromatography, such as cation exchange chromatography, and tangential flow filtration. See FIG. 15 (depicting production of SpyCatcher fused to an antibody or Fab) and FIG. 16 (depicting production of AAV-SpyT).

FIG. 18 schematically depicts the effects of different concentrations of additives on conjugation. The selected additives were dimethylsulfoxide (DMSO), L-glutamic acid monosodium salt (L-glutamic acid), arginine, sorbitol, urea, isopropyl alcohol (IPA) and sodium thiocyanate (NaSCN).

FIG. 19 depicts data showing the effects of the additives used in FIG. 18.

FIGS. 20A to 20H are as follows: FIG. 20A schematically depicts a hypothetical readout on the impact that mosaicism has on transduction efficiency (green) and percent (%) conjugation (red) with Fab, bispecific Ab and mAb. In this hypothetical, 80% is the desired limit for conjugated capsids. Mosaicisms selected for testing were 1/5, 1/7.5, 1/10, 1/15 and 1/30. FIG. 20B is a bar graph comparing upstream bioreactor titer and process yield for five different mosaicisms (“M”), produced at 2 L scale for 1:5 (M1), 1:7.5 (M2), 1:15 (M4) and 1:30 (M5), and at 50 L scale for 1:10 (M3). FIG. 20C is a bar graph comparing percent (%) conjugation vs. time for the five different mosaicisms, as measured using size-exclusion chromatography. The data indicates a maximum conjugation for the 1:7.5 and 1:10 mosaicisms.

FIGS. 20D to 20H are graphs schematically depicting data of size-exclusion chromatography overlays showing conjugation process for different mosaicisms M1 (1:5), M2 (1:7.5), M3 (1:10), M4 (1:15), and M5 (1:30). The FM is SpyT and the SCM is SpyC here. FIG. 20D depicts an overlay of M1-M5 prior to addition of SpyC-Abs, showing increasing size with increasing level of mosaicism (M1>M2>M3>M4>M5). The X-axis designates 7.60 to 10.40 minutes and the Y-axis designates 0.00 to 800.00 EU. FIG. 20E depicts an overlay of conjugation reactions between M1 and anti-CACNG1 single-SpyC mAb at 2, 8, 24 and 36 h, showing increasing conjugation level over time via reduction of the peak at about 8.5 min RT. The X-axis designates 3.00 to 14.50 minutes and the Y-axis designates 0.00 to 200.00 EU. FIG. 20F depicts an overlay of conjugation reaction between M2 and anti-CACNG1 single-SpyC Fab at 2, 8, 24 and 36 h, showing increasing conjugation level over time via reduction of the peak shoulder at about 8.5 min RT. The X-axis designates 1.50 to 14.50 minutes and the Y-axis designates 0.00 to 450.00 EU. FIG. 20G schematically depicts an overlay of conjugated species of M1-M5 and anti-CACNG1 single-SpyC Fab after 48 h of reaction, showing increasing conjugation level for higher mosaicisms. The X-axis designates 0.00 to 15.00 minutes and the Y-axis designates 0.00 to 1600.00 EU. FIG. 20H depicts in the Final Concentration Pool (FCP) an overlay of conjugated species with mosaicisms M1-M5 and anti-CACNG1 single-SpyC mAb after 48 h of reaction and removal of majority of excess free SpyC-Ab using preparative size exclusion chromatography, which showed increasing conjugation level for higher mosaicisms. The X-axis designates 0.00 to 15.00 minutes and the Y-axis designates 0.00 to 160.00 EU.

FIG. 21A depicts production purification trains. The top train uses a batch tangential flow filtration unit where repeated passes are required to exchange buffer and concentrate the retentate. The bottom section replaces the batch tangential flow filtration unit with a single pass tangential flow filtration unit. Ionic exchange chromatography of different modalities can be used following TFF, and anion exchange is depicted as an exemplar. FIG. 21B depicts a process for purifying retargeted AAV at the 500 L production scale.

FIG. 22A schematically shows a batch tangential flow filtration (Batch TFF) (top), where the retentate is repeatedly cycled through a feed tank and pump to repeatedly passed through a membrane, with the concentrated permeate being removed after repeated cycles. A single pass tangential flow unit (Single-Pass TFF or SPTFF) removes material from the feed tank through a pump to a multi-stage membrane module that separate the retentate from the permeate, while concentrating the permeate. FIG. 22B is a graph comparing Batch TFF and Single-Pass TFF. Single-Pass TFF achieves higher concentration and is faster as compared to Batch TFF. Single-Pass TFF continuously delivers biological material (such as AAV) to the next operation in the purification train, whereas Batch TFF does not deliver biological material (such as AAV) until the end of the batch cycle.

FIG. 23 schematically compares the batch operation to a continuous operation in terms of Cell lysis, Clarification, TFF (Batch or Single-Pass) and Affinity Capture. The continuous process can be completed in less than a day, whereas the batch process can be multi-day.

FIG. 24 schematically depicts exemplary arrangements for multi-stage membrane module cassettes to be used with Single-Pass TFF. The configurations depict four to seven tiers of membrane module cassettes where the initial tiers (left side) contain more or same number of membrane module cassettes as the succeeding tiers (moving towards the right side). Total area and path length of the membrane module cassettes also are set forth.

FIG. 25 is a graph depicting volumetric concentration factor (VCF) versus transmembrane pressure (TMP) using the 4-in-series, 5-in-series, 6-in-series and 7-in-series exemplary configurations depicted in FIG. 24 with a feed comprising an exemplary AAV, here AAV9 comprising a SpyTag insert.

FIG. 26 depicts data from a 5-in-series configuration according to FIG. 24 at flow rates of 90 ml/minute, 120 ml/minute and 150 ml/minute. The log best-fit equation of VCF=A In (TMP-B) using the values at each flow rate set forth near the plot (and rounded off in the included table) can be used to parameterize the data. At the right side of the figure, there is a graph of parameter value (A, B) and feed flow rate in liters per square meter of membrane per hour (LMH) for 4-in-series and 5-in-series exemplary configurations of FIG. 24 and allows optimized conditions to be selected in silico using an exemplary AAV, here AAV9 comprising a SpyTag insert. This model can be used to predict the VCF for any flow rate and TMP for an in-series configuration of interest.

FIG. 27A is a design space model based on FIGS. 25 and 26 using the 5-in-series configuration of FIG. 24. Here, the process target was 35 LMH, and the intersecting lines indicate a VCF of 8 and a TMP of 10 psi. An exemplary acceptable zone would be a VCF of 6-10 and a TMP of 7.5 to 12.5 psi.

FIG. 27B is an exemplary comparison of process parameters between SPTFF and Batch TFF. With Batch TFF, typically there would be one batch before the next operation. However, depending on the scheduling of upstream production bioreactors and bioreactor titers, there could be pooling of multiple batches before the next operation

FIG. 28 depicts data from a bench-scale trial to determine the number of buffer washes need to attain about a 90% recovery of AAV, here AAV9 with integrated SpyTag, in a low-TMP process. On average, the AAV9 here contained an average of 6 SpyTag peptides per capsid. Capsid titer in retentate (cp/ml) versus SPTFF operating time (minutes) was measured using four buffer flushes. As the right side of the figure shows, it was determined that only two buffer flushes were required to achieve about a 90% recovery with a VCF of 8λ.

FIG. 29 is a graph depicting Permeate Flux (LMH), Throughput (L/m²), Feed Flow Rate (L/hr) and TMP (psi) in a pilot-scale trial. The data showed flux decline and TMP build up. To mitigate TMP increase beyond 12.5 psi, feed flow rate was slowed. This resulted in a longer process time of 180 minutes rather than the expected 90 minutes and an overall VCF of 5× was achieved rather than the target of 8λ.

FIG. 30 schematically depicts a tween micelle build-up on the TFF membrane, which is believed to be the cause of an unexpected flux decline of about 50%. This figure also set forth the approximate size of AAV, Host Cell protein aggregates (HCP) and Tween-20 micelles. Detergents, such as Tweens, are a common component of cell lysis buffers used in the purification of AAV.

FIG. 31 is a graph depicting fold presence of Tween-20 on the retentate side of membrane and the Permeate side of the membrane for both Batch TFF and SPTFF.

FIG. 32 is a graph depicting the flux decline after two hours with varying percentages of Tween-20 in the lysis buffer. In addition to Tween-20, the buffer contained 20 mM Tris, 2 mM MgCl₂ at a pH of 7.4. The feed flow rate was 35 LMH and the TMP was about 5 to 10 psi.

FIG. 33 compares control with the retentate valve to control with a permeate pump. Option 1 with the retentate valve found that TMP reached 22 psi, and after which the flow had to be reduced from 40 LMH to 30 LMH. VCF dropped from about 10× to about 6λ. Option 2 with the permeate pump was superior. TMP was controlled to well under 10 psi and a VCF of 8× was maintained. At the right side of the figure, Option 1 (SPTFF with retentate valve) and Option 2 (SPTFF with permeate pump) were compared to a Batch TFF. Option 1 did not perform as well as Option 2 and Batch TFF. Option 2 was superior to Batch TFF and Option 1 in terms of capsid yield and percent aggregation.

FIG. 34 depicts an overall pilot scale process.

FIG. 35 compares VCFs (1-14), SPTFF retentate flow rates and residence time in affinity capture. VCFs of 7 to 13 and SPTFF retentate flow rates of 75-40 provided an exemplary range of residence time suitable for affinity loading.

FIG. 36 depicts how UV280 profile of affinity capture flow can be used for process monitoring of VCF and process stability using SPTFF for continuous processing. Three different runs were performed for comparison purposes. Run 1 was performed without a permeate pump and achieved a VCF of only 5λ. Run 2 was performed with a permeate pump with a feed to retentate flush (with recirculation) and achieved a VCF of 8λ. Run 3 was performed with a permeate pump with a feed to retentate flush (with recirculation) and a permeate to retentate flush and achieved a VCF of 1 Ox.

FIG. 37A depicts vector viral titer and viral genome to capsid ratio (Vg:Cp) of different capsid types: a wild type (WT) AAV9, a detargeted AAV9 and a detargeted AAV9 comprising the SpyTag peptide in the viral capsid. FIG. 37B depicts virus vector viral titer and viral genome to capsid ratio (Vg:Cp) at different bioreactor scales: 0.2 liters, 2 liters and 50 liters.

FIG. 38 is a graph depicting conjugation reactions between AAV9-SpyT capsids with N272A detargeting mutation, with two different SpyCatcher antibodies, SpyC-ASGR1 mAb and SpyC-FELD1 mAb. Successful conjugation was achieved comparable to results using AAV9-SpyT capsids with W503A detargeting mutations (FIG. 5A).

### DETAILED DESCRIPTION OF THE INVENTIONS

### Definitions

Unless defined otherwise, all technical and scientific terms used herein have the same meaning as commonly understood by one of ordinary skill in the art to which the inventions belong.

The term “about” in the context of numerical values and ranges refers to values or ranges that approximate or are close to the recited values or ranges such that the invention can perform as intended, such as having a desired rate, amount, density, degree, increase, decrease, percentage, ratio, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDRI, LCDR2 and LCDR3. The term “high affinity” antibody refers to those antibodies having a binding affinity to their target of at least 10-9 M, at least 10⁻¹⁰ M; at least 10⁻¹¹ M; or at least 10⁻¹² M, as measured by surface plasmon resonance, for example, BIACORE™ or solution-affinity ELISA.

The phrase “bispecific antibody” includes, but is not limited to, an antibody capable of selectively binding two or more epitopes, and can be used as retargeting molecules. In one approach, bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. Predominantly, a bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes. In another approach, a bispecific antibody can be an antibody where there are alterations to one or both of the heavy chains on either the variable or constant region. For example, there can be a bispecific antibody where one of the heavy chains is joined, modified to include, or genetically fused to another molecule, such as a SpyCatcher protein or fragment/derivative thereof, while the other heavy chain is not .Techniques and components can be referenced in International Patent Publication Nos. WO2010/151792, WO2014/047231, WO2016/018740, WO2016/161010, WO2019/006046 and U.S. Pat. No. 8,586,713.

The term “components” refers to the constituent molecules needed to produce recombinant AAV, such as covalently surface modified adeno-associated viruses and includes, but is not limited to, promoters, polyadenylation signals, transgenes, polynucleotides encoding retargeting molecules, AAV cap genes, AAV rep genes, ITRs, helper polynucleotide sequence(s), polynucleotides encoding a first member of a specific binding pair and a second cognate member of a specific binding pair (for covalently surface modified AAV), as well as peptides encoded by the genes and polynucleotide sequences. Optional components include detargeting mutation sequences, introns, IRESs, RRSs, operators and enhancers.

The phrase “assembly of components” refers to components that assemble together by way of bonds, forces, interactions and/or attractions. Examples include the assembly of heavy and light chains to form antibodies, capsid proteins, and isopeptide bonds formed during conjugation of specific binding pairs.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

“Recombinase recognition sites” (RRS), also known as “heterospecific recombination sites,” are used in recombinase mediated cassette exchange (RMCE). Cre/Lox Dre/Rox, Vre/Vlox, SCre/Slox and FIp/Frt are suitable systems, for example. Suitable RRSs for use according to the inventions include Lox P, Lox 66, Lox 71, Lox 511, Lox 2272, Lox 2372, Lox 5171, Lox M2, Lox M3, lox M7 and Lox M11. These sites can be referred to generically as first (1), second (2), third (3), fourth (4), fifth (5), sixth (6), seventh (7), eighth (8), ninth (9), tenth (10), etc., as is apparent from the context of usage.

An “intron” is a section of DNA that is not protein encoding, and typically is located between “exons”, which encode protein regions. An intron is removed to form a mature messenger RNA, which is translated to form protein. Some introns are those that can affect the starting point of translation, and exemplars are the hCMV-IE intron (Human cytomegalovirus immediate early protein) and FMDV intron (Foot and Mouth Disease Virus).

“Intronic selection” refers to the optional use of recombinase recognition sites located in intronic regions to allow for integration of multiple cassettes to form a construct. See Published applications US 2019/0263937 A1 and US 2019/0233544 A1. For example, selection markers and reporter genes can be engineered to include introns with RRSs contained therein. Intronic selection can be used to create constructs sectionally. For instance, a large construct containing multiple cassettes can be created by using smaller, constituent constructs.

“Antibody derivatives and fragments” include, but are not limited to: antibody fragments (for example, Fab, ScFv-Fc, dAB-Fc, half antibodies and other combinations of heavy and/or light chains), multispecifics (for example, bispecifics, IgG-ScFv, IgG-dab, ScFV-Fc-ScFV, trispecifics).

The phrase “Fc-containing protein” includes antibodies, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region can create charge variation.

“Fc-fusion proteins” comprise part or all of two or more proteins, one of which is an Fc portion of an immunoglobulin molecule, that are not fused in their natural state. Fc-fusion proteins include Fc-Fusion (N-terminal), Fc-Fusion (C-terminal), Mono Fc-Fusion and Bi-specific Fc-Fusion. Preparation of fusion proteins comprising certain heterologous polypeptides fused to various portions of antibody-derived polypeptides (including the Fc domain) has been described, for example, by Ashkenazi et al., Proc. Natl. Acad. Sci. USA 88: 10535-39 (1991); Byrn et al., Nature 344:677-70, 1990; and Hollenbaugh et al., “Construction of Immunoglobulin Fusion Proteins”, in Current Protocols in Immunology, Suppl. 4, pages 10.19.1-10.19.11 (1992). “Receptor Fc-fusion proteins” comprise one or more of one or more extracellular domain(s) of a receptor coupled to an Fc moiety, which in some aspects comprise a hinge region followed by a CH2 and CH3 domain of an immunoglobulin. In some aspects, the Fc-fusion protein contains two or more distinct receptor chains that bind to a single or more than one ligand(s). Some receptor Fc-fusion proteins may contain ligand binding domains of multiple different receptors. Receptor Fc-fusion proteins are also referred to as “traps,” “trap molecules” or “trap proteins.” For example, such trap proteins include an IL-1 trap (for example, Rilonacept, which contains the IL-IRAcP ligand binding region fused to the IL-1 R1 extracellular region fused to Fc of hlgGI; see U.S. Pat. No. 6,927,044, or a VEGF Trap (for example, Aflibercept, which contains the Ig domain 2 of the VEGF receptor FItl fused to the Ig domain 3 of the VEGF receptor FIkl fused to Fc of hlgG1 See U.S. Pat. Nos. 7,087,411 and 7,279,159.

“Polynucleotide” includes a sequence of nucleotides covalently joined, and includes RNA and DNA. Oligonucleotides are considered shorter polynucleotides. Genes are DNA polynucleotides (polydeoxyribonucleic acid) that ultimately encode polypeptides, which are translated from RNA (polyribonucleic acid) that was typically transcribed from DNA. DNA polynucleotides also can encode RNA polynucleotides that is not translated, but rather function as RNA “products”. The type of polynucleotide (that is, DNA or RNA) is apparent from the context of the usage of the term. A polynucleotide referred to or identified by the polypeptide it encodes sets forth and covers all suitable sequences in accordance with codon degeneracy. Polynucleotides, including those disclosed herein, include percent identity sequences and homologous sequences when indicated.

“Polypeptide” or “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

A “gene of interest” (GOI) encodes a “protein of interest” or “polypeptide of interest” and optionally can include other associated sequences. The sequences can be natural, semi-synthetic or synthetic. Native sequences, mutant sequences and degenerate sequences can be GOIs. A gene of interest also can be referred to as a “transgene.”

A “nucleotide of interest” includes GOIs and sequences encoding non-translated RNAs/non-coding RNAs (such as, but not limited to, antisense RNA, micro RNA, small interfering RNA, catalytic RNA and ribozymes). NOIs and GOIs also can be referred to as “payloads.”

“Protein of interest” or “polypeptide of interest” (POI) can have any amino acid sequence, and includes any protein, polypeptide, or peptide that is desired to be expressed, typically for gene therapy purposes. Protein types can include, but are not limited to, receptors, fusion proteins, agonists, antagonists, activators, inhibitors, enzymes (such as those used in enzyme replacement therapy), factors and co-factors, repressors, activators, ligands, protein hormones, therapeutic proteins, suicide proteins, structural proteins, storage proteins, transport proteins, signal proteins, neurotransmitters and contractile proteins. Derivatives, components, domains, chains and fragments of the above also are included. The sequences can be natural, semi-synthetic or synthetic.

“Purification” in its various grammatical forms includes, but is not limited to, the use of one or more procedures such as depth filtration, tangential flow filtration, affinity capture, ionic exchange and the like.

The term “recombinant capsid protein” includes a capsid protein that has at least one mutation in comparison to the corresponding capsid protein of the wild-type virus, which wild-type may be a reference and/or control virus for comparative study. A recombinant capsid protein includes a capsid protein that comprises a heterologous amino acid sequence, which may be inserted into and/or displayed by the capsid protein. “Heterologous” in a general context means heterologous as compared to the virus, from which the capsid protein is derived. The inserted amino acids can simply be inserted between two given amino acids of the capsid protein. An insertion of amino acids can also go along with a deletion of given amino acids of the capsid protein at the site of insertion, for example, 1 or more capsid protein amino acids are substituted by 5 or more heterologous amino acids). An example of a heterologous amino acid sequence that can be inserted is a member of a specific binding pair, such SpyTag.

“Detargeting” refers to reducing or abolishing AAV natural preferential transduction, primarily of the liver, by mutating Cap proteins. For example, mutations in the galactose binding domain of VP1 assist in detargeting the liver. Other mutations include, but are not limited to, Cap mutations that alter heparin binding or sialic acid binding. These mutations are optional and can be referred to as “detargeting mutations.”

By way of example, different AAV serotypes are known to preferentially transduce the cells of different tissues. Tissue specificity is limited, and AAV is known to preferentially transduce the liver, which can be a safety and efficacy concern in some contexts. The inventions further provide mutations in the VP1 Protein of AAV9, for example, to lower the AAV preferential transduction of the liver. The AAV9 mutations include N272A and W503A substitutions, where alanine replaces both asparagine at position 272 of VP1 and tryptophan at position 503 of VP1. One or both of the mutations can be undertaken in the VP1 protein. Optionally, other amino acids, such as glutamic acid, serine or others, can be used instead of alanine for substitution. Additional detargeting mutation sites include, but are not limited to, N470, D271, and Y446. The inventions provide exemplary mutations for use with AAVs as follows, which for each serotype can be utilized singly or in combinations
AAV1-N500E;
AAV2-R585A and R588A;
AAV5-T571 S;
AAV6-N500E, K531A and K531 E;
AAV9-N272A, W503A, T568P, Q590L, N498Y, L602F, S414N, G453D, K557E, T5821, and combinations include, but are not limited to, T568P+Q590L, N498Y+L602F, S414N+G453D+K557E+T5821.

These and others are set forth in the chart below:

| AAV | Insertion | |
|---|---|---|
| serotype | Sites | Exemplary Mutations |
| AAV1 | 447, 472, 473, | N477, S472, V473, N500, N502, W503, |
| | 500, 502, 503 | N500E, S268, D270, N271, Y445, G470, |
| | 587, 589 | K531, and K531, K531A and K531E |
| AAV2 | 1, 34, 138, | R484, R485, R487, R532, R585, R585A, |
| | 139, 161, | R588, T589, R588A, K532 |
| | 261, 266, | R484A, R487A, R487G, K532A, K532D, |
| | 381, 447, | R585A, R585S, R585Q, R588A, R588T, |
| | 448, 453, | G453, N587, E499 |
| | 459, 471, | Q464V, A467P, D469N, 1470M, R471A, |
| | 484, 487, | D472V, S474G, Y500F, S501A, R432A, |
| | 520, 532, | L510A, N511R, R404A, R295A, R294A, |
| | 534, 570, | Q297A, R298A, R294A/R298A, T331A, |
| | 573, 584, | K692A, W694A, P696A, R389A, Y441A, |
| | 585, 587, | L510A N511R, P602A, R432A, P622A, |
| | 588, 591, | D625A, V221A/S224A, H255/K258A, |
| | 657, 664, | Y413A, F415A, D416A, M402A, E415A, |
| | 713, 716 | H229/D231A, K321A/E322A, N334W, |
| | | V221W, V221C, V221Y, L336C |
| AAV3 | 585, 594 | R594, G594, N588A, N588S, A590Q, |
| | | S384A, T717V |
| AAV4 | 492, 499, | K492, S499, K503, T504, M523, M524, |
| | 503, 504, | G581, N583, Q583, Q585, N585, S587 |
| | 523, 524, | |
| | 581, 583, | |
| | 584, 585, 587 | |
| AAV5 | 531, 569, | K531A, K531E, T571S, M569, A570, |
| | 570, 571, | T571, V580, A581, V582, G583, T584, |
| | 575, 580, | Y585, N586, L587, A592, G594, D595, |
| | 581, 582, | V596, H597, A598, T571, T571S |
| | 583, 584, | |
| | 585, 586, | |
| | 587, 592, | |
| | 594, 595, | |
| | 596, 597, 598 | |
| AAV6 | 447, 459, | K459, K493, N500E, K531, K531A, |
| | 472, 473, | K531E, R576, N500, Q585 |
| | 493, 500, | |
| | 502, 503, | |
| | 531, 576 | |
| AAV7 | | S157, N254, N460, A2, K61, T252 |
| AAV8 | | Y6, A2, N521, Q588, T591, N590 |
| AAV9 | 270, 271, | D270, D271, N271, N272, N272A, Y445, |
| | 272, 445, | Y446, N470, G470, W503, W503A, |
| | 446, 453, | S469, A742, V473, P504 and Q590, |
| | 470, 503, | K532A, K532D, R484A, R487A, R585A, |
| | 587, 589 | R585S, R585Q, R487G, R588A, R588T, |
| | | G453, A587, A589, E500, S586, A589 |
| AAV10 | | A589T |
| AAV13 | 528, 532 | K528, E531 |
| Avian | 444, 580 | G444, K580 |
| AAV | | |
| Sea lion | 429, 430, | |
| AAV | 431, 432, | |
| | 433, 434, | |
| | 436, 437, 565 | |
| Bearded | 573, 436 | G436, T573 |
| Dragon | | |
| AAV | | |
| AAV-DJ | | K137R, T251A, S503A |
| rAAVrh.10 | | K259, K333, S453, S501, S559, Q589, |
| | | N590, A592, S671, T674, Y708, T719, |
| | | AS453, K259L, K333V, AS453, S501A, |
| | | S559A, Q589N, N590S, A592Q, S671A, |
| | | T674V, Y708A, T719V |
| AAVrh10 | | A2, Y90, S149, S157, T252, N306, T332, |
| (AAV10) | | K333, S449, K652, K709 |

Other mutations and detargeting approaches, such as variable loop swaps, are disclosed in publications or otherwise available See, for example, Shen et al., Molecular Therapy 15: 1955-62 (2007). These technologies can be used according to the inventions. Detargeting mutations are optional.

“Retargeting” or “redirecting” may include a situations in which the wildtype vector targets several cells within a tissue and/or several organs within an organism, which general targeting of the tissue or organs is reduced or abolished by provision of a retargeting molecule, which retargets the covalently surface modified AAV to a different, and optionally more specific cell in the tissue or a specific organ in the organism.

The term “retargeting molecule” (Rm) is a molecule useful for targeting an antigen, receptor protein, including glycoproteins, and/or ligand (“collectively targets”) found on the surface of a cell, referred to as a “target cell.” The retargeting molecule can be bound to a polypeptide that is part of a specific binding pair. For example, a retargeting molecule could be bound to SpyCatcher (or fragments/derivatives thereof) in order to utilize the SpyTag-SpyCatcher system. The retargeting molecule can target the cell that has the antigen, receptor and/or ligand that the retargeting molecule can bind to, and thereby direct a recombinant AAV to that cell. Fc-containing proteins, such as antibodies, monoclonal antibodies (including derivatives, fragments, half antibodies and other heavy chain and/or light chain combinations), multispecific antibodies (for example, bispecifics and trispecifics), IgG-ScFv, IgG-dab, ScFV-Fc-ScFV, Fc-fusion proteins, receptor-Fc fusion proteins, and trap proteins, are useful as retargeting molecules. Mini-trap proteins also can be used as retargeting molecules. In the literature, the phrase “targeting ligand” has been used for molecules that are useful for targeting. See Yan et al., Pharmaceutics 16: 248 (2024).

All human and non-human antibody classes can be used as retargeting molecules. IgA, IgD IgE, IgG and IgM can be used as retargeting molecules. IgG is a preferred class, and includes subclasses IgG1 (including IgG1A and IgG1 K), IgG2, IgG3, and IgG4. Further antibody types include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody, tetrabody, Fab, F(ab′), F(ab′)2 and a half antibody.

“Specific binding pair”, also referred to a “protein:protein binding pair” and the like include two proteins (that is, a first member, such as a first polypeptide, and a second cognate member, such as a second polypeptide) that interact to form a covalent isopeptide bond under conditions that enable or facilitate isopeptide bond formation, wherein the term “cognate” refers to components that function together by to reacting together to form an isopeptide bond. Thus, two proteins that react together efficiently to form an isopeptide bond under conditions that enable or facilitate isopeptide bond formation can also be referred to as being a “complementary” pair of peptide linkers. Specific binding pairs capable of interacting to form a covalent isopeptide bond are reviewed in Veggiani et al. (2014) Trends Biotechnol. 32:506, and include, for example, peptide:peptide binding pairs such as SpyTag:SpyCatcher, SpyTag002:SpyCatcher002, SpyTag:KTag, isopeptag:pilin C, SnoopTag:SnoopCatcher and others. Spy Tag002:SpyCatcher002 and SpyTag003:SpyCatcher003 are different iterations of Spy Tag:Spy Catcher.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly, that is between two peptide/protein molecules, such as between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. An isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

“Mosaicism” refers to the ratio of AAV cap sequence containing plasmids that also contain a sequence encoding a first member as compared to the cap plasmids without the first member sequence. Typically, a cap plasmid will contain both the AAV cap gene and the AAV rep gene. An example is plasmid pRC as taught herein. An example of a plasmid that also encodes a first member is pRC-SpyT as taught herein. Plasmid pRC-SpyT serves as an exemplar of a cap plasmid that comprises a nucleotide sequence of a first member.

For example, when 1/10 of all the pRC plasmids transfected are pRC-SpyT plasmids, the AAV capsid is expected to have an average of 6 SpyT, as 1/10 of the 60 viral proteins are expected to have a SpyT (1 pRC-SpyT/10 total cap-containing plasmids)×60=6). Similarly, a transfection ratio of 1/4 is expected to result in 15 SpyT per AAV capsid (1/4×60=15), and a transfection ratio of 1/30 is expected to result in 2 SpyT per AAV capsid (1/30×60=2), as the capsid is stoichiometrically assembled inside the cell using the available viral proteins. The ratio of pRC-SpyT to total pRC plasmids during transfection is referred to as the “mosaicism” of the AAV capsid, with “higher mosaicisms” such as 1/4 resulting in higher SpyT on the AAV capsids on average, and “lower mosaicisms” such as 1/30 resulting in fewer SpyT on the AAV capsids on average. Thus, where the ratio of pRC-FM to pRC plasmids is a larger fractional value, the mosaicism will be higher. As explained above, 1/4 is a larger fractional value than 1/30.

Mosaicisms can be selected by the person skilled in the art in view of the teachings contained herein. Mosaicisms can be ratios as set forth below and those thereabout and therebetween: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, or 1/60.

Ranges of ratios of 1/1 to 1/60 can be employed, and subranges as set forth below.

Preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

More preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of ratios are 1/4 to 1/13; 1/5 to 1/12; 1/6 to 1/11, 1/7 to 1/10.5 or 1/7.5 to 1/10.

“Reporter proteins” as used herein, refers to any protein capable of generating directly or indirectly a detectable signal. Reporter proteins typically fluoresce, or catalyze a colorimetric, bioluminescence, or fluorescent reaction, and often are referred to as “color proteins,” “bioluminescent proteins” or “fluorescent proteins.” However, a reporter protein also can be non-enzymatic and non-fluorescent as long as it can be detected by another protein or moiety, such as a cell surface protein detected with a fluorescent ligand. A reporter protein also can be an inactive protein that is made functional through interaction with another protein that is fluorescent or catalyzes a reaction. Accordingly, any suitable reporter protein, as understood by one of skill in the art, could be used. The reporter protein can be selected from fluorescent protein, luciferase, alkaline phosphatase, β-galactosidase, β-lactamase, dihydrofolate reductase, ubiquitin, and variants thereof. Fluorescent proteins are useful for the recognition of gene cassettes that have or have not been successfully inserted and/or replaced, as the case may be. Fluid cytometry and fluorescence-activated cell sorting are suitable for detection. Examples of fluorescent proteins are well-known in the art, including, but not limited to Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP) and far-red fluorescent protein (e.g. mKate, mKate2, mPlum, mRaspberry or E2-crimson. See, for example, U.S. Pat. No. 9,816,110. Reporter proteins are encoded by polynucleotides, and are referred to herein as “reporter genes” or “reporter protein genes.” Reporter genes and proteins can be referred to generically as first (1), second (2), third (3), fourth (4), fifth (5), sixth (6), seventh (7), eighth (8), ninth (9), tenth (10), etc., as is apparent from the context of usage. Reporters can be considered a type of marker. “Color” or “fluorescent,” in their various grammatical forms, also can be used the more specifically refer to a reporter protein or gene. Where multiple plasmids are used in a transfection, the plasmids can collectively comprise the same reporter genes or different reporter genes.

“Selectable” or “selection” marker proteins include proteins conferring certain traits, including but not limited to drug resistance or other selective advantages. Selection markers can give the cell receiving the selectable marker gene resistance towards a certain toxin, drug, antibiotic or other compound and permit the cell to produce protein and propagate in the presence of the toxin, drug, antibiotic or other compound, and are often referred to as “positive selectable markers.” Suitable examples of antibiotic resistance markers include, but are not limited to, proteins that impart resistance to various antibiotics, such as kanamycin, spectinomycin, neomycin, gentamycin (G418), ampicillin, tetracycline, chloramphenicol, puromycin, hygromycin, zeocin, and/or blasticidin. There are other selectable markers, often referred to as “negative selectable markers,” which cause a cell to stop propagating, stop protein production and/or are lethal to the cell in the presence of the negative selectable marker proteins. Thymidine kinase and certain fusion proteins can serve as negative selectable markers, including but not limited to GyrB-PKR. See White et al., Biotechniques, 50: 303-309 (May 2011). Selectable marker proteins and corresponding genes can be referred to generically as first (1), second (2), third (3), fourth (4), fifth (5), sixth (6), seventh (7), eighth (8), ninth (9), tenth (10), etc., as is apparent from the context of usage. Where multiple plasmids are used in a transfection, the plasmids can collectively comprise the same selection marker genes or different selection marker genes.

The term “target cells” includes any cells in which expression of a nucleotide of interest is desired or tolerated. Preferably, target cells exhibit a “target,” such as a receptor, ligand, protein, including glycoproteins, and/or antigen, including complexes thereof, on their surface that allows the cell to be targeted. Exemplary targets are calcium voltage-gated channel auxiliary subunit gamma 1 (CACNG1), asialoglycoprotein receptor 1 (ASGR1), Fel d 1, ENTPD3, PTPRA, CD20, CD63 and Her2. Additional targets include GAB A, transferrin receptor, CD3, CD34, integrin, adipophilin, AIM-2, ALDHIAI, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIAI, CTAGI, CTAG2, cyclin DI, Cyclin-AI, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, E6, E7, FGF5, FLT3-ITD, FN1, G250/MN/CAIX, GAGE-1,2,8, GAGE-3,4,5,6,7, GAS7, glypican-3, GnTV, gplOO/Pmel 17, GPNMB, HAUS3, Hepsin, HER-2/neu, HERV-K-MEL, HLA-A11, HLA-A2, HLA-DOB, hsp70-2, IDOI, IGF2B3, IL13Ralpha2, Intestinal carboxyl esterase, K-ras, Kallikrein 4, KIF20A, KK-LC-1, KKLC1, KM-HN-1, KMHN1 also known as CCDCl10, LAGE-1, LDLR-fucosyltransferase AS fusion protein, Lengsin, M-CSF, MAGE-A1, MAGE-A 10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Mel an-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-I/LAGE-2, OA1, OGT, OS-9, P polypeptide, p53, PAP, PAX5, PBF, pml-RARalpha fusion protein, polymorphic epithelial mucin (“PEM”), PPP1 R3B, PRAME, PRDX5, PSA, PSMA, PTPRK, RAB 38/N Y-MEL-1, RAGE-1, RBAF600, RGS5, RhoC, R F43, RU2AS, SAGE, secernin 1, SIRT2, SNRPDi, SOX10, Sp17, SPA17, SSX-2, SSX-4, STEAPI, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a, Kras, NY-ESOI, MAGE-A3, HPV E2, HPV E6, HPV E7, WT-1 antigen (in lymphoma and other solid tumors), ErbB receptors, Melan A [MARTI], gp 100, tyrosinase, TRP-1/gp 75, and TRP-2 (in melanoma); MAGE-1 and MAGE-3 (in bladder, head and neck, and non-small cell carcinoma); HPV EG and E7 proteins (in cervical cancer); Mucin [MUC-1](in breast, pancreas, colon, and prostate cancers); prostate-specific antigen [PSA](in prostate cancer); carcinoembryonic antigen [CEA](in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-I/LAGE-2, NA-88, GnTV, TRP2-INT2, E6, E7, human glucagon receptor (hGCGR) and. I human ectonucleoside triphosphate diphosphohydrolase 3 (hENTPD3). Other targets can be selected by the person skilled in the art. See WO 2019/006046.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are intended to serve as a way of referring individually to each separate value falling within the range, unless otherwise indicated herein, and each separate value is incorporated into the specification as if it were individually recited herein. All recited numbers or values expressly set forth and denominate all ranges created by the recited number or values.

### DESCRIPTION

### Challenges in Producing Covalently Surface Modified AAVs

The Spy:Tag:SpyCatcher system is used to engineer many covalently surface modified AAVs, and is used as an exemplar here.

In vitro conjugation reactions can occur when (1) AAV having SpyTag sequences in its capsid (“AAV-SpyTag”) and (2) SpyCatcher sequences bound to retargeting molecules (“SpyCatcher-Rm”), such as monoclonal antibodies (“SpyCatcher-Ab”) are mixed to allow formation of a joining isopeptide bond. Typically, the in vitro conjugation reaction is carried out after both the AAV-SpyTag and the SpyCatcher-Rm have been each separately and fully purified to remove contaminants, such as host cell proteins, which typically includes clarification, affinity capture chromatography, non-AAV viral inactivation, polishing chromatography, viral retentive filtration, and/or final formulation. However, following conjugation it is necessary to include an additional processing step to remove the unreacted SpyCatcher-Rm species to prevent them from being present in the final conjugated drug product. These challenges are met and overcome by the present inventions.

To achieve optimum conjugation (also referred to as “complete conjugation” depending on the purpose) of all available SpyTags on an AAV, such as AAV9, capsid surface with a SpyC-Rm species, reaction conditions should be optimized. Parameters affecting the speed and extent of the reaction are pH, temperature, reaction time, reactant concentration, and molar ratio of reactants. Suboptimal parameters can result in lower conjugation efficiency or degradative effects including aggregation or fragmentation of the AAV9-SpyT, SpyC-Rm, or conjugated AAV9-SpyT-SpyC-Rm species. Thus, there is a need to develop optimal reaction conditions as well as to be able to track the formation of conjugated species.

Another challenge is the variability in the AAV-SpyTag population in terms of the number of SpyTag peptides on the capsid. This is because AAV capsids are assembled stoichiometrically from 60 total subunits of viral proteins VP1, VP2 and VP3, and a subset of each of VP1, VP2 and VP3 proteins will have a SpyTag insertion. This leads to a distribution in the number of SpyTag-VP proteins present in each assembled capsid. The distribution of species can be either normal or non-normal. For example, if 1/10 of the VP proteins have SpyTag insertions, the assembled AAV molecules are expected to have a SpyTag on an estimated 60/10=6 VP proteins on average, but the actual number of SpyTags expressed can range from 0 to 20 or more. See FIG. 1A. This variability is compounded by the variability of the conjugation reaction itself, as all or a subset of the SpyTags can be conjugated to SpyCatcher-Rm species, depending on the conjugation reaction conditions (See, for example, FIG. 1A).

Thus, there are multiple species in the post-processing conjugation reaction mixture with significantly different size and charge characteristics which complicates purification: AAV-SpyTag is about 3700 kDa and each SpyCatcher-Ab, for example, is about 160 kDa (when the Ab is a monoclonal antibody), so a molecule with ten surface conjugations would be of size of about 5300 kDa. FIG. 1A schematically depicts the species heterogeneity involved in in-vitro conjugation reactions between rAAV9-SpyT and SpyC-Ab species. This figure depicts the heterogeneity at the level of the AAV9-SpyT capsid, depicting the “average” species with 6 SpyTags, as well as species with lower and higher number of SpyTags expressed on the surface. The figure also depicts the heterogeneity at the level of the conjugated AAV9-SpyT-SpyC-ASGR1 mAb species, where the number of SpyC-ASGR1 mAb species during the in-vitro conjugation reaction were not in significant molar excess compared to the number of AAV9-SpyT species, which results in incomplete conjugation (showing species with average, low and high levels of conjugation in the middle row). The figure further depicts the heterogeneity at the level of the conjugated AAV9-SpyT-SpyC-ASGR1 mAb species, where the number of SpyC-ASGR1 mAb species during the in-vitro conjugation reaction were in significant molar excess compared to the number of AAV9-SpyT species, resulting in optimal conjugation (showing species with average, low and high levels of conjugation in the bottom row). Differences in size and charge are a result of different amounts of antibody conjugation.

FIG. 1B is a graph that depicts retention time (minutes) of high molecular weight (HMV) variants comprising conjugated AAV9-SpyT-SpyC-Ab species, conjugated AAV9-SpyT-SpyC-Ab, unconjugated AAV9-SpyT and unconjugated SpyC-mAb. Size exclusion chromatography (SEC) was employed. Table 1 below provides percentages of HMW fractions, conjugated fractions and unconjugated fractions for various ratios of AAV to antibody (Fabs also can be used, for example). The data shows that 1:300 ratio of AAV capsid to antibody performs best. Notably, the ratios of 1:6 and 1:15 provided the greatest percentages of HWM variants.

TABLE 1

| Molar ratio of | | | |
|---|---|---|---|
| AAV: Antibody | HMV (%) | Conjugated (%) | Unconjugated (%) |
| 1:0 | 0 | 0 | 100 |
| 1:1 | 4.5 | 15.7 | 79.8 |
| 1:6 | 19.4 | 51.1 | 29.6 |
| 1:15 | 25.2 | 52.4 | 22.4 |
| 1:30 | 4.6 | 81.6 | 13.9 |
| 1:300 | 3.6 | 88.2 | 8.2 |

Thus, there is a need to establish new approaches suitable for removal of unreacted SpyCatcher-Rm species. Challenges include making the methods applicable to removal of different SpyCatcher-Rm species (including but not limited to mAb, Fab, ScFv, bispecific mAb, trispecific mAb, Fc-fusion proteins and other Ab-related formats, such as half antibodies and other heavy chain and/or light chain combinations), which differ greatly in size, charge, isoelectric point (pI) and structure. A challenge with charge-based methods is that molecules with a high level of surface conjugation typically are more “Rm-like” in terms of their surface charge, and thus tend to co-elute with the SpyCatcher-Rm species in many different types of ion-exchange chromatography. Increasing similarity between the surface charge characteristics of conjugated AAV9-SpyT-SpyC-Rm to the unconjugated SpyC-Rm species is expected with increasing number of conjugations. This presents a significant purification challenge, as applying fractionation criteria to limit the presence of unreacted SpyCatcher-Rm in the elution pool would tend to lose the most heavily conjugated species as well, in addition to reducing the product yield. Thus, anion exchange chromatography methods, which are traditionally useful for AAV separations cannot be applied to this separation in a straightforward manner. Optimization of the ion-exchange process on different modalities (such as cation exchange or multimodal separations) and with different pH and salt conditions is useful to achieve partitioning between the free SpyC-Rm species and the conjugated AAV9-SpyT-SpyC-Rm species without significant co-elution.

Size-based methods would seem to be an option, particularly due to the apparently large difference in size between the conjugated AAV species (>3700 kDa) compared to the Ab species (<200 kDa). However, despite this difference, the tightly packed structure of AAV makes it very small compared to other viruses, and it has an effective radius of about 15 to 25 nm, while the SpyCatcher-Rm species are around 10-20 nm in radius. Thus, the difference in actual size is not very significant, leading to ineffectiveness of membrane-based separations on 100, 300 or 500 kDa membranes. Though large-scale size exclusion chromatography is a potential option, the method is difficult to scale due to very long column lengths and long processing times required. Other size-based chromatography resins developed for large-scale purification of viruses include CaptoCore400 and CaptoCore700 resins, but these cannot be directly used in AAV separations due to the small size of AAV (about 15-25 nm) compared to other viruses (such as Adenoviruses or Retroviruses which are greater than 60 nm). The small size of AAV makes it enter the pores of the resins, after which it can only be recovered in denatured form in the strip.

According to the inventions, buffers comprising at least one buffering agent, for example Bis-Tris-Propane, Bis-Tris, Tris, Glycine, Bicine, Tricine, Acetate, Borate, Citrate, Carbonate, Phosphate, Formate, Sulfate, Succinic acid, Sulfonic acid and variants thereof (for example, MES, PIPES, HEPES, CHES, CAPS, MMS, PBMS), Diethanolamine, or Imidazole can be used. Buffers also may comprise one or more organic or inorganic salts, such as NaCl, KCl, MgCl₂, CaCl₂), NH₄Cl, Na₂SO₄, CaSO4, K₂SO₄, MgSO₄, (NH₄)₂SO₄, sodium citrate, and tetramethylammonium chloride (TMAC). The buffer may also comprise additives such as antibiotics and bacteriostatics (for example, sodium azide or AEBSF), protease inhibitors (for example E64), detergents and chaotropes (for example poloxamer, CHAPS, SDS, Triton, Tween, Urea), saturating agents (for example, bovine serum albumin), organic solvents (for example ethanol, isopropanol, acetonitrile), and other additives or excipients including chelatants, stabilizers, reducers (for example, DTT, TCEP, EDTA, Glycerol, Sucrose), and amino acids (for example, Histidine or Arginine).

The inventions are amenable to the use of anion exchange (AEX) media, which include monoliths, resins and membranes. AEX media includes AEX monoliths (for example, CIM QA, and CIM DEAE), AEX resins (for example, Capto Q, Capto Q ImpRes, CAPTO DEAE, POROS HQ, POROS XQ, POROS PI, POROS D, Fractogel EMD TMAE, Fractogel EMD DEAE, Nuvia Q, Nuvia HP-Q) and AEX membranes (for example, Sartobind STIC PA, Sartobind Q, Natrix Q).

The inventions also are amenable to the use of cation exchange (CEX) media, which include monoliths, resins and membranes. CEX media includes monoliths (for example CIM SO3), CEX resins (for example Capto S, Capto S ImpRes, Capto S ImpAct, Capto SP, POROS HS, POROS XS, CM Sepharose, Nuvia S, Nuvia HR-S), and CEX membranes (for example, Sartobind S, Natrix CH).

The inventions are amenable to the use of multimodal media, which includes monoliths and resins. Multimodal monoliths (for example PRIMA T, PRIMA S), multimodal resins (for example Capto Adhere, Capto MMC, CaptoCore400, Capto Adhere ImpRes, Capto MMC ImpRes, HEA HyperCel, PPA HyperCel, CMM HyperCel, MEP HyperCel, HA Ultrogel).

### Specific Binding Pairs

The inventions advantageously employ one or more “specific binding pairs,” also referred to as “protein:protein binding pairs.” An example is the SpyTag:SpyCatcher system. The SpyTag:SpyCatcher system was developed using the Streptococcus pyogenes second immunoglobulin-like collagen adhesion domain (CnaB2) from the fibronectin binding protein FbaB. An isopeptide bond can be formed spontaneously between the SpyTag protein and the SpyCatcher protein. The SpyCatcher peptide weighs about 15 kD. However, the SpyTag protein is only 13 amino acids long. The small size of the SpyTag protein makes it amenable for insertion into the AAV genome, which has a total packing capacity of only about 4.7 kilobases. These systems, such as SpyTag:SpyCatcher, allow a retargeting molecule to be bound to an AAV. See Examples 34, 35 and 36.

Other systems to facilitate retargeting include the SpyTag:SpyCatcher system is described in U.S. Pat. No. 9,547,003 and Zakeri et al. (2012) PNAS 109:E690-E697, is derived from the CnaB2 domain of the Streptococcus pyogenes fibronecting-binding protein FbaB. See WO 2019/006046.

SpyTag002:SpyCatcher002 system is described in Keeble et al (2017) Angew. Chem. Int. Ed. Engl 56:16521-25. See WO 2019/006046.

SpyTag003:Spy Catcher003 also has been created. Spy Tag002:SpyCatcher002 and SpyTag003:SpyCatcher003 are different iterations of Spy Tag:Spy Catcher.

The SnoopTag:SnoopCatcher system is described in Veggiani (2016) PNAS 113:1202-07. The D4 Ig-like domain of RrgA, an adhesion from Streptococcus pneumoniae, was split to form SnoopTag. Incubation of SnoopTag and SnoopCatcher results in a spontaneous isopeptide bond that is specific between the complementary proteins. Veggiani (2016), cited above. See WO 2019/006046.

The Isopeptag:Pilin-C specific binding pair was derived from the major pilin protein Spy0128 from Streptococcus pyogenes. (Zakeir and Howarth (2010) J. Am. Chem. Soc. 132:4526-27). See WO 2019/006046.

Other systems to facilitate retargeting can be based upon the splitting and engineering of RegA domain 4. These have led to SnoopTagJr:SnoopCatcher, DogTag:DogCatcher and Snoop Ligase. Other systems include Isopeptag:Pilin-N, SdyTg:SdyCatcher, Jo:In, 3kptTag: 3kptCatcher, 4oq1Taq/4oq1 Catcher, NGTag/Catcher, Rumtrunk/Mooncake, GalacTag, Cpe, Ececo, Corio and all others based upon isopeptide binding pairs or other covalent bonds.

Systems based upon truncated and/or other variations/derivatives have been developed to lessen the possibility of encountering neutralizing antibodies when the covalently surface modified AAV is administered to a subject. For example, the use of KTag (SEQ ID NO: 85) with SpyTag (SEQ ID NOS: 54 and 86) can be employed. The bonding between KTag and SpyTag can be undertaken using SpyLigase (SEQ ID NO: 61).

KTag is made by expressing the β-strand of CnaB2 containing a reactive Lys, which was separately expressed. (10 amino acids). SpyLigase (11 kDa) is based on SpyCatcher and made by removing amino acids from the β-strand that has the reactive Lys and using a circular permutation to replace amino acids from the C-terminus of CnaB2 with a Gly/Ser linker, which is followed by N-terminal CnaB2 amino acids. See Fierer et al., Proc. Nat'l Acad. Sci. 131577611.

### Retargeting Molecules

Retargeting molecules (Rm) include Fc-containing proteins, such as antibodies and Fc-fusion proteins (including receptor Fc fusion proteins such as trap proteins). Fragments and derivatives of antibodies and Fc-fusion proteins also can be used as retargeting molecules.

All major antibody classes, namely IgG, IgA, IgM, IgD and IgE, can be used as retargeting molecules. IgG is a preferred class, and includes subclasses IgG1 (including IgG1λ and IgG1κ), IgG2, IgG3, and IgG4. Further antibody types include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody.

Retargeting molecules bind to targets, which are antigens, receptors and/or ligands found on the surface of a target cell. Exemplary targets are calcium voltage-gated channel auxiliary subunit gamma 1 (CACNG1), asialoglycoprotein receptor 1 (ASGR1), Fel d 1, ENTPD3, PTPRA, CD20, CD63 and Her2. Additional targets include GAB A, transferrin. CD3, CD34, integrin, adipophilin, AIM-2, ALDHIAI, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIAI, CTAGI, CTAG2, cyclin DI, Cyclin-AI, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, E6, E7, FGF5, FLT3-ITD, FN1, G250/MN/CAIX, GAGE-1,2,8, GAGE-3,4,5,6,7, GAS7, glypican-3, GnTV, gplOO/Pmel 17, GPNMB, HAUS3, Hepsin, HER-2/neu, HERV-K-MEL, HLA-A11, HLA-A2, HLA-DOB, hsp70-2, IDOI, IGF2B3, IL13Ralpha2, Intestinal carboxyl esterase, K-ras, Kallikrein 4, KIF20A, KK-LC-1, KKLC1, KM-HN-1, KMHN1 also known as CCDCl10, LAGE-1, LDLR-fucosyltransferase AS fusion protein, Lengsin, M-CSF, MAGE-A1, MAGE-A 10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Mel an-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-I/LAGE-2, OA1, OGT, OS-9, P polypeptide, p53, PAP, PAX5, PBF, pml-RARalpha fusion protein, polymorphic epithelial mucin (“PEM”), PPP1 R3B, PRAME, PRDX5, PSA, PSMA, PTPRK, RAB 38/N Y-MEL-1, RAGE-1, RBAF600, RGS5, RhoC, R F43, RU2AS, SAGE, secernin 1, SIRT2, SNRPD1, SOX10, Spl7, SPA17, SSX-2, SSX-4, STEAPI, survivin, SYT-SSX1 or -SSX2 fusion protein, TAG-1, TAG-2, Telomerase, TGF-betaRll, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a, Kras, NY-ESOI, MAGE-A3, HPV E2, HPV E6, HPV E7, WT-1 antigen (in lymphoma and other solid tumors), ErbB receptors, Melan A [MARTI], gp 100, tyrosinase, TRP-1/gp 75, and TRP-2 (in melanoma); MAGE-1 and MAGE-3 (in bladder, head and neck, and non-small cell carcinoma); HPV EG and E7 proteins (in cervical cancer); Mucin [MUC-1](in breast, pancreas, colon, and prostate cancers); prostate-specific antigen [PSA](in prostate cancer); carcinoembryonic antigen [CEA](in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-I/LAGE-2, NA-88, GnTV, TRP2-INT2, E6, E7, human glucagon receptor (hGCGR) and. I human ectonucleoside triphosphate diphosphohydrolase 3 (hENTPD3). Other targets can be selected by the person skilled in the art. See WO 2019/006046.

### Detargeting Mutations

The inventions further provide mutations in the VP1 Protein to lower the AAV preferential transduction of the liver. For AAV9, preferred The mutations include N272A and W503A substitutions, where alanine replaces both asparagine at position 272 of VP1 and tryptophan at position 503 of VP1. One or both of the mutations can be undertaken in the VP1 protein. Optionally, other amino acids, such as glutamic acid, serine or others, can be used instead of alanine for substitution. Other detargeting mutation sites include, but are not limited to, N470, D271, and Y446.

The inventions provide exemplary mutations for use with AAVs as follows, which for each serotype can be utilized singly or in combinations
AAV1-N500E;
AAV2-R585A and R588A;
AAV5-T571 S;
AAV6-N500E, K531 A and K531E;
AAV9-N272A, W503A, T568P, R590L, N498Y, 8602F, 8414N, G453D, K557E, T5821, and combinations include, but are not limited to, T568P+4590L, N498Y+L602F, S414N+G453D+K557E+T5821.

These and others are set forth in the chart below:

| AAV | Insertion | |
|---|---|---|
| serotype | Sites | Exemplary Mutations |
| AAV1 | 447, 472, 473, | N477, S472, V473, N500, N502, W503, |
| | 500, 502, 503 | N500E, S268, D270, N271, Y445, G470, |
| | 587, 589 | K531, and K531, K531A and K531E. |
| AAV2 | 1, 34, 138, | R484, R485, R487, R532, R585, R585A, |
| | 139, 161, | R588, T589, R588A, K532, |
| | 261, 266, | R484A, R487A, R487G, K532A, K532D, |
| | 381, 447, | R585A, R585S, R585Q, R588A, R588T, |
| | 448, 453, | G453, N587, E499, |
| | 459, 471, | Q464V, A467P, D469N, 1470M, R471A, |
| | 484, 487, | D472V, S474G, Y500F, S501A, R432A, |
| | 520, 532, | L510A, N511R, R404A, R295A, R294A, |
| | 534, 570, | Q297A, R298A, R294A/R298A, T331A, |
| | 573, 584, | K692A, W694A, P696A, R389A, Y441A, |
| | 585, 587, | L510A N511R, P602A, R432A, P622A, |
| | 588, 591, | D625A, V221A/S224A, H255/K258A, |
| | 657, 664, | Y413A, F415A, D416A, M402A, E415A, |
| | 713, 716 | H229/D231A, K321A/E322A, N334W, |
| | | V221W, V221C, V221Y, L336C. |
| AAV3 | 585, 594 | R594, G594, N588A, N588S, A590Q, |
| | | S384A, T717V. |
| AAV4 | 492, 499, | K492, S499, K503, T504, M523, M524, |
| | 503, 504, | G581, N583, Q583, Q585, N585, S587. |
| | 523, 524, | |
| | 581, 583, | |
| | 584, 585, 587 | |
| AAV5 | 531, 569, | K531A, K531E, T571S, M569, A570, |
| | 570, 571, | T571, V580, A581, V582, G583, T584, |
| | 575, 580, | Y585, N586, L587, A592, G594, D595, |
| | 581, 582, | V596, H597, A598, T571, T571S. |
| | 583, 584, | |
| | 585, 586, | |
| | 587, 592, | |
| | 594, 595, | |
| | 596, 597, 598 | |
| AAV6 | 447, 459, | K459, K493, N500E, K531, K531A, |
| | 472, 473, | K531E, R576, N500, Q585. |
| | 493, 500, | |
| | 502, 503, | |
| | 531, 576 | |
| AAV7 | | S157, N254, N460, A2, K61, T252 |
| AAV8 | | Y6, A2, N521, Q588, T591, N590. |
| AAV9 | 270, 271, | D270, D271, N271, N272, N272A, Y445, |
| | 272, 445, | Y446, N470, G470, W503, W503A, |
| | 446, 453, | S469, A742, V473, P504 and Q590, |
| | 470, 503, | K532A, K532D, R484A, R487A, R585A, |
| | 587, 589 | R585S, R585Q, R487G, R588A, R588T, |
| | | G453, A587, A589, E500, S586, A589. |
| AAV10 | | A589T. |
| AAV13 | 528, 532 | K528, E531. |
| Avian | 444, 580 | G444, K580. |
| AAV | | |
| Sea lion | 429, 430, | |
| AAV | 431, 432, | |
| | 433, 434, | |
| | 436, 437, 565 | |
| Bearded | 573, 436 | G436, T573. |
| Dragon | | |
| AAV | | |
| AAV-DJ | | K137R, T251A, S503A. |
| rAAVrh.10 | | K259, K333, S453, S501, S559, Q589, |
| | | N590, A592, S671, T674, Y708, T719, |
| | | AS453, K259L, K333V, AS453, S501A, |
| | | S559A, Q589N, N590S, A592Q, S671A, |
| | | T674V, Y708A, T719V. |
| AAVrh10 | | A2, Y90, S149, S157, T252, N306, T332, |
| (AAV10) | | K333, S449, K652, K709. |

Other mutations and detargeting approaches, such as variable loop swaps, are disclosed in publications or otherwise available See, for example, Shen et al., Molecular Therapy 15: 1955-62 (2007). These technologies can be used according to the inventions. Detargeting mutations are optional.

### Host Cells

The present inventions are amenable for production in mammalian cell culture. Exemplary cell lines are CHO, Per.C6 cells, Sp2/0 cells, HeLa and HEK293 cells. CHO cells include, but are not limited to, CHO-ori, CHO-K₁, CHO-s, CHO-DHB11, CHO-DXB11, CHO-K1 SV, and mutants and variants thereof. HEK293 cells include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof. Adherent HEK 293 cells also can be used for production of covalently surface modified AAV according to the inventions. HEK 293 suspension cultured cells were derived from HEK 293 adherent cells. See Maim et al., Scientific Reports 10: 18996 (2020). Other suitable cells include, but are not limited to BHK (baby hamster kidney) cells, HeLa cells and Human Amniotic cells, such as Human Amniotic Epithelial cells. Other cell types for production include insect cells, such as Sf9.

### Production

As described above, FIG. 2 schematically depicts and describes quad transfection systems and methods for producing covalently surface modified AAV in a eukaryotic cell, such as HEK 293F. As described in FIG. 2, the quad transfection system utilizes four plasmids (schematically depicted) to produce AAV comprising SpyTag sequences in the capsids. The plasmids are as follows:
(i) pGOI—a plasmid comprising a GOI (for example, a transgene) flanked by two AAV inverted terminal repeats (ITRs), and optionally a selection marker gene, such as an antibiotic resistance gene (for example, ampicillin resistance);
(ii) pRC—a plasmid comprising AAV (here AAV9) rep and cap genes, and optionally a selection marker gene, such as an antibiotic resistance gene (for example, ampicillin resistance);
(iii) pRC-SpyT—a plasmid as above in (ii) and further comprising a polynucleotide sequence encoding a 13 amino acid Spy Tag peptide (a first member of a specific binding pair), and optionally a selection marker gene, such as an antibiotic resistance gene (for example, ampicillin resistance). This is an exemplary plasmid pRC-first member (pRC-FM);
and
(iv) pHELP—a plasmid comprising adenovirus helper genes E4 and E2, and VA RNA, (exemplary helper genes) and a optionally selection marker gene, such as an antibiotic resistance gene (for example, ampicillin resistance). The following chart provides a brief summation of this example:

| Plasmid type | Specifics |
|---|---|
| pGOI | Any gene(s) of interest flanked by |
| | AAV ITRs. See, e.g., Example 32. |
| pRC | For example, comprises AAV 9 rep |
| | and cap genes. See, e.g., Example |
| | 32. |
| pRC-FM | pRC-Spy Tag. Provides VP1, VP2 |
| | and VP3 proteins fused to Spy Tag |
| | protein. See, e.g., Examples 35 and |
| | 36. |
| pHELP | Comprises one or more adenovirus |
| | helper genes. See, e.g., Example |
| | 33. |

Promoters that are functional in eukaryotic cells, such as AAV P5, CMV, lac, CAG, CAGG, and SV40 promoters, are provided where needed to initiate transcription, but are not shown. Additionally, internal ribosome entry sites (IRESs), recombinase recognition sites (RRSs), enhancers and operators optionally also can be included, but are not shown. For constructing plasmids having large polynucleotides, such as those encoding antibody chains, intronic selection can be employed, preferably with a second selection marker gene that is different from the first selection marker gene.

FIG. 3 schematically depicts at the top a pRC-SpyTag plasmid comprising the rep and cap genes and the p40 promoter, and at the bottom schematically shows the insertion of the SpyTag 13 amino acid peptide sequence to form a fusion protein comprising SpyTag and Cap peptide sequences, thereby resulting in mutant VP1, VP2 and VP3 proteins fused to the SpyTag peptide sequence. The AAV still possesses the 5:5:50 stoichiometry of the VP1, 2 and 3 proteins.

Reaction time for the conjugation should be an incubation of at least 0.5, 1, 1.5, 2, 2.5, 3, 3.5 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 2 9.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 54.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72 or more hours (4 to 72 hours or more). Reaction temperature should be about 4° C. to about 40° C., preferably room temperature (19° C. to 25° C., preferably about 22° C.). Molar ratio of the reactants, namely (AAV-first member) to (retargeting molecule-second cognate member) should be 1:20, 1:25 or preferably greater differences. Preferably, the molar ratio should be 1:30 to 1:1000 (includes all ratios there between) to maximize conjugation efficiency. Ratios of 1:30 to 1:300 (includes all ratios there between) are more preferred. See Example 12. Other parameters are set forth below in Table 2:

TABLE 2

| | Preferred | Acceptable | Parameters to |
|---|---|---|---|
| Variable | parameters | Parameters | avoid |
| pH | 5.0-6.5 | 3.0-9.5 | Greater than 9.5 |
| Temperature | 22° C. | 4-40° C. | Greater 40° C. |
| Molar ratio of | 1:300 | 1:20-1:1000 | Differences less |
| AAV-first | | | than 1:20 |
| member (e.g., | | | |
| AV-SpyT) to | | | |
| RM-second | | | |
| cognate member | | | |
| (e.g., SpyC-Ab) | | | |
| Incubation Time | 24-72 hours | 1 hour to 7 days | Less than 0.5 hours |
| Concentration | 1 × 1013 to | 1 × 1010 to | Less than |
| of AAV9-first | 5 × 10¹³ | 2 × 10¹⁴ | 1 × 10¹⁰ |
| member | cp/mL | cp/mL | cp/mL |
| Conductivity of | 20 mS/cm | 2-100 mS/cm | Less than 2 mS/cm |
| Reaction Mixture | | | |
| Stirring RPM | 0 | 0-600 RPM | |

Significant molar excess of SpyCatcher-Rm is typically beneficial to ensure optimum conjugation of SpyTag-AAV species, where the molar excess can range from 20 to 1000-fold, preferably 30 to 300 fold, and most preferably 300 fold. The inventions provide for complete conjugation of first members of a specific binding pair are bound to second cognate members of the specific binding pair. The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. Optimal conjugation (also known as complete conjugation based on the purpose) is typically desired by the skilled person and preferably will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 90%, 70% to 90%, 75% to 90%, 75% to 85%, 75% to 80%, or 80% to 85%, and any ranges made by combining these ranges, such as 65% to 85%, for example. More preferably, the range is 65% to 95%.

The percentages of conjugation can be ascertained by a variety of separation techniques, including size exclusion chromatography (SEC) or asymmetrical flow field-flow fractionation (A4F). SEC and A4F would be combined with a detector, such as UV, fluorescence or a multi-angle light scattering (MALS) detector. Due to the molar excess of SpyCatcher-Rm, a significant amount of SpyCatcher-Rm will need to be removed following the conjugation reaction.

When starting the conjugation reaction, the AAV-SpyT (FM) should be added to the vessel prior to the SpyC (SCM)-RM. AAV-SpyT should be added first but not in ratio excess due to low concentration of AAV to Ab.

In order to drive the conjugation reaction to completion, it can be beneficial to provide the SpyC-RM in molar excess of the AAV-SpyT, where the excess can be up to 1000-fold. In such cases, once the conjugation reaction is completed, there will remain a significant amount of excess of unconjugated SpyCatcher-Rm following conjugation. This can be due to the SpyCatcher-Rm being added in molar excess to the number of available SpyTag binding sites on the AAV and/or due to incomplete reactions.

Additives can be included in the conjugation reaction to modify conditions and enhance conjugation efficiency. Additives include, but are not limited to, dimethylsulfoxide (DMSO), L-glutamic acid monosodium salt (L-glutamic acid), arginine, sorbitol, urea, isopropyl alcohol, (IPA) and sodium thiocyanate (NaSCN).

The inventions are further described by the following examples, which do not limit the inventions in any manner. The order of performance of the below examples can be altered or combined as determined by the person of skill in the art in view of the teachings and data contained herein.

The AAV serotype sequences and component sequence types used according to the inventions used in the Description, Summary, Examples and Figures are representative and not limiting. Any AAV serotype sequence and component sequence can be used according to the inventions. These include but are not limited to sequences for promoters, markers, specific binding pairs, helper proteins, AAV sequences (Cap, Rep and ITRs), retargeting molecules, detargeting mutations, IRESs, RRSs, introns, operators, enhancers and polydenylation signals.

### Example 1—In Vitro Conjugation Using SpyTag:SpyCatcher

In vitro conjugation can use a four plasmid transfection system (quad transfection) for cells to produce for rAAV comprising SpyTag. For example, the plasmids could be: (1) plasmid containing a GOI flanked by AAV inverted terminal repeats (pGOI); (2) plasmid containing AAV rep and cap genes (pRC); (3) plasmid containing AAV rep gene and cap gene with a polynucleotide encoding SpyTag peptide inserted in frame into the cap gene (pRC-SpyT); and (4) a plasmid comprising helper polynucleotide sequences, such as adenovirus helper genes, in a first bioreactor (pHELP). See FIG. 2. Helper genes also can be obtained from herpesviruses and papilloma viruses.

For retargeting molecules, such as antibodies, cells (for example, HEK 293) can be transfected with two plasmids, such as: plasmid encoding an antibody heavy chain sequence in frame with a SpyCatcher sequence and (2) a plasmid encoding an antibody light chain sequence in a second bioreactor. The cells will produce an antibody bound to SpyCatcher.

In addition to the use of monospecific monoclonal antibodies as retargeting molecules, antibody derivatives and fragments can also be employed as retargeting molecules. Antibody fragments and derivatives include, but are not limited to, Fab, ScFv-Fc, dAB-Fc, half antibodies bispecifics, tri-specifics, IgG-ScFv, IgG-dab, ScFV-Fc-ScFV and other combinations of heavy and/or light chains. Fc-fusion proteins, such as trap proteins, and well as mini-trap proteins also can be used a retargeting molecules.

Eukaryotic cells, preferably mammalian cells, such as human and rodent cells, can be used to produce the rAAV and the retargeting molecules, such as antibodies. The cell types can be the same or different. For example, rAAV can be produced in human cells, such as HEK 293 cells and retargeting molecules in rodent cells, such as CHO cells, and conversely rAAV can be produced in rodent cells (for example, CHO cells) and retargeting molecules in human cells (for example, HEK 293 cells). In other approaches, both rAAV and retargeting molecules can be both produced in human cells or both produced in rodent cells (for example, CHO cells), depending on the preference of the person skilled in the art. Insect cells such as Sf9 can alternatively be used for producing the rAAV, retargeting molecules, or both.

The rAAV and retargeting antibody produced as described above can each be purified by one or more of depth filtration, tangential flow filtration (TFF), affinity capture polishing, and viral retentive filtration. TFF is a generic term and includes, but is not limited to, ultrafiltration/diafiltration (UF/DF) and newer approaches such as single-pass tangential flow filtration (SPTFF).

Conventional TFF can be employed, which uses a batch approach and repeated cycling through the membrane to create a permeate and a retentate. Alternatively, single-pass TFF can be employed to allow for a continuous process. According to the inventions, TFF can advantageously employ permeate pumps to reduce TMP buildup and flux decline. Although not to be bound by any hypothesis or theory, it is believed that detergents, such as Tweens, build up on the retentate side of a TFF membrane and cause TMP buildup and flux decline. Single-Pass TFF units are available from Pall/Cytiva, Repligen and Millipore. Thereafter, the conjugation reaction can be undertaken between the rAAV and the retargeting antibody.

FIG. 1A depicts the species heterogeneity involved in in-vitro conjugation reactions between AAV9 comprising a SpyTag peptide (rAAV9-SpyT) and SpyCatcher fused to a monoclonal antibody (SpyC-mAb species). This figure depicts the heterogeneity at the level of the AAV9-SpyT capsid, depicting the “average” AAV species with 6 SpyTags, as well as AAV species with lower (3) and higher number (10) of SpyTags expressed on the capsid surface. Heterogeneity at the level of the conjugated AAV9-SpyT-SpyC-ASGR1 monoclonal antibody Ab species (ASGR1 mAb) also is shown where the number of SpyC-ASGR1 mAb species during the in-vitro conjugation reaction were not in a significant molar excess compared to the number of AAV9-SpyT species (1:1 to 1:15 in terms of AAV-SpyT to SpyC-mAb). These 1:1 to 1:15 ratios resulted in incomplete conjugation (species with average, low and high levels of conjugation are depicted). Heterogeneity at the conjugated level also is shown where the number of SpyC-ASGR1 mAb species during the in-vitro conjugation reaction were in significant molar excess compared to the number of AAV9-SpyT species (1:30 to 1:300 in terms of AAV-SpyT to SpyC-mAb). These 1:30 to 1:300 ratios resulted in optimum (complete) conjugation (showing species with average, low and high levels of conjugation).

Optimal (complete) conjugation occurs when most available first members of a specific binding pair are bound to second cognate members of the specific binding pair. For example, FIG. 1A (bottom row) schematically depicts instances of all and nearly all SpyTags bound by SpyCatcher on covalently surface modified AAVs.

The inventions provide for Optimal conjugation (also referred to as complete conjugation) of first members of a specific binding pair are bound to second cognate members of the specific binding pair. Preferably, optimal conjugation (also known as complete conjugation based on the purpose of the skilled person) is typically desired and preferably will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 90%, 70% to 90%, 75% to 90%, 75% to 85%, 75% to 80%, or 80% to 85%, and any ranges made by combining these ranges, such as 65% to 85%, for example. More preferably, the range is 65% to 95%.

The percentages of conjugation can be ascertained by a variety of separation techniques, including size exclusion chromatography (SEC) or asymmetrical flow field-flow fractionation (A4F). SEC and A4F would be combined with a detector, such as UV, fluorescence, or a multi-angle light scattering (MALS) detector.

### Example 2—Separating a Covalently Surface Modified Adeno-Associated Virus Species from Unconjugated Antibody Species Using Size Exclusion Chromatography

FIG. 4 depicts size exclusion chromatograms (SEC) of in vitro conjugation reaction mixtures of rAAV9 to antibody in ratios of 1:1, 1:6, 1:18, 1:30 and 1:300 at pH 8.0 and using rAAV concentration of 5×10¹⁰ to 1×10¹² capsids (cp) per milliliter on a size exclusion chromatography column with pore size of 1000 Å. The shift in retention time of the AAV peak is highlighted with an arrow showing shifting of the peak maximum to the left indicating conjugation reaction completion. The x-axis is minutes for elution and the y-axis is emission units (EU) for fluorescence.

FIG. 5A depicts size exclusion chromatograms of in-vitro conjugation reaction mixtures of rAAV9 to antibody in ratios of 1:0, 1:1, 1:6, 1:15, 1:30 and 1:300 at pH 5.0, 6.5, 8.0 and 8.5 and using rAAV concentration of 2.3×10¹³ cp/mL on a size exclusion chromatography column with pore size of 450 Å. Reaction conditions included brief mixing followed by overnight incubation, resulting in maximum conjugation as shown by the almost complete disappearance of the AAV-SpyT peak and formation of a new Conjugated AAV peak at ratios of 1:30 and 1:300. FIG. 5B is a preparative scale SEC on a 100 ml SEPAX SRT 500A column to remove unconjugated antibody.

FIGS. 6A and 6B depict size exclusion chromatograms of in-vitro conjugation reaction mixtures of rAAV9 to antibody in ratios of 1:0, 1:1, 1:6, 1:10, 1:20 (FIG. 6A), and 1:30 at pH 5.0, 6.5, 8.0, 8.5, 9.0, 9.5 and 10.0 (FIG. 6B), and using rAAV concentration of 2.3×10¹³ cp/mL on a 450 Å size exclusion chromatography column. Reaction conditions included brief mixing followed by 1 hour incubation prior to freezing, resulting in incomplete conjugation shown by the presence of AAV-SpyT peak at all molar ratios.

### Example 3—Separating a Covalently Surface Modified Adeno-Associated Virus Species from Unconjugated Antibody Species Using Anion Exchange or Size Exclusion Chromatography

FIG. 7A depicts exemplary elution profiles for AAV9 and AAV9-SpyT for empty, partially filled and full capsids, and serves as a model for other AAVs. There will exist empty and partially-filled capsid populations can have isoelectric points (pI) that are above full capsids and other empty and partially-filled capsid populations will have isoelectric points that are below full capsids. FIG. 7B is a graph depicting separation empty, partially filled and full AAV9-SpyT capsids using a complex hybrid gradient. The column used to obtain the data in FIG. 7B was a CIM QA monolith loaded with 5×10¹³ to 5×10¹⁴ capsids (cp/)ml. The buffer was 20 mM Bis-Tris-Propane, 0.001% P188, pH 9.6 and 1 M NaCl. FIG. 7C depicts data from a two-pass anion exchange chromatogram for a 500 L purification of AAV-SpyTag. First pass and second pass microsteps. This process achieved a recovery of 96% full capsids. Partially-filled capsids were only 1% of total and empty capsids were only 3% of total. FIGS. 7D to 7F depict data from an experiment using anion exchange to separate a covalently surface modified adeno-associated virus species from unconjugated antibody species. A CIM QA 1 mL monolith was used as the anion exchange column, and the column was loaded with an in-vitro conjugation reaction mixture of AAV9-SpyT and SpyC-ASGR1 mAb mixed in a 1:30 molar ratio. In FIG. 7D, the AAV9-SpyT capsids were 10% full, whereas in FIG. 7D the AAV9-SpyT capsids were 70% full. FIG. 7F depicts the size exclusion chromatograms combined with multi-angle light scattering (MALS) data of six elution fractions collected from FIG. 7E, showing the increasing conjugation level, and increasing mass distribution of the later eluting fractions.

FIG. 7G is a graph depicting the retention time of conjugated AAV9 using SpyTag-SpyCatcher with an antibody, unconjugated AAV-SpyTag and unconjugated SpyCatcher-antibody. Conjugation reaction was studied by tracking percent conjugation over time using size exclusion chromatography according to the following:

$$\frac{Peak area of conjugated AAV}{Peak area of conjugated plus unconjugated AAV} \times 100$$

FIG. 7H is a graph depicting the effect of AAV capsid concentration at 1×10¹² cp/ml, 1×10¹³ cp/ml and 3×10¹³ cp/ml on percent conjugation. Higher capsid concentration resulted in a greater percentage of conjugation.

FIG. 7I is a graph depicting the effect of incubation temperature at 4° C., 20° C. and 37° C. on percent conjugation. Higher temperature resulted in a greater percentage of conjugation.

FIG. 7J is a graph depicting the effect of pH on percent conjugation. Strongly basic conditions reduce conjugation percentages.

FIG. 7K depicts data for conjugation of AAV-SpyT conjugated to Spy C-Fab (Construct 1) and AAV-SpyT conjugated to Spy C-mAb (Construct 2). Both AAVs have the W503A detargeting mutation and HA-MTM1 as a transgene.

The data for the experiment of FIG. 7K is set forth below in Table 3.

TABLE 3

| | | | | Percent (%) | Estimated % | % Residual | | |
|---|---|---|---|---|---|---|---|---|
| | Estimated | | | Full/Partial/ | Conjugation by | free SpyC- | % HMW by | |
| | titer | | Endotoxin | Empty | SEC | Ab by SEC | SEC | % Monomer |
| Material | (vg/ml) | Total vg | (EU/mg) | Capsids by Mass | (FIG. 1) | (FIG. 1) | (FIG. 1) | by DLS |
| Conjugate 1 | ~3.5 × 10¹³ | 7.5 × 10¹⁵ | <0.119 | 95.6% Full | 85-90% | Not | Not | 100% |
| | | | | 1.4% Partial | | detected | detected | |
| | | | | 3.0% Empty | | | | |
| Conjugate 2 | ~3.5 × 10¹³ | 7.5 × 10¹⁵ | 0.222 | (measured on | 90.4% | 1.6% | Not | 100% |
| | | | | AAV9-SpyT prior | | | detected | |
| | | | | to conjugations) | | | | |

### Example 4—Separating a Covalently Surface Modified Adeno-Associated Virus Species from Unconjugated Antibody Species Using Cation Exchange

FIGS. 8A and 8B depict data from an experiment using cation exchange to separate a covalently surface modified adeno-associated virus species from unconjugated antibody species. In FIG. 8A, a CIM SO3 1 mL monolith was used as the cation exchange column, and the column was loaded with an in-vitro conjugation reaction mixture of AAV9-SpyT and SpyC-ASGR1 mAb mixed in a 1:30 molar ratio. FIG. 8A depicts a linear gradient elution and highlights the peaks corresponding to the SpyC-Ab and the conjugated AAV9-SpyT-SpyC-Ab species. Binding was at pH 5, less than 4 mS/cm and elution was at pH 5, 4-80 mS/cm. FIG. 8B depicts a step elution in which the in-vitro conjugation reaction mixture was adjusted with sodium chloride solution to a conductivity of 15 mS/cm and the mixture was loaded onto a Poros XS cation exchange column, leading to all SpyC-Ab species not binding into the column and being removed in the flow-through, while all AAV species bound and were removed later in an isocratic elution at 40 mS/cm conductivity.

FIG. 8C depicts data showing that Poros XS cation exchange can separate free SpyC-Ab species following the conjugation reaction. FIGS. 8D and 8E compare CaptoCore400 flow through (size exclusion and anion exchange) (FIG. 8D) to Poros XS elution (cation exchange) (FIG. 8E). Both provide a large conjugated peak detected with dynamic light scattering (DLS). CaptoCore400 flow though did exhibit aggregates, whereas Poros XS elution did not.

### Example 5—Separating a Covalently Surface Modified Adeno-Associated Virus Species from Unconjugated Antibody Species Using Multimodal Multimodal Binding Chromatography

Multimodal binding and size separation bead chromatography exchange to separate a covalently surface modified adeno-associated virus species from unconjugated antibody species. More specifically, the multimodal binding and size separation bead chromatography are functionalized with an octyl amine that is hydrophobic and positively charged, and also include a matrix that provides size exclusion, such as Capto™ Core 400 or Capto™ Core 700.

Covalently surface modified adeno-associated virus species are expected in the flow through and wash (FTW). Unconjugated antibody species are expected to enter resin and be bound tightly, and removed during the strip phase. Loading was 5×10¹³ to 1×10¹⁴ cp/ml beads over 3 minutes and room temperature. FIG. 9 depicts an experiment using multimodal anion exchange to separate a covalently surface modified adeno-associated virus species from unconjugated antibody species. Table 4 below sets forth the relative percentage of SpyC-Ab in the flow through and the percentage yield of AAV species in the flow through as measured using size exclusion chromatography when the load material is treated with different amounts of NaCl and adjusted to different levels of pH. The green row in the figure highlights the optimized condition of adjusting the load material to pH 6.0 with 1 M NaCl, enabling a complete separation of SpyC-Ab and AAV species in the flow through with 0% SpyC-Ab, along with high yield of AAV species of 85%.

TABLE 4

| | | | % Ab in | % Yield | % Yield |
|---|---|---|---|---|---|
| | | | first | rAAV in | rAAV in |
| | Load | Load | FTW | first FTW | Elution |
| Column | NaCl | pH | (SEC) | (SEC) | (SEC) |
| Capto | 300 mM | 8 | 0 | 20 | 80 |
| Adhere | 1M | 8 | 0 | 70 | 30 |
| | 1.5M | 8 | 0 | 70 | 30 |
| | 1M | 6 | 0 | 85 | 5 |
| Capto MMC | 50 mM | 5 | 50 | 60 | 40 |
| | 1M | 5 | 50 | 95 | 5 |

FIG. 10 depicts data from an experiment using multimodal binding and size separation bead chromatography exchange on CaptoCore400 resin to separate a covalently surface modified adeno-associated virus species from unconjugated antibody species. The relative size of species was ˜8 nm for the SpyC-Ab, about 15 nm for the AAV9-SpyT, and about 17 nm for the conjugated AAV9-SpyT-SpyC-Ab. The figure includes a size exclusion chromatogram showing that the load material included two peaks (AAV and SpyC-Ab), but the flow through included only one peak (only AAV, showing removal of free SpyC-Ab species using this approach.

### Example 6—Multivariate Modeling

FIG. 11 depicts a multivariate model from experiments using different salt and pH conditions to induce a change in the size and surface characteristics of AAVs, leading to improved yields in the flow through of CaptoCore400 chromatography. The model is a yield profiler and predicts an optimum condition of adjusting the load material with sodium citrate to a final concentration of 150 mM at pH 5.5, resulting in a predicted yield of 100% and an experimental yield of 107% as measured by ddPCR quantification.

### Example 7—Dynamic Light Scattering of Conjugation Reactions

FIGS. 12A-12C depict dynamic light scattering (DLS) data from AAV9-SpyT, SpyC-ASGR1 mAb (FIG. 12A), and in-vitro conjugation reactions between these two species under stirred and unstirred conditions in sodium chloride and sodium citrate based buffers. The DLS data shows the formation of large aggregates in the conjugation reaction under stirred conditions (FIG. 12B), but no such aggregates in the unstirred condition (FIG. 12C) (incubation only).

### Example 8—Size Exclusion Analysis

FIG. 13A shows size exclusion chromatography data tracking the rate of reaction between AAV9-SpyT and SpyC-ASGR1 mAb for three different batches of AAV9-SpyT with different percentages of full capsids. The molar ratio of AAV9-SpyT to SpyC-ASGR1 mAb was 1:300. The conjugation was performed at a pH of 5.8 with an overnight hold and light mixing. Here, 80% conjugation was selected as the optimal level.

The percentage of conjugated species is measured by relative peak area between the AAV9-SpyT peak at about 8.3 min RT and the conjugated species peaks at 6.5-8.0 RT on the size exclusion chromatograms, and tracked in the chart on the right. All batches reached 80% conjugation, but Batch 1 took 48 hours to reach 80% conjugation while Batches 2 and 3 took 24 hours.

FIG. 13B depicts conjugation data with AAV9-SpyT and SpyC-mAb (anti-CACNG1). The monospecific mAb has two SpyC.

FIG. 13C depicts conjugation data with AAV9-SpyT and SpyC-bispecific Ab (anti-CACNG1). The bispecific mAb has one SpyC.

FIG. 13D depicts conjugation data with AAV9-SpyT and SpyC-Fab (anti-CACNG1). The Fab has one SpyC.

Molar AAV:Ab (antibody with two SpyC, bispecific antibody with one SpyC, or Fab with one SpyC, as described above) ratios of 1:1, 1:6, 1:15, 1:30, 1:300 and 1:1000 were tested. The results showed that ratios of 1:30 to 1:1000 of AAV to Ab resulted in greater levels of conjugation. Table 5 below shows that ratios of 1:30 to 1:1000 of AAV to Ab also resulted in lower percentages of high molecular weight species, and that the use of antibodies with only one SpyC (FIGS. 13C and 13D) resulted in less high molecular weight species than use of an antibody with two SpyC (FIG. 13B). See Table 5.

TABLE 5

| | HMW (%) | | |
|---|---|---|---|
| | mAb | Bispecific mAb | |
| Molar Ratio of | (Two | (Single | |
| AAV:Ab | SpyC) | SpyC) | Fab |
| 1:1 | 9 | 4 | 4 |
| 1:6 | 13 | 2 | 4 |
| 1:15 | 5 | 0 | 3 |
| 1:30 | 4 | 0 | 2 |
| 1:300 | 0 | 0 | 0 |
| 1 1000 | 0 | 0 | 0 |

### Example 9—SEC MALS and Anion Exchange Analysis

FIG. 14A is a graph depicting how anion exchange chromatography separates unconjugated AAV from more conjugated species, which are schematically depicted underneath the graph. Increasing alt gradients lead to more conjugated AAV. Anion exchange uses positively charged ligands to bind to negatively charged species (at pH greater than pI).

FIG. 14B depicts SEC-MALS data of samples with different levels of conjugation. The samples were obtained from an anion exchange chromatography method similar to the one described in FIGS. 7A-7D that can be used to separate populations of differently conjugated species. The chart in the lower section of FIG. 14B summarizes the SEC-MALS analysis of the samples which is used to estimate the number of mAbs conjugated on average in each sample, ranging from less than 1 mAb in the first fraction to greater than 5 mAbs in the last fraction.

FIG. 14C shows the results of a transduction assay where these differently conjugated species were compared for transduction ability using cells expressing ASGR1 receptor. It was observed that lower conjugated species (F1-F2) resulted in higher transduction efficiency than the combined pool (Load) as well as later fractions (F3-F5), suggesting a negative impact of higher conjugation levels on transduction ability, despite potential advantages in targeting ability.

FIG. 14D and FIG. 14E collectively concern transduction efficiency at different pHs and percent conjugation. FIG. 14D depicts green fluorescent protein (GFP) percentage of bispecific antibody with one SpyC, a monospecific monoclonal antibody (mAb) with two SpyCs, or an antigen-binding antibody fragment (Fab) with one SpyC, conjugated at a reaction pH of 5 or 8. See FIGS. 13A to 13C and Table 5. FIG. 14E depicts mean fluorescence intensities (MFI) for the bsAb, mAb and Fab at pH 5 or 8. Data is depicted for multiplicity of infection (MOI) at 2×10⁵ (2E5) and 4×10⁴ (4E4).

The MOI refers to the ratio of infectious agents (in this case, AAV9-SpyT-SpyC-Ab molecules) to target cells used in the assay. AAV particles are added to the cells in different amounts so that the transduction effect can be compared at different levels of infection, as there may be some levels where no signals are observed. The data in this figure shows results from the 4E4 and 2E5 MOI conditions on this transduction assay, showing that all conjugated viruses performed comparably at both these conditions regardless of antibody format or conjugation reaction pH.

Additional data is provided in Table 6.

TABLE 6

| SpyC-Ab | Conjugation | Endotoxin | Estimated % |
|---|---|---|---|
| Type | pH | (EU/ml) | Conjugation |
| mAb | 8 | <0.100 | 67% |
| Fab | 8 | <0.107 | ~60% |
| mAb | 5 | <0.100 | 82% |
| Fab | 5 | <0.100 | ~80% |
| bsAb | 5 | <0.100 | 81% |

The low endotoxin results show that the endotoxin is not confounding the results of cell-based assays (high endotoxin can be detrimental to cells). The data indicate that transduction efficiency post-conjugation was not significantly affected by different pH (5 or 8). However, a higher percentage of conjugation was achieved using pH 5 during the conjugation reaction.

### Example 10—Large-Scale Production of Covalently Surface Modified AAV

FIGS. 15-17 schematically depict exemplary end-to-end processes for production of Covalently Surface Modified AAVs using the in-vitro conjugation reaction and free SpyC-Ab removal steps post-production of a Spy Tagged AAV capsid.

FIG. 15 schematically depicts an exemplary production of retargeting molecules (Rm), here an antibody or Fab fused to SpyCatcher. The antibodies/Fab fused to SpyCatcher are produced in CHO cells in this figure.

Cell lysis is an early part of AAV production and purification. Detergents can be used to lyse cells to release proteins and viruses contained with the cell. Detergents to lyse cells are usually considered mild detergents and include: sodium dodecyl sulphate (SDS), NP-40, Tweens (for example 20 and 80), Tritons (for example X-100 and X-114), CHAPS, CHAPSO, Brij (for example, 35 and 58), Octyl thioglucoside, Octyl Glucoside, deoxycholate, and alkyl sulfates, for example.

Depth filtration typically follows lysis. Downstream processing starts with depth filtration/chromatographic clarification to remove cellular debris after cell lysis, which is followed by affinity capture and non-AAV viral inactivation. Next, polishing 1 and 2 occurs using ion exchange. This is followed by viral retentive filtration and then tangential flow filtration. Here, SpyC-mAb/Fab is formed, which also is depicted in FIG. 1A.

FIG. 16 schematically depicts the formation of AAV comprising SpyTag on the capsid surface, specifically AAV9-SpyT. AAV can be produced in human cells (for example, HEK293 or HeLa cells). Cell lysis is undertaken, preferably without Benzonase. Chromatic clarification is performed to remove cell debris following lysis. Next, tangential flow filtration 1 is performed, followed by affinity capture and polishing, here using one, two or three passes on anion exchange chromatographic columns. This is followed by viral retentive filtration and then tangential flow filtration 2. Here, AAV-SpyT is formed. See also FIG. 1A.

FIG. 17 schematically depicts the conjugation of SpyCatcher-Rm (for example, mAb/Fab as per FIG. 15) to AAV-SpyT (FIG. 16). When starting the conjugation reaction, the SpyC-Rm should be added to the vessel prior to the AAV-SpyT. In order to drive the conjugation reaction to completion, it can be beneficial to provide the SpyC-Rm in molar excess of the AAV9-SpyT, where the excess can be up to 1000-fold. In such cases, once the conjugation reaction is finished, there will remain a significant amount of excess of unconjugated SpyCatcher-Rm following conjugation. This can be due to the SpyCatcher-Rm being added in molar excess to the number of available SpyTag binding sites on the AAV and/or due to incomplete reactions.

Next, unconjugated retargeting molecules (here mAb or Fab, are removed via chromatography, and then TFF for enrichment of the covalently surface modified AAV. Enrichment can result in greater than 50%, 55%, 60%, 65%, 70%, 75%, 80%, 85%, 90%, 95%, 96%, 97%, 98%, 99% or more of unconjugated retargeting molecule species being separated from the covalently surface modified AAV species. Enrichment can result in reducing the presence of unconjugated retargeting molecule species to undetectable levels. The covalently surface modified AAV is schematically depicted in FIG. 1A.

An exemplary downstream protocol for a 500 L bioreactor would be as follows:
1. Culturing, cell lysis, clarification and affinity capture with a yield of 71%;
2. Two pass AEX with a yield of 80%;
3. Formulation of AAV-SpyT with a yield of 75%—exemplary formulation processes would include ultrafiltration/diafiltration for concentration and buffer exchange to increase the concentration of AAV capsids to 3 to 8×10¹³ cp/mL and to place it into a stable buffer suitable for extended storage, freeze-thaw and reaction incubation;
4. In vitro conjugation AAV-SpyT with SpyC-Rm at 4.0×10¹³ vg/ml and using preparative SEC to remove free SpyC-Rm with a yield of 108%; and
5. Final formulation AAV-SpyT-SpyC-Rm at 3-3.5×10¹³ vg/ml at a yield of 81%. The yield percentages are the % of viral genomes (i.e. full capsids) recovered at the end of each unit operation compared to the amounts going into the unit operation. Typically, a yield about 15% is considered acceptable, 30% is considered good and 50% is considered very good.

Table 7 below identifies titers and yields that would results in worst case for doses per batch. Table 8 below identifies titers and yields per batch under different parameters and conditions that would be expected.

TABLE 7

| | | | | Lowest |
|---|---|---|---|---|
| | | | Purification | Expected Doses |
| Component | Batch Size | Titer | Yield | per batch² |
| SpyT-AAV | 500 L | 3 × 10¹⁴ | 20% | 34 |
| | | vg/L | | |
| SpyC-mAb¹ | 500 L | 5.0 g/L | 50% | about 100, 000 |

¹Assuming 50-fold excess of SpyC-mAb required
²Assuming systemic 1 × 10¹³ vg/kg dosing in 80 kg adults

TABLE 8

| | | | | Lowest |
|---|---|---|---|---|
| | | | Purification | Expected Doses |
| Component¹ | Batch Size | Titer | Yield | per batch² |
| SpyT-AAV | 500 L | 1 × 10¹⁵ | 20% | 250 |
| | | vg/L | | |
| SpyC-mAb1 | 500 L | 3.0 g/L | 50% | about 25, 000 |

¹Assuming 300-fold excess of SpyC-mAb required
²Assuming systemic 5 × 10¹² vg/kg dosing in 80 kg adults

### Example 11—the Effects of Additives on Conjugation

The following additives were selected and tested to determine their effects on conjugation. The additives were selected due to potential effects on the charge or hydrophobicity of the reaction molecules or the ionic characteristics of the reaction mixture:
1) 0.5% or 2.5% DMSO.
2) 50 mM or 250 mM L-glutamic acid monosodium salt.
3) 50 mM or 250 mM Arginine.
4) 0.5% or 2.5% w/v Sorbitol.
5) 50 mM or 250 mM Urea.
6) 5% or 25% Isopropyl alcohol (IPA).
7) 5 mM or 25 mM Sodium thiocyanate (NaSCN).

FIG. 18 schematically depicts the effects on conjugation of each of the 7 additives identified above at different concentrations. The addition of 25% IPA during the conjugation reaction yielded 80% conjugation in 4 hours, which was higher than that achieved by a control in 72 hours. The other additives at 4 hours achieved about 70% conjugation in 4 hours, which was the same on controls 1 and 2. Urea at 50 mM achieved about 68% conjugation after 4 hours. The use of additives is optional.

Isopropyl alcohol should preferably be added at a concentration above 5%. Concentrations include about 5.5%. 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25% or more.

FIG. 19 depicts data from a conjugation reaction between AAV-SpyT and SpyC-mAb using the 7 additives. The magnified plot shows the effectiveness of 25% isopropyl alcohol.

### Example 12—Optimization of Mosaicism Level and Rm Format in Covalently Surface Modified Adeno-Associated Viruses

The following discussion concerns Covalently Surface Modified Adeno-Associated Viruses and uses AAV with the SpyTag-Spy-Catcher System and having monoclonal antibodies (or fragments or derivative thereof) as retargeting molecules (AAV-SpyT-SpyC-Ab) as an exemplar. Any specific binding pair (that is, a first member and second cognate member) can be used according to the inventions.

When producing AAV-SpyT-SpyC-Ab, three important aspects are (1) the number of SpyT (an exemplary FM) on the AAV capsid, which directly affects the number of SpyC-Abs that can be covalently attached to the AAV capsid, (2) the format of SpyC-Ab to be covalently attached. Formats include, but are not limited to (i) a mAb with two SpyC in the constant regions, (ii) an antibody with a single SpyC on a constant region (referred to as a “bispecific” here), (iii) a Fab with a single SpyC on the variable heavy chain, or (iv) other options (such as ScFv), and (3) the molar ratio of AAV to retargeting molecule during conjugation.

To further explain the first aspect, the number of SpyT (an example of a first member (FM)) on the AAV capsid can be modified by adjusting the ratio of the pRC and the pRC-SpyT plasmids that are co-transfected into the bioreactor along with the pGOI and pHelper plasmids. For example, when 1/10 of all the pRC plasmids transfected are pRC-SpyT plasmids, the AAV capsid is expected to have an average of 6 SpyT, as 1/10 of the 60 viral proteins are expected to have a SpyT (1/10×60=6). Similarly, a transfection ratio of 1/4 is expected to result in 15 SpyT per AAV capsid (1/4×60=15), and a transfection ratio of 1/30 is expected to result in 2 SpyT per AAV capsid (1/30×60=2), as the capsid is stoichiometrically assembled inside the cell using the available viral proteins. The ratio of pRC-SpyT to pRC plasmids during transfection is referred to as the “mosaicism” of the AAV capsid, with “higher mosaicisms” such as 1/4 resulting in higher SpyT on the AAV capsids on average, and “lower mosaicisms” such as 1/30 resulting in fewer SpyT on the AAV capsids on average. Thus, where the ratio of pRC-FM to pRC plasmids is a larger fractional value, the mosaicism will be higher. As explained above, 1/4 is a larger fractional value than 1/30.

It is desirable to optimize the mosaicism when producing AAV-SpyT-SpyC-Ab molecules. Firstly, it has shown that when an AAV has too many Abs conjugated on its surface, it can result in a negative impact on transduction efficiency and can lower the potency of the therapeutic (See Example 9 showing how higher levels of Ab conjugation resulted in lower transduction efficiency). However, since the capsid is stoichiometrically assembled, it is desirable for the mosaicism to not be too low in order to prevent the formation of a high proportion of AAV capsids without even a single SpyT, which prevents conjugation. For example, with a mosaicism of 1/10, it is difficult to achieve optimal (i.e., ‘complete’ conjugation), which suggests that there is a proportion of AAV capsids without any or without accessible SpyT sites. See, for example, FIG. 13A. Lowering the mosaicism yet further to 1/20 or 1/30 is expected to increase the proportion of capsids that cannot be conjugated at all. Thus, there is a need to optimize mosaicism to achieve both a therapeutic product with maximum potency, as well as a product that does not have a significant proportion of unconjugated molecules. See FIGS. 20A to 20H (showing the effect of different mosaicisms on conjugation percentage).

The second aspect requiring optimization is selecting the format of SpyC-Ab to be conjugated to the AAV capsids. There are advantages and disadvantages to various formats and a combination of in vitro and in vivo data can help to make the decision. For the mAb format, due to the presence of two SpyC, there is the potential for each SpyC to bind to a different AAV capsid, resulting in the formation of “daisy-chain” species such as AAV-SpyT-SpyC-mAb-SpyC-SpyT-AAV-, etc. These species have high molecular weight, are undesirable, and have been observed to form particularly when carrying out the in vitro conjugation reaction between AAV-SpyT and SpyC-mAb in a limiting molar ratio.

A way to avoid the formation of these species is to use a bispecific format with only one SpyC. However, due to the potential negative effect of high levels of surface conjugation on viral potency, the bispecific format also may not be ideal due to increasing the number of SpyC-Abs conjugated on the surface of each capsid. The Fab format may be beneficial to achieve higher levels of conjugation with maximum potency due to the small size of Fabs (˜50 kDa) compared to mAbs (˜150 kDa).

FIGS. 20A to 20H are as follows: FIG. 20A schematically depicts a hypothetical readout on the impact that mosaicism has on transduction efficiency (green) and percent (%) conjugation (red) with Fab, bispecific Ab and mAb. In this hypothetical, 80% is the desired limit for conjugated capsids. Mosaicisms selected were 1/5, 1/7.5, 1/10, 1/15 and 1/30. FIG. 20B is a bar graph comparing upstream bioreactor titer and process yield for five different mosaicisms (“M”), produced at 2 L scale for 1/5 (M1), 1/7.5 (M2), 1/15 (M4) and 1/30 (M5), and at 50 L scale for 1/10 (M3). FIG. 20C is a bar graph comparing percent (%) conjugation vs. time for the five different mosaicisms, as measured using size-exclusion chromatography. The data indicates a maximum conjugation for the 1/7.5 and 1/10 mosaicisms.

FIGS. 20D to 20H are graphs schematically depicting data of size-exclusion chromatography overlays showing conjugation process for different mosaicisms M1 (1/5), M2 (1/7.5), M3 (1/10), M4 (1/15), and M5 (1/30). The FM is SpyT and the SCM is SpyC here. FIG. 20D depicts an overlay of M1-M5 prior to addition of SpyC-Abs, showing increasing size with increasing level of mosaicism (M1>M2>M3>M4>M5). The X-axis designates 7.60 to 10.40 minutes and the Y-axis designates 0.00 to 800.00 EU. FIG. 20E depicts an overlay of conjugation reactions between M1 and anti-CACNG1 bispecific single-SpyC mAb at 2, 8, 24 and 36 h, showing increasing conjugation level over time via reduction of the peak at about 8.5 min RT. The X-axis designates 3.00 to 14.50 minutes and the Y-axis designates 0.00 to 200.00 EU. FIG. 20F depicts an overlay of conjugation reaction between M2 and anti-CACNG1 single-SpyC Fab at 2, 8, 24 and 36 h, showing increasing conjugation level over time via reduction of the peak shoulder at about 8.5 min RT. The X-axis designates 1.50 to 14.50 minutes and the Y-axis designates 0.00 to 450.00 EU. FIG. 20G schematically depicts an overlay of conjugated species of M1-M5 and anti-CACNG1 single-SpyC Fab after 48 h of reaction, showing increasing conjugation level for higher mosaicisms. The X-axis designates 0.00 to 15.00 minutes and the Y-axis designates 0.00 to 1600.00 EU. FIG. 20H depicts an overlay of conjugated species with mosaicisms M1-M5 and anti-CACNG1 single-SpyC bispecific mAb after 48 h of reaction and removal of majority of excess free SpyC-Ab using preparative size exclusion chromatography, which showed increasing conjugation level for higher mosaicisms. The X-axis designates 0.00 to 15.00 minutes and the Y-axis designates 0.00 to 160.00 EU.

The third aspect for optimization is the molar ratio of AAV to Retargeting Molecule (for example, an antibody. FIG. 5A shows that significant high molecular weight species can be observed in the SEC chromatograms corresponding to molar ratios of 1:6 and 1:15 compared to 1:30 and 1:300. In particular, FIG. 5A shows the impact of AAV:antibody molar ratio on a AAV capsid with fixed number of SpyTags (1/10 mosaicism level=about 6 SpyTags), while FIGS. 20A to 20H use a fixed AAV:antibody molar ratio (1:300) but vary the mosaicism, that is the number of SpyTags on the AAV capsid (1/30=about 2 SpyTags to 1/5=about 12 SpyTags).

FIG. 5B is a preparative scale SEC on a 100 ml SEPAX SRT 500A column to remove unconjugated antibody. FIG. 5B is an overlay of UV280 profiles over 20 column injections, and clearly depicts the separation of free antibody species (in other words, unconjugated antibody species) from AAV species. The multiple overlaid curves are different injections on the same column, as about 30 injections were required to process all of the material.

### Example 13—Process Analytical Technology and Improved Purification

FIG. 21A depicts exemplary production purification trains for AAV, such as recombinant AAV. The top train uses a batch process where repeated passes are required to exchange buffer and concentrate the retentate, which contains the desired biological material, such as AAV. See Adams et al., Biotech. Bioeng. 117: 3199-3211 (2020).

The bottom section of FIG. 21A replaces the batch tangential flow filtration unit with a single-pass tangential flow filtration unit (SPTFF unit), which permits a continuous process. It was surprising how well SPTFF performed with AAV, as taught herein,

The Batch TFF approach can take multiple days (for example, 2 days) due to the repeated cycling through the conventional TFF unit to achieve concentration prior to further purification. The SPTFF approach is a continuous approach, and is significantly faster than the Batch TFF approach, and can be performed in several hours, such as 3 to 5 hours. The SPTFF approach provides faster concentration, while minimizing sheer stress and damage to AAVs. The SPTFF approach also is amenable to the use of Process Analytical Technology (PAT) and automation. FIG. 21B depicts a process for purifying retargeted AAV at the 500 L production scale.

For further comparison, FIG. 22A schematically shows a Batch TFF (top), where the retentate is repeatedly cycled through a feed tank and pump to repeatedly passed through a membrane, with the concentrated retentate being removed after repeated cycles. A Single-Pass TFF removes biological material from the feed tank through a pump to a multi-stage membrane module that separate the retentate from the permeate, while concentrating the retentate.

FIG. 22B is a graph comparing Batch TFF and Single-Pass TFF. Single-Pass TFF achieves higher concentration and is faster as compared to Batch TFF. Single-Pass TFF continuously sends biological material to the next operation in the purification train, whereas Batch TFF does not send biological material until the end of the batch cycle.

FIG. 23 schematically and qualitatively compares the batch operation to a continuous operation for AAV purification in terms of Cell lysis, Clarification (depth filtration), TFF (Batch or Single-Pass) and Affinity Capture. The continuous process (SPTFF) can be finished in less than a day, whereas the batch process can be multi-day.

The cell lysis step, typically using a detergent such as Tween-20, typically takes up to about two hours, and is depicted as the same for both the Batch and the SPTFF (continuous) process. Following lysis, clarification takes about 1 hour. The processes then diverge at the TFF step.

For the Batch process, TFF takes about 3 hours per batch due to the repeated cycling. Not until a batch is complete can the concentrated biological material in a buffer be passed on the affinity capture, which takes about 2 to 3 hours per batch. Because multiple batches are required, the affinity chromatography is typically not completed until the next day.

For the Continuous process, clarification, SPTFF and affinity capture can take place substantially simultaneously. Biological material continuously flows to clarification (about 1 hour), SPTFF (about 1½ hours) and affinity capture (about 2 hours to 3 hour). Accordingly, when an early portion of biological material is in affinity capture, later portions of biological material are in SPTFF or clarification.

FIG. 24 schematically depicts exemplary arrangements for multi-stage membrane module cassettes to be used with Single-Pass TFF. The configurations depict four to seven tiers of membrane module cassettes where the initial tiers (left side) contain more or same number of membrane module cassettes as the succeeding tiers (moving towards the right side), in the manner suggested by the manufacturer, here Pall/Cytiva. Total area and path length of the membrane module cassettes also are set forth. Other arrangement of membranes, flow rates and transmembrane pressure (TMP) can be selected by the person skilled in the art.

FIG. 25 is a graph depicting volumetric concentration factor (VCF) versus transmembrane pressure (TMP) using the 4-in-series, 5-in-series, 6-in-series and 7-in-series exemplary configurations depicted in FIG. 24 with a feed comprising an exemplary AAV, here AAV9 comprising a SpyTag insert. A Batch process target would be 8-10×VCF at a TMP of 5 to 10 psi.

FIG. 26 depicts data from a 5-in-series configuration according to FIG. 24 at flow rates of 90 ml/minute, 120 ml/minute and 150 ml/minute. The log best-fit equation of VCF=A In (TMP-B) using the values at each flow rate set forth near the plot (and rounded off in the included table) can be used to parameterize the data. At the right side of the figure, there is a graph of parameter value (A, B) and feed flow rate in liters per square meter of membrane per hour (LMH) for 4-in-series and 5-in-series exemplary configurations of FIG. 24 and allows optimized conditions to be selected in silico using an exemplary AAV, here AAV9 comprising a SpyTag insert. This model can be used to predict the VCF for any flow rate and TMP for an in-series configuration of interest.

FIG. 27A is a design space model based on FIGS. 25 and 26 using the 5-in-series configuration of FIG. 24. Here, the process target was 35 LMH, and the intersecting lines indicate a VCF of 8 and a TMP of 10 psi. An exemplary acceptable zone would be a VCF of 6-10 and a TMP of 7.5 to 12.5 psi. FIG. 27B is an exemplary comparison of process parameters between SPTFF and Batch TFF. With Batch TFF, typically there would be one batch before the next operation. However, depending on the scheduling of upstream production bioreactors and bioreactor titers, there could be pooling of multiple batches before the next operation. Effective residence time of a given portion of biological material in the SPTFF is only about 10 minutes, and the overall time is for all biological material to pass thought the SPTFF.

FIG. 28 depicts data from a bench-scale trial to determine the number of buffer washes need to attain about a 90% recovery of AAV, here AAV9 with integrated SpyTag, in a low-TMP process. On average, the AAV9 here contained 6 SpyTag peptides per viral capsid. The load concentration was 1.7×10¹² capsids/ml (cp/ml). The steady state concentration using SPTFF was 1.6-1.9×10¹³ cp/ml, yielding a steady state VCF of 10 to 11λ. Capsid titer in retentate (cp/ml) versus SPTFF operating time (minutes) was measured using four buffer flushes. The final pool flush (1 and 2) achieved 1.4×10¹³ cp/ml. As the right side of the figure shows, 71% of capsids were recovered in the retentate pool, 11% of capsids were recovered by flush 1 and 6% of capsids were recovered with flush 2. It was determined that only two buffer flushes were required to achieved about a 90% recovery with a VCF of 8λ.

FIG. 29 is a graph depicting Permeate Flux (LMH), Throughput (L/m²), Feed Flow Rate (L/hr) and TMP (psi) in a pilot-scale trial. Using continuous SPTFF, the data showed flux decline and TMP build up. To mitigate TMP increase beyond 12.5 psi, feed flow rate was slowed. This resulted in a longer process time of 180 minutes rather than the expected 90 minutes and an overall VCF of 5× was achieved rather than the target VCF of 8λ.

FIG. 30 depicts a tween micelle build-up on the TFF membrane. Without being bound by any theory or hypothesis, it is believed that detergent micelle buildup (here, Tween-20) is the cause of an unexpected flux decline of about 50% using SPTFF to concentrate AAV. Typically, a 20% flux decline is expected when concentrating antibodies. This figure also set forth the approximate size of AAV, Host Cell Protein aggregates (HCP) and Tween-20 micelles. The micelle concentration of Tween-20 was determined to be about 0.7%. See Basheva et al, J. Physical Chemistry Chemical Physics, Issue 38 (2007) (discusses properties of films formed by Brij 35 and Tween 20). Detergents, such as Tweens, are a common component of cell lysis buffers used in the production of AAV.

FIG. 31 is a graph depicting fold presence of Tween-20 on the retentate side of membrane and the permeate side of the membrane for both Batch TFF and SPTFF. Most Tween-20 is on the retentate side.

FIG. 32 is a graph depicting the flux decline after two hours with varying percentages of Tween-20 in the lysis buffer. The lower the percentage of Tween-20, the lower the percentage of flux decline encountered. In addition to Tween-20, the buffer contained 20 mM Tris, 2 mM MgCl₂ at a pH of 7.4. The feed flow rate was 35 LMH and the TMP was about 5 to 10 psi.

With Batch TFF, flux decline can be address by increasing processing time. However, with SPTFF immediate control is desired.

FIG. 33 compares control with the retentate valve to control with a Permeate pump. Option 1 with the retentate valve found that TMP reached 22 psi, and after which the flow had to be reduced from 40 LMH to 30 LMH. VCF dropped from about 10× to about 6λ. Option 2 with the permeate pump was superior, which acts as a suction pump. TMP was controlled to well under 10 psi and a VCF of 8× was maintained. At the right side to the figure, Option 1 (SPTFF with retentate valve) and Option 2 (SPTFF with permeate pump) were compared to a Batch TFF. Option 1 did not perform as well as Option 2 and Batch TFF. Option 2 was superior to Batch TFF and Option 1 in terms of capsid yield and percent aggregation. The permeate pump flow should be set within the VCF design space to avoid negative permeate pressure buildup. Thus, the flow rate of operation of the permeate pump should be within the range established in FIG. 27A.

FIG. 34 depicts an overall pilot scale process.

FIG. 35 compares VCFs (1-14), SPTFF retentate flow rates and residence time in affinity capture. VCFs of 7 to 13 and SPTFF retentate flow rates of 75-40 provided an exemplary range of residence time suitable for affinity loading. The flow rate should be selected to avoid depleting or overwhelming the affinity column. This calculation was based on a pilot-scale trial with a 525 ml/minute feed flow in a 5-in-one series SPTFF module and then loaded on to a 200 ml POROS CaptureSelect AAV9 column.

FIG. 36 depicts how UV280 profile of affinity capture flow can be used for process monitoring of VCF and process stability using SPTFF for continuous processing. Three different runs were performed for comparison purposes. Run 1 was performed without a permeate pump and achieved a VCF of only 5λ. Run 2 was performed with a permeate pump with a feed to retentate flush (with recirculation) and achieved a VCF of 8λ. Run 3 was performed with a permeate pump with a feed to retentate flush (with recirculation) and a permeate to retentate flush, which achieved a VCF of 10λ. Most chromatography systems have built-in UV280 sensors that can detect load concentration, and provide an indication of VCF and process stability. Any needed correction, such as pump and/or valve control, can be based upon the data received through process analytical technology. See Thakur et aL., J. Membrane. Sci. 613: 118492 (2020) discuss the use of process analytical technology with SPTFF.

Advantages and Aspects of SPTFF include:
Greater efficiency in AAV manufacturing from harvest to final capture and purification;
Use of a permeate pump provides real-time control over TMP and maximizes AAV yield and minimizes AAV aggregation;
Detergents, such as Tween-20, can decrease permeate flux, which can be best managed through use of a permeate pump; and
Volumetric concentration factor (VCF) depends on flow rate, TMP and SPTFF membrane module configuration, which can be addressed by the empirical modeling of VCF vs. TMP curves for optimization based upon the teachings contained herein.

### Example 14—Effect of Retargeting Conditions and Bioreactor Scale on Vector Titer

FIG. 37A depicts vector viral titer and viral genome to capsid ratio (Vg:Cp) of different capsid types: a wild type (WT) AAV9, a detargeted AAV9 and a detargeted AAV9 comprising the SpyTag peptide in the viral capsid. Below each column there is provided a schematic diagram of each capsid type. Vector genome titer was consistent amongst the three capsid types.

Viral genome to capsid percentage exhibited slightly more variation, and ranged from 11% to 17% full capsids. amongst the three capsid types.

FIG. 37B depicts virus vector viral titer and viral genome to capsid ratio (Vg:Cp) at different bioreactor scales: 0.2 liters, 2 liters and 50 liters. The 0.2 liter exhibited the highest vector genome titer, and was in the middle in terms of viral genome to capsid percentage. The range overall was 13% to 24% viral genome to capsid percentage amongst the three sizes of bioreactors The 2 liter reactor exhibited the largest viral genome to capsid percentage, and the 50 liter reactor exhibited the lowest viral genome to capsid percentage.

FIG. 38 is a graph depicting conjugated AAV9-SpyT-SpyC-Rm species with a detargeting mutation compared to an unconjugated antibody. The detargeting mutation was N272A The retargeting molecules were an ASGR1 monoclonal antibody and a FELD1 monoclonal antibody. The conjugated AAV9-SpyT-SpyC-Rm species were tested.

### Examples of Covalently Surface Modified AAV

The following examples provide teachings on how to produce covalently surface modified AAV of all serotypes using exemplary sequences according to the inventions. Pertinent polynucleotide and amino acid sequences are widely available in the published literature, and therefore the inventions are not limited to the polynucleotide and amino acid sequences set forth in the specification and the sequence listing.

The below examples set forth rAAV comprising VP1, VP2 and/or VP3 protein fused to a first member of a specific binding pair. The rAAV can be later conjugated to a retargeting molecule fused to a second cognate member of the specific binding pair. Use of SpyTag and SpyCatcher as specific binding pairs as disclosed herein provides teachings and data that is applicable to the use of other specific binding pairs, including fragments and derivative of specific binding pairs, disclosed herein or otherwise known in the field. Conjugation is disclosed throughout, and an exemplary overview can be found at FIGS. 12A-C, 13A-B, 14A-E, 15, 16, 17, 18, 19, 20 A-H and 21A-B and associated text. Rep and cap genes from the same serotype are exemplified below, but the inventions also provide for use or rep and cap genes from different serotypes.

### Example 15—AAV1

Covalently surface modified AAV1 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 1 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV1.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween.
Ranges of ratios of 1/1 to 1/60 can be employed, and subranges as set forth below. More preferred mosaicisms are:
1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.
Further preferred mosaicisms are:
1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.
Still more preferred mosaicisms are:
1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.
Preferred ranges of mosaicisms are 1/4 to 1/13; 1/5 to 1/12; 1/6 to 1/11, 1/7 to 1/10.5. or 1/7.5 to 1/10.
(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid Type | Specifics |
|---|---|
| pRC | AAV 1 rep and cap genes. See, |
| | e.g., Example 32. |
| pRC-FM | See, e.g., Examples 34-36 to |
| | obtain VP1, VP2 and VP3 |
| | proteins fused to an FM (e.g., |
| | tag-type protein). |
| pHELP | See, e.g., Examples 33 and 37- |
| | 39 disclosing helper genes from |
| | herpes simplex virus (HSV), |
| | human papilloma virus (HPV), |
| | bocavirus, and baculovirus. |

### Example 16—AAV2

Covalently surface modified AAV2 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 2 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV2.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.
More preferred mosaicisms are:
1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV2 rep and cap genes. See, e.g., |
| | Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

Variant AAV2 7m8: AAV2 7m8 (also referred to as AAV2.7m8) can be used and is characterized by a 10-amino acid peptide ‘LALGETTRPA’, referred to as ‘7m8’, inserted at position 588 of the AAV2 capsid protein sequence. (Dalkara, D., et al. “In vivo-directed evolution of a new adeno-associated virus for therapeutic outer retinal gene delivery from the vitreous. Sci. Transl. Med. 2013; 5: 189ra76.” Gene therapy restores vision in a canine model of childhood blindness. Nat Genet 28.1 (2001): 92-5).

Variant AAV2-4Y-F: AAV2 Quad Y-F can be used and is a modified AAV2 comprises a mutated AAV2 VP3 capsid protein comprising phenylalanines (F) at each of the positions corresponding to Y272, Y444, Y500, and Y730 in a wild type AAV2 VP3 capsid protein (Petrs-Silva, Hilda, et aL. “High-efficiency transduction of the mouse retina by tyrosine-mutant AAV serotype vectors.” Molecular therapy 17.3 (2009): 463-471).

### Example 17—AAV3

Covalently surface modified AAV3 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 3 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV3.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.
(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV 3 rep and cap genes. |
| | See, e.g., Example 32. |
| pRC-FM | See, e.g., Examples 34-36 to |
| | obtain VP1, VP2 and VP3 |
| | proteins fused to an FM (e.g., |
| | tag-type protein). |
| pHELP | See, e.g., Examples 33 and |
| | 37-39 concerning helper genes |
| | from herpes simplex virus |
| | (HSV), human papilloma virus |
| | (HPV), bocavirus, and |
| | baculovirus. |

### Example 18—AAV4

Covalently surface modified AAV4 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 4 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV4.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV 4 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 19—AAV5

Covalently surface modified AAV5 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 5 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV5.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are: 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV 5 rep and cap genes. See, |
| | e.g., Examples 32. |
| pRC-FM | See, e.g., Examples 34-36 to |
| | obtain VP1, VP2 and VP3 |
| | proteins fused to an FM (e.g., |
| | tag-type protein). |
| pHELP | See, e.g., Examples 33 and 37- |
| | 39 concerning helper genes from |
| | herpes simplex virus (HSV), |
| | human papilloma virus (HPV), |
| | bocavirus, and baculovirus. |

### Example 20—AAV6

Covalently surface modified AAV6 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 6 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV6.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See, e.g., Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid types | Specifics |
|---|---|
| pRC | AAV 6 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 21—AAV 7

Covalently surface modified AAV 7 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 7 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV7.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid types | Specifics |
|---|---|
| pRC | AAV 7 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 22—AAV8

Covalently surface modified AAV8 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV8 rep and cap genes. See, e.g., |
| | Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV8.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid types | Specifics |
|---|---|
| pRC | AAV 8 rep and cap genes. See, |
| | e.g., Examples 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 23—AAV9

Covalently surface modified AAV9 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV9 rep and cap genes. See, e.g., |
| | Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV9.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV9 rep and cap genes. See, e.g., |
| | Examples 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 24—AAV10

Covalently surface modified AAV10 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV10 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Example 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV10.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV10 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 25—AAV11

Covalently surface modified AAV11 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 11 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Example 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV11.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV 11 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 26—AAV12

Covalently surface modified AAV12 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. |
| pRC | AAV 12 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV12.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV 12 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 37-39 |
| | concerning helper genes from |
| | herpes simplex virus (HSV), human |
| | papilloma virus (HPV), bocavirus, |
| | and baculovirus. |

### Example 27—AAV13

Covalently surface modified AAV13 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV 13 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34- 36. |
| pHELP | Comprises one or more adenovirus |
| | helper genes. See, e.g., Example |
| | 33. |

(1) Plasmid pGOI can comprise one or more genes that are desired to be expressed in covalently surface modified AAV13.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| | |
|---|---|
| pRC | AAV 13 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 28—AAV rh10

Covalently surface modified AAV rhp0 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV rh10 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV rh10.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid types | Specifics |
|---|---|
| pRC | AAV rh10 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 29—AAV rh39

Covalently surface modified AAV rh39 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV rh39 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV rh39.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid types | Specifics |
|---|---|
| pRC | AAV rh39 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 30—AAV rh43

Covalently surface modified AAV rh43 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV rh43 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV rh43.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred ratios mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid types | Specifics |
|---|---|
| pRC | AAV rh43 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Example 31—AAV rh74

Covalently surface modified AAV rh74 can be made by transfecting a cell with plasmids as set forth below, followed by culturing the transfected cells and harvesting the resulting AAV, and then conjugating the resulting AAV with a second cognate member (SCM) that is fused to a retargeting molecule The first chart below uses SpyTag as the first member of a specific binding pair, and Adenovirus Helper Genes. Modifications can be undertaken as disclosed below:

| Plasmid type | Specifics |
|---|---|
| pGOI | Any gene(s) of interest flanked by |
| | AAV ITRs. See, e.g., Example 32. |
| pRC | AAV rh74 rep and cap genes. See, |
| | e.g., Example 32. |
| pRC-FM | pRC-Spy Tag. Provides VP1, VP2 |
| | and VP3 proteins fused to Spy Tag |
| | protein. See, e.g., Examples 34-36. |
| pHELP | Comprises one or more adenovirus |
| | helper genes. See, e.g., Example |
| | 33. |

(1) Plasmid pGOI can comprise one or more genes that are desired to be expressed in covalently surface modified AAV rh74.
(2) Plasmids pRC-FM can be modified for other specific binding pairs. The first member (FM) should be complementary to a second cognate member (SCM) that is fused to a retargeting molecule (for example, an antibody, as well derivatives and fragments thereof, and still other molecules). See Definitions and Description sections of the Detailed Description, the Figures and associated text for “retargeting” and “retargeting molecules”. The populations of FMs and SCMs can be homogenous or heterogeneous as long as the selected FMs can come in contact with appropriate SCMs so that binding can occur.
(3) Detargeting mutations are optional and can be undertaken as disclosed herein and in publications. See Definitions and Description sections of the Detailed Description, the Figures and associated text for “detargeting” and “detargeting mutations”.
(4) To lessen the possibility of encountering neutralizing antibodies when the covalently surface modified AAV is administered to a subject, KTag can be used as an SCM to bind with SpyTag (FM) (SEQ ID NOS: 54 and 86). KTag and SpyLigase are based upon SpyCatcher. KTag (SEQ ID NO: 85) has only 10 amino acids, which renders it less immunogenic. KTag can be bound to SpyTag in the presence of SpyLigase (SEQ ID NO: 61). The SpyLigase can be removed by subsequent purification, such as by chromatography and/or filtration so that the SpyLigase will not have an appreciable presence in the final drug substance.
(5) Preferred mosaicisms are as follows: 1/1, 1/1.1, 1/1.2, 1/1.3, 1/1.4, 1/1.5, 1/1.6, 1/1.7, 1/1.8, 1/1.9, 1/2, 1/2.1, 1/2.2, 1/2.3, 1/2.4, 1/2.5, 1/2.6, 1/2.7, 1/2.8, 1/2.9, 1/3, 1/3.1, 1/3.2, 1/3.3, 1/3.4, 1/3.5, 1/3.6, 1/3.7, 1/3.8, 1/3.9, 1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9, 1/13, 1/13.1, 1/13.2, 1/13.3, 1/13.4, 1/13.5, 1/13.6, 1/13.7, 1/13.8, 1/13.9, 1/14, 1/14.1, 1/14.2, 1/14.3, 1/14.4, 1/14.5, 1/14.6, 1/14.7, 1/14.8, 1/14.9, 1/15, 1/15.1, 1/15.2, 1/15.3, 1/15.4, 1/15.5, 1/15.6, 1/15.7, 1/15.8, 1/15.9, 1/16, 1/16.1, 1/16.2, 1/16.3, 1/16.4, 1/16.5, 1/16.6, 1/16.7, 1/16.8, 1/16.9, 1/17, 1/17.1, 1/17.2, 1/17.3, 1/17.4, 1/17.5, 1/17.6, 1/17.7, 1/17.8, 1/17.9, 1/18, 1/18.1, 1/18.2, 1/18.3, 1/18.4, 1/18.5, 1/18.6, 1/18.7, 1/18.8, 1/18.9, 1/19, 11/9.1, 1/19.2, 1/19.3, 1/19.4, 1/91.5, 1/19.6, 1/19.7, 1/19.8, 1/19.9, 1/20, 1/21, 1/21.1, 1/21.2, 1/21.3, 1/21.4, 1/21.5, 1/21.6, 1/21.7, 1/21.8, 1/21.9, 1/22, 1/22.1, 1/22.2, 1/22.3, 1/22.4, 1/22.5, 1/22.6, 1/22.7, 1/22.8, 1/22.9, 1/23, 1/23.1, 1/23.2, 1/23.3, 1/23.4, 1/23.5, 1/23.6, 1/23.7, 1/23.8, 1/23.9, 1/24, 1/24.1, 1/24.2, 1/24.3, 1/24.4, 1/24.5, 1/24.6, 1/24.7, 1/24.8, 1/24.9, 1/25, 1/25.1, 1/25.2, 1/25.3, 1/25.4, 1/25.5, 1/25.6, 1/25.7, 1/25.8, 1/25.9, 1/26, 1/26.1, 1/26.2, 1/26.3, 1/26.4, 1/26.5, 1/26.6, 1/26.7, 1/26.8, 12/6.9, 1/27, 1/27.1, 1/27.2, 1/27.3, 1/27.4, 1/27.5, 1/27.6, 1/27.7, 1/27.8, 1/27.9, 1/28, 1/28.1, 1/28.2, 1/28.3, 1/28.4, 1/28.5, 1/28.6, 1/28.7, 1/28.8, 1/28.9, 1/29, 1/29.1, 1/29.2, 1/29.3, 1/29.4, 1/29.5, 1/29.6, 1/29.7, 1/29.8, 1/29.9, 1/30, 1/30.1, 1/30.2, 1/30.3, 1/30.4, 1/30.5, 1/30.6, 1/30.7, 1/30.8, 1/30.9, 1/31, 1/31.1, 1/31.2, 1/31.3, 1/31.4, 1/31.5, 1/31.6, 1/31.7, 1/31.8, 1/31.9, 1/32, 1/32.1, 1/32.2, 1/32.3, 1/32.4, 1/32.5, 1/32.6, 1/32.7, 1/32.8, 1/32.9, 1/33, 1/33.1, 1/33.2, 1/33.3, 1/33.4, 1/33.5, 1/33.6, 1/33.7, 1/33.8, 1/33.9, 1/34, 1/34.1, 1/34.2, 1/34.3, 1/34.4, 1/34.5, 1/34.6, 1/34.7, 1/34.8, 1/34.9, 1/35, 1/35.1, 1/35.2, 1/35.3, 1/35.4, 1/35.5, 1/35.6, 1/35.7, 1/35.8, 1/35.9, 1/36, 1/36.1, 1/36.2, 1/36.3, 1/36.4, 1/36.5, 1/36.6, 1/36.7, 1/36.8, 1/36.9, 1/37, 1/37.1, 1/37.2, 1/37.3, 1/37.4, 1/37.5, 1/37.6, 1/37.7, 1/37.8, 1/37.9, 1/38, 1/38.1, 1/38.2, 1/38.3, 1/38.4, 1/38.5, 1/38.6, 1/38.7, 1/38.8, 1/38.9, 1/39, 1/39.1, 1/39.2, 1/39.3, 1/39.4, 1/39.5, 1/39.6, 1/39.7, 1/39.8, 1/39.9, 1/40, 1/40.1, 1/40.2, 1/40.3, 1/40.4, 1/40.5, 1/40.6, 1/40.7, 1/40.8, 1/40.9, 1/41, 1/41.1, 1/41.2, 1/41.3, 1/41.4, 1/41.5, 1/41.6, 1/41.7, 1/41.8, 1/41.9, 1/42, 1/42.1, 1/42.2, 1/42.3, 1/42.4, 1/42.5, 1/42.6, 1/42.7, 1/42.8, 1/42.9, 1/43, 1/43.1, 1/43.2, 1/43.3, 1/43.4, 1/43.5, 1/43.6, 1/43.7, 1/43.8, 1/43.9, 1/44, 1/44.1, 1/44.2, 1/44.3, 1/44.4, 1/44.5, 1/44.6, 1/44.7, 1/44.8, 1/44.9, 1/45, 1/45.1, 1/45.2, 1/45.3, 1/45.4, 1/45.5, 1/45.6, 1/45.7, 1/45.8, 1/45.9, 1/46, 1/46.1, 1/46.2, 1/46.3, 1/46.4, 1/46.5, 1/46.6, 1/46.7, 1/46.8, 1/46.9, 1/47, 1/47.1, 1/47.2, 1/47.3, 1/47.4, 1/47.5, 1/47.6, 1/47.7, 1/47.8, 1/47.9, 1/48, 1/48.1, 1/48.2, 1/48.3, 1/48.4, 1/48.5, 1/48.6, 1/48.7, 1/48.8, 1/48.9, 1/49, 1/49.1, 1/49.2, 1/49.3, 1/49.4, 1/49.5, 1/49.6, 1/49.7, 1/49.8, 1/49.9, 1/50, 1/50.1, 1/50.2, 1/50.3, 1/50.4, 1/50.5, 1/50.6, 1/50.7, 1/50.8, 1/50.9, 1/51, 1/51.1, 1/51.2, 1/51.3, 1/51.4, 1/51.5, 1/51.6, 1/51.7, 1/51.8, 1/51.9, 1/52, 1/52.1, 1/52.2, 1/52.3, 1/52.4, 1/52.5, 1/52.6, 1/52.7, 1/52.8, 1/52.9, 1/53, 1/53.1, 1/53.2, 1/53.3, 1/53.4, 1/53.5, 1/53.6, 1/53.7, 1/53.8, 1/53.9, 1/54, 1/54.1, 1/54.2, 1/54.3, 1/54.4, 1/54.5, 1/54.6, 1/54.7, 1/54.8, 1/54.9, 1/55, 1/55.1, 1/55.2, 1/55.3, 1/55.4, 1/55.5, 1/55.6, 1/55.7, 1/55.8, 1/55.9, 1/56, 1/56.1, 1/56.2, 1/56.3, 1/56.4, 1/56.5, 1/56.6, 1/56.7, 1/56.8, 1/56.9, 1/57, 1/57.1, 1/57.2, 1/57.3, 1/57.4, 1/57.5, 1/57.6, 1/57.7, 1/57.8, 1/57.9, 1/58, 1/58.1, 1/58.2, 1/58.3, 1/58.4, 1/58.5, 1/58.6, 1/58.7, 1/58.8, 1/58.9, 1/59, 1/59.1, 1/59.2, 1/59.3, 1/59.4, 1/59.5, 1/59.6, 1/59.7, 1/59.8, 1/59.9, 1/60 or ratios thereabout or therebetween. Ranges of ratios of 1/1 to 1/60 can be employed, and subranges as set forth below.

More preferred mosaicisms are:

1/4, 1/4.1, 1/4.2, 1/4.3, 1/4.4, 1/4.5, 1/4.6, 1/4.7, 1/4.8, 1/4.9, 1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, 1/12, 1/12.1, 1/12.2, 1/12.3, 1/12.4, 1/12.5, 1/12.6, 1/12.7, 1/12.8, 1/12.9 or 1/13.

Further preferred mosaicisms are:

1/5, 1/5.1, 1/5.2, 1/5.3, 1/5.4, 1/5.5, 1/5.6, 1/5.7, 1/5.8, 1/5.9, 1/6, 1/6.1, 1/6.2, 1/6.3, 1/6.4, 1/6.5, 1/6.6, 1/6.7, 1/6.8, 1/6.9, 1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, 1/11, 1/11.1, 1/11.2, 1/11.3, 1/11.4, 1/11.5, 1/11.6, 1/11.7, 1/11.8, 1/11.9, or 1/12.

Still more preferred mosaicisms are:

1/7, 1/7.1, 1/7.2, 1/7.3, 1/7.4, 1/7.5, 1/7.6, 1/7.7, 1/7.8, 1/7.9, 1/8, 1/8.1, 1/8.2, 1/8.3, 1/8.4, 1/8.5, 1/8.6, 1/8.7, 1/8.8, 1/8.9, 1/9, 1/9.1, 1/9.2, 1/9.3, 1/9.4, 1/9.5, 1/9.6, 1/9.7, 1/9.8, 1/9.9, 1/10, 1/10.1, 1/10.2, 1/10.3, 1/10.4, 1/10.5, 1/10.6, 1/10.7, 1/10.8, 1/10.9, or 1/11.

Preferred ranges of mosaicisms are 1/4 to 1/13; 1/5 to 1/12; 1/6 to 1/11, 1/7 to 1/10.5. or 1/7.5 to 1/10.

(6) Preferred molar ratios of AAV-FM to SCM-RM are as follows: 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300, 1:305, 1:310, 1:315, 1:320, 1:325, 1:330, 1:335, 1:340, 1:345, 1:350, 1:355, 1:360, 1:365, 1:370, 1:375, 1:380, 1:385, 1:390, 1:395, 1:400, 1:405, 1:410, 1:415, 1:420, 1:425, 1:430, 1:435, 1:440, 1:445, 1:450, 1:455, 1:460, 1:465, 1:470, 1:475, 1:480, 1:485, 1:490, 1:495, 1:500, 1:505, 1:510, 1:515, 1:520, 1:525, 1:530, 1:535, 1:540, 1:545, 1:550, 1:555, 1:560, 1:565, 1:570, 1:575, 1:580, 1:585, 1:590, 1:595, 1:600, 1:605, 1:610, 1:615, 1:620, 1:625, 1:630, 1:635, 1:640, 1:645, 1:650, 1:655, 1:660, 1:665, 1:670, 1:675, 1:680, 1:685, 1:690, 1:695, 1:700, 1:705, 1:710, 1:715, 1:720, 1:725, 1:730, 1:735, 1:740, 1:745, 1:750, 1:755, 1:760, 1:765, 1:770, 1:775, 1:780, 1:785, 1:790, 1:795, 1:800, 1:805, 1:810, 1:815, 1:820, 1:825, 1:830, 1:835, 1:840, 1:845, 1:850, 1:855, 1:860, 1:865, 1:870, 1:875, 1:880, 1:885, 1:890, 1:895, 1:900, 1:905, 1:910, 1:915, 1:920, 1:925, 1:930, 1:935, 1:940, 1:945, 1:950, 1:955, 1:960, 1:965, 1:970, 1:975, 1:980, 1:985, 1:990, 1:995, 1:1000, 1:>1000 or any ratio thereabout or therebetween.

More preferred ratios AAV-FM to SCM-RM are: 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200, 1:205, 1:210, 1:215, 1:220, 1:225, 1:230, 1:235, 1:240, 1:245, 1:250, 1:255, 1:260, 1:265, 1:270, 1:275, 1:280, 1:285, 1:290, 1:295, 1:300 or any ratio thereabout or therebetween.

Preferred ranges of AAV-FM to SCM-RM are 1:20 to 1:1000, and more preferred ranges are: 1:30 to 1:100, 1:40 to 1:100, 1:50 to 1:100 1:60 to 1:100, 1:70 to 1:100, 1:80 to 1:100, 1:90:1:100, 1:30 to 1:150, 1:40 to 1:150, 1:50 to 1:150 1:60 to 1:150, 1:70 to 1:150, 1:80 to 1:150, 1:90 to :150, 1:100 to 1:150, 1:30 to 1:200, 1:40 to 1:200, 1:50 to 1:200 1:60 to 1:200, 1:70 to 1:200, 1:80 to 1:200, 1:90:1:200, 1:100 to 1:200, 1:30 to 1:250, 1:40 to 1:250, 1:50 to 1:250 1:60 to 1:250, 1:70 to 1:250, 1:80 to 1:250, 1:90 to :250, 1:100 to 1:250, 1:30 to 1:300, 1:40 to 1:300, 1:50 to 1:300 1:60 to 1:300, 1:70 to 1:300, 1:80 to 1:300, 1:90:1:300, or 1:100 to 1:300.

(7) The inventions provide for a desired conjugation of a level of conjugation of at least 10%, at least 20%, at least 30%, and at least 40%. Preferred levels are at least 50%, at least 60%, at least 70%, at least 80%, at least 90%, or up to 100%, any of which can be considered complete depending on the purpose. More preferably, the desired level of conjugation typically will be about 40%, 41%, 42%, 43%, 44%, 45%, 46%, 47%, 48%, 49%, 50%, 51%, 52%, 53%, 54%, 55%, 56%, 57%, 58%, 59%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Ranges of desired conjugation levels can be 10% to 100%, 20% to 99%, 30% to 99%, 40% to 99%, 50% to 99%, 60% to 99%, 70% to 99%, 80% to 99%, or 90% to 99%. Preferred ranges of desired conjugation levels can be 40% to 100%, 40% to 99%, 45% to 95%, 50% to 95%, 55% to 90%, 60% to 90%, 65% to 95%, 65% to 85%, 65% to 90%, 70% to 90%, 75% to 90%, 75% to 85%, 75% to 80%, or 80% to 85%. More preferably, the range is 65% to 95%.

The second chart below cites to exemplary modifications to the above chart based upon the descriptions and sequence examples contained herein. Points 1-7 above also apply to the second chart below:

| Plasmid type | Specifics |
|---|---|
| pRC | AAV rh74 rep and cap genes. See, |
| | e.g., Example 32. |
| pRC-FM | See, e.g., Examples 34-36 to obtain |
| | VP1, VP2 and VP3 proteins fused to |
| | an FM (e.g., tag-type protein). |
| pHELP | See, e.g., Examples 33 and 37-39 |
| | concerning helper genes from |
| | herpes simplex virus (HSV), human |
| | papilloma virus (HPV), bocavirus, |
| | and baculovirus. |

### Sequence Examples

The following examples set forth exemplary sequences, which are optional for use and do not limit the inventions in any manner. Other AAV sequences, helper sequences, and Specific Binding Pair sequences are available and accessible. Gene of interest sequences and retargeting molecule sequences can be selected based upon the purpose of the covalently surface modified AAV and the cell/tissue to be targeted. Sequences that can be used according to the inventions are set for the below.

### Example 32—AAV Polynucleotide Sequences

AAV Rep, Cap and ITR sequences are known in the art. The present inventions are amenable to all AAV serotypes. AAV sequences from various AAV serotypes are set forth below. Many of these sequences are available from the National Center for Biotechnology Information (NCBI).

AAV-1
Full Genome: NC_002077
CapVP1: (SEQ ID NO: 1)
ATGGCTGCCGATGGTTATCTTCCAGATTGGCTCGAGGACAACCTCTCTGAGGGCATTCGCGAGTGGTGGGACTTG
AAACCTGGAGCCCCGAAGCCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAAAGAAACGTCCGGTAGAGCAGTCGCCA
CAAGAGCCAGACTCCTCCTCGGGCATCGGCAAGACAGGCCAGCAGCCCGCTAAAAAGAGACTCAATTTTGGTCAG
ACTGGCGACTCAGAGTCAGTCCCCGATCCACAACCTCTCGGAGAACCTCCAGCAACCCCCGCTGCTGTGGGACCT
ACTACAATGGCTTCAGGCGGTGGCGCACCAATGGCAGACAATAACGAAGGCGCCGACGGAGTGGGTAATGCCTCA
GGAAATTGGCATTGCGATTCCACATGGCTGGGCGACAGAGTCATCACCACCAGCACCCGCACCTGGGCCTTGCCC
ACCTACAATAACCACCTCTACAAGCAAATCTCCAGTGCTTCAACGGGGGCCAGCAACGACAACCACTACTTCGGC
TACAGCACCCCCTGGGGGTATTTTGATTTCAACAGATTCCACTGCCACTTTTCACCACGTGACTGGCAGCGACTC
ATCAACAACAATTGGGGATTCCGGCCCAAGAGACTCAACTTCAAACTCTTCAACATCCAAGTCAAGGAGGTCACG
ACGAATGATGGCGTCACAACCATCGCTAATAACCTTACCAGCACGGTTCAAGTCTTCTCGGACTCGGAGTACCAG
CTTCCGTACGTCCTCGGCTCTGCGCACCAGGGCTGCCTCCCTCCGTTCCCGGCGGACGTGTTCATGATTCCGCAA
TACGGCTACCTGACGCTCAACAATGGCAGCCAAGCCGTGGGACGTTCATCCTTTTACTGCCTGGAATATTTCCCT
TCTCAGATGCTGAGAACGGGCAACAACTTTACCTTCAGCTACACCTTTGAGGAAGTGCCTTTCCACAGCAGCTAC
GCGCACAGCCAGAGCCTGGACCGGCTGATGAATCCTCTCATCGACCAATACCTGTATTACCTGAACAGAACTCAA
AATCAGTCCGGAAGTGCCCAAAACAAGGACTTGCTGTTTAGCCGTGGGTCTCCAGCTGGCATGTCTGTTCAGCCC
AAAAACTGGCTACCTGGACCCTGTTATCGGCAGCAGCGCGTTTCTAAAACAAAAACAGACAACAACAACAGCAAT
TTTACCTGGACTGGTGCTTCAAAATATAACCTCAATGGGCGTGAATCCATCATCAACCCTGGCACTGCTATGGCC
TCACACAAAGACGACGAAGACAAGTTCTTTCCCATGAGCGGTGTCATGATTTTTGGAAAAGAGAGCGCCGGAGCT
TCAAACACTGCATTGGACAATGTCATGATTACAGACGAAGAGGAAATTAAAGCCACTAACCCTGTGGCCACCGAA
AGATTTGGGACCGTGGCAGTCAATTTCCAGAGCAGCAGCACAGACCCTGCGACCGGAGATGTGCATGCTATGGGA
GCATTACCTGGCATGGTGTGGCAAGATAGAGACGTGTACCTGCAGGGTCCCATTTGGGCCAAAATTCCTCACACA
GATGGACACTTTCACCCGTCTCCTCTTATGGGCGGCTTTGGACTCAAGAACCCGCCTCCTCAGATCCTCATCAAA
AACACGCCTGTTCCTGCGAATCCTCCGGCGGAGTTTTCAGCTACAAAGTTTGCTTCATTCATCACCCAATACTCC
ACAGGACAAGTGAGTGTGGAAATTGAATGGGAGCTGCAGAAAGAAAACAGCAAGCGCTGGAATCCCGAAGTGCAG
TACACATCCAATTATGCAAAATCTGCCAACGTTGATTTTACTGTGGACAACAATGGACTTTATACTGAGCCTCGC
CCCATTGGCACCCGTTACCTTACCCGTCCCCTGTAA
Rep78: (SEQ ID NO: 2)
ATGCCGGGCTTCTACGAGATCGTGATCAAGGTGCCGAGCGACCTGGACGAGCACCTGCCGGGCATTTCTGACTCG
TTTGTGAGCTGGGTGGCCGAGAAGGAATGGGAGCTGCCCCCGGATTCTGACATGGATCTGAATCTGATTGAGCAG
GCACCCCTGACCGTGGCCGAGAAGCTGCAGCGCGACTTCCTGGTCCAATGGCGCCGCGTGAGTAAGGCCCCGGAG
GCCCTCTTCTTTGTTCAGTTCGAGAAGGGCGAGTCCTACTTCCACCTCCATATTCTGGTGGAGACCACGGGGGTC
AAATCCATGGTGCTGGGCCGCTTCCTGAGTCAGATTAGGGACAAGCTGGTGCAGACCATCTACCGCGGGATCGAG
CCGACCCTGCCCAACTGGTTCGCGGTGACCAAGACGCGTAATGGCGCCGGAGGGGGGAACAAGGTGGTGGACGAG
TGCTACATCCCCAACTACCTCCTGCCCAAGACTCAGCCCGAGCTGCAGTGGGCGTGGACTAACATGGAGGAGTAT
ATAAGCGCCTGTTTGAACCTGGCCGAGCGCAAACGGCTCGTGGCGCAGCACCTGACCCACGTCAGCCAGACCCAG
GAGCAGAACAAGGAGAATCTGAACCCCAATTCTGACGCGCCTGTCATCCGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCTTCCAACTCGCGGTCCCAGATCAAGGCCGCTCTGGACAATGCCGGCAAGATCATGGCG
CTGACCAAATCCGCGCCCGACTACCTGGTAGGCCCCGCTCCGCCCGCGGACATTAAAACCAACCGCATCTACCGC
ATCCTGGAGCTGAACGGCTACGAACCTGCCTACGCCGGCTCCGTCTTTCTCGGCTGGGCCCAGAAAAGGTTCGGG
AAGCGCAACACCATCTGGCTGTTTGGGCCGGCCACCACGGGCAAGACCAACATCGCGGAAGCCATCGCCCACGCC
GTGCCCTTCTACGGCTGCGTCAACTGGACCAATGAGAACTTTCCCTTCAATGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGACCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCATGACTTTGGCAAGGTGACAAAGCAGGAAGTCAAAGAGTTCTTCCGCTGGGCGCAGGAT
CACGTGACCGAGGTGGCGCATGAGTTCTACGTCAGAAAGGGTGGAGCCAACAAAAGACCCGCCCCCGATGACGCG
GATAAAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGCTTCAGATGCTGTTTCCCTGCAAGACA
TGCGAGAGAATGAATCAGAATTTCAACATTTGCTTCACGCACGGGACGAGAGACTGTTCAGAGTGCTTCCCCGGC
GTGTCAGAATCTCAACCGGTCGTCAGAAAGAGGACGTATCGGAAACTCTGTGCCATTCATCATCTGCTGGGGCGG
GCTCCCGAGATTGCTTGCTCGGCCTGCGATCTGGTCAACGTGGACCTGGATGACTGTGTTTCTGAGCAATAA
AAV-2
Full Genome: NC_001401
Rep78: (SEQ ID NO: 3)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTTCTGACGGAATGGCGCCGTGTGAGTAAGGCCCCGGAG
GCCCTTTTCTTTGTGCAATTTGAGAAGGGAGAGAGCTACTTCCACATGCACGTGCTCGTGGAAACCACCGGGGTG
AAATCCATGGTTTTGGGACGTTTCCTGAGTCAGATTCGCGAAAAACTGATTCAGAGAATTTACCGCGGGATCGAG
CCGACTTTGCCAAACTGGTTCGCGGTCACAAAGACCAGAAATGGCGCCGGAGGCGGGAACAAGGTGGTGGATGAG
TGCTACATCCCCAATTACTTGCTCCCCAAAACCCAGCCTGAGCTCCAGTGGGCGTGGACTAATATGGAACAGTAT
TTAAGCGCCTGTTTGAATCTCACGGAGCGTAAACGGTTGGTGGCGCAGCATCTGACGCACGTGTCGCAGACGCAG
GAGCAGAACAAAGAGAATCAGAATCCCAATTCTGATGCGCCGGTGATCAGATCAAAAACTTCAGCCAGGTACATG
GAGCTGGTCGGGTGGCTCGTGGACAAGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTTGGACAATGCGGGAAAGATTATGAGC
CTGACTAAAACCGCCCCCGACTACCTGGTGGGCCAGCAGCCCGTGGAGGACATTTCCAGCAATCGGATTTATAAA
ATTTTGGAACTAAACGGGTACGATCCCCAATATGCGGCTTCCGTCTTTCTGGGATGGGCCACGAAAAAGTTCGGC
AAGAGGAACACCATCTGGCTGTTTGGGCCTGCAACTACCGGGAAGACCAACATCGCGGAGGCCATAGCCCACACT
GTGCCCTTCTACGGGTGCGTAAACTGGACCAATGAGAACTTTCCCTTCAACGACTGTGTCGACAAGATGGTGATC
TGGTGGGAGGAGGGGAAGATGACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCAAATTTGAACTC
ACCCGCCGTCTGGATCATGACTTTGGGAAGGTCACCAAGCAGGAAGTCAAAGACTTTTTCCGGTGGGCAAAGGAT
CACGTGGTTGAGGTGGAGCATGAATTCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATGGGAAAGGTG
CCAGACGCTTGCACTGCCTGCGATCTGGTCAATGTGGATTTGGATGACTGCATCTTTGAACAATAA
Rep52: (SEQ ID NO: 4)
ATGGAGCTGGTCGGGTGGCTCGTGGACAAGGGGATTACCTCGGAGAAGCAGTGGATCCAGGAGGACCAGGCCTCA
TACATCTCCTTCAATGCGGCCTCCAACTCGCGGTCCCAAATCAAGGCTGCCTTGGACAATGCGGGAAAGATTATG
AGCCTGACTAAAACCGCCCCCGACTACCTGGTGGGCCAGCAGCCCGTGGAGGACATTTCCAGCAATCGGATTTAT
AAAATTTTGGAACTAAACGGGTACGATCCCCAATATGCGGCTTCCGTCTTTCTGGGATGGGCCACGAAAAAGTTC
GGCAAGAGGAACACCATCTGGCTGTTTGGGCCTGCAACTACCGGGAAGACCAACATCGCGGAGGCCATAGCCCAC
ACTGTGCCCTTCTACGGGTGCGTAAACTGGACCAATGAGAACTTTCCCTTCAACGACTGTGTCGACAAGATGGTG
ATCTGGTGGGAGGAGGGGAAGATGACCGCCAAGGTCGTGGAGTCGGCCAAAGCCATTCTCGGAGGAAGCAAGGTG
CGCGTGGACCAGAAATGCAAGTCCTCGGCCCAGATAGACCCGACTCCCGTGATCGTCACCTCCAACACCAACATG
TGCGCCGTGATTGACGGGAACTCAACGACCTTCGAACACCAGCAGCCGTTGCAAGACCGGATGTTCAAATTTGAA
CTCACCCGCCGTCTGGATCATGACTTTGGGAAGGTCACCAAGCAGGAAGTCAAAGACTTTTTCCGGTGGGCAAAG
GATCACGTGGTTGAGGTGGAGCATGAATTCTACGTCAAAAAGGGTGGAGCCAAGAAAAGACCCGCCCCCAGTGAC
GCAGATATAAGTGAGCCCAAACGGGTGCGCGAGTCAGTTGCGCAGCCATCGACGTCAGACGCGGAAGCTTCGATC
AACTACGCAGACAGGTACCAAAACAAATGTTCTCGTCACGTGGGCATGAATCTGATGCTGTTTCCCTGCAGACAA
TGCGAGAGAATGAATCAGAATTCAAATATCTGCTTCACTCACGGACAGAAAGACTGTTTAGAGTGCTTTCCCGTG
TCAGAATCTCAACCCGTTTCTGTCGTCAAAAAGGCGTATCAGAAACTGTGCTACATTCATCATATCATGGGAAAG
GTGCCAGACGCTTGCACTGCCTGCGATCTGGTCAATGTGGATTTGGATGACTGCATCTTTGAACAATAA
CapVP1: (SEQ ID NO: 5)
ATGGCTGCCGATGGTTATCTTCCAGATTGGCTCGAGGACACTCTCTCTGAAGGAATAAGACAGTGGTGGAAGCTC
AAACCTGGCCCACCACCACCAAAGCCCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCGACGCGGAGTTT
CAGGAGCGCCTTAAAGAAGATACGTCTTTTGGGGGCAACCTCGGACGAGCAGTCTTCCAGGCGAAAAAGAGGGTT
CTTGAACCTCTGGGCCTGGTTGAGGAACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGCAAGAAAAAGATTGAATTTTGGTCAG
ACTGGAGACGCAGACTCAGTACCTGACCCCCAGCCTCTCGGACAGCCACCAGCAGCCCCCTCTGGTCTGGGAACT
AATACGATGGCTACAGGCAGTGGCGCACCAATGGCAGACAATAACGAGGGCGCCGACGGAGTGGGTAATTCCTCG
GGAAATTGGCATTGCGATTCCACATGGATGGGCGACAGAGTCATCACCACCAGCACCCGAACCTGGGCCCTGCCC
ACCTACAACAACCACCTCTACAAACAAATTTCCAGCCAATCAGGAGCCTCGAACGACAATCACTACTTTGGCTAC
AGCACCCCTTGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACTTTCCTTCT
CAGATGCTGCGTACCGGAAACAACTTTACCTTCAGCTACACTTTTGAGGACGTTCCTTTCCACAGCAGCTACGCT
CACAGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCTGCGGATAACAACAACAGTGAATAC
TCGTGGACTGGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCAGAGAAAACA
AATGTGGACATTGAAAAGGTCATGATTACAGACGAAGAGGAAATCAGGACAACCAATCCCGTGGCTACGGAGCAG
TATGGTTCTGTATCTACCAACCTCCAGAGAGGCAACAGACAAGCAGCTACCGCAGATGTCAACACACAAGGCGTT
CTTCCAGGCATGGTCTGGCAGGACAGAGATGTGTACCTTCAGGGGCCCATCTGGGCAAAGATTCCACACACGGAC
GGACATTTTCACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACAGTACTCCACG
GGACAGGTCAGCGTGGAGATCGAGTGGGAGCTGCAGAAGGAAAACAGCAAACGCTGGAATCCCGAAATTCAGTAC
ACTTCCAACTACAACAAGTCTGTTAATGTGGACTTTACTGTGGACACTAATGGCGTGTATTCAGAGCCTCGCCCC
ATTGGCACCAGATACCTGACTCGTAATCTGTAA
CapVP2: (SEQ ID NO: 6)
ACGGCTCCGGGAAAAAAGAGGCCGGTAGAGCACTCTCCTGTGGAGCCAGACTCCTCCTCGGGAACCGGAAAGGCG
GGCCAGCAGCCTGCAAGAAAAAGATTGAATTTTGGTCAGACTGGAGACGCAGACTCAGTACCTGACCCCCAGCCT
CTCGGACAGCCACCAGCAGCCCCCTCTGGTCTGGGAACTAATACGATGGCTACAGGCAGTGGCGCACCAATGGCA
GACAATAACGAGGGCGCCGACGGAGTGGGTAATTCCTCGGGAAATTGGCATTGCGATTCCACATGGATGGGCGAC
AGAGTCATCACCACCAGCACCCGAACCTGGGCCCTGCCCACCTACAACAACCACCTCTACAAACAAATTTCCAGC
CAATCAGGAGCCTCGAACGACAATCACTACTTTGGCTACAGCACCCCTTGGGGGTATTTTGACTTCAACAGATTC
CACTGCCACTTTTCACCACGTGACTGGCAAAGACTCATCAACAACAACTGGGGATTCCGACCCAAGAGACTCAAC
TTCAAGCTCTTTAACATTCAAGTCAAAGAGGTCACGCAGAATGACGGTACGACGACGATTGCCAATAACCTTACC
AGCACGGTTCAGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACCGGAAACAACTTTACCTTCAGC
TACACTTTTGAGGACGTTCCTTTCCACAGCAGCTACGCTCACAGCCAGAGTCTGGACCGTCTCATGAATCCTCTC
ATCGACCAGTACCTGTATTACTTGAGCAGAACAAACACTCCAAGTGGAACCACCACGCAGTCAAGGCTTCAGTTT
TCTCAGGCCGGAGCGAGTGACATTCGGGACCAGTCTAGGAACTGGCTTCCTGGACCCTGTTACCGCCAGCAGCGA
GTATCAAAGACATCTGCGGATAACAACAACAGTGAATACTCGTGGACTGGAGCTACCAAGTACCACCTCAATGGC
AGAGACTCTCTGGTGAATCCGGGCCCGGCCATGGCAAGCCACAAGGACGATGAAGAAAAGTTTTTTCCTCAGAGC
GGGGTTCTCATCTTTGGGAAGCAAGGCTCAGAGAAAACAAATGTGGACATTGAAAAGGTCATGATTACAGACGAA
GAGGAAATCAGGACAACCAATCCCGTGGCTACGGAGCAGTATGGTTCTGTATCTACCAACCTCCAGAGAGGCAAC
AGACAAGCAGCTACCGCAGATGTCAACACACAAGGCGTTCTTCCAGGCATGGTCTGGCAGGACAGAGATGTGTAC
CTTCAGGGGCCCATCTGGGCAAAGATTCCACACACGGACGGACATTTTCACCCCTCTCCCCTCATGGGTGGATTC
GGACTTAAACACCCTCCTCCACAGATTCTCATCAAGAACACCCCGGTACCTGCGAATCCTTCGACCACCTTCAGT
GCGGCAAAGTTTGCTTCCTTCATCACACAGTACTCCACGGGACAGGTCAGCGTGGAGATCGAGTGGGAGCTGCAG
AAGGAAAACAGCAAACGCTGGAATCCCGAAATTCAGTACACTTCCAACTACAACAAGTCTGTTAATGTGGACTTT
ACTGTGGACACTAATGGCGTGTATTCAGAGCCTCGCCCCATTGGCACCAGATACCTGACTCGTAATCTGTAA
CapVP3: (SEQ ID NO: 7)
ATGGCTACAGGCAGTGGCGCACCAATGGCAGACAATAACGAGGGCGCCGACGGAGTGGGTAATTCCTCGGGAAAT
TGGCATTGCGATTCCACATGGATGGGCGACAGAGTCATCACCACCAGCACCCGAACCTGGGCCCTGCCCACCTAC
AACAACCACCTCTACAAACAAATTTCCAGCCAATCAGGAGCCTCGAACGACAATCACTACTTTGGCTACAGCACC
CCTTGGGGGTATTTTGACTTCAACAGATTCCACTGCCACTTTTCACCACGTGACTGGCAAAGACTCATCAACAAC
AACTGGGGATTCCGACCCAAGAGACTCAACTTCAAGCTCTTTAACATTCAAGTCAAAGAGGTCACGCAGAATGAC
GGTACGACGACGATTGCCAATAACCTTACCAGCACGGTTCAGGTGTTTACTGACTCGGAGTACCAGCTCCCGTAC
GTCCTCGGCTCGGCGCATCAAGGATGCCTCCCGCCGTTCCCAGCAGACGTCTTCATGGTGCCACAGTATGGATAC
CTCACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAGAACAAACACTCCAAGT
GGAACCACCACGCAGTCAAGGCTTCAGTTTTCTCAGGCCGGAGCGAGTGACATTCGGGACCAGTCTAGGAACTGG
CTTCCTGGACCCTGTTACCGCCAGCAGCGAGTATCAAAGACATCTGCGGATAACAACAACAGTGAATACTCGTGG
ACTGGAGCTACCAAGTACCACCTCAATGGCAGAGACTCTCTGGTGAATCCGGGCCCGGCCATGGCAAGCCACAAG
GACGATGAAGAAAAGTTTTTTCCTCAGAGCGGGGTTCTCATCTTTGGGAAGCAAGGCTCAGAGAAAACAAATGTG
GACATTGAAAAGGTCATGATTACAGACGAAGAGGAAATCAGGACAACCAATCCCGTGGCTACGGAGCAGTATGGT
TCTGTATCTACCAACCTCCAGAGAGGCAACAGACAAGCAGCTACCGCAGATGTCAACACACAAGGCGTTCTTCCA
GGCATGGTCTGGCAGGACAGAGATGTGTACCTTCAGGGGCCCATCTGGGCAAAGATTCCACACACGGACGGACAT
TTTCACCCCTCTCCCCTCATGGGTGGATTCGGACTTAAACACCCTCCTCCACAGATTCTCATCAAGAACACCCCG
GTACCTGCGAATCCTTCGACCACCTTCAGTGCGGCAAAGTTTGCTTCCTTCATCACACAGTACTCCACGGGACAG
GTCAGCGTGGAGATCGAGTGGGAGCTGCAGAAGGAAAACAGCAAACGCTGGAATCCCGAAATTCAGTACACTTCC
AACTACAACAAGTCTGTTAATGTGGACTTTACTGTGGACACTAATGGCGTGTATTCAGAGCCTCGCCCCATTGGC
ACCAGATACCTGACTCGTAATCTGTAA
CapAAP: (SEQ ID NO: 8)
CTGGAGACGCAGACTCAGTACCTGACCCCCAGCCTCTCGGACAGCCACCAGCAGCCCCCTCTGGTCTGGGAACTA
ATACGATGGCTACAGGCAGTGGCGCACCAATGGCAGACAATAACGAGGGCGCCGACGGAGTGGGTAATTCCTCGG
GAAATTGGCATTGCGATTCCACATGGATGGGCGACAGAGTCATCACCACCAGCACCCGAACCTGGGCCCTGCCCA
CCTACAACAACCACCTCTACAAACAAATTTCCAGCCAATCAGGAGCCTCGAACGACAATCACTACTTTGGCTACA
GCACCCCTTGGGGGTATTTTGACTTCAACAGATTCCACTGCCACTTTTCACCACGTGACTGGCAAAGACTCATCA
ACAACAACTGGGGATTCCGACCCAAGAGACTCAACTTCAAGCTCTTTAACATTCAAGTCAAAGAGGTCACGCAGA
ATGACGGTACGACGACGATTGCCAATAACCTTACCAGCACGGTTCAGGTGTTTACTGACTCGGAGTACCAGCTCC
CGTACGTCCTCGGCTCGGCGCATCAAGGATGCCTCCCGCCGTTCCCAGCAGACGTCTTCATGGTGCCACAGTATG
GATACCTCACCCTGA
AAV-3
Full Genome: NC_001729
Rep78: (SEQ ID NO: 9)
ATGCCGGGGTTCTACGAGATTGTCCTGAAGGTCCCGAGTGACCTGGACGAGCGCCTGCCGGGCATTTCTAACTCG
TTTGTTAACTGGGTGGCCGAGAAGGAATGGGACGTGCCGCCGGATTCTGACATGGATCCGAATCTGATTGAGCAG
GCACCCCTGACCGTGGCCGAAAAGCTTCAGCGCGAGTTCCTGGTGGAGTGGCGCCGCGTGAGTAAGGCCCCGGAG
GCCCTCTTTTTTGTCCAGTTCGAAAAGGGGGAGACCTACTTCCACCTGCACGTGCTGATTGAGACCATCGGGGTC
AAATCCATGGTGGTCGGCCGCTACGTGAGCCAGATTAAAGAGAAGCTGGTGACCCGCATCTACCGCGGGGTCGAG
CCGCAGCTTCCGAACTGGTTCGCGGTGACCAAAACGCGAAATGGCGCCGGGGGCGGGAACAAGGTGGTGGACGAC
TGCTACATCCCCAACTACCTGCTCCCCAAGACCCAGCCCGAGCTCCAGTGGGCGTGGACTAACATGGACCAGTAT
TTAAGCGCCTGTTTGAATCTCGCGGAGCGTAAACGGCTGGTGGCGCAGCATCTGACGCACGTGTCGCAGACGCAG
GAGCAGAACAAAGAGAATCAGAACCCCAATTCTGACGCGCCGGTCATCAGGTCAAAAACCTCAGCCAGGTACATG
GAGCTGGTCGGGTGGCTGGTGGACCGCGGGATCACGTCAGAAAAGCAATGGATTCAGGAGGACCAGGCCTCGTAC
ATCTCCTTCAACGCCGCCTCCAACTCGCGGTCCCAGATCAAGGCCGCGCTGGACAATGCCTCCAAGATCATGAGC
CTGACAAAGACGGCTCCGGACTACCTGGTGGGCAGCAACCCGCCGGAGGACATTACCAAAAATCGGATCTACCAA
ATCCTGGAGCTGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCGGAAGCCATCGCCCACGCC
GTGCCCTTCTACGGCTGCGTAAACTGGACCAATGAGAACTTTCCCTTCAACGATTGCGTCGACAAGATGGTGATC
TGGTGGGAGGAGGGCAAGATGACGGCCAAGGTCGTGGAGAGCGCCAAGGCCATTCTGGGCGGAAGCAAGGTGCGC
GTGGACCAAAAGTGCAAGTCATCGGCCCAGATCGAACCCACTCCCGTGATCGTCACCTCCAACACCAACATGTGC
GCCGTGATTGACGGGAACAGCACCACCTTCGAGCATCAGCAGCCGCTGCAGGACCGGATGTTTGAATTTGAACTT
ACCCGCCGTTTGGACCATGACTTTGGGAAGGTCACCAAACAGGAAGTAAAGGACTTTTTCCGGTGGGCTTCCGAT
CACGTGACTGACGTGGCTCATGAGTTCTACGTCAGAAAGGGTGGAGCTAAGAAACGCCCCGCCTCCAATGACGCG
GATGTAAGCGAGCCAAAACGGGAGTGCACGTCACTTGCGCAGCCGACAACGTCAGACGCGGAAGCACCGGCGGAC
TACGCGGACAGGTACCAAAACAAATGTTCTCGTCACGTGGGCATGAATCTGATGCTTTTTCCCTGTAAAACATGC
GAGAGAATGAATCAAATTTCCAATGTCTGTTTTACGCATGGTCAAAGAGACTGTGGGGAATGCTTCCCTGGAATG
TCAGAATCTCAACCCGTTTCTGTCGTCAAAAAGAAGACTTATCAGAAACTGTGTCCAATTCATCATATCCTGGGA
AGGGCACCCGAGATTGCCTGTTCGGCCTGCGATTTGGCCAATGTGGACTTGGATGACTGTGTTTCTGAGCAATAA
CapVP1: (SEQ ID NO: 10)
ATGGCTGCTGACGGTTATCTTCCAGATTGGCTCGAGGACAACCTTTCTGAAGGCATTCGTGAGTGGTGGGCTCTG
AAACCTGGAGTCCCTCAACCCAAAGCGAACCAACAACACCAGGACAACCGTCGGGGTCTTGTGCTTCCGGGTTAC
AAATACCTCGGACCCGGTAACGGACTCGACAAAGGAGAGCCGGTCAACGAGGCGGACGCGGCAGCCCTCGAACAC
GACAAAGCTTACGACCAGCAGCTCAAGGCCGGTGACAACCCGTACCTCAAGTACAACCACGCCGACGCCGAGTTT
CAGGAGCGTCTTCAAGAAGATACGTCTTTTGGGGGCAACCTTGGCAGAGCAGTCTTCCAGGCCAAAAAGAGGATC
CTTGAGCCTCTTGGTCTGGTTGAGGAAGCAGCTAAAACGGCTCCTGGAAAGAAGGGGGCTGTAGATCAGTCTCCT
CAGGAACCGGACTCATCATCTGGTGTTGGCAAATCGGGCAAACAGCCTGCCAGAAAAAGACTAAATTTCGGTCAG
ACTGGAGACTCAGAGTCAGTCCCAGACCCTCAACCTCTCGGAGAACCACCAGCAGCCCCCACAAGTTTGGGATCT
AATACAATGGCTTCAGGCGGTGGCGCACCAATGGCAGACAATAACGAGGGTGCCGATGGAGTGGGTAATTCCTCA
GGAAATTGGCATTGCGATTCCCAATGGCTGGGCGACAGAGTCATCACCACCAGCACCAGAACCTGGGCCCTGCCC
ACTTACAACAACCATCTCTACAAGCAAATCTCCAGCCAATCAGGAGCTTCAAACGACAACCACTACTTTGGCTAC
AGCACCCCTTGGGGGTATTTTGACTTTAACAGATTCCACTGCCACTTCTCACCACGTGACTGGCAGCGACTCATT
AACAACAACTGGGGATTCCGGCCCAAGAAACTCAGCTTCAAGCTCTTCAACATCCAAGTTAGAGGGGTCACGCAG
AACGATGGCACGACGACTATTGCCAATAACCTTACCAGCACGGTTCAAGTGTTTACGGACTCGGAGTATCAGCTC
CCGTACGTGCTCGGGTCGGCGCACCAAGGCTGTCTCCCGCCGTTTCCAGCGGACGTCTTCATGGTCCCTCAGTAT
GGATACCTCACCCTGAACAACGGAAGTCAAGCGGTGGGACGCTCATCCTTTTACTGCCTGGAGTACTTCCCTTCG
CAGATGCTAAGGACTGGAAATAACTTCCAATTCAGCTATACCTTCGAGGATGTACCTTTTCACAGCAGCTACGCT
CACAGCCAGAGTTTGGATCGCTTGATGAATCCTCTTATTGATCAGTATCTGTACTACCTGAACAGAACGCAAGGA
ACAACCTCTGGAACAACCAACCAATCACGGCTGCTTTTTAGCCAGGCTGGGCCTCAGTCTATGTCTTTGCAGGCC
AGAAATTGGCTACCTGGGCCCTGCTACCGGCAACAGAGACTTTCAAAGACTGCTAACGACAACAACAACAGTAAC
TTTCCTTGGACAGCGGCCAGCAAATATCATCTCAATGGCCGCGACTCGCTGGTGAATCCAGGACCAGCTATGGCC
AGTCACAAGGACGATGAAGAAAAATTTTTCCCTATGCACGGCAATCTAATATTTGGCAAAGAAGGGACAACGGCA
AGTAACGCAGAATTAGATAATGTAATGATTACGGATGAAGAAGAGATTCGTACCACCAATCCTGTGGCAACAGAG
CAGTATGGAACTGTGGCAAATAACTTGCAGAGCTCAAATACAGCTCCCACGACTGGAACTGTCAATCATCAGGGG
GCCTTACCTGGCATGGTGTGGCAAGATCGTGACGTGTACCTTCAAGGACCTATCTGGGCAAAGATTCCTCACACG
GATGGACACTTTCATCCTTCTCCTCTGATGGGAGGCTTTGGACTGAAACATCCGCCTCCTCAAATCATGATCAAA
AATACTCCGGTACCGGCAAATCCTCCGACGACTTTCAGCCCGGCCAAGTTTGCTTCATTTATCACTCAGTACTCC
ACTGGACAGGTCAGCGTGGAAATTGAGTGGGAGCTACAGAAAGAAAACAGCAAACGTTGGAATCCAGAGATTCAG
TACACTTCCAACTACAACAAGTCTGTTAATGTGGACTTTACTGTAGACACTAATGGTGTTTATAGTGAACCTCGC
CCTATTGGAACCCGGTATCTCACACGAAACTTGTGA
AAV-4
Full Genome: NC_001829
Rep78: (SEQ ID NO: 11)
ATGCCGGGGTTCTACGAGATCGTGCTGAAGGTGCCCAGCGACCTGGACGAGCACCTGCCCGGCATTTCTGACTCT
TTTGTGAGCTGGGTGGCCGAGAAGGAATGGGAGCTGCCGCCGGATTCTGACATGGACTTGAATCTGATTGAGCAG
GCACCCCTGACCGTGGCCGAAAAGCTGCAACGCGAGTTCCTGGTCGAGTGGCGCCGCGTGAGTAAGGCCCCGGAG
GCCCTCTTCTTTGTCCAGTTCGAGAAGGGGGACAGCTACTTCCACCTGCACATCCTGGTGGAGACCGTGGGCGTC
AAATCCATGGTGGTGGGCCGCTACGTGAGCCAGATTAAAGAGAAGCTGGTGACCCGCATCTACCGCGGGGTCGAG
CCGCAGCTTCCGAACTGGTTCGCGGTGACCAAGACGCGTAATGGCGCCGGAGGCGGGAACAAGGTGGTGGACGAC
TGCTACATCCCCAACTACCTGCTCCCCAAGACCCAGCCCGAGCTCCAGTGGGCGTGGACTAACATGGACCAGTAT
ATAAGCGCCTGTTTGAATCTCGCGGAGCGTAAACGGCTGGTGGCGCAGCATCTGACGCACGTGTCGCAGACGCAG
GAGCAGAACAAGGAAAACCAGAACCCCAATTCTGACGCGCCGGTCATCAGGTCAAAAACCTCCGCCAGGTACATG
GAGCTGGTCGGGTGGCTGGTGGACCGCGGGATCACGTCAGAAAAGCAATGGATCCAGGAGGACCAGGCGTCCTAC
ATCTCCTTCAACGCCGCCTCCAACTCGCGGTCACAAATCAAGGCCGCGCTGGACAATGCCTCCAAAATCATGAGC
CTGACAAAGACGGCTCCGGACTACCTGGTGGGCCAGAACCCGCCGGAGGACATTTCCAGCAACCGCATCTACCGA
ATCCTCGAGATGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCGGAAGCCATCGCCCACGCC
GTGCCCTTCTACGGCTGCGTGAACTGGACCAATGAGAACTTTCCGTTCAACGATTGCGTCGACAAGATGGTGATC
TGGTGGGAGGAGGGCAAGATGACGGCCAAGGTCGTAGAGAGCGCCAAGGCCATCCTGGGCGGAAGCAAGGTGCGC
GTGGACCAAAAGTGCAAGTCATCGGCCCAGATCGACCCAACTCCCGTGATCGTCACCTCCAACACCAACATGTGC
GCGGTCATCGACGGAAACTCGACCACCTTCGAGCACCAACAACCACTCCAGGACCGGATGTTCAAGTTCGAGCTC
ACCAAGCGCCTGGAGCACGACTTTGGCAAGGTCACCAAGCAGGAAGTCAAAGACTTTTTCCGGTGGGCGTCAGAT
CACGTGACCGAGGTGACTCACGAGTTTTACGTCAGAAAGGGTGGAGCTAGAAAGAGGCCCGCCCCCAATGACGCA
GATATAAGTGAGCCCAAGCGGGCCTGTCCGTCAGTTGCGCAGCCATCGACGTCAGACGCGGAAGCTCCGGTGGAC
TACGCGGACAGGTACCAAAACAAATGTTCTCGTCACGTGGGTATGAATCTGATGCTTTTTCCCTGCCGGCAATGC
GAGAGAATGAATCAGAATGTGGACATTTGCTTCACGCACGGGGTCATGGACTGTGCCGAGTGCTTCCCCGTGTCA
GAATCTCAACCCGTGTCTGTCGTCAGAAAGCGGACGTATCAGAAACTGTGTCCGATTCATCACATCATGGGGAGG
GCGCCCGAGGTGGCCTGCTCGGCCTGCGAACTGGCCAATGTGGACTTGGATGACTGTGACATGGAACAATAA
CapVP1: (SEQ ID NO: 12)
ATGACTGACGGTTACCTTCCAGATTGGCTAGAGGACAACCTCTCTGAAGGCGTTCGAGAGTGGTGGGCGCTGCAA
CCTGGAGCCCCTAAACCCAAGGCAAATCAACAACATCAGGACAACGCTCGGGGTCTTGTGCTTCCGGGTTACAAA
TACCTCGGACCCGGCAACGGACTCGACAAGGGGGAACCCGTCAACGCAGCGGACGCGGCAGCCCTCGAGCACGAC
AAGGCCTACGACCAGCAGCTCAAGGCCGGTGACAACCCCTACCTCAAGTACAACCACGCCGACGCGGAGTTCCAG
CAGCGGCTTCAGGGCGACACATCGTTTGGGGGCAACCTCGGCAGAGCAGTCTTCCAGGCCAAAAAGAGGGTTCTT
GAACCTCTTGGTCTGGTTGAGCAAGCGGGTGAGACGGCTCCTGGAAAGAAGAGACCGTTGATTGAATCCCCCCAG
CAGCCCGACTCCTCCACGGGTATCGGCAAAAAAGGCAAGCAGCCGGCTAAAAAGAAGCTCGTTTTCGAAGACGAA
ACTGGAGCAGGCGACGGACCCCCTGAGGGATCAACTTCCGGAGCCATGTCTGATGACAGTGAGATGCGTGCAGCA
GCTGGCGGAGCTGCAGTCGAGGGCGGACAAGGTGCCGATGGAGTGGGTAATGCCTCGGGTGATTGGCATTGCGAT
TCCACCTGGTCTGAGGGCCACGTCACGACCACCAGCACCAGAACCTGGGTCTTGCCCACCTACAACAACCACCTC
TACAAGCGACTCGGAGAGAGCCTGCAGTCCAACACCTACAACGGATTCTCCACCCCCTGGGGATACTTTGACTTC
AACCGCTTCCACTGCCACTTCTCACCACGTGACTGGCAGCGACTCATCAACAACAACTGGGGCATGCGACCCAAA
GCCATGCGGGTCAAAATCTTCAACATCCAGGTCAAGGAGGTCACGACGTCGAACGGCGAGACAACGGTGGCTAAT
AACCTTACCAGCACGGTTCAGATCTTTGCGGACTCGTCGTACGAACTGCCGTACGTGATGGATGCGGGTCAAGAG
GGCAGCCTGCCTCCTTTTCCCAACGACGTCTTTATGGTGCCCCAGTACGGCTACTGTGGACTGGTGACCGGCAAC
ACTTCGCAGCAACAGACTGACAGAAATGCCTTCTACTGCCTGGAGTACTTTCCTTCGCAGATGCTGCGGACTGGC
AACAACTTTGAAATTACGTACAGTTTTGAGAAGGTGCCTTTCCACTCGATGTACGCGCACAGCCAGAGCCTGGAC
CGGCTGATGAACCCTCTCATCGACCAGTACCTGTGGGGACTGCAATCGACCACCACCGGAACCACCCTGAATGCC
GGGACTGCCACCACCAACTTTACCAAGCTGCGGCCTACCAACTTTTCCAACTTTAAAAAGAACTGGCTGCCCGGG
CCTTCAATCAAGCAGCAGGGCTTCTCAAAGACTGCCAATCAAAACTACAAGATCCCTGCCACCGGGTCAGACAGT
CTCATCAAATACGAGACGCACAGCACTCTGGACGGAAGATGGAGTGCCCTGACCCCCGGACCTCCAATGGCCACG
GCTGGACCTGCGGACAGCAAGTTCAGCAACAGCCAGCTCATCTTTGCGGGGCCTAAACAGAACGGCAACACGGCC
ACCGTACCCGGGACTCTGATCTTCACCTCTGAGGAGGAGCTGGCAGCCACCAACGCCACCGATACGGACATGTGG
GGCAACCTACCTGGCGGTGACCAGAGCAACAGCAACCTGCCGACCGTGGACAGACTGACAGCCTTGGGAGCCGTG
CCTGGAATGGTCTGGCAAAACAGAGACATTTACTACCAGGGTCCCATTTGGGCCAAGATTCCTCATACCGATGGA
CACTTTCACCCCTCACCGCTGATTGGTGGGTTTGGGCTGAAACACCCGCCTCCTCAAATTTTTATCAAGAACACC
CCGGTACCTGCGAATCCTGCAACGACCTTCAGCTCTACTCCGGTAAACTCCTTCATTACTCAGTACAGCACTGGC
CAGGTGTCGGTGCAGATTGACTGGGAGATCCAGAAGGAGCGGTCCAAACGCTGGAACCCCGAGGTCCAGTTTACC
TCCAACTACGGACAGCAAAACTCTCTGTTGTGGGCTCCCGATGCGGCTGGGAAATACACTGAGCCTAGGGCTATC
GGTACCCGCTACCTCACCCACCACCTGTAA
AAV-5
Full Genome: NC_006152
Rep78: (SEQ ID NO: 13)
ATGGCTACCTTCTATGAAGTCATTGTTCGCGTCCCATTTGACGTGGAGGAACATCTGCCTGGAATTTCTGACAGC
TTTGTGGACTGGGTAACTGGTCAAATTTGGGAGCTGCCTCCAGAGTCAGATTTAAATTTGACTCTGGTTGAACAG
CCTCAGTTGACGGTGGCTGATAGAATTCGCCGCGTGTTCCTGTACGAGTGGAACAAATTTTCCAAGCAGGAGTCC
AAATTCTTTGTGCAGTTTGAAAAGGGATCTGAATATTTTCATCTGCACACGCTTGTGGAGACCTCCGGCATCTCT
TCCATGGTCCTCGGCCGCTACGTGAGTCAGATTCGCGCCCAGCTGGTGAAAGTGGTCTTCCAGGGAATTGAACCC
CAGATCAACGACTGGGTCGCCATCACCAAGGTAAAGAAGGGCGGAGCCAATAAGGTGGTGGATTCTGGGTATATT
CCCGCCTACCTGCTGCCGAAGGTCCAACCGGAGCTTCAGTGGGCGTGGACAAACCTGGACGAGTATAAATTGGCC
GCCCTGAATCTGGAGGAGCGCAAACGGCTCGTCGCGCAGTTTCTGGCAGAATCCTCGCAGCGCTCGCAGGAGGCG
GCTTCGCAGCGTGAGTTCTCGGCTGACCCGGTCATCAAAAGCAAGACTTCCCAGAAATACATGGCGCTCGTCAAC
TGGCTCGTGGAGCACGGCATCACTTCCGAGAAGCAGTGGATCCAGGAAAATCAGGAGAGCTACCTCTCCTTCAAC
TCCACCGGCAACTCTCGGAGCCAGATCAAGGCCGCGCTCGACAACGCGACCAAAATTATGAGTCTGACAAAAAGC
GCGGTGGACTACCTCGTGGGGAGCTCCGTTCCCGAGGACATTTCAAAAAACAGAATCTGGCAAATTTTTGAGATG
AATGGCTACGACCCGGCCTACGCGGGATCCATCCTCTACGGCTGGTGTCAGCGCTCCTTCAACAAGAGGAACACC
GTCTGGCTCTACGGACCCGCCACGACCGGCAAGACCAACATCGCGGAGGCCATCGCCCACACTGTGCCCTTTTAC
GGCTGCGTGAACTGGACCAATGAAAACTTTCCCTTTAATGACTGTGTGGACAAAATGCTCATTTGGTGGGAGGAG
GGAAAGATGACCAACAAGGTGGTTGAATCCGCCAAGGCCATCCTGGGGGGCTCAAAGGTGCGGGTCGATCAGAAA
TGTAAATCCTCTGTTCAAATTGATTCTACCCCTGTCATTGTAACTTCCAATACAAACATGTGTGTGGTGGTGGAT
GGGAATTCCACGACCTTTGAACACCAGCAGCCGCTGGAGGACCGCATGTTCAAATTTGAACTGACTAAGCGGCTC
CCGCCAGATTTTGGCAAGATTACTAAGCAGGAAGTCAAGGACTTTTTTGCTTGGGCAAAGGTCAATCAGGTGCCG
GTGACTCACGAGTTTAAAGTTCCCAGGGAATTGGCGGGAACTAAAGGGGCGGAGAAATCTCTAAAACGCCCACTG
GGTGACGTCACCAATACTAGCTATAAAAGTCTGGAGAAGCGGGCCAGGCTCTCATTTGTTCCCGAGACGCCTCGC
AGTTCAGACGTGACTGTTGATCCCGCTCCTCTGCGACCGCTCAATTGGAATTCAAGGTATGATTGCAAATGTGAC
TATCATGCTCAATTTGACAACATTTCTAACAAATGTGATGAATGTGAATATTTGAATCGGGGCAAAAATGGATGT
ATCTGTCACAATGTAACTCACTGTCAAATTTGTCATGGGATTCCCCCCTGGGAAAAGGAAAACTTGTCAGATTTT
GGGGATTTTGACGATGCCAATAAAGAACAGTAA
CapVP1: (SEQ ID NO: 14)
ATGTCTTTTGTTGATCACCCTCCAGATTGGTTGGAAGAAGTTGGTGAAGGTCTTCGCGAGTTTTTGGGCCTTGAA
GCGGGCCCACCGAAACCAAAACCCAATCAGCAGCATCAAGATCAAGCCCGTGGTCTTGTGCTGCCTGGTTATAAC
TATCTCGGACCCGGAAACGGTCTCGATCGAGGAGAGCCTGTCAACAGGGCAGACGAGGTCGCGCGAGAGCACGAC
ATCTCGTACAACGAGCAGCTTGAGGCGGGAGACAACCCCTACCTCAAGTACAACCACGCGGACGCCGAGTTTCAG
GAGAAGCTCGCCGACGACACATCCTTCGGGGGAAACCTCGGAAAGGCAGTCTTTCAGGCCAAGAAAAGGGTTCTC
GAACCTTTTGGCCTGGTTGAAGAGGGTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGACGCCGAAGCTGGACCCAGCGGATCC
CAGCAGCTGCAAATCCCAGCCCAACCAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGATTGGCATTGCGATTCCACGTGGATG
GGGGACAGAGTCGTCACCAAGTCCACCCGAACCTGGGTGCTGCCCAGCTACAACAACCACCAGTACCGAGAGATC
AAAAGCGGCTCCGTCGACGGAAGCAACGCCAACGCCTACTTTGGATACAGCACCCCCTGGGGGTACTTTGACTTT
AACCGCTTCCACAGCCACTGGAGCCCCCGAGACTGGCAAAGACTCATCAACAACTACTGGGGCTTCAGACCCCGG
TCCCTCAGAGTCAAAATCTTCAACATTCAAGTCAAAGAGGTCACGGTGCAGGACTCCACCACCACCATCGCCAAC
AACCTCACCTCCACCGTCCAAGTGTTTACGGACGACGACTACCAGCTGCCCTACGTCGTCGGCAACGGGACCGAG
GGATGCCTGCCGGCCTTCCCTCCGCAGGTCTTTACGCTGCCGCAGTACGGTTACGCGACGCTGAACCGCGACAAC
ACAGAAAATCCCACCGAGAGGAGCAGCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTTCGCTCCCAGTCAGAACCTGTTCAAG
CTGGCCAACCCGCTGGTGGACCAGTACTTGTACCGCTTCGTGAGCACAAATAACACTGGCGGAGTCCAGTTCAAC
AAGAACCTGGCCGGGAGATACGCCAACACCTACAAAAACTGGTTCCCGGGGCCCATGGGCCGAACCCAGGGCTGG
AACCTGGGCTCCGGGGTCAACCGCGCCAGTGTCAGCGCCTTCGCCACGACCAATAGGATGGAGCTCGAGGGCGCG
AGTTACCAGGTGCCCCCGCAGCCGAACGGCATGACCAACAACCTCCAGGGCAGCAACACCTATGCCCTGGAGAAC
ACTATGATCTTCAACAGCCAGCCGGCGAACCCGGGCACCACCGCCACGTACCTCGAGGGCAACATGCTCATCACC
AGCGAGAGCGAGACGCAGCCGGTGAACCGCGTGGCGTACAACGTCGGCGGGCAGATGGCCACCAACAACCAGAGC
TCCACCACTGCCCCCGCGACCGGCACGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCACTTTCACCCCTCTCCGGCCATGGGC
GGATTCGGACTCAAACACCCACCGCCCATGATGCTCATCAAGAACACGCCTGTGCCCGGAAATATCACCAGCTTC
TCGGACGTGCCCGTCAGCAGCTTCATCACCCAGTACAGCACCGGGCAGGTCACCGTGGAGATGGAGTGGGAGCTC
AAGAAGGAAAACTCCAAGAGGTGGAACCCAGAGATCCAGTACACAAACAACTACAACGACCCCCAGTTTGTGGAC
TTTGCCCCGGACAGCACCGGGGAATACAGAACCACCAGACCTATCGGAACCCGATACCTTACCCGACCCCTTTAA
AAV-6
Full Genome: AF028704
Rep78: (SEQ ID NO: 15)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTCCTGGTCCAGTGGCGCCGCGTGAGTAAGGCCCCGGAG
GCCCTCTTCTTTGTTCAGTTCGAGAAGGGCGAGTCCTACTTCCACCTCCATATTCTGGTGGAGACCACGGGGGTC
AAATCCATGGTGCTGGGCCGCTTCCTGAGTCAGATTAGGGACAAGCTGGTGCAGACCATCTACCGCGGGATCGAG
CCGACCCTGCCCAACTGGTTCGCGGTGACCAAGACGCGTAATGGCGCCGGAGGGGGGAACAAGGTGGTGGACGAG
TGCTACATCCCCAACTACCTCCTGCCCAAGACTCAGCCCGAGCTGCAGTGGGCGTGGACTAACATGGAGGAGTAT
ATAAGCGCGTGTTTAAACCTGGCCGAGCGCAAACGGCTCGTGGCGCACGACCTGACCCACGTCAGCCAGACCCAG
GAGCAGAACAAGGAGAATCTGAACCCCAATTCTGACGCGCCTGTCATCCGGTCAAAAACCTCCGCACGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCTCTGGACAATGCCGGCAAGATCATGGCG
CTGACCAAATCCGCGCCCGACTACCTGGTAGGCCCCGCTCCGCCCGCCGACATTAAAACCAACCGCATTTACCGC
ATCCTGGAGCTGAACGGCTACGACCCTGCCTACGCCGGCTCCGTCTTTCTCGGCTGGGCCCAGAAAAGGTTCGGA
AAACGCAACACCATCTGGCTGTTTGGGCCGGCCACCACGGGCAAGACCAACATCGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGATCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCATGACTTTGGCAAGGTGACAAAGCAGGAAGTCAAAGAGTTCTTCCGCTGGGCGCAGGAT
CACGTGACCGAGGTGGCGCATGAGTTCTACGTCAGAAAGGGTGGAGCCAACAAGAGACCCGCCCCCGATGACGCG
GATAAAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGCTTCAGATGCTGTTTCCCTGCAAAACA
TGCGAGAGAATGAATCAGAATTTCAACATTTGCTTCACGCACGGGACCAGAGACTGTTCAGAATGTTTCCCCGGC
GTGTCAGAATCTCAACCGGTCGTCAGAAAGAGGACGTATCGGAAACTCTGTGCCATTCATCATCTGCTGGGGCGG
GCTCCCGAGATTGCTTGCTCGGCCTGCGATCTGGTCAACGTGGATCTGGATGACTGTGTTTCTGAGCAATAA
CapVP1: (SEQ ID NO: 16)
ATGGCTGCCGATGGTTATCTTCCAGATTGGCTCGAGGACAACCTCTCTGAGGGCATTCGCGAGTGGTGGGACTTG
AAACCTGGAGCCCCGAAACCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGATGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGAGGGTT
CTCGAACCTTTTGGTCTGGTTGAGGAAGGTGCTAAGACGGCTCCTGGAAAGAAACGTCCGGTAGAGCAGTCGCCA
CAAGAGCCAGACTCCTCCTCGGGCATTGGCAAGACAGGCCAGCAGCCCGCTAAAAAGAGACTCAATTTTGGTCAG
ACTGGCGACTCAGAGTCAGTCCCCGACCCACAACCTCTCGGAGAACCTCCAGCAACCCCCGCTGCTGTGGGACCT
ACTACAATGGCTTCAGGCGGTGGCGCACCAATGGCAGACAATAACGAAGGCGCCGACGGAGTGGGTAATGCCTCA
GGAAATTGGCATTGCGATTCCACATGGCTGGGCGACAGAGTCATCACCACCAGCACCCGAACATGGGCCTTGCCC
ACCTATAACAACCACCTCTACAAGCAAATCTCCAGTGCTTCAACGGGGGCCAGCAACGACAACCACTACTTCGGC
TACAGCACCCCCTGGGGGTATTTTGATTTCAACAGATTCCACTGCCATTTCTCACCACGTGACTGGCAGCGACTC
ATCAACAACAATTGGGGATTCCGGCCCAAGAGACTCAACTTCAAGCTCTTCAACATCCAAGTCAAGGAGGTCACG
ACGAATGATGGCGTCACGACCATCGCTAATAACCTTACCAGCACGGTTCAAGTCTTCTCGGACTCGGAGTACCAG
TTGCCGTACGTCCTCGGCTCTGCGCACCAGGGCTGCCTCCCTCCGTTCCCGGCGGACGTGTTCATGATTCCGCAG
TACGGCTACCTAACGCTCAACAATGGCAGCCAGGCAGTGGGACGGTCATCCTTTTACTGCCTGGAATATTTCCCA
TCGCAGATGCTGAGAACGGGCAATAACTTTACCTTCAGCTACACCTTCGAGGACGTGCCTTTCCACAGCAGCTAC
GCGCACAGCCAGAGCCTGGACCGGCTGATGAATCCTCTCATCGACCAGTACCTGTATTACCTGAACAGAACTCAG
AATCAGTCCGGAAGTGCCCAAAACAAGGACTTGCTGTTTAGCCGGGGGTCTCCAGCTGGCATGTCTGTTCAGCCC
AAAAACTGGCTACCTGGACCCTGTTACCGGCAGCAGCGCGTTTCTAAAACAAAAACAGACAACAACAACAGCAAC
TTTACCTGGACTGGTGCTTCAAAATATAACCTTAATGGGCGTGAATCTATAATCAACCCTGGCACTGCTATGGCC
TCACACAAAGACGACAAAGACAAGTTCTTTCCCATGAGCGGTGTCATGATTTTTGGAAAGGAGAGCGCCGGAGCT
TCAAACACTGCATTGGACAATGTCATGATCACAGACGAAGAGGAAATCAAAGCCACTAACCCCGTGGCCACCGAA
AGATTTGGGACTGTGGCAGTCAATCTCCAGAGCAGCAGCACAGACCCTGCGACCGGAGATGTGCATGTTATGGGA
GCCTTACCTGGAATGGTGTGGCAAGACAGAGACGTATACCTGCAGGGTCCTATTTGGGCCAAAATTCCTCACACG
GATGGACACTTTCACCCGTCTCCTCTCATGGGCGGCTTTGGACTTAAGCACCCGCCTCCTCAGATCCTCATCAAA
AACACGCCTGTTCCTGCGAATCCTCCGGCAGAGTTTTCGGCTACAAAGTTTGCTTCATTCATCACCCAGTATTCC
ACAGGACAAGTGAGCGTGGAGATTGAATGGGAGCTGCAGAAAGAAAACAGCAAACGCTGGAATCCCGAAGTGCAG
TATACATCTAACTATGCAAAATCTGCCAACGTTGATTTCACTGTGGACAACAATGGACTTTATACTGAGCCTCGC
CCCATTGGCACCCGTTACCTCACCCGTCCCCTGTAA
AAV-7
Full Genome: NC_006260
Rep78: (SEQ ID NO: 17)
ATGCCGGGTTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCTGAATCTGATCGAGCAG
GCACCCCTGACCGTGGCCGAGAAGCTGCAGCGCGACTTCCTGGTCCAATGGCGCCGCGTGAGTAAGGCCCCGGAG
GCCCTGTTCTTTGTTCAGTTCGAGAAGGGCGAGAGCTACTTCCACCTTCACGTTCTGGTGGAGACCACGGGGGTC
AAGTCCATGGTGCTAGGCCGCTTCCTGAGTCAGATTCGGGAGAAGCTGGTCCAGACCATCTACCGCGGGGTCGAG
CCCACGCTGCCCAACTGGTTCGCGGTGACCAAGACGCGTAATGGCGCCGGCGGGGGGAACAAGGTGGTGGACGAG
TGCTACATCCCCAACTACCTCCTGCCCAAGACCCAGCCCGAGCTGCAGTGGGCGTGGACTAACATGGAGGAGTAT
ATAAGCGCGTGTTTGAACCTGGCCGAACGCAAACGGCTCGTGGCGCAGCACCTGACCCACGTCAGCCAGACGCAG
GAGCAGAACAAGGAGAATCTGAACCCCAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGCAAGATCATGGCG
CTGACCAAATCCGCGCCCGACTACCTGGTGGGGCCCTCGCTGCCCGCGGACATTAAAACCAACCGCATCTACCGC
ATCCTGGAGCTGAACGGGTACGATCCTGCCTACGCCGGCTCCGTCTTTCTCGGCTGGGCCCAGAAAAAGTTCGGG
AAGCGCAACACCATCTGGCTGTTTGGGCCCGCCACCACCGGCAAGACCAACATTGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGACCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCACGACTTTGGCAAGGTGACGAAGCAGGAAGTCAAAGAGTTCTTCCGCTGGGCCAGTGAT
CACGTGACCGAGGTGGCGCATGAGTTCTACGTCAGAAAGGGCGGAGCCAGCAAAAGACCCGCCCCCGATGACGCG
GATATAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGATTCAGATGCTGTTTCCCTGCAAAACG
TGCGAGAGAATGAATCAGAATTTCAACATTTGCTTCACACACGGGGTCAGAGACTGTTTAGAGTGTTTCCCCGGC
GTGTCAGAATCTCAACCGGTCGTCAGAAAAAAGACGTATCGGAAACTCTGCGCGATTCATCATCTGCTGGGGCGG
GCGCCCGAGATTGCTTGCTCGGCCTGCGACCTGGTCAACGTGGACCTGGACGACTGCGTTTCTGAGCAATAA
CapVP1: (SEQ ID NO: 18)
ATGGCTGCCGATGGTTATCTTCCAGATTGGCTCGAGGACAACCTCTCTGAGGGCATTCGCGAGTGGTGGGACCTG
AAACCTGGAGCCCCGAAACCCAAAGCCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCATTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGCAAAGAAGAGACCGGTAGAGCCGTCACCT
CAGCGTTCCCCCGACTCCTCCACGGGCATCGGCAAGAAAGGCCAGCAGCCCGCCAGAAAGAGACTCAATTTCGGT
CAGACTGGCGACTCAGAGTCAGTCCCCGACCCTCAACCTCTCGGAGAACCTCCAGCAGCGCCCTCTAGTGTGGGA
TCTGGTACAGTGGCTGCAGGCGGTGGCGCACCAATGGCAGACAATAACGAAGGTGCCGACGGAGTGGGTAATGCC
TCAGGAAATTGGCATTGCGATTCCACATGGCTGGGCGACAGAGTCATTACCACCAGCACCCGAACCTGGGCCCTG
CCCACCTACAACAACCACCTCTACAAGCAAATCTCCAGTGAAACTGCAGGTAGTACCAACGACAACACCTACTTC
GGCTACAGCACCCCCTGGGGGTATTTTGACTTTAACAGATTCCACTGCCACTTCTCACCACGTGACTGGCAGCGA
CTCATCAACAACAACTGGGGATTCCGGCCCAAGAAGCTGCGGTTCAAGCTCTTCAACATCCAGGTCAAGGAGGTC
ACGACGAATGACGGCGTTACGACCATCGCTAATAACCTTACCAGCACGATTCAGGTATTCTCGGACTCGGAATAC
CAGCTGCCGTACGTCCTCGGCTCTGCGCACCAGGGCTGCCTGCCTCCGTTCCCGGCGGACGTCTTCATGATTCCT
CAGTACGGCTACCTGACTCTCAACAATGGCAGTCAGTCTGTGGGACGTTCCTCCTTCTACTGCCTGGAGTACTTC
CCCTCTCAGATGCTGAGAACGGGCAACAACTTTGAGTTCAGCTACAGCTTCGAGGACGTGCCTTTCCACAGCAGC
TACGCACACAGCCAGAGCCTGGACCGGCTGATGAATCCCCTCATCGACCAGTACTTGTACTACCTGGCCAGAACA
CAGAGTAACCCAGGAGGCACAGCTGGCAATCGGGAACTGCAGTTTTACCAGGGCGGGCCTTCAACTATGGCCGAA
CAAGCCAAGAATTGGTTACCTGGACCTTGCTTCCGGCAACAAAGAGTCTCCAAAACGCTGGATCAAAACAACAAC
AGCAACTTTGCTTGGACTGGTGCCACCAAATATCACCTGAACGGCAGAAACTCGTTGGTTAATCCCGGCGTCGCC
ATGGCAACTCACAAGGACGACGAGGACCGCTTTTTCCCATCCAGCGGAGTCCTGATTTTTGGAAAAACTGGAGCA
ACTAACAAAACTACATTGGAAAATGTGTTAATGACAAATGAAGAAGAAATTCGTCCTACTAATCCTGTAGCCACG
GAAGAATACGGGATAGTCAGCAGCAACTTACAAGCGGCTAATACTGCAGCCCAGACACAAGTTGTCAACAACCAG
GGAGCCTTACCTGGCATGGTCTGGCAGAACCGGGACGTGTACCTGCAGGGTCCCATCTGGGCCAAGATTCCTCAC
ACGGATGGCAACTTTCACCCGTCTCCTTTGATGGGCGGCTTTGGACTTAAACATCCGCCTCCTCAGATCCTGATC
AAGAACACTCCCGTTCCCGCTAATCCTCCGGAGGTGTTTACTCCTGCCAAGTTTGCTTCGTTCATCACACAGTAC
AGCACCGGACAAGTCAGCGTGGAAATCGAGTGGGAGCTGCAGAAGGAAAACAGCAAGCGCTGGAACCCGGAGATT
CAGTACACCTCCAACTTTGAAAAGCAGACTGGTGTGGACTTTGCCGTTGACAGCCAGGGTGTTTACTCTGAGCCT
CGCCCTATTGGCACTCGTTACCTCACCCGTAATCTGTAA
AAV-8
Full Genome: NC_006261
Rep78: (SEQ ID NO: 19)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCAATGGCGCCGCGTGAGTAAGGCCCCGGAG
GCCCTCTTCTTTGTTCAGTTCGAGAAGGGCGAGAGCTACTTTCACCTGCACGTTCTGGTCGAGACCACGGGGGTC
AAGTCCATGGTGCTAGGCCGCTTCCTGAGTCAGATTCGGGAAAAGCTTGGTCCAGACCATCTACCCGCGGGGTCG
AGCCCCACCTTGCCCAACTGGTTCGCGGTGACCAAAGACGCGGTAATGGCGCCGGCGGGGGGGAACAAGGTGGTG
GACGAGTGCTACATCCCCAACTACCTCCTGCCCAAGACTCAGCCCGAGCTGCAGTGGGCGTGGACTAACATGGAG
GAGTATATAAGCGCGTGCTTGAACCTGGCCGAGCGCAAACGGCTCGTGGCGCAGCACCTGACCCACGTCAGCCAG
ACGCAGGAGCAGAACAAGGAGAATCTGAACCCCAATTCTGACGCGCCCGTGATCAGGTCAAAAACCTCCGCGCGC
TATATGGAGCTGGTCGGGTGGCTGGTGGACCGGGGCATCACCTCCGAGAAGCAGTGGATCCAGGAGGACCAGGCC
TCGTACATCTCCTTCAACGCCGCCTCCAACTCGCGGTCCCAGATCAAGGCCGCGCTGGACAATGCCGGCAAGATC
ATGGCGCTGACCAAATCCGCGCCCGACTACCTGGTGGGGCCCTCGCTGCCCGCGGACATTACCCAGAACCGCATC
TACCGCATCCTCGCTCTCAACGGCTACGACCCTGCCTACGCCGGCTCCGTCTTTCTCGGCTGGGCTCAGAAAAAG
TTCGGGAAACGCAACACCATCTGGCTGTTTGGACCCGCCACCACCGGCAAGACCAACATTGCGGAAGCCATCGCC
CACGCCGTGCCCTTCTACGGCTGCGTCAACTGGACCAATGAGAACTTTCCCTTCAATGATTGCGTCGACAAGATG
GTGATCTGGTGGGAGGAGGGCAAGATGACGGCCAAGGTCGTGGAGTCCGCCAAGGCCATTCTCGGCGGCAGCAAG
GTGCGCGTGGACCAAAAGTGCAAGTCGTCCGCCCAGATCGACCCCACCCCCGTGATCGTCACCTCCAACACCAAC
ATGTGCGCCGTGATTGACGGGAACAGCACCACCTTCGAGCACCAGCAGCCTCTCCAGGACCGGATGTTTAAGTTC
GAACTCACCCGCCGTCTGGAGCACGACTTTGGCAAGGTGACAAAGCAGGAAGTCAAAGAGTTCTTCCGCTGGGCC
AGTGATCACGTGACCGAGGTGGCGCATGAGTTTTACGTCAGAAAGGGCGGAGCCAGCAAAAGACCCGCCCCCGAT
GACGCGGATAAAAGCGAGCCCAAGCGGGCCTGCCCCTCAGTCGCGGATCCATCGACGTCAGACGCGGAAGGAGCT
CCGGTGGACTTTGCCGACAGGTACCAAAACAAATGTTCTCGTCACGCGGGCATGCTTCAGATGCTGTTTCCCTGC
AAAACGTGCGAGAGAATGAATCAGAATTTCAACATTTGCTTCACACACGGGGTCAGAGACTGCTCAGAGTGTTTC
CCCGGCGTGTCAGAATCTCAACCGGTCGTCAGAAAGAGGACGTATCGGAAACTCTGTGCGATTCATCATCTGCTG
GGGCGGGCTCCCGAGATTGCTTGCTCGGCCTGCGATCTGGTCAACGTGGACCTGGATGACTGTGTTTCTGAGCAA
TAA
CapVP1: (SEQ ID NO: 20)
ATGGCTGCCGATGGTTATCTTCCAGATTGGCTCGAGGACAACCTCTCTGAGGGCATTCGCGAGTGGTGGGCGCTG
AAACCTGGAGCCCCGAAGCCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTGCAGGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAAAGAAGAGACCGGTAGAGCCATCACCC
CAGCGTTCTCCAGACTCCTCTACGGGCATCGGCAAGAAAGGCCAACAGCCCGCCAGAAAAAGACTCAATTTTGGT
CAGACTGGCGACTCAGAGTCAGTTCCAGACCCTCAACCTCTCGGAGAACCTCCAGCAGCGCCCTCTGGTGTGGGA
CCTAATACAATGGCTGCAGGCGGTGGCGCACCAATGGCAGACAATAACGAAGGCGCCGACGGAGTGGGTAGTTCC
TCGGGAAATTGGCATTGCGATTCCACATGGCTGGGCGACAGAGTCATCACCACCAGCACCCGAACCTGGGCCCTG
CCCACCTACAACAACCACCTCTACAAGCAAATCTCCAACGGGACATCGGGAGGAGCCACCAACGACAACACCTAC
TTCGGCTACAGCACCCCCTGGGGGTATTTTGACTTTAACAGATTCCACTGCCACTTTTCACCACGTGACTGGCAG
CGACTCATCAACAACAACTGGGGATTCCGGCCCAAGAGACTCAGCTTCAAGCTCTTCAACATCCAGGTCAAGGAG
GTCACGCAGAATGAAGGCACCAAGACCATCGCCAATAACCTCACCAGCACCATCCAGGTGTTTACGGACTCGGAG
TACCAGCTGCCGTACGTTCTCGGCTCTGCCCACCAGGGCTGCCTGCCTCCGTTCCCGGCGGACGTGTTCATGATT
CCCCAGTACGGCTACCTAACACTCAACAACGGTAGTCAGGCCGTGGGACGCTCCTCCTTCTACTGCCTGGAATAC
TTTCCTTCGCAGATGCTGAGAACCGGCAACAACTTCCAGTTTACTTACACCTTCGAGGACGTGCCTTTCCACAGC
AGCTACGCCCACAGCCAGAGCTTGGACCGGCTGATGAATCCTCTGATTGACCAGTACCTGTACTACTTGTCTCGG
ACTCAAACAACAGGAGGCACGGCAAATACGCAGACTCTGGGCTTCAGCCAAGGTGGGCCTAATACAATGGCCAAT
CAGGCAAAGAACTGGCTGCCAGGACCCTGTTACCGCCAACAACGCGTCTCAACGACAACCGGGCAAAACAACAAT
AGCAACTTTGCCTGGACTGCTGGGACCAAATACCATCTGAATGGAAGAAATTCATTGGCTAATCCTGGCATCGCT
ATGGCAACACACAAAGACGACGAGGAGCGTTTTTTTCCCAGTAACGGGATCCTGATTTTTGGCAAACAAAATGCT
GCCAGAGACAATGCGGATTACAGCGATGTCATGCTCACCAGCGAGGAAGAAATCAAAACCACTAACCCTGTGGCT
ACAGAGGAATACGGTATCGTGGCAGATAACTTGCAGCAGCAAAACACGGCTCCTCAAATTGGAACTGTCAACAGC
CAGGGGGCCTTACCCGGTATGGTCTGGCAGAACCGGGACGTGTACCTGCAGGGTCCCATCTGGGCCAAGATTCCT
CACACGGACGGCAACTTCCACCCGTCTCCGCTGATGGGCGGCTTTGGCCTGAAACATCCTCCGCCTCAGATCCTG
ATCAAGAACACGCCTGTACCTGCGGATCCTCCGACCACCTTCAACCAGTCAAAGCTGAACTCTTTCATCACGCAA
TACAGCACCGGACAGGTCAGCGTGGAAATTGAATGGGAGCTGCAGAAGGAAAACAGCAAGCGCTGGAACCCCGAG
ATCCAGTACACCTCCAACTACTACAAATCTACAAGTGTGGACTTTGCTGTTAATACAGAAGGCGTGTACTCTGAA
CCCCGCCCCATTGGCACCCGTTACCTCACCCGTAATCTGTAA
AAV-9
Cap only: AY530579
CapVP1: (SEQ ID NO: 21)
ATGGCTGCCGATGGTTATCTTCCAGATTGGCTCGAGGACAACCTTAGTGAAGGAATTCGCGAGTGGTGGGCTTTG
AAACCTGGAGCCCCTCAACCCAAGGCAAATCAACAACATCAAGACAACGCTCGAGGTCTTGTGCTTCCGGGTTAC
AAATACCTTGGACCCGGCAACGGACTCGACAAGGGGGAGCCGGTCAACGCAGCAGACGCGGCGGCCCTCGAGCAC
GACAAGGCCTACGACCAGCAGCTCAAGGCCGGAGACAACCCGTACCTCAAGTACAACCACGCCGACGCCGAGTTC
CAGGAGCGGCTCAAAGAAGATACGTCTTTTGGGGGCAACCTCGGGCGAGCAGTCTTCCAGGCCAAAAAGAGGCTT
CTTGAACCTCTTGGTCTGGTTGAGGAAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGCTAAAAAGAGACTCAATTTCGGTCAG
ACTGGCGACACAGAGTCAGTCCCAGACCCTCAACCAATCGGAGAACCTCCCGCAGCCCCCTCAGGTGTGGGATCT
CTTACAATGGCTTCAGGTGGTGGCGCACCAGTGGCAGACAATAACGAAGGTGCCGATGGAGTGGGTAGTTCCTCG
GGAAATTGGCATTGCGATTCCCAATGGCTGGGGGACAGAGTCATCACCACCAGCACCCGAACCTGGGCCCTGCCC
ACCTACAACAATCACCTCTACAAGCAAATCTCCAACAGCACATCTGGAGGATCTTCAAATGACAACGCCTACTTC
GGCTACAGCACCCCCTGGGGGTATTTTGACTTCAACAGATTCCACTGCCACTTCTCACCACGTGACTGGCAGCGA
CTCATCAACAACAACTGGGGATTCCGGCCTAAGCGACTCAACTTCAAGCTCTTCAACATTCAGGTCAAAGAGGTT
ACGGACAACAATGGAGTCAAGACCATCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTTCCCAGCGGACGTTTTCATGATTCCT
CAGTACGGGTATCTGACGCTTAATGATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTTTGAGAACGTACCTTTCCATAGCAGC
TACGCTCACAGCCAAAGCCTGGACCGACTAATGAATCCACTCATCGACCAATACTTGTACTATCTCTCAAAGACT
ATTAACGGTTCTGGACAGAATCAACAAACGCTAAAATTCAGTGTGGCCGGACCCAGCAACATGGCTGTCCAGGGA
AGAAACTACATACCTGGACCCAGCTACCGACAACAACGTGTCTCAACCACTGTGACTCAAAACAACAACAGCGAA
TTTGCTTGGCCTGGAGCTTCTTCTTGGGCTCTCAATGGACGTAATAGCTTGATGAATCCTGGACCTGCTATGGCC
AGCCACAAAGAAGGAGAGGACCGTTTCTTTCCTTTGTCTGGATCTTTAATTTTTGGCAAACAAGGAACTGGAAGA
GACAACGTGGATGCGGACAAAGTCATGATAACCAACGAAGAAGAAATTAAAACTACTAACCCGGTAGCAACGGAG
TCCTATGGACAAGTGGCCACAAACCACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACCCATTTGGGCCAAAATTCCTCACACG
GACGGCAACTTTCACCCTTCTCCGCTGATGGGAGGGTTTGGAATGAAGCACCCGCCTCCTCAGATCCTCATCAAA
AACACACCTGTACCTGCGGATCCTCCAACGGCCTTCAACAAGGACAAGCTGAACTCTTTCATCACCCAGTATTCT
ACTGGCCAAGTCAGCGTGGAGATCGAGTGGGAGCTGCAGAAGGAAAACAGCAAGCGCTGGAACCCGGAGATCCAG
TACACTTCCAACTATTACAAGTCTAATAATGTTGAATTTGCTGTTAATACTGAAGGTGTATATAGTGAACCCCGC
CCCATTGGCACCAGATACCTGACTCGTAATCTGTAA
AAV-10
Partial Genome: AY631965
Rep78: (SEQ ID NO: 22)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCACTGGCGCCGCGTGAGTAAGGCCCCGGAG
GCCCTCTTCTTTGTTCAGTTCGAGAAGGGCGAGTCCTACTTTCACCTGCACGTTCTGGTCGAGACCACGGGGGTC
AAGTCCATGGTCCTGGGCCGCTTCCTGAGTCAGATCAGAGACAGGCTGGTGCAGACCATCTACCGCGGGGTAGAG
CCCACGCTGCCCAACTGGTTCGCGGTGACCAAGACGCGAAATGGCGCCGGCGGGGGGAACAAGGTGGTGGACGAG
TGCTACATCCCCAACTACCTCCTGCCCAAGACGCAGCCCGAGCTGCAGTGGGCGTGGACTAACATGGAGGAGTAT
ATAAGCGCGTGTCTGAACCTCGCGGAGCGTAAACGGCTCGTGGCGCAGCACCTGACCCACGTCAGCCAGACGCAG
GAGCAGAACAAGGAGAATCTGAACCCGAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGAAAGATCATGGCG
CTGACCAAATCCGCGCCCGACTACCTGGTAGGCCCGTCCTTACCCGCGGACATTAAGGCCAACCGCATCTACCGC
ATCCTGGAGCTCAACGGCTACGACCCCGCCTACGCCGGCTCCGTCTTCCTGGGCTGGGCGCAGAAAAAGTTCGGT
AAAAGGAATACAATTTGGCTGTTCGGGCCCGCCACCACCGGCAAGACCAACATCGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACCGCCAAGGTCGTGGAGTCCGCCAAGGCCATTCTGGGCGGAAGCAAGGTGCGC
GTCGACCAAAAGTGCAAGTCCTCGGCCCAGATCGACCCCACGCCCGTGATCGTCACCTCCAACACCAACATGTGC
GCCGTGATCGACGGGAACAGCACCACCTTCGAGCACCAGCAGCCCCTGCAGGACCGCATGTTCAAGTTCGAGCTC
ACCCGCCGTCTGGAGCACGACTTTGGCAAGGTGACCAAGCAGGAAGTCAAAGAGTTCTTCCGCTGGGCTCAGGAT
CACGTGACTGAGGTGACGCATGAGTTCTACGTCAGAAAGGGCGGAGCCACCAAAAGACCCGCCCCCAGTGACGCG
GATATAAGCGAGCCCAAGCGGGCCTGCCCCTCAGTTGCGGAGCCATCGACGTCAGACGCGGAAGCACCGGTGGAC
TTTGCGGACAGGTACCAAAACAAATGTTCTCGTCACGCGGGCATGCTTCAGATGCTGTTTCCCTGCAAGACATGC
GAGAGAATGAATCAGAATTTCAACGTCTGCTTCACGCACGGGGTCAGAGACTGCTCAGAGTGCTTCCCCGGCGCG
TCAGAATCTCAACCTGTCGTCAGAAAAAAGACGTATCAGAAACTGTGCGCGATTCATCATCTGCTGGGGGGGGCA
CCCGAGATTGCGTGTTCGGCCTGCGATCTCGTCAACGTGGACTTGGATGACTGTGTTTCTGAGCAATAA
CapVP1: (SEQ ID NO: 23)
ATGGCTGCTGACGGTTATCTTCCAGATTGGCTCGAGGACAACCTCTCTGAGGGCATTCGCGAGTGGTGGGACCTG
AAACCTGGAGCCCCCAAGCCCAAGGCCAACCAGCAGAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGCTGCTAAGACGGCTCCTGGAAAGAAGAGACCGGTAGAACCGTCACCT
CAGCGTTCCCCCGACTCCTCCACGGGCATCGGCAAGAAAGGCCAGCAGCCCGCTAAAAAGAGACTGAACTTTGGG
CAGACTGGCGAGTCAGAGTCAGTCCCCGACCCTCAACCAATCGGAGAACCACCAGCAGGCCCCTCTGGTCTGGGA
TCTGGTACAATGGCTGCAGGCGGTGGCGCTCCAATGGCAGACAATAACGAAGGCGCCGACGGAGTGGGTAGTTCC
TCAGGAAATTGGCATTGCGATTCCACATGGCTGGGCGACAGAGTCATCACCACCAGCACCCGAACCTGGGCCCTG
CCCACCTACAACAACCACCTCTACAAGCAAATCTCCAACGGGACATCGGGAGGAAGCACCAACGACAACACCTAC
TTCGGCTACAGCACCCCCTGGGGGTATTTTGACTTCAACAGATTCCACTGCCACTTCTCACCACGTGACTGGCAG
CGACTCATCAACAACAACTGGGGATTCCGGCCAAAAAGACTCAGCTTCAAGCTCTTCAACATCCAGGTCAAGGAG
GTCACGCAGAATGAAGGCACCAAGACCATCGCCAATAACCTTACCAGCACGATTCAGGTATTTACGGACTCGGAA
TACCAGCTGCCGTACGTCCTCGGCTCCGCGCACCAGGGCTGCCTGCCTCCGTTCCCGGCGGATGTCTTCATGATT
CCCCAGTACGGCTACCTGACACTGAACAATGGAAGTCAAGCCGTAGGCCGTTCCTCCTTCTACTGCCTGGAATAT
TTTCCATCTCAAATGCTGCGAACTGGAAACAATTTTGAATTCAGCTACACCTTCGAGGACGTGCCTTTCCACAGC
AGCTACGCACACAGCCAGAGCTTGGACCGACTGATGAATCCTCTCATTGACCAGTACCTGTACTACTTATCCAGA
ACTCAGTCCACAGGAGGAACTCAAGGTACCCAGCAATTGTTATTTTCTCAAGCTGGGCCTGCAAACATGTCGGCT
CAGGCCAAGAACTGGCTGCCTGGACCTTGCTACCGGCAGCAGCGAGTCTCCACGACACTGTCGCAAAACAACAAC
AGCAACTTTGCTTGGACTGGTGCCACCAAATATCACCTGAACGGAAGAGACTCTCTGGTGAATCCCGGTGTCGCC
ATGGCAACCCACAAGGACGACGAGGAACGCTTCTTCCCGTCGAGCGGAGTCCTGATGTTTGGAAAACAGGGTGCT
GGAAGAGACAATGTGGACTACAGCAGCGTTATGCTAACAAGCGAAGAAGAAATTAAAACCACTAACCCTGTAGCC
ACAGAACAATACGGCGTGGTGGCTGACAACTTGCAGCAAGCCAATACAGGGCCTATTGTGGGAAATGTCAACAGC
CAAGGAGCCTTACCTGGCATGGTCTGGCAGAACCGAGACGTGTACCTGCAGGGTCCCATCTGGGCCAAGATTCCT
CACACGGACGGCAACTTTCACCCGTCTCCTCTGATGGGCGGCTTTGGACTTAAACACCCGCCTCCACAGATCCTG
ATCAAGAACACGCCGGTACCTGCGGATCCTCCAACAACGTTCAGCCAGGCGAAATTGGCTTCCTTCATCACGCAG
TACAGCACCGGACAGGTCAGCGTGGAAATCGAGTGGGAGCTGCAGAAGGAGAACAGCAAACGCTGGAACCCAGAG
ATTCAGTACACTTCAAACTACTACAAATCTACAAATGTGGACTTTGCTGTCAATACAGAGGGAACTTATTCTGAG
CCTCGCCCCATTGGTACTCGTTATCTGACACGTAATCTGTAA
AAV-11
Partial Genome: AY631966
Rep78: (SEQ ID NO: 24)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCACTGGCGCCGCGTGAGTAAGGCCCCGGAG
GCCCTCTTCTTTGTTCAGTTCGAGAAGGGCGAGTCCTACTTCCACCTCCACGTTCTCGTCGAGACCACGGGGGTC
AAGTCCATGGTCCTGGGCCGCTTCCTGAGTCAGATCAGAGACAGGCTGGTGCAGACCATCTACCGCGGGGTCGAG
CCCACGCTGCCCAACTGGTTCGCGGTGACCAAGACGCGAAATGGCGCCGGCGGGGGGAACAAGGTGGTGGACGAG
TGCTACATCCCCAACTACCTCCTGCCCAAGACCCAGCCCGAGCTGCAGTGGGCGTGGACTAACATGGAGGAGTAT
ATAAGCGCGTGTCTAAACCTCGCGGAGCGTAAACGGCTCGTGGCGCAGCACCTGACCCACGTCAGCCAGACGCAG
GAGCAGAACAAGGAGAATCTGAACCCGAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGAAAGATCATGGCG
CTGACCAAATCCGCGCCCGACTACCTGGTAGGCCCGTCCTTACCCGCGGACATTAAGGCCAACCGCATCTACCGC
ATCCTGGAGCTCAACGGCTACGACCCCGCCTACGCCGGCTCCGTCTTCCTGGGCTGGGCGCAGAAAAAGTTCGGT
AAACGCAACACCATCTGGCTGTTTGGGCCCGCCACCACCGGCAAGACCAACATCGCGGAAGCCATAGCCCACGCC
GTGCCCTTCTACGGCTGCGTGAACTGGACCAATGAGAACTTTCCCTTCAACGATTGCGTCGACAAGATGGTGATC
TGGTGGGAGGAGGGCAAGATGACCGCCAAGGTCGTGGAGTCCGCCAAGGCCATTCTGGGCGGAAGCAAGGTGCGC
GTGGACCAAAAGTGCAAGTCCTCGGCCCAGATCGACCCCACGCCCGTGATCGTCACCTCCAACACCAACATGTGC
GCCGTGATCGACGGGAACAGCACCACCTTCGAGCACCAGCAGCCGCTGCAGGACCGCATGTTCAAGTTCGAGCTC
ACCCGCCGTCTGGAGCACGACTTTGGCAAGGTGACCAAGCAGGAAGTCAAAGAGTTCTTCCGCTGGGCTCAGGAT
CACGTGACTGAGGTGGCGCATGAGTTCTACGTCAGAAAGGGCGGAGCCACCAAAAGACCCGCCCCCAGTGACGCG
GATATAAGCGAGCCCAAGCGGGCCTGCCCCTCAGTTCCGGAGCCATCGACGTCAGACGCGGAAGCACCGGTGGAC
TTTGCGGACAGGTACCAAAACAAATGTTCTCGTCACGCGGGCATGCTTCAGATGCTGTTTCCCTGCAAGACATGC
GAGAGAATGAATCAGAATTTCAACGTCTGCTTCACGCACGGGGTCAGAGACTGCTCAGAGTGCTTCCCCGGCGCG
TCAGAATCTCAACCCGTCGTCAGAAAAAAGACGTATCAGAAACTGTGCGCGATTCATCATCTGCTGGGGGGGGCA
CCCGAGATTGCGTGTTCGGCCTGCGATCTCGTCAACGTGGACTTGGATGACTGTGTTTCTGAGCAATAA
CapVP1: (SEQ ID NO: 25)
ATGGCTGCTGACGGTTATCTTCCAGATTGGCTCGAGGACAACCTCTCTGAGGGCATTCGCGAGTGGTGGGACCTG
AAACCTGGAGCCCCGAAGCCCAAGGCCAACCAGCAGAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGAGGGTA
CTCGAACCTCTGGGCCTGGTTGAAGAAGGTGCTAAAACGGCTCCTGGAAAGAAGAGACCGTTAGAGTCACCACAA
GAGCCCGACTCCTCCTCGGGCATCGGCAAAAAAGGCAAACAACCAGCCAGAAAGAGGCTCAACTTTGAAGAGGAC
ACTGGAGCCGGAGACGGACCCCCTGAAGGATCAGATACCAGCGCCATGTCTTCAGACATTGAAATGCGTGCAGCA
CCGGGCGGAAATGCTGTCGATGCGGGACAAGGTTCCGATGGAGTGGGTAATGCCTCGGGTGATTGGCATTGCGAT
TCCACCTGGTCTGAGGGCAAGGTCACAACAACCTCGACCAGAACCTGGGTCTTGCCCACCTACAACAACCACTTG
TACCTGCGTCTCGGAACAACATCAAGCAGCAACACCTACAACGGATTCTCCACCCCCTGGGGATATTTTGACTTC
AACAGATTCCACTGTCACTTCTCACCACGTGACTGGCAAAGACTCATCAACAACAACTGGGGACTACGACCAAAA
GCCATGCGCGTTAAAATCTTCAATATCCAAGTTAAGGAGGTCACAACGTCGAACGGCGAGACTACGGTCGCTAAT
AACCTTACCAGCACGGTTCAGATATTTGCGGACTCGTCGTATGAGCTCCCGTACGTGATGGACGCTGGACAAGAG
GGGAGCCTGCCTCCTTTCCCCAATGACGTGTTCATGGTGCCTCAATATGGCTACTGTGGCATCGTGACTGGCGAG
AATCAGAACCAAACGGACAGAAACGCTTTCTACTGCCTGGAGTATTTTCCTTCGCAAATGTTGAGAACTGGCAAC
AACTTTGAAATGGCTTACAACTTTGAGAAGGTGCCGTTCCACTCAATGTATGCTCACAGCCAGAGCCTGGACAGA
CTGATGAATCCCCTCCTGGACCAGTACCTGTGGCACTTACAGTCGACTACCTCTGGAGAGACTCTGAATCAAGGC
AATGCAGCAACCACATTTGGAAAAATCAGGAGTGGAGACTTTGCCTTTTACAGAAAGAACTGGCTGCCTGGGCCT
TGTGTTAAACAGCAGAGATTCTCAAAAACTGCCAGTCAAAATTACAAGATTCCTGCCAGCGGGGGCAACGCTCTG
TTAAAGTATGACACCCACTATACCTTAAACAACCGCTGGAGCAACATCGCGCCCGGACCTCCAATGGCCACAGCC
GGACCTTCGGATGGGGACTTCAGTAACGCCCAGCTTATATTCCCTGGACCATCTGTTACCGGAAATACAACAACT
TCAGCCAACAATCTGTTGTTTACATCAGAAGAAGAAATTGCTGCCACCAACCCAAGAGACACGGACATGTTTGGC
CAGATTGCTGACAATAATCAGAATGCTACAACTGCTCCCATAACCGGCAACGTGACTGCTATGGGAGTGCTGCCT
GGCATGGTGTGGCAAAACAGAGACATTTACTACCAAGGGCCAATTTGGGCCAAGATCCCACACGCGGACGGACAT
TTTCATCCTTCACCGCTGATTGGTGGGTTTGGACTGAAACACCCGCCTCCCCAGATATTCATCAAGAACACTCCC
GTACCTGCCAATCCTGCGACAACCTTCACTGCAGCCAGAGTGGACTCTTTCATCACACAATACAGCACCGGCCAG
GTCGCTGTTCAGATTGAATGGGAAATTGAAAAGGAACGCTCCAAACGCTGGAATCCTGAAGTGCAGTTTACTTCA
AACTATGGGAACCAGTCTTCTATGTTGTGGGCTCCTGATACAACTGGGAAGTATACAGAGCCGCGGGTTATTGGC
TCTCGTTATTTGACTAATCATTTGTAA
AAV-12
Partial Genome: DQ813647
Rep78: (SEQ ID NO: 26)
ATGCCGGGGTTCTACGAGGTGGTGATCAAGGTGCCCAGCGACCTGGACGAGCACCTGCCCGGCATTTCTGACTCC
TTTGTGAACTGGGTGGCCGAGAAGGAATGGGAGTTGCCCCCGGATTCTGACATGGATCAGAATCTGATTGAGCAG
GCACCCCTGACCGTGGCCGAGAAGCTGCAGCGCGAGTTCCTGGTGGAATGGCGCCGAGTGAGTAAATTTCTGGAG
GCCAAGTTTTTTGTGCAGTTTGAAAAGGGGGACTCGTACTTTCATTTGCATATTCTGATTGAAATTACCGGCGTG
AAATCCATGGTGGTGGGCCGCTACGTGAGTCAGATTAGGGATAAACTGATCCAGCGCATCTACCGCGGGGTCGAG
CCCCAGCTGCCCAACTGGTTCGCGGTCACAAAGACCCGAAATGGCGCCGGAGGCGGGAACAAGGTGGTGGACGAG
TGCTACATCCCCAACTACCTGCTCCCCAAGGTCCAGCCCGAGCTTCAGTGGGCGTGGACTAACATGGAGGAGTAT
ATAAGCGCCTGTTTGAACCTCGCGGAGCGTAAACGGCTCGTGGCGCAGCACCTGACGCACGTCTCCCAGACCCAG
GAGGGCGACAAGGAGAATCTGAACCCGAATTCTGACGCGCCGGTGATCCGGTCAAAAACCTCCGCCAGGTACATG
GAGCTGGTCGGGTGGCTGGTGGACAAGGGCATCACGTCCGAGAAGCAGTGGATCCAGGAGGACCAGGCCTCGTAC
ATCTCCTTCAACGCGGCCTCCAACTCCCGGTCGCAGATCAAGGCGGCCCTGGACAATGCCTCCAAAATCATGAGC
CTCACCAAAACGGCTCCGGACTATCTCATCGGGCAGCAGCCCGTGGGGGACATTACCACCAACCGGATCTACAAA
ATCCTGGAACTGAACGGGTACGACCCCCAGTACGCCGCCTCCGTCTTTCTCGGCTGGGCCCAGAAAAAGTTTGGA
AAGCGCAACACCATCTGGCTGTTTGGGCCCGCCACCACCGGCAAGACCAACATCGCGGAAGCCATCGCCCACGCG
GTCCCCTTCTACGGCTGCGTCAACTGGACCAATGAGAACTTTCCCTTCAACGACTGCGTCGACAAAATGGTGATT
TGGTGGGAGGAGGGCAAGATGACCGCCAAGGTCGTAGAGTCCGCCAAGGCCATTCTGGGCGGCAGCAAGGTGCGC
GTGGACCAAAAATGCAAGGCCTCTGCGCAGATCGACCCCACCCCCGTGATCGTCACCTCCAACACCAACATGTGC
GCCGTGATTGACGGGAACAGCACCACCTTCGAGCACCAGCAGCCCCTGCAGGACCGGATGTTCAAGTTTGAACTC
ACCCGCCGCCTCGACCACGACTTTGGCAAGGTCACCAAGCAGGAAGTCAAGGACTTTTTCCGGTGGGCGGCTGAT
CACGTGACTGACGTGGCTCATGAGTTTTACGTCACAAAGGGTGGAGCTAAGAAAAGGCCCGCCCCCTCTGACGAG
GATATAAGCGAGCCCAAGCGGCCGCGCGTGTCATTTGCGCAGCCGGAGACGTCAGACGCGGAAGCTCCCGGAGAC
TTCGCCGACAGGTACCAAAACAAATGTTCTCGTCACGCGGGTATGCTGCAGATGCTCTTTCCCTGCAAGACGTGC
GAGAGAATGAATCAGAATTCCAACGTCTGCTTCACGCACGGTCAGAAAGATTGCGGGGAGTGCTTTCCCGGGTCA
GAATCTCAACCGGTTTCTGTCGTCAGAAAAACGTATCAGAAACTGTGCATCCTTCATCAGCTCCGGGGGGCACCC
GAGATCGCCTGCTCTGCTTGCGACCAACTCAACCCCGATTTGGACGATTGCCAATTTGAGCAATAA
CapVP1: (SEQ ID NO: 27)
ATGGCTGCTGACGGTTATCTTCCAGATTGGCTCGAGGACAACCTCTCTGAAGGCATTCGCGAGTGGTGGGCGCTG
AAACCTGGAGCTCCACAACCCAAGGCCAACCAACAGCATCAGGACAACGGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAGGCCTACGACAAGCAGCTCGAGCAGGGGGACAACCCGTATCTCAAGTACAACCACGCCGACGCCGAGTTC
CAGCAGCGCTTGGCGACCGACACCTCTTTTGGGGGCAACCTCGGGCGAGCAGTCTTCCAGGCCAAAAAGAGGATT
CTCGAGCCTCTGGGTCTGGTTGAAGAGGGCGTTAAAACGGCTCCTGGAAAGAAACGCCCATTAGAAAAGACTCCA
AATCGGCCGACCAACCCGGACTCTGGGAAGGCCCCGGCCAAGAAAAAGCAAAAAGACGGCGAACCAGCCGACTCT
GCTAGAAGGACACTCGACTTTGAAGACTCTGGAGCAGGAGACGGACCCCCTGAGGGATCATCTTCCGGAGAAATG
TCTCATGATGCTGAGATGCGTGCGGCGCCAGGCGGAAATGCTGTCGAGGCGGGACAAGGTGCCGATGGAGTGGGT
AATGCCTCCGGTGATTGGCATTGCGATTCCACCTGGTCAGAGGGCCGAGTCACCACCACCAGCACCCGAACCTGG
GTCCTACCCACGTACAACAACCACCTGTACCTGCGAATCGGAACAACGGCCAACAGCAACACCTACAACGGATTC
TCCACCCCCTGGGGATACTTTGACTTTAACCGCTTCCACTGCCACTTTTCCCCACGCGACTGGCAGCGACTCATC
AACAACAACTGGGGACTCAGGCCGAAATCGATGCGTGTTAAAATCTTCAACATACAGGTCAAGGAGGTCACGACG
TCAAACGGCGAGACTACGGTCGCTAATAACCTTACCAGCACGGTTCAGATCTTTGCGGATTCGACGTATGAACTC
CCATACGTGATGGACGCCGGTCAGGAGGGGAGCTTTCCTCCGTTTCCCAACGACGTCTTTATGGTTCCCCAATAC
GGATACTGCGGAGTTGTCACTGGAAAAAACCAGAACCAGACAGACAGAAATGCCTTTTACTGCCTGGAATACTTT
CCATCCCAAATGCTAAGAACTGGCAACAATTTTGAAGTCAGTTACCAATTTGAAAAAGTTCCTTTCCATTCAATG
TACGCGCACAGCCAGAGCCTGGACAGAATGATGAATCCTTTACTGGATCAGTACCTGTGGCATCTGCAATCGACC
ACTACCGGAAATTCCCTTAATCAAGGAACAGCTACCACCACGTACGGGAAAATTACCACTGGAGACTTTGCCTAC
TACAGGAAAAACTGGTTGCCTGGAGCCTGCATTAAACAACAAAAATTTTCAAAGAATGCCAATCAAAACTACAAG
ATTCCCGCCAGCGGGGGAGACGCCCTTTTAAAGTATGACACGCATACCACTCTAAATGGGCGATGGAGTAACATG
GCTCCTGGACCTCCAATGGCAACCGCAGGTGCCGGGGACTCGGATTTTAGCAACAGCCAGCTGATCTTTGCCGGA
CCCAATCCGAGCGGTAACACGACCACATCTTCAAACAATTTGTTGTTTACCTCAGAAGAGGAGATTGCCACAACA
AACCCACGAGACACGGACATGTTTGGACAGATTGCAGATAATAATCAAAATGCCACCACCGCCCCTCACATCGCT
AACCTGGACGCTATGGGAATTGTTCCCGGAATGGTCTGGCAAAACAGAGACATCTACTACCAGGGCCCTATTTGG
GCCAAGGTCCCTCACACGGACGGACACTTTCACCCTTCGCCGCTGATGGGAGGATTTGGACTGAAACACCCGCCT
CCACAGATTTTCATCAAAAACACCCCCGTACCCGCCAATCCCAATACTACCTTTAGCGCTGCAAGGATTAATTCT
TTTCTGACGCAGTACAGCACCGGACAAGTTGCCGTTCAGATCGACTGGGAAATTCAGAAGGAGCATTCCAAACGC
TGGAATCCCGAAGTTCAATTTACTTCAAACTACGGCACTCAAAATTCTATGCTGTGGGCTCCCGACAATGCTGGC
AACTACCACGAACTCCGGGCTATTGGGTCCCGTTTCCTCACCCACCACTTGTAA
AAV-13
Partial Genome: EU285562
Rep78: (SEQ ID NO: 28)
ATGCCGGGATTCTACGAGATTGTCCTGAAGGTGCCCAGCGACCTGGACGAGCACCTGCCTGGCATTTCTGACTCT
TTTGTAAACTGGGTGGCGGAGAAGGAATGGGAGCTGCCGCCGGATTCTGACATGGATCTGAATCTGATTGAGCAG
GCACCCCTAACCGTGGCCGAAAAGCTGCAACGCGAATTCCTGGTCGAGTGGCGCCGCGTGAGTAAGGCCCCGGAG
GCCCTCTTCTTTGTTCAGTTCGAGAAGGGGGACAGCTACTTCCACCTACACATTCTGGTGGAGACCGTGGGCGTG
AAATCCATGGTGGTGGGCCGCTACGTGAGCCAGATTAAAGAGAAGCTGGTGACCCGCATCTACCGCGGGGTCGAG
CCGCAGCTTCCGAACTGGTTCGCGGTGACCAAGACGCGTAATGGCGCCGGAGGCGGGAACAAGGTGGTGGACGAC
TGCTACATCCCCAACTACCTGCTCCCCAAGACCCAGCCCGAGCTCCAGTGGGCGTGGACTAATATGGACCAGTAT
TTAAGCGCCTGTTTGAATCTCGCGGAGCGTAAACGGCTGGTGGCGCAGCATCTGACGCACGTGTCGCAGACGCAG
GAGCAGAACAAAGAGAACCAGAATCCCAATTCTGACGCGCCGGTGATCAGATCAAAAACCTCCGCGAGGTACATG
GAGCTGGTCGGGTGGCTGGTGGACCGCGGGATCACGTCAGAAAAGCAATGGATCCAGGAGGACCAGGCCTCTTAC
ATCTCCTTCAACGCCGCCTCCAACTCGCGGTCACAAATCAAGGCCGCACTGGACAATGCCTCCAAATTTATGAGC
CTGACAAAAACGGCTCCGGACTACCTGGTGGGAAACAACCCGCCGGAGGACATTACCAGCAACCGGATCTACAAA
ATCCTCGAGATGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCTGAAGCTATCGCCCACGCC
GTGCCCTTTTACGGCTGCGTGAACTGGACCAATGAGAACTTTCCGTTCAACGATTGCGTCGACAAGATGGTGATC
TGGTGGGAGGAGGGCAAGATGACGGCCAAGGTCGTGGAGTCCGCCAAGGCCATTCTGGGCGGAAGCAAGGTGCGC
GTGGACCAAAAGTGCAAGTCATCGGCCCAGATCGACCCAACTCCCGTCATCGTCACCTCCAACACCAACATGTGC
GCGGTCATCGACGGAAATTCCACCACCTTCGAGCACCAACAACCACTCCAAGACCGGATGTTCAAGTTCGAGCTC
ACCAAGCGCCTGGAGCACGACTTTGGCAAGGTCACCAAGCAGGAAGTCAAGGACTTTTTCCGGTGGGCGTCAGAT
CACGTGACTGAGGTGTCTCACGAGTTTTACGTCAGAAAGGGTGGAGCTAGAAAGAGGCCCGCCCCCAATGACGCA
GATATAAGTGAGCCCAAGCGGGCCTGTCCGTCAGTTGCGCAGCCATCGACGTCAGACGCGGAAGCTCCGGTGGAC
TACGCGGACAGGTACCAAAACAAATGTTCTCGTCACGTGGGCATGAATCTGATGCTTTTTCCCTGCCGGCAATGC
GAGAGAATGAATCAGAATGTGGACATTTGCTTCACGCACGGGGTCATGGACTGTGCCGAGTGCTTCCCCGTGTCA
GAATCTCAACCCGTGTCTGTCGTCAGAAAGCGGACATATCAGAAACTGTGTCCGATTCATCACATCATGGGGAGG
GCGCCCGAGGTGGCTTGTTCGGCCTGCGATCTGGCCAATGTGGACTTGGATGACTGTGACATGGAGCAATAA
CapVP1: (SEQ ID NO: 29)
ATGACTGACGGTTACCTTCCAGATTGGCTAGAGGACAACCTCTCTGAAGGCGTTCGAGAGTGGTGGGCGCTGCAA
CCTGGAGCCCCTAAACCCAAGGCAAATCAACAACATCAGGACAACGCTCGGGGTCTTGTGCTTCCGGGTTACAAA
TACCTCGGACCCGGCAACGGACTTGACAAGGGGGAACCCGTCAACGCAGCGGACGCGGCAGCCCTCGAACACGAC
AAGGCCTACGACCAGCAGCTCAAGGCCGGTGACAACCCCTACCTCAAGTACAACCACGCCGACGCCGAGTTTCAG
GAGCGTCTTCAAGAAGATACGTCTTTTGGGGGCAACCTCGGACGAGCAGTCTTCCAGGCCAAAAAGAGGATCCTT
GAGCCTCTGGGTCTGGTTGAGGAAGCGGCTAAGACGGCTCCTGGAAAAAAGAGACCTGTAGAGCAATCTCCAGCA
GAACCGGACTCCTCTTCGGGCATCGGCAAATCAGGCCAGCAGCCCGCTAGAAAAAGACTGAATTTTGGTCAGACT
GGCGACACAGAGTCAGTCCCAGACCCTCAACCACTCGGACAACCTCCCGCAGCCCCCTCTGGTGTGGGATCTACT
ACAATGGCTTCAGGCGGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGCACCTGGGCCCTGCCCACC
TACAACAATCACCTCTACAAGCAAATCTCCAGCCAATCAGGAGCCACCAACGACAACCACTACTTTGGCTACAGC
ACCCCCTGGGGGTATTTTGACTTCAACAGATTCCACTGCCACTTTTCACCACGTGACTGGCAAAGACTCATCAAC
AACAACTGGGGATTCCGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCCGAGTACCAGCTCCCG
TACGTCCTCGGCTCGGCGCATCAGGGATGCCTCCCGCCGTTCCCAGCAGACGTCTTCATGGTCCCACAGTATGGA
TACCTCACCCTGAACAACGGGAGTCAGGCGGTAGGACGCTCTTCCTTTTACTGCCTGGAGTACTTTCCTTCTCAG
ATGCTGCGTACTGGAAACAACTTTCAGTTTAGCTACACTTTTGAAGACGTGCCTTTCCACAGCAGCTACGCTCAC
AGCCAAAGTCTGGACCGTCTCATGAATCCTCTGATCGACCAGTACCTGTACTATCTGAACAGGACACAAACAGCC
AGTGGAACTCAGCAGTCTCGGCTACTGTTTAGCCAAGCTGGACCCACCAGTATGTCTCTTCAAGCTAAAAACTGG
CTGCCTGGACCTTGCTACAGACAGCAGCGTCTGTCAAAGCAGGCAAACGACAACAACAACAGCAACTTTCCCTGG
ACTGGTGCCACCAAATATCATCTGAATGGCCGGGACTCATTGGTGAACCCGGGCCCTGCTATGGCCAGTCACAAG
GATGACAAAGAAAAGTTTTTCCCCATGCATGGAACCCTGATATTTGGTAAAGAAGGAACAAATGCCAACAACGCG
GATTTGGAAAATGTCATGATTACAGATGAAGAAGAAATCCGCACCACCAATCCCGTGGCTACGGAGCAGTACGGG
ACTGTGTCAAATAATTTGCAAAACTCAAACGCTGGTCCAACTACTGGAACTGTCAATCACCAAGGAGCGTTACCT
GGTATGGTGTGGCAGGATCGAGACGTGTACCTGCAGGGACCCATTTGGGCCAAGATTCCTCACACCGATGGACAC
TTTCATCCTTCTCCACTGATGGGAGGTTTTGGGCTCAAACACCCGCCTCCTCAGATCATGATCAAAAACACTCCC
GTTCCAGCCAATCCTCCCACAAACTTTAGTGCGGCAAAGTTTGCTTCCTTCATCACACAGTACTCCACGGGGCAG
GTCAGCGTGGAGATCGAGTGGGAGCTGCAGAAGGAGAACAGCAAACGCTGGAATCCCGAAATTCAGTACACTTCC
AACTACAACAAATCTGTTAATGTGGACTTTACTGTGGACACTAATGGTGTGTATTCAGAGCCTCGCCCCATTGGC
ACCAGATACCTGACTCGTAATCTGTAA
AAV rh10 (SEQ ID NO: 30)
GenBank: AY243015.1-Non-human primate Adeno-associated virus isolate
AAVrh. 10 capsid protein (VP1) gene, complete cds

| | |
|---|---|
| 1 | ATGGCTGCCG ATGGTTATCT TCCAGATTGG CTCGAGGACA ACCTCTCTGA GGGCATTCGC |
| 61 | GAGTGGTGGG ACTTGAAACC TGGAGCCCCG AAACCCAAAG CCAACCAGCA AAAGCAGGAC |
| 121 | GACGGCCGGG GTCTGGTGCT TCCTGGCTAC AAGTACCTCG GACCCTTCAA CGGACTCGAC |
| 181 | AAGGGGGAGC CCGTCAACGC GGCGGACGCA GCGGCCCTCG AGCACGACAA GGCCTACGAC |
| 241 | CAGCAGCTCA AAGCGGGTGA CAATCCGTAC CTGCGGTATA ACCACGCCGA CGCCGAGTTT |
| 301 | CAGGAGCGTC TGCAAGAAGA TACGTCTTTT GGGGGCAACC TCGGGCGAGC AGTCTTCCAG |
| 361 | GCCAAGAAGC GGGTTCTCGA ACCTCTCGGT CTGGTTGAGG AAGGCGCTAA GACGGCTCCT |
| 421 | GGAAAGAAGA GACCGGTAGA GCCATCACCC CAGCGTTCTC CAGACTCCTC TACGGGCATC |
| 481 | GGCAAGAAAG GCCAGCAGCC CGCGAAAAAG AGACTCAACT TTGGGCAGAC TGGCGACTCA |
| 541 | GAGTCAGTGC CCGACCCTCA ACCAATCGGA GAACCCCCCG CAGGCCCCTC TGGTCTGGGA |
| 601 | TCTGGTACAA TGGCTGCAGG CGGTGGCGCT CCAATGGCAG ACAATAACGA AGGCGCCGAC |
| 661 | GGAGTGGGTA GTTCCTCAGG AAATTGGCAT TGCGATTCCA CATGGCTGGG CGACAGAGTC |
| 721 | ATCACCACCA GCACCCGAAC CTGGGCCCTC CCCACCTACA ACAACCACCT CTACAAGCAA |
| 781 | ATCTCCAACG GGACTTCGGG AGGAAGCACC AACGACAACA CCTACTTCGG CTACAGCACC |
| 841 | CCCTGGGGGT ATTTTGACTT TAACAGATTC CACTGCCACT TCTCACCACG TGACTGGCAG |
| 901 | CGACTCATCA ACAACAACTG GGGATTCCGG CCCAAGAGAC TCAACTTCAA GCTCTTCAAC |
| 961 | ATCCAGGTCA AGGAGGTCAC GCAGAATGAA GGCACCAAGA CCATCGCCAA TAACCTTACC |
| 1021 | AGCACGATTC AGGTCTTTAC GGACTCGGAA TACCAGCTCC CGTACGTCCT CGGCTCTGCG |
| 1081 | CACCAGGGCT GCCTGCCTCC GTTCCCGGCG GACGTCTTCA TGATTCCTCA GTACGGGTAC |
| 1141 | CTGACTCTGA ACAATGGCAG TCAGGCCGTG GGCCGTTCCT CCTTCTACTG CCTGGAGTAC |
| 1201 | TTTCCTTCTC AAATGCTGAG AACGGGCAAC AACTTTGAGT TCAGCTACCA GTTTGAGGAC |
| 1261 | GTGCCTTTTC ACAGCAGCTA CGCGCACAGC CAAAGCCTGG ACCGGCTGAT GAACCCCCTC |
| 1321 | ATCGACCAGT ACCTGTACTA CCTGTCTCGG ACTCAGTCCA CGGGAGGTAC CGCAGGAACT |
| 1381 | CAGCAGTTGC TATTTTCTCA GGCCGGGCCT AATAACATGT CGGCTCAGGC CAAAAACTGG |
| 1441 | CTACCCGGGC CCTGCTACCG GCAGCAACGC GTCTCCACGA CACTGTCGCA AAATAACAAC |
| 1501 | AGCAACTTTG CCTGGACCGG TGCCACCAAG TATCATCTGA ATGGCAGAGA CTCTCTGGTA |
| 1561 | AATCCCGGTG TCGCTATGGC AACCCACAAG GACGACGAAG AGCGATTTTT TCCGTCCAGC |
| 1621 | GGAGTCTTAA TGTTTGGGAA ACAGGGAGCT GGAAAAGACA ACGTGGACTA TAGCAGCGTT |
| 1681 | ATGCTAACCA GTGAGGAAGA AATTAAAACC ACCAACCCAG TGGCCACAGA ACAGTACGGC |
| 1741 | GTGGTGGCCG ATAACCTGCA ACAGCAAAAC GCCGCTCCTA TTGTAGGGGC CGTCAACAGT |
| 1801 | CAAGGAGCCT TACCTGGCAT GGTCTGGCAG AACCGGGACG TGTACCTGCA GGGTCCTATC |
| 1861 | TGGGCCAAGA TTCCTCACAC GGACGGAAAC TTTCATCCCT CGCCGCTGAT GGGAGGCTTT |
| 1921 | GGACTGAAAC ACCCGCCTCC TCAGATCCTG ATTAAGAATA CACCTGTTCC CGCGGATCCT |
| 1981 | CCAACTACCT TCAGTCAAGC TAAGCTGGCG TCGTTCATCA CGCAGTACAG CACCGGACAG |
| 2041 | GTCAGCGTGG AAATTGAATG GGAGCTGCAG AAAGAAAACA GCAAACGCTG GAACCCAGAG |
| 2101 | ATTCAATACA CTTCCAACTA CTACAAATCT ACAAATGTGG ACTTTGCTGT TAACACAGAT |
| 2161 | GGCACTTATT CTGAGCCTCG CCCCATCGGC ACCCGTTACC TCACCCGTAA TCTGTAA |

AAV rh39: (SEQ ID NO: 31)
GENBANK: EU368921.1 ADENO-ASSOCIATED VIRUS ISOLATE RH. 39 CAPSID PROTEIN VP1
GENE, PARTIAL CDS

| | |
|---|---|
| 1 | ATGGCTGCCG ATGGTTATCT TCCAGATTGG CTCGAGGACA ACCTCTCTGA GGGCATTCGC |
| 61 | GAGTGGTGGG CGCTGAAACC TGGAGCCCCG AAGCCCAAAG CCAACCAGCA AAAGCAGGAC |
| 121 | GACGGCCGGG GTCTGGTGCT TCCTGGCTAC AAGTACCTCG GACCCTTCAA CGGACTCGAC |
| 181 | AAGGGGGAGC CCGTCAACGC GGCGGACGCA GCGGCCCTCG AGCACGACAA GGCCTACGAC |
| 241 | CAGCAGCTCA AAGCGGGTGA CAATCCGTAC CTGCGGTATA ACCACGCCGA CGCCGAGTTT |
| 301 | CAGGAGCGTC TGCAAGAAGA TACGTCTTTT GGGGGCAACC TCGGGCGAGC AGTCTTCCAG |
| 361 | GCCAAGAAGC GGGTTCTCGA ACCTCTCGGT CTGGTTGAGG AAGCTGCTAA GACGGCTCCT |
| 421 | GGAAAGAAGA GACCGGTAGA ACCGTCACCT CAGCGTTCCC CCGACTCCTC CACGGGCATC |
| 481 | GGCAAGAAAG GCCAGCAGCC CGCTAAAAAG AGACTGAACT TTGGTCAGAC TGGCGACTCA |
| 541 | GAGTCAGTCC CCGACCCTCA ACCAATCGGA GAACCACCAG CAGGCCCCTC TGGTCTGGGA |
| 601 | TCTGGTACAA TGGCTGCAGG CGGTGGCGCT CCAATGGCAG ACAATAACGA AGGCGCCGAC |
| 661 | GGAGTGGGTA GTTCCTCAGG AAATTGGCAT TGCGATTCCA CATGGCTGGG CGACAGAGTC |
| 721 | ATCACCACCA GCACCCGAAC CTGGGCCCTG CCCACCTACA ACAACCACCT CTACAAGCAA |
| 781 | ATATCCAATG GGACATCGGG AGGAAGCACC AACGACAACA CCTACTTCGG CTACAGCACC |
| 841 | CCCTGGGGGT ATTTTGACTT CAACAGATTC CACTGCCACT TCTCACCACG TGACTGGCAG |
| 901 | CGACTCATCA ACAACAACTG GGGATTCCGG CCAAAAAGAC TCAGCTTCAA GCTCTTCAAC |
| 961 | ATCCAGGTCA AGGAGGTCAC GCAGAATGAA GGCACCAAGA CCATCGCCAA TAACCTTACC |
| 1021 | AGCACGATTC AGGTATTTAC GGACTCGGAA TACCAGCTGC CGTACGTCCT CGGCTCCGCG |
| 1081 | CACCAGGGCT GCCTGCCTCC GTTCCCGGCG GACGTCTTCA TGATTCCCCA GTACGGCTAC |
| 1141 | CTTACACTGA ACAATGGAAG TCAAGCCGTA GGCCGTTCCT CCTTCTACTG CCTGGAATAT |
| 1201 | TTTCCATCTC AAATGCTGCG AACTGGAAAC AATTTTGAAT TCAGCTACAC CTTCGAGGAC |
| 1261 | GTGCCTTTCC ACAGCAGCTA CGCACACAGC CAGAGCTTGG ACCGACTGAT GAATCCTCTC |
| 1321 | ATCGACCAGT ACCTGTACTA CTTATCCAGA ACTCAGTCCA CAGGAGGAAC TCAAGGTACC |
| 1381 | CAGCAATTGT TATTTTCTCA AGCTGGGCCT GCAAACATGT CGGCTCAGGC TAAGAACTGG |
| 1441 | CTACCTGGAC CTTGCTACCG GCAGCAGCGA GTCTCTACGA CACTGTCGCA AAACAACAAC |
| 1501 | AGCAACTTTG CTTGGACTGG TGCCACCAAA TATCACCTGA ACGGAAGAGA CTCTTTGGTA |
| 1561 | AATCCCGGTG TCGCCATGGC AACCCACAAG GACGACGAGG AACGCTTCTT CCCGTCGAGT |
| 1621 | GGAGTCCTGA TGTTTGGAAA ACAGGGTGCT GGAAGAGACA ATGTGGACTA CAGCAGCGTT |
| 1681 | ATGCTAACCA GCGAAGAAGA AATTAAAACC ACTAACCCTG TAGCCACAGA ACAATACGGT |
| 1741 | GTGGTGGCTG ATAACTTGCA GCAAACCAAT ACGGGGCCTA TTGTGGGAAA TGTCAACAGC |
| 1801 | CAAGGAGCCT TACCTGGCAT GGTCTGGCAG AACCGAGACG TGTACCTGCA GGGTCCCATC |
| 1861 | TGGGCCAAGA TTCCTCACAC GGACGGCAAC TTCCACCCTT CACCGCTAAT GGGAGGATTT |
| 1921 | GGACTGAAGC ACCCACCTCC TCAGATCCTG ATCAAGAACA CGCCGGTACC TGCGGATCCT |
| 1981 | CCAACAACGT TCAGCCAGGC GAAATTGGCT TCCTTCATTA CGCAGTACAG CACCGGACAG |
| 2041 | GTCAGCGTGG AAATCGAGTG GGAGCTGCAG AAGGAGAACA GCAAACGCTG GAACCCAGAG |
| 2101 | ATTCAGTACA CTTCAAACTA CTACAAATCT ACAAATGTGG ACTTTGCTGT CAATACAGAG |
| 2161 | GGAACTTATT CTGAGCCTCG CCCCATTGGT ACTCGTTACC TCACCCGTAA TCTG |

AAV rh43: (SEQ ID NO: 32)
GENBANK: JA400154.1 AAV serotype, clone rh. 43

| | |
|---|---|
| 1 | ATGGCTGCCG ATGGTTATCT TCCAGATTGG CTCGAGGACA ACCTCTCTGA GGGCATTCGC |
| 61 | GAGTGGTGGG ACTTGAAACC TGGAGCCCCG AAACCCAAAG CCAACCAGCA AAAGCAGGAC |
| 121 | GACGGCCGGG GCCTGGTGCT TCCTGGCTAC AAGTACCTCG GACCCTTCAA CGGACTCGAC |
| 181 | AAGGGGGAGC CCGTCAACGC GGCGGACGCA GCGGCCCTCG AGCACGACAA GGCCTACGAC |
| 241 | CAGCAGCTCG AAGCGGGTGA CAATCCGTAC CTGCGGTATA ACCACGCCGA CGCCGAGTTT |
| 301 | CAGGAGCGTC TGCAAGAAGA TACGTCTTTT GGGGGCAACC TCGGGCGAGC AGTCTTCCAG |
| 361 | GCCAAGAAGC GGGTTCTCGA ACCTCTCGGT CTGGTTGAGG AAGGCGCTAA GACGGCTCCT |
| 421 | GGAAAGAAGA GACCAGTAGA GCAGTCACCC CAAGAACCAG ACTCCTCCTC GGGCATCGGC |
| 481 | AAGAAAGGCC AACAGCCCGC CAGAAAAAGA CTCAATTTTG GCCAGACTGG CGACTCAGAG |
| 541 | TCAGTTCCAG ACCCTCAACC TCTCGGAGAA CCTCCAGCAG CGCCCTCTGG TGTGGGACCT |
| 601 | AATACAATGG CTGCAGGCGG TGGCGCACCA ATGGCAGACA ATAACGAAGG CGCCGACGGA |
| 661 | GTGGGTAGTT CCTCGGGAAA TTGGCATTGC GATTCCACAT GGCTGGGCGA CAGAGTCATC |
| 721 | ACCACCAGCA CCCGAACCTG GGCCCTGCCC ACCTACAACA ACCACCTCTA CAAGCAAATC |
| 781 | TCCAACGGGA CATCGGGAGG AGCCACCAAC GACAACACCT ACTTCGGCTA CAGCACCCCC |
| 841 | TGGGGGTATT TTGACTTTAA CAGATTCCAC TGCCACTTTT CACCACGTGA CTGGCAGCGA |
| 901 | CTCATCAACA ACAACTGGGG ATTCCGGCCC AAGAGACTCA GCTTCAAGCT CTTCAACATC |
| 961 | CAGGTCAAGG AGGTCACGCA GAATGAAGGC ACCAAGACCA TCGCCAATAA CCTCACCAGC |
| 1021 | ACCATCCAGG TGTTTACGGA CTCGGAGTAC CAGCTGCCGT ACGTTCTCGG CTCTGCCCAC |
| 1081 | CAGGGCTGCC TGCCTCCGTT CCCGGCGGAC GTGTTCATGA TTCCCCAGTA CGGCTACCTA |
| 1141 | ACACTCAACA ACGGTAGTCA GGCCGTGGGA CGCTCCTCCT TCTACTGCCT GGAATACTTT |
| 1201 | CCTTCGCAGA TGCTGAGAAC CGGCAACAAC TTCCAGTTTA CTTACACCTT CGAGGACGTG |
| 1261 | CCTTTCCACA GCAGCTACGC CCACAGCCAG AGCTTGGACC GGCTGATGAA TCCTCTGATT |
| 1321 | GACCAGTACC TGTACTACTT GTCTCGGACT CAAACAACAG GAGGCACGGC AAATACGCAG |
| 1381 | ACTCTGGGCT TCAGCCAAGG TGGGCCTAAT ACAATGGCCA ATCAGGCAAA GAACTGGCTG |
| 1441 | CCAGGACCCT GTTACCGCCA ACAACGCGTC TCAACGACAA CCGGGCAAAA CAACAATAGC |
| 1501 | AACTTTGCCT GGACTGCTGG GACCAAATAC CATCTGAATG GAAGAAATTC ATTGGCTAAT |
| 1561 | CCTGGCATCG CTATGGCAAC ACACAAAGAC GACGAGGAGC GTTTTTTCCC AGTAACGGGA |
| 1621 | TCCTGTTTTT GGCAACAAAA TGCTGCCAGA GACAATGCGG ATTACAGCGA TGTCATGCTC |
| 1681 | ACCAGCGAGG AAGAAATCAA AACCACTAAC CCTGTGGCTA CAGAGGAATA CGGTATCGTG |
| 1741 | GCAGATAACT TGCAGCAGCA AAACACGGCT CCTCAAATTG GAACTGTCAA CAGCCAGGGG |
| 1801 | GCCTTACCCG GTATGGTCTG GCAGAACCGG GACGTGTACC TGCAGGGTCC CATCTGGGCC |
| 1861 | AAGATTCCTC ACACGGACGG CAACTTCCAC CCGTCTCCGC TGATGGGCGG CTTTGGCCTG |
| 1921 | AAACATCCTC CGCCTCAGAT CCTGATCAAG AACACGCCTG TACCTGCGGA TCCTCCGACC |
| 1981 | ACCTTCAACC AGTCAAAGCT GAACTCTTTC ATCACGCAAT ACAGCACCGG ACAGGTCAGC |
| 2041 | GTGGAAATTG AATGGGAGCT ACAGAAGGAA AACAGCAAGC GCTGGAACCC CGAGATCCAG |
| 2101 | TACACCTCCA ACTACTACAA ATCTACAAGT GTGGACTTTG CTGTTAATAC AGAAGGCGTG |
| 2161 | TACTCTGAAC CCCGCCCCAT TGGCACCCGT TACCTCACCC GTAATCTGTA A |

AAVrh. 74: (SEQ ID NO: 33)
Nucleotide sequence encoding AAV rh74 capsid protein:
ATGGCTGCCGATGGTTATCTTCCAGATTGGCTCGAGGACAACCTCTCTGAGGGCATTCGCGAGTGGTGGGACCTG
AA
ACCTGGAGCCCCGAAACCCAAAGCCAACCAGCAAAAGCAGGACAACGGCCGGGGTCTGGTGCTTCCTGGCTACAA
GT
ACCTCGGACCCTTCAACGGACTCGACAAGGGGGAGCCCGTCAACGCGGCGGACGCAGCGGCCCTCGAGCACGACA
AG
GCCTACGACCAGCAGCTCCAAGCGGGTGACAATCCGTACCTGCGGTATAATCACGCCGACGCCGAGTTTCAGGAG
CG
TCTGCAAGAAGATACGTCTTTTGGGGGCAACCTCGGGCGCGCAGTCTTCCAGGCCAAAAAGCGGGTTCTCGAACC
TC
TGGGCCTGGTTGAATCGCCGGTTAAGACGGCTCCTGGAAAGAAGAGACCGGTAGAGCCATCACCCCAGCGCTCTC
CA
GACTCCTCTACGGGCATCGGCAAGAAAGGCCAGCAGCCCGCAAAAAAGAGACTCAATTTTGGGCAGACTGGCGAC
TC
AGAGTCAGTCCCCGACCCTCAACCAATCGGAGAACCACCAGCAGGCCCCTCTGGTCTGGGATCTGGTACAATGGC
TG
CAGGCGGTGGCGCTCCAATGGCAGACAATAACGAAGGCGCCGACGGAGTGGGTAGTTCCTCAGGAAATTGGCATT
GC
GATTCCACATGGCTGGGCGACAGAGTCATCACCACCAGCACCCGCACCTGGGCCCTGCCCACCTACAACAACCAC
CT
CTACAAGCAAATCTCCAACGGGACCTCGGGAGGAAGCACCAACGACAACACCTACTTCGGCTACAGCACCCCCTG
GG
GGTATTTTGACTTCAACAGATTCCACTGCCACTTTTCACCACGTGACTGGCAGCGACTCATCAACAACAACTGGG
GA
TTCCGGCCCAAGAGGCTCAACTTCAAGCTCTTCAACATCCAAGTCAAGGAGGTCACGCAGAATGAAGGCACCAAG
AC
CATCGCCAATAACCTTACCAGCACGATTCAGGTCTTTACGGACTCGGAATACCAGCTCCCGTACGTGCTCGGCTC
GG
CGCACCAGGGCTGCCTGCCTCCGTTCCCGGCGGACGTCTTCATGATTCCTCAGTACGGGTACCTGACTCTGAACA
AT
GGCAGTCAGGCTGTGGGCCGGTCGTCCTTCTACTGCCTGGAGTACTTTCCTTCTCAAATGCTGAGAACGGGCAAC
AA
CTTTGAATTCAGCTACAACTTCGAGGACGTGCCCTTCCACAGCAGCTACGCGCACAGCCAGAGCCTGGACCGGCT
GA
TGAACCCTCTCATCGACCAGTACTTGTACTACCTGTCCCGGACTCAAAGCACGGGCGGTACTGCAGGAACTCAGC
AG
TTGCTATTTTCTCAGGCCGGGCCTAACAACATGTCGGCTCAGGCCAAGAACTGGCTACCCGGTCCCTGCTACCGG
CA
GCAACGCGTCTCCACGACACTGTCGCAGAACAACAACAGCAACTTTGCCTGGACGGGTGCCACCAAGTATCATCT
GA
ATGGCAGAGACTCTCTGGTGAATCCTGGCGTTGCCATGGCTACCCACAAGGACGACGAAGAGCGATTTTTTCCAT
CC
AGCGGAGTCTTAATGTTTGGGAAACAGGGAGCTGGAAAAGACAACGTGGACTATAGCAGCGTGATGCTAACCAGC
GA
GGAAGAAATAAAGACCACCAACCCAGTGGCCACAGAACAGTACGGCGTGGTGGCCGATAACCTGCAACAGCAAAA
CG
CCGCTCCTATTGTAGGGGCCGTCAATAGTCAAGGAGCCTTACCTGGCATGGTGTGGCAGAACCGGGACGTGTACC
TG
CAGGGTCCCATCTGGGCCAAGATTCCTCATACGGACGGCAACTTTCATCCCTCGCCGCTGATGGGAGGCTTTGGA
CT
GAAGCATCCGCCTCCTCAGATCCTGATTAAAAACACACCTGTTCCCGCCGATCCTCCGACCACCTTCAATCAGGC
CA
AGCTGGCTTCTTTCATCACGCAGTACAGTACCGGTCAGGTCAGCGTGGAGATCGAGTGGGAGCTGCAGAAGGAGA
AC
AGCAAACGCTGGAACCCAGAGATTCAGTACACTTCCAACTACTACAAATCTACAAATGTGGACTTTGCTGTCAAT
AC TGAGGGTACTTATTCCGAGCCTCGCCCCATTGGCACCCGTTACCTCACCCGTAATCTGTAA
AAV2 7M8: AAV2 7m8 is characterized by a 10-amino acid peptide (SEQ ID NO: 34)
LALGETTRPA
ITR Sequence (SEQ ID NO: 35)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCT
Rep2 Sequence-Contains Rep78 and Rep52 (start codon underlined) (SEQ ID NO: 36)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTTCTGACGGAATGGCGCCGTGTGAGTAAGGCCCCGGAG
GCCCTTTTCTTTGTGCAATTTGAGAAGGGAGAGAGCTACTTCCACATGCACGTGCTCGTGGAAACCACCGGGGTG
AAATCCATGGTTTTGGGACGTTTCCTGAGTCAGATTCGCGAAAAACTGATTCAGAGAATTTACCGCGGGATCGAG
CCGACTTTGCCAAACTGGTTCGCGGTCACAAAGACCAGAAATGGCGCCGGAGGCGGGAACAAGGTGGTGGATGAG
TGCTACATCCCCAATTACTTGCTCCCCAAAACCCAGCCTGAGCTCCAGTGGGCGTGGACTAATATGGAACAGTAT
TTAAGCGCCTGTTTGAATCTCACGGAGCGTAAACGGTTGGTGGCGCAGCATCTGACGCACGTGTCGCAGACGCAG
GAGCAGAACAAAGAGAATCAGAATCCCAATTCTGATGCGCCGGTGATCAGATCAAAAACTTCAGCCAGGTACATG
GAGCTGGTCGGGTGGCTCGTGGACAAGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTTGGACAATGCGGGAAAGATTATGAGC
CTGACTAAAACCGCCCCCGACTACCTGGTGGGCCAGCAGCCCGTGGAGGACATTTCCAGCAATCGGATTTATAAA
ATTTTGGAACTAAACGGGTACGATCCCCAATATGCGGCTTCCGTCTTTCTGGGATGGGCCACGAAAAAGTTCGGC
AAGAGGAACACCATCTGGCTGTTTGGGCCTGCAACTACCGGGAAGACCAACATCGCGGAGGCCATAGCCCACACT
GTGCCCTTCTACGGGTGCGTAAACTGGACCAATGAGAACTTTCCCTTCAACGACTGTGTCGACAAGATGGTGATC
TGGTGGGAGGAGGGGAAGATGACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCAAATTTGAACTC
ACCCGCCGTCTGGATCATGACTTTGGGAAGGTCACCAAGCAGGAAGTCAAAGACTTTTTCCGGTGGGCAAAGGAT
CACGTGGTTGAGGTGGAGCATGAATTCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATGGGAAAGGTG
CCAGACGCTTGCACTGCCTGCGATCTGGTCAATGTGGATTTGGATGACTGCATCTTTGAACAATAA
Cap2 Sequence-contains sequentially VP1, VP2, AAP, VP3 (start codons
underlined) (SEQ ID NO: 37)
ATGGCTGCCGATGGTTATCTTCCAGATTGGCTCGAGGACACTCTCTCTGAAGGAATAAGACAGTGGTGGAAGCTC
AAACCTGGCCCACCACCACCAAAGCCCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCGACGCGGAGTTT
CAGGAGCGCCTAAAGAAGATACGTCTTTTGGGGGCAACCTCGGACGAGCAGTCTTCCAGGCGAAAAAGAGGGTT
CTTGAACCTCTGGGCCTGGTTGAGGAACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGCAAGAAAAAGATTGAATTTTGGTCAG
ACTGGAGACGCAGACTCAGTACCTGACCCCCAGCCTCTCGGACAGCCACCAGCAGCCCCCTCTGGTCTGGGAACT
AATACGATGGCTACAGGCAGTGGCGCACCAATGGCAGACAATAACGAGGGCGCCGACGGAGTGGGTAATTCCTCG
GGAAATTGGCATTGCGATTCCACATGGATGGGCGACAGAGTCATCACCACCAGCACCCGAACCTGGGCCCTGCCC
ACCTACAACAACCACCTCTACAAACAAATTTCCAGCCAATCAGGAGCCTCGAACGACAATCACTACTTTGGCTAC
AGCACCCCTTGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACTTTCCTTCT
CAGATGCTGCGTACCGGAAACAACTTTACCTTCAGCTACACTTTTGAGGACGTTCCTTTCCACAGCAGCTACGCT
CACAGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCTGCGGATAACAACAACAGTGAATAC
TCGTGGACTGGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCAGAGAAAACA
AATGTGGACATTGAAAAGGTCATGATTACAGACGAAGAGGAAATCAGGACAACCAATCCCGTGGCTACGGAGCAG
TATGGTTCTGTATCTACCAACCTCCAGAGAGGCAACAGACAAGCAGCTACCGCAGATGTCAACACACAAGGCGTT
CTTCCAGGCATGGTCTGGCAGGACAGAGATGTGTACCTTCAGGGGCCCATCTGGGCAAAGATTCCACACACGGAC
GGACATTTTCACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACAGTACTCCACG
GGACAGGTCAGCGTGGAGATCGAGTGGGAGCTGCAGAAGGAAAACAGCAAACGCTGGAATCCCGAAATTCAGTAC
ACTTCCAACTACAACAAGTCTGTTAATGTGGACTTTACTGTGGACACTAATGGCGTGTATTCAGAGCCTCGCCCC
ATTGGCACCAGATACCTGACTCGTAATCTGTAA
Cap5 Sequence-contains sequentially VP1, VP2, AAP, VP3 (start codons
underlined) (SEQ ID NO: 38)
ATGGCTTTTGTTGATCACCCTCCAGATTGGTTGGAAGAAGTTGGTGAAGGTCTTCGCGAGTTTTTGGGCCTTGAA
GCGGGCCCACCGAAACCAAAACCCAATCAGCAGCATCAAGATCAAGCCCGTGGTCTTGTGCTGCCTGGTTATAAC
TATCTCGGACCCGGAAACGGTCTCGATCGAGGAGAGCCTGTCAACAGGGCAGACGAGGTCGCGCGAGAGCACGAC
ATCTCGTACAACGAGCAGCTTGAGGCGGGAGACAACCCCTACCTCAAGTACAACCACGCGGACGCCGAGTTTCAG
GAGAAGCTCGCCGACGACACATCCTTCGGGGGAAACCTCGGAAAGGCAGTCTTTCAGGCCAAGAAAAGGGTTCTC
GAACCTTTTGGCCTGGTTGAAGAGGGTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGACGCCGAAGCTGGACCCAGCGGATCC
CAGCAGCTGCAAATCCCAGCCCAACCAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGATTGGCATTGCGATTCCACGTGGATG
GGGGACAGAGTCGTCACCAAGTCCACCCGAACCTGGGTGCTGCCCAGCTACAACAACCACCAGTACCGAGAGATC
AAAAGCGGCTCCGTCGACGGAAGCAACGCCAACGCCTACTTTGGATACAGCACCCCCTGGGGGTACTTTGACTTT
AACCGCTTCCACAGCCACTGGAGCCCCCGAGACTGGCAAAGACTCATCAACAACTACTGGGGCTTCAGACCCCGG
TCCCTCAGAGTCAAAATCTTCAACATTCAAGTCAAAGAGGTCACGGTGCAGGACTCCACCACCACCATCGCCAAC
AACCTCACCTCCACCGTCCAAGTGTTTACGGACGACGACTACCAGCTGCCCTACGTCGTCGGCAACGGGACCGAG
GGATGCCTGCCGGCCTTCCCTCCGCAGGTCTTTACGCTGCCGCAGTACGGTTACGCGACGCTGAACCGCGACAAC
ACAGAAAATCCCACCGAGAGGAGCAGCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTTCGCTCCCAGTCAGAACCTCTTCAAG
CTGGCCAACCCGCTGGTGGACCAGTACTTGTACCGCTTCGTGAGCACAAATAACACTGGCGGAGTCCAGTTCAAC
AAGAACCTGGCCGGGAGATACGCCAACACCTACAAAAACTGGTTCCCGGGGCCCATGGGCCGAACCCAGGGCTGG
AACCTGGGCTCCGGGGTCAACCGCGCCAGTGTCAGCGCCTTCGCCACGACCAATAGGATGGAGCTCGAGGGCGCG
AGTTACCAGGTGCCCCCGCAGCCGAACGGCATGACCAACAACCTCCAGGGCAGCAACACCTATGCCCTGGAGAAC
ACTATGATCTTCAACAGCCAGCCGGCGAACCCGGGCACCACCGCCACGTACCTCGAGGGCAACATGCTCATCACC
AGCGAGAGCGAGACGCAGCCGGTGAACCGCGTGGCGTACAACGTCGGCGGGCAGATGGCCACCAACAACCAGAGC
TCCACCACTGCCCCCGCGACCGGCACGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCACTTTCACCCCTCTCCGGCCATGGGC
GGATTCGGACTCAAACACCCACCGCCCATGATGCTCATCAAGAACACGCCTGTGCCCGGAAATATCACCAGCTTC
TCGGACGTGCCCGTCAGCAGCTTCATCACCCAGTACAGCACCGGGCAGGTCACCGTGGAGATGGAGTGGGAGCTC
AAGAAGGAAAACTCCAAGAGGTGGAACCCAGAGATCCAGTACACAAACAACTACAACGACCCCCAGTTTGTGGAC
TTTGCCCCGGACAGCACCGGGGAATACAGAAGCACCAGACCTATCGGAACCCGATACCTTACCCGACCCCTTTAA

### Example 33—Adenovirus Polynucleotide Sequences

Adenovirus (Ad) polynucleotides can be selected from any serotype, and representative polynucleotides are exemplified below.

E2A Full Sequence
(SEQ ID NO: 39)
CGACCGCACCCTGTGACGAAAGCCGCCCGCAAGCTGCGCCCCTGAGTTAGTCATCTGAACTTCGGCCTGGGCGT
CTCTGGGAAGTACCACAGTGGTGGGAGCGGGACTTTCCTGGTACACCAGGGCAGCGGGCCAACTACGGGGATTAA
GGTTATTACGAGGTGTGGTGGTAATAGCCGCCTGTTCGAGGAGAATTCGGTTTCGGTGGGCGCGGATTCCGTTGA
CCCGGGATATCATGTGGGGTCCCGCGCTCATGTAGTTTATTCGGGTTGAGTAGTCTTGGGCAGCTCCAGCCGCAA
GTCCCATTTGTGGCTGGTAACTCCACATGTAGGGCGTGGGAATTTCCTTGCTCATAATGGCGCTGACGACAGGTG
CTGGCGCCGGGTGTGGCCGCTGGAGATGACGTAGTTTTCGCGCTTAAATTTGAGAAAGGGCGCGAAACTAGTCCT
TAAGAGTCAGCGCGCAGTATTTGCTGAAGAGAGCCTCCGCGTCTTCCAGCGTGCGCCGAAGCTGATCTTCGCTTT
TGTGATACAGGCAGCTGCGGGTGAGGGAGCGCAGAGACCTGTTTTTTATTTTCAGCTCTTGTTCTTGGCCCCTGC
TTTGTTGAAATATAGCATACAGAGTGGGAAAAATCCTATTTCTAAGCTCGCGGGTCGATACGGGTTCGTTGGGCG
CCAGACGCAGCGCTCCTCCTCCTGCTGCTGCCGCCGCTGTGGATTTCTTGGGCTTTGTCAGAGTCTTGCTATCCG
GTCGCCTTTGCTTCTGTGTGACCGCTGCTGTTGCTGCCGCTGCCGCTGCCGCCGGTGCAGTAGGGGCTGTAGAGA
TGACGGTAGTAATGCAGGATGTTACGGGGGAAGGCCACGCCGTGATGGTAGAGAAGAAAGCGGCGGGCGAAGGAG
ATGTTGCCCCCACAGTCTTGCAAGCAAGCAACTATGGCGTTCTTGTGCCCGCGCCACGAGCGGTAGCCTTGGCGC
TGTTGTTGCTCTTGGGCTAACGGCGGCGGCTGCTTAGACTTACCGGCCCTGGTTCCAGTGGTGTCCCATCTACGG
TTGGGTCGGCGAACAGGCAGTGCCGGCGGCGCCTGAGGAGCGGAGGTTGTAGCGATGCTGGGAACGGTTGCCAAT
TTCTGGGGCGCCGGCGAGGGGAATGCGACCGAGGGTGACGGTGTTTCGTCTGACACCTCTTCGGCCTCGGAAGCT
TCGTCTAGGCTGTCCCAGTCTTCCATCATCTCCTCCTCCTCGTCCAAAACCTCCTCTGCCTGACTGTCCCAGTAT
TCCTCCTCGTCCGTGGGTGGCGGCGGCGGCAGCTGCAGCTTCTTTTTGGGTGCCATCCTGGGAAGCAAGGGCCCG
CGGCTGCTGATAGGGCTGCGGCGGCGGGGGGATTGGGTTGAGCTCCTCGCCGGACTGGGGGTCCAGGTAAACCCC
CCGTCCCTTTCGTAGCAGAAACTCTTGGCGGGCTTTGTTGATGGCTTGCAATTGGCCAAGGATGTGGCCCTGGGT
AATGACGCAGGCGGTAAGCTCCGCATTTGGCGGGCGGGATTGGTCTTCGTAGAACCTAATCTCGTGGGCGTGGTA
GTCCTCAGGTACAAATTTGCGAAGGTAAGCCGACGTCCACAGCCCCGGAGTGAGTTTCAACCCCGGAGCCGCGGA
CTTTTCGTCAGGCGAGGGACCCTGCAGCTCAAAGGTACCGATAATTTGACTTTCGCTAAGCAGTTGCGAATTGCA
GACCAGGGAGCGGTGCGGGGTGCATAGGTTGCAGCGACAGTGACACTCCAGTAGGCCGTCACCGCTCACGTCTTC
CATGATGTCGGAGTGGTAGGCAAGGTAGTTGGCTAGCTGCAGAAGGTAGCAGTGACCCCAAAGCGGCGGAGGGCA
TTCACGGTACTTAATGGGCACAAAGTCGCTAGGAAGCGCACAGCAGGTGGCGGGCAGAATTCCTGAACGCTCTAG
GATAAAGTTCCTAAAGTTTTGCAACATGCTTTGACTGGTGAAGTCTGGCAGACCCTGTTGCAGGGTTTTAAGCAG
GCGTTCGGGGAAGATAATGTCCGCCAGGTGCGCGGCCACGGAGCGCTCGTTGAAGGCCGTCCATAGGTCCTTCAA
GTTTTGCTTTAGCAGCTTCTGCAGCTCCTTTAGGTTGCGCTCCTCCAGGCATTGCTGCCACACGCCCATGGCCGT
TTGCCAGGTGTAGCACAGAAATAAGTAAACGCAGTCGCGGACGTAGTCGCGGCGCGCCTCGCCCTTGAGCGTGGA
ATGAAGCACGTTTTGCCCGAGGCGGTTTTCGTGCAAAATTCCAAGGTAGGAGACCAGGTTGCAGAGCTCCACGTT
GGAAATTTTGCAGGCCTGGCGCACGTAGCCCTGGCGAAAGGTGTAGTGCAACGTTTCCTCTAGCTTGCGCTGCAT
CTCCGGGTCAGCAAAGAACCGCTGCATGCACTCAAGCTCCACGGTAACAAGCACTGCGGCCATCATTAGCTTGCG
TCGCTCCTCCAAGTCGGCAGGCTCGCGCGTCTCAAGCCAGCGCGCCAGCTGCTCATCGCCAACTGCGGGTAGGCC
CTCCTCGGTTTGTTCTTGCAAGTTTGCATCCCTCTCCAGGGGTCGTGCACGGCGCACGATCAGCTCGCTCATGAC
TGTGCTCATAACCTTGGGGGGTAGGTTAAGTGCCGGGTAGGCAAAGTGGGTGACCTCGATGCTGCGTTTCAGCAC
GGCTAGGCGCGCGTTGTCACCCTCAAGTTCCACCAGCACTCCACAGTGACTTTCATTTTCGCTGTTTTCTTGTTG
CAGAGCGTTTGCCGCGCGTTTCTCGTCGCGTCCAAGACCCTCAAAGATTTTTGGCACTTCGTCGAGCGAGGCGAT
ATCAGGTATGACAGCGCCCTGCCGCAAGGCCAGCTGCTTGTCCGCTCGGCTGCGGTTGGCACGGCAGGATAGGGG
TATCTTGCAGTTTTGGAAAAAGATGTGATAGGTGGCAAGCACCTCTGGCACGGCAAATACGGGGTAGAAGTTGAG
GCGCGGGTTGGGCTCGCATGTGCCGTTTTCTTGGCGTTTGGGGGGTACGCGCGGTGAGAACAGGTGGCGTTCGTA
GGCAAGGCTGACATCCGCTATGGCGAGGGGCACATCGCTGCGCTCTTGCAACGCGTCGCAGATAATGGCGCACTG
GCGCTGCAGATGCTTCAACAGCACGTCGTCTCCCACATCTAGGTAGTCGCCATGCCTTTGGTCCCCCCGCCCGAC
TTGTTCCTCGTTTGCCTCTGCGTCGTCCTGGTCTTGCTTTTTATCCTCTGTTGGTACTGAGCGATCCTCGTCGTC
TTCGCTTACAAAACCTGGGTCCTGCTCGATAATCACTTCCTCCTCCTCAAGCGGGGGTGCCTCGACGGGGAAGGT
GGTAGGCGCGTTGGCGGCATCGGTGGAGGCGGTGGTGGCGAACTCAAAGGGGGCGGTTAGGCTGTCCTCCTTCTC
GACTGACTCCATGATCTTTTTCTGCCTATAGGAGAAGGAAATGGCCAGTCGGGAAGAGGAGCAGCGCGAAACCAC
CCCCGAGCGCGGACGCGGTGCGGCGCGACGTCCACCAACCATGGAGGACGTGTCGTCCCCGTCGCCGTCGCCGCC
GCCTCCCCGCGCGCCCCCAAAAAAGCGGCTGAGGCGGCGTCTCGAGTCCGAGGACGAAGAAGACTCGTCACAAGA
TGCGCTGGTGCCGCGCACACCCAGCCCGCGGCCATCGACCTCGACGGCGGATTTGGCCATTGCGTCCAAAAAGAA
AAAGAAGCGCCCCTCTCCCAAGCCCGAGCGCCCGCCATCCCCAGAGGTGATCGTGGACAGCGAGGAAGAAAGAGA
AGATGTGGCGCTACAAATGGTGGGTTTCAGCAACCCACCGGTGCTAATCAAGCACGGCAAGGGAGGTAAGCGCAC
GGTGCGGCGGCTGAATGAAGACGACCCAGTGGCGCGGGGTATGCGGACGCAAGAGGAAAAGGAAGAGTCCAGTGA
AGCGGAAAGTGAAAGCACGGTGATAAACCCGCTGAGCCTGCCGATCGTGTCTGCGTGGGAGAAGGGCATGGAGGC
TGCGCGCGCGTTGATGGACAAGTACCACGTGGATAACGATCTAAAGGCAAACTTCAAGCTACTGCCTGACCAAGT
GGAAGCTCTGGCGGCCGTATGCAAGACCTGGCTAAACGAGGAGCACCGCGGGTTGCAGCTGACCTTCACCAGCAA
CAAGACCTTTGTGACGATGATGGGGCGATTCCTGCAGGCGTACCTGCAGTCGTTTGCAGAGGTAACCTACAAGCA
CCACGAGCCCACGGGCTGCGCGTTGTGGCTGCACCGCTGCGCTGAGATCGAAGGCGAGCTTAAGTGTCTACACGG
GAGCATTATGATAAATAAGGAGCACGTGATTGAAATGGATGTGACGAGCGAAAACGGGCAGCGCGCGCTGAAGGA
GCAGTCTAGCAAGGCCAAGATCGTGAAGAACCGGTGGGGCCGAAATGTGGTGCAGATCTCCAACACCGACGCAAG
GTGCTGCGTGCATGACGCGGCCTGTCCGGCCAATCAGTTTTCCGGCAAGTCTTGCGGCATGTTCTTCTCTGAAGG
CGCAAAGGCTCAGGTGGCTTTTAAGCAGATCAAGGCTTTCATGCAGGCGCTGTATCCTAACGCCCAGACCGGGCA
CGGTCACCTTCTGATGCCACTACGGTGCGAGTGCAACTCAAAGCCTGGGCATGCACCCTTTTTGGGAAGGCAGCT
ACCAAAGTTGACTCCGTTCGCCCTGAGCAACGCGGAGGACCTGGACGCGGATCTGATCTCCGACAAGAGCGTGCT
GGCCAGCGTGCACCACCCGGCGCTGATAGTGTTCCAGTGCTGCAACCCTGTGTATCGCAACTCGCGCGCGCAGGG
CGGAGGCCCCAACTGCGACTTCAAGATATCGGCGCCCGACCTGCTAAACGCGTTGGTGATGGTGCGCAGCCTGTG
GAGTGAAAACTTCACCGAGCTGCCGCGGATGGTTGTGCCTGAGTTTAAGTGGAGCACTAAACACCAGTATCGCAA
CGTGTCCCTGCCAGTGGCGCATAGCGATGCGCGGCAGAACCCCTTTGATTTTTAAACGGCGCAGACGGCAAGGGT
GGGGGGTAAATAATCACCCGAGAGTGTACAAATAAAAACATTTGCCTTTATTGAAAGTGTCTCCTAGTACATTAT
TTTTACATGTTTTTCAAGTGACAAAAAGAAGTGGCGCTCCTAATCTGCGCACTGTGGCTGCGGAAGTAGGGCGAG
TGGCGCTCCAGGAAGCTGTAGAGCTGTTCCTGGTTGCGACGCAGGGTGGGCTGTACCTGGGGACTGTTAAGCATG
GAGTTGGGTACC
E2A ORF Sequence
(SEQ ID NO: 40)
ATGGCCAGTCGGGAAGAGGAGCAGCGCGAAACCACCCCCGAGCGCGGACGCGGTGCGGCGCGACGTCCACCAACC
ATGGAGGACGTGTCGTCCCCGTCGCCGTCGCCGCCGCCTCCCCGCGCGCCCCCAAAAAAGCGGCTGAGGCGGCGT
CTCGAGTCCGAGGACGAAGAAGACTCGTCACAAGATGCGCTGGTGCCGCGCACACCCAGCCCGCGGCCATCGACC
TCGACGGCGGATTTGGCCATTGCGTCCAAAAAGAAAAAGAAGCGCCCCTCTCCCAAGCCCGAGCGCCCGCCATCC
CCAGAGGTGATCGTGGACAGCGAGGAAGAAAGAGAAGATGTGGCGCTACAAATGGTGGGTTTCAGCAACCCACCG
GTGCTAATCAAGCACGGCAAGGGAGGTAAGCGCACGGTGCGGCGGCTGAATGAAGACGACCCAGTGGCGCGGGGT
ATGCGGACGCAAGAGGAAAAGGAAGAGTCCAGTGAAGCGGAAAGTGAAAGCACGGTGATAAACCCGCTGAGCCTG
CCGATCGTGTCTGCGTGGGAGAAGGGCATGGAGGCTGCGCGCGCGTTGATGGACAAGTACCACGTGGATAACGAT
CTAAAGGCAAACTTCAAGCTACTGCCTGACCAAGTGGAAGCTCTGGCGGCCGTATGCAAGACCTGGCTAAACGAG
GAGCACCGCGGGTTGCAGCTGACCTTCACCAGCAACAAGACCTTTGTGACGATGATGGGGCGATTCCTGCAGGCG
TACCTGCAGTCGTTTGCAGAGGTAACCTACAAGCACCACGAGCCCACGGGCTGCGCGTTGTGGCTGCACCGCTGC
GCTGAGATCGAAGGCGAGCTTAAGTGTCTACACGGGAGCATTATGATAAATAAGGAGCACGTGATTGAAATGGAT
GTGACGAGCGAAAACGGGCAGCGCGCGCTGAAGGAGCAGTCTAGCAAGGCCAAGATCGTGAAGAACCGGTGGGGC
CGAAATGTGGTGCAGATCTCCAACACCGACGCAAGGTGCTGCGTGCATGACGCGGCCTGTCCGGCCAATCAGTTT
TCCGGCAAGTCTTGCGGCATGTTCTTCTCTGAAGGCGCAAAGGCTCAGGTGGCTTTTAAGCAGATCAAGGCTTTC
ATGCAGGCGCTGTATCCTAACGCCCAGACCGGGCACGGTCACCTTCTGATGCCACTACGGTGCGAGTGCAACTCA
AAGCCTGGGCATGCACCCTTTTTGGGAAGGCAGCTACCAAAGTTGACTCCGTTCGCCCTGAGCAACGCGGAGGAC
CTGGACGCGGATCTGATCTCCGACAAGAGCGTGCTGGCCAGCGTGCACCACCCGGCGCTGATAGTGTTCCAGTGC
TGCAACCCTGTGTATCGCAACTCGCGCGCGCAGGGCGGAGGCCCCAACTGCGACTTCAAGATATCGGCGCCCGAC
CTGCTAAACGCGTTGGTGATGGTGCGCAGCCTGTGGAGTGAAAACTTCACCGAGCTGCCGCGGATGGTTGTGCCT
GAGTTTAAGTGGAGCACTAAACACCAGTATCGCAACGTGTCCCTGCCAGTGGCGCATAGCGATGCGCGGCAGAAC
CCCTTTGATTTTTAA
E4 Full Sequence
(SEQ ID NO: 41)
CCCGGGCGTTTTAGGGCGGAGTAACTTGCATGTATTGGGAATTGTAGTTTTTTTAAAATGGGAAGTGACGTATCG
TGGGAAAACGGAAGTGAAGATTTGAGGAAGTTGTGGGTTTTTTGGCTTTCGTTTCTGGGCGTAGGTTCGCGTGCG
GTTTTCTGGGTGTTTTTTGTGGACTTTAACCGTTACGTCATTTTTTAGTCCTATATATACTCGCTCTGTACTTGG
CCCTTTTTACACTGTGACTGATTGAGCTGGTGCCGTGTCGAGTGGTGTTTTTTAATAGGTTTTTTTACTGGTAAG
GCTGACTGTTATGGCTGCCGCTGTGGAAGCGCTGTATGTTGTTCTGGAGCGGGAGGGTGCTATTTTGCCTAGGCA
GGAGGGTTTTTCAGGTGTTTATGTGTTTTTCTCTCCTATTAATTTTGTTATACCTCCTATGGGGGCTGTAATGTT
GTCTCTACGCCTGCGGGTATGTATTCCCCCGGGCTATTTCGGTCGCTTTTTAGCACTGACCGATGTTAACCAACC
TGATGTGTTTACCGAGTCTTACATTATGACTCCGGACATGACCGAGGAACTGTCGGTGGTGCTTTTTAATCACGG
TGACCAGTTTTTTTACGGTCACGCCGGCATGGCCGTAGTCCGTCTTATGCTTATAAGGGTTGTTTTTCCTGTTGT
AAGACAGGCTTCTAATGTTTAAATGTTTTTTTTTTTGTTATTTTATTTTGTGTTTAATGCAGGAACCCGCAGACA
TGTTTGAGAGAAAAATGGTGTCTTTTTCTGTGGTGGTTCCGGAACTTACCTGCCTTTATCTGCATGAGCATGACT
ACGATGTGCTTGCTTTTTTGCGCGAGGCTTTGCCTGATTTTTTGAGCAGCACCTTGCATTTTATATCGCCGCCCA
TGCAACAAGCTTACATAGGGGCTACGCTGGTTAGCATAGCTCCGAGTATGCGTGTCATAATCAGTGTGGGTTCTT
TTGTCATGGTTCCTGGCGGGGAAGTGGCCGCGCTGGTCCGTGCAGACCTGCACGATTATGTTCAGCTGGCCCTGC
GAAGGGACCTACGGGATCGCGGTATTTTTGTTAATGTTCCGCTTTTGAATCTTATACAGGTCTGTGAGGAACCTG
AATTTTTGCAATCATGATTCGCTGCTTGAGGCTGAAGGTGGAGGGCGCTCTGGAGCAGATTTTTACAATGGCCGG
ACTTAATATTCGGGATTTGCTTAGAGACATATTGATAAGGTGGCGAGATGAAAATTATTTGGGCATGGTTGAAGG
TGCTGGAATGTTTATAGAGGAGATTCACCCTGAAGGGTTTAGCCTTTACGTCCACTTGGACGTGAGGGCAGTTTG
CCTTTTGGAAGCCATTGTGCAACATCTTACAAATGCCATTATCTGTTCTTTGGCTGTAGAGTTTGACCACGCCAC
CGGAGGGGAGCGCGTTCACTTAATAGATCTTCATTTTGAGGTTTTGGATAATCTTTTGGAATAAAAAAAAAAAAA
CATGGTTCTTCCAGCTCTTCCCGCTCCTCCCGTGTGTGACTCGCAGAACGAATGTGTAGGTTGGCTGGGTGTGGC
TTATTCTGCGGTGGTGGATGTTATCAGGGCAGCGGCGCATGAAGGAGTTTACATAGAACCCGAAGCCAGGGGGCG
CCTGGATGCTTTGAGAGAGTGGATATACTACAACTACTACACAGAGCGAGCTAAGCGACGAGACCGGAGACGCAG
ATCTGTTTGTCACGCCCGCACCTGGTTTTGCTTCAGGAAATATGACTACGTCCGGCGTTCCATTTGGCATGACAC
TACGACCAACACGATCTCGGTTGTCTCGGCGCACTCCGTACAGTAGGGATCGCCTACCTCCTTTTGAGACAGAGA
CCCGCGCTACCATACTGGAGGATCATCCGCTGCTGCCCGAATGTAACACTTTGACAATGCACAACGTGAGTTACG
TGCGAGGTCTTCCCTGCAGTGTGGGATTTACGCTGATTCAGGAATGGGTTGTTCCCTGGGATATGGTTCTGACGC
GGGAGGAGCTTGTAATCCTGAGGAAGTGTATGCACGTGTGCCTGTGTTGTGCCAACATTGATATCATGACGAGCA
TGATGATCCATGGTTACGAGTCCTGGGCTCTCCACTGTCATTGTTCCAGTCCCGGTTCCCTGCAGTGCATAGCCG
GCGGGCAGGTTTTGGCCAGCTGGTTTAGGATGGTGGTGGATGGCGCCATGTTTAATCAGAGGTTTATATGGTACC
GGGAGGTGGTGAATTACAACATGCCAAAAGAGGTAATGTTTATGTCCAGCGTGTTTATGAGGGGTCGCCACTTAA
TCTACCTGCGCTTGTGGTATGATGGCCACGTGGGTTCTGTGGTCCCCGCCATGAGCTTTGGATACAGCGCCTTGC
ACTGTGGGATTTTGAACAATATTGTGGTGCTGTGCTGCAGTTACTGTGCTGATTTAAGTGAGATCAGGGTGCGCT
GCTGTGCCCGGAGGACAAGGCGTCTCATGCTGCGGGCGGTGCGAATCATCGCTGAGGAGACCACTGCCATGTTGT
ATTCCTGCAGGACGGAGCGGCGGCGGCAGCAGTTTATTCGCGCGCTGCTGCAGCACCACCGCCCTATCCTGATGC
ACGATTATGACTCTACCCCCATGTAGGCGTGGACTTCCCCTTCGCCGCCCGTTGAGCAACCGCAAGTTGGACAGC
AGCCTGTGGCTCAGCAGCTGGACAGCGACATGAACTTAAGCGAGCTGCCCGGGGAGTTTATTAATATCACTGATG
AGCGTTTGGCTCGACAGGAAACCGTGTGGAATATAACACCTAAGAATATGTCTGTTACCCATGATATGATGCTTT
TTAAGGCCAGCCGGGGAGAAAGGACTGTGTACTCTGTGTGTTGGGAGGGAGGTGGCAGGTTGAATACTAGGGTTC
TGTGAGTTTGATTAAGGTACGGTGATCAATATAAGCTATGTGGTGGTGGGGCTATACTACTGAATGAAAAATGAC
TTGAAATTTTCTGCAATTGAAAAATAAACACGTTGAAACATAACATGCAACAGGTTCACGATTCTTTATTCCTGG
GCAATGTAGGAGAAGGTGTAAGAGTTGGTAGCAAAAGTTTCAGTGGTGTATTTTCCACTTTCCCAGGACCATGTA
AAAGACATAGAGTAAGTGCTTACCTCGCTAGTTTCTGTGGATTCACTAGAA
E4 Orf6 Sequence
(SEQ ID NO: 42)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGTGAGTTACGTGCGAGGTCTTCCCTGCAGTGTGGGATTTACGCTGATTCAG
GAATGGGTTGTTCCCTGGGATATGGTTCTGACGCGGGAGGAGCTTGTAATCCTGAGGAAGTGTATGCACGTGTGC
CTGTGTTGTGCCAACATTGATATCATGACGAGCATGATGATCCATGGTTACGAGTCCTGGGCTCTCCACTGTCAT
TGTTCCAGTCCCGGTTCCCTGCAGTGCATAGCCGGCGGGCAGGTTTTGGCCAGCTGGTTTAGGATGGTGGTGGAT
GGCGCCATGTTTAATCAGAGGTTTATATGGTACCGGGAGGTGGTGAATTACAACATGCCAAAAGAGGTAATGTTT
ATGTCCAGCGTGTTTATGAGGGGTCGCCACTTAATCTACCTGCGCTTGTGGTATGATGGCCACGTGGGTTCTGTG
GTCCCCGCCATGAGCTTTGGATACAGCGCCTTGCACTGTGGGATTTTGAACAATATTGTGGTGCTGTGCTGCAGT
TACTGTGCTGATTTAAGTGAGATCAGGGTGCGCTGCTGTGCCCGGAGGACAAGGCGTCTCATGCTGCGGGCGGTG
CGAATCATCGCTGAGGAGACCACTGCCATGTTGTATTCCTGCAGGACGGAGCGGCGGCGGCAGCAGTTTATTCGC
GCGCTGCTGCAGCACCACCGCCCTATCCTGATGCACGATTATGACTCTACCCCCATGTAG
VA Sequence (VA transcripts I and II are underlined)
(SEQ ID NO: 43)
CGTAATCCGTAGATGTACCTGGACATCCAGGTGATGCCGGCGGCGGTGGTGGAGGCGCGCGGAAAGTCGCGGAC
GCGGTTCCAGATGTTGCGCAGCGGCAAAAAGTGCTCCATGGTCGGGACGCTCTGGCCGGTGAGGCGTGCGCAGTC
GTTGACGCTCTAGACCGTGCAAAAGGAGAGCCTGTAAGCGGGCACTCTTCCGTGGTCTGGTGGATAAATTCGCAA
GGGTATCATGGCGGACGACCGGGGTTCGAACCCCGGATCCGGCCGTCCGCCGTGATCCATGCGGTTACCGCCCGC
GTGTCGAACCCAGGTGTGCGACGTCAGACAACGGGGGAGCGCTCCTTTTGGCTTCCTTCCAGGCGCGGCGGCTGC
TGCGCTAGCTTTTTTGGCCACTGGCCGCGCGCGGCGTAAGCGGTTAGGCTGGAAAGCGAAAGCATTAAGTGGCTC
GCTCCCTGTAGCCGGAGGGTTATTTTCCAAGGGTTGAGTCGCAGGACCCCCGGTTCGAGTCTCGGGCCGGCCGGA
CTGCGGCGAACGGGGGTTTGCCTCCCCGTCATGCAAGACCCCGCTTGCAAATTCCTCCGGAAACAGGGACGAGCC
CCTTTTTTGCTTTTCCCAGATGCATCCGGTGCTGCGGCAGATGCGCCCCCCTCCTCAGCAGCGGCAAGAGCAAGA
GCAGCGGCAGACATGCAGGGCACCCTCCCCTTCTCCTACCGCGTCAGGAGGGGCAACATCCTACATCGA
Sequences for E1A and E1B are both contained within Accession AY339865.1
Ad5 E1A
Two proteins can be transcribed, a 32 kDa protein (first accession number)
and a 27 kDa protein (second accession number). These are both splice
variants from the transcript:
Accession 1: AAQ19284.1
Accession 2: AAQ19285.1
(SEQ ID NO: 44)
ATGAGACATATTATCTGCCACGGAGGTGTTATTACCGAAGAAATGGCCGCCAGTCTTTTGGACCAGCTGATCGAA
GAGGTACTGGCTGATAATCTTCCACCTCCTAGCCATTTTGAACCACCTACCCTTCACGAACTGTATGATTTAGAC
GTGACGGCCCCCGAAGATCCCAACGAGGAGGCGGTTTCGCAGATTTTTCCCGACTCTGTAATGTTGGCGGTGCAG
GAAGGGATTGACTTACTCACTTTTCCGCCGGCGCCCGGTTCTCCGGAGCCGCCTCACCTTTCCCGGCAGCCCGAG
CAGCCGGAGCAGAGAGCCTTGGGTCCGGTTTCTATGCCAAACCTTGTACCGGAGGTGATCGATCTTACCTGCCAC
GAGGCTGGCTTTCCACCCAGTGACGACGAGGATGAAGAGGGTGAGGAGTTTGTGTTAGATTATGTGGAGCACCCC
GGGCACGGTTGCAGGTCTTGTCATTATCACCGGAGGAATACGGGGGACCCAGATATTATGTGTTCGCTTTGCTAT
ATGAGGACCTGTGGCATGTTTGTCTACAGTCCTGTGTCTGAACCTGAGCCTGAGCCCGAGCCAGAACCGGAGCCT
GCAAGACCTACCCGCCGTCCTAAAATGGCGCCTGCTATCCTGAGACGCCCGACATCACCTGTGTCTAGAGAATGC
AATAGTAGTACGGATAGCTGTGACTCCGGTCCTTCTAACACACCTCCTGAGATACACCCGGTGGTCCCGCTGTGC
CCCATTAAACCAGTTGCCGTGAGAGTTGGTGGGCGTCGCCAGGCTGTGGAATGTATCGAGGACTTGCTTAACGAG
CCTGGGCAACCTTTGGACTTGAGCTGTAAACGCCCCAGGCCATAA
(SEQ ID NO: 45)
ATGAGACATATTATCTGCCACGGAGGTGTTATTACCGAAGAAATGGCCGCCAGTCTTTTGGACCAGCTGATCGAA
GAGGTACTGGCTGATAATCTTCCACCTCCTAGCCATTTTGAACCACCTACCCTTCACGAACTGTATGATTTAGAC
GTGACGGCCCCCGAAGATCCCAACGAGGAGGCGGTTTCGCAGATTTTTCCCGACTCTGTAATGTTGGCGGTGCAG
GAAGGGATTGACTTACTCACTTTTCCGCCGGCGCCCGGTTCTCCGGAGCCGCCTCACCTTTCCCGGCAGCCCGAG
CAGCCGGAGCAGAGAGCCTTGGGTCCGGTTTCTATGCCAAACCTTGTACCGGAGGTGATCGATCTTACCTGCCAC
GAGGCTGGCTTTCCACCCAGTGACGACGAGGATGAAGAGGGTCCTGTGTCTGAACCTGAGCCTGAGCCCGAGCCA
GAACCGGAGCCTGCAAGACCTACCCGCCGTCCTAAAATGGCGCCTGCTATCCTGAGACGCCCGACATCACCTGTG
TCTAGAGAATGCAATAGTAGTACGGATAGCTGTGACTCCGGTCCTTCTAACACACCTCCTGAGATACACCCGGTG
GTCCCGCTGTGCCCCATTAAACCAGTTGCCGTGAGAGTTGGTGGGCGTCGCCAGGCTGTGGAATGTATCGAGGAC
TTGCTTAACGAGCCTGGGCAACCTTTGGACTTGAGCTGTAAACGCCCCAGGCCATAA
Ad5 E1B_19K
Accession: AAQ19286.1
(SEQ ID NO: 46)
ATGGAGGCTTGGGAGTGTTTGGAAGATTTTTCTGCTGTGCGTAACTTGCTGGAACAGAGCTCTAACAGTACCTCT
TGGTTTTGGAGGTTTCTGTGGGGCTCATCCCAGGCAAAGTTAGTCTGCAGAATTAAGGAGGATTACAAGTGGGAA
TTTGAAGAGCTTTTGAAATCCTGTGGTGAGCTGTTTGATTCTTTGAATCTGGGTCACCAGGCGCTTTTCCAAGAG
AAGGTCATCAAGACTTTGGATTTTTCCACACCGGGGCGCGCTGCGGCTGCTGTTGCTTTTTTGAGTTTTATAAAG
GATAAATGGAGCGAAGAAACCCATCTGAGCGGGGGGTACCTGCTGGATTTTCTGGCCATGCATCTGTGGAGAGCG
GTTGTGAGACACAAGAATCGCCTGCTACTGTTGTCTTCCGTCCGCCCGGCGATAATACCGACGGAGGAGCAGCAG
CAGCAGCAGGAGGAAGCCAGGCGGCGGCGGCAGGAGCAGAGCCCATGGAACCCGAGAGCCGGCCTGGACCCTCGG
GAATGA
Ad5 E1B_55K
Accession: AAQ19287.1
(SEQ ID NO: 47)
ATGGAGCGAAGAAACCCATCTGAGCGGGGGGTACCTGCTGGATTTTCTGGCCATGCATCTGTGGAGAGCGGTTGT
GAGACACAAGAATCGCCTGCTACTGTTGTCTTCCGTCCGCCCGGCGATAATACCGACGGAGGAGCAGCAGCAGCA
GCAGGAGGAAGCCAGGCGGCGGCGGCAGGAGCAGAGCCCATGGAACCCGAGAGCCGGCCTGGACCCTCGGGAATG
AATGTTGTACAGGTGGCTGAACTGTATCCAGAACTGAGACGCATTTTGACAATTACAGAGGATGGGCAGGGGCTA
AAGGGGGTAAAGAGGGAGCGGGGGGCTTGTGAGGCTACAGAGGAGGCTAGGAATCTAGCTTTTAGCTTAATGACC
AGACACCGTCCTGAGTGTATTACTTTTCAACAGATCAAGGATAATTGCGCTAATGAGCTTGATCTGCTGGCGCAG
AAGTATTCCATAGAGCAGCTGACCACTTACTGGCTGCAGCCAGGGGATGATTTTGAGGAGGCTATTAGGGTATAT
GCAAAGGTGGCACTTAGGCCAGATTGCAAGTACAAGATCAGCAAACTTGTAAATATCAGGAATTGTTGCTACATT
TCTGGGAACGGGGCCGAGGTGGAGATAGATACGGAGGATAGGGTGGCCTTTAGATGTAGCATGATAAATATGTGG
CCGGGGGTGCTTGGCATGGACGGGGTGGTTATTATGAATGTAAGGTTTACTGGCCCCAATTTTAGCGGTACGGTT
TTCCTGGCCAATACCAACCTTATCCTACACGGTGTAAGCTTCTATGGGTTTAACAATACCTGTGTGGAAGCCTGG
ACCGATGTAAGGGTTCGGGGCTGTGCCTTTTACTGCTGCTGGAAGGGGGTGGTGTGTCGCCCCAAAAGCAGGGCT
TCAATTAAGAAATGCCTCTTTGAAAGGTGTACCTTGGGTATCCTGTCTGAGGGTAACTCCAGGGTGCGCCACAAT
GTGGCCTCCGACTGTGGTTGCTTCATGCTAGTGAAAAGCGTGGCTGTGATTAAGCATAACATGGTATGTGGCAAC
TGCGAGGACAGGGCCTCTCAGATGCTGACCTGCTCGGACGGCAACTGTCACCTGCTGAAGACCATTCACGTAGCC
AGCCACTCTCGCAAGGCCTGGCCAGTGTTTGAGCATAACATACTGACCCGCTGTTCCTTGCATTTGGGTAACAGG
AGGGGGGTGTTCCTACCTTACCAATGCAATTTGAGTCACACTAAGATATTGCTTGAGCCCGAGAGCATGTCCAAG
GTGAACCTGAACGGGGTGTTTGACATGACCATGAAGATCTGGAAGGTGCTGAGGTACGATGAGACCCGCACCAGG
TGCAGACCCTGCGAGTGTGGCGGTAAACATATTAGGAACCAGCCTGTGATGCTGGATGTGACCGAGGAGCTGAGG
CCCGATCACTTGGTGCTGGCCTGCACCCGCGCTGAGTTTGGCTCTAGCGATGAAGATACAGATTGA
Sequences for E2A and E4A are both contained within Accession MN088492 Ad5 E2A
orf:
Accession: QHX41645.1
(SEQ ID NO: 48)
ATGGCCAGTCGGGAAGAGGAGCAGCGCGAAACCACCCCCGAGCGCGGACGCGGTGCGGCGCGACGTCCACCAACC
ATGGAGGACGTGTCGTCCCCGTCGCCGTCGCCGCCGCCTCCCCGCGCGCCCCCAAAAAAGCGGCTGAGGCGGCGT
CTCGAGTCCGAGGACGAAGAAGACTCGTCACAAGATGCGCTGGTGCCGCGCACACCCAGCCCGCGGCCATCGACC
TCGACGGCGGATTTGGCCATTGCGTCCAAAAAGAAAAAGAAGCGCCCCTCTCCCAAGCCCGAGCGCCCGCCATCC
CCAGAGGTGATCGTGGACAGCGAGGAAGAAAGAGAAGATGTGGCGCTACAAATGGTGGGTTTCAGCAACCCACCG
GTGCTAATCAAGCACGGCAAGGGAGGTAAGCGCACGGTGCGGCGGCTGAATGAAGACGACCCAGTGGCGCGGGGT
ATGCGGACGCAAGAGGAAAAGGAAGAGTCCAGTGAAGCGGAAAGTGAAAGCACGGTGATAAACCCGCTGAGCCTG
CCGATCGTGTCTGCGTGGGAGAAGGGCATGGAGGCTGCGCGCGCGTTGATGGACAAGTACCACGTGGATAACGAT
CTAAAGGCAAACTTCAAGCTACTGCCTGACCAAGTGGAAGCTCTGGCGGCCGTATGCAAGACCTGGCTAAACGAG
GAGCACCGCGGGTTGCAGCTGACCTTCACCAGCAACAAGACCTTTGTGACGATGATGGGGCGATTCCTGCAGGCG
TACCTGCAGTCGTTTGCAGAGGTAACCTACAAGCACCACGAGCCCACGGGCTGCGCGTTGTGGCTGCACCGCTGC
GCTGAGATCGAAGGCGAGCTTAAGTGTCTACACGGGAGCATTATGATAAATAAGGAGCACGTGATTGAAATGGAT
GTGACGAGCGAAAACGGGCAGCGCGCGCTGAAGGAGCAGTCTAGCAAGGCCAAGATCGTGAAGAACCGGTGGGGC
CGAAATGTGGTGCAGATCTCCAACACCGACGCAAGGTGCTGCGTGCATGACGCGGCCTGTCCGGCCAATCAGTTT
TCCGGCAAGTCTTGCGGCATGTTCTTCTCTGAAGGCGCAAAGGCTCAGGTGGCTTTTAAGCAGATCAAGGCTTTC
ATGCAGGCGCTGTATCCTAACGCCCAGACCGGGCACGGTCACCTTCTGATGCCACTACGGTGCGAGTGCAACTCA
AAGCCTGGGCATGCACCCTTTTTGGGAAGGCAGCTACCAAAGTTGACTCCGTTCGCCCTGAGCAACGCGGAGGAC
CTGGACGCGGATCTGATCTCCGACAAGAGCGTGCTGGCCAGCGTGCACCACCCGGCGCTGATAGTGTTCCAGTGC
TGCAACCCTGTGTATCGCAACTCGCGCGCGCAGGGCGGAGGCCCCAACTGCGACTTCAAGATATCGGCGCCCGAC
CTGCTAAACGCGTTGGTGATGGTGCGCAGCCTGTGGAGTGAAAACTTCACCGAGCTGCCGCGGATGGTTGTGCCT
GAGTTTAAGTGGAGCACTAAACACCAGTATCGCAACGTGTCCCTGCCAGTGGCGCATAGCGATGCGCGGCAGAAC
CCCTTTGATTTTTAA
Ad5 E4A:
Two proteins are present in this ORF. The first is a splice variant contained
within the ORF. The second is a non-spliced transcript present in the ORF.
Accession 1: QHX41659.1
Accession 2: QHX41660.1
(SEQ ID NO: 49)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGCGTGGACTTCCCCTTCGCCGCCCGTTGAGCAACCGCAAGTTGGACAGCAG
CCTGTGGCTCAGCAGCTGGACAGCGACATGAACTTAAGCGAGCTGCCCGGGGAGTTTATTAATATCACTGATGAG
CGTTTGGCTCGACAGGAAACCGTGTGGAATATAACACCTAAGAATATGTCTGTTACCCATGATATGATGCTTTTT
AAGGCCAGCCGGGGAGAAAGGACTGTGTACTCTGTGTGTTGGGAGGGAGGTGGCAGGTTGAATACTAGGGTTCTG
TGA
(SEQ ID NO: 50)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGTGAGTTACGTGCGAGGTCTTCCCTGCAGTGTGGGATTTACGCTGATTCAG
GAATGGGTTGTTCCCTGGGATATGGTTCTGACGCGGGAGGAGCTTGTAATCCTGAGGAAGTGTATGCACGTGTGC
CTGTGTTGTGCCAACATTGATATCATGACGAGCATGATGATCCATGGTTACGAGTCCTGGGCTCTCCACTGTCAT
TGTTCCAGTCCCGGTTCCCTGCAGTGCATAGCCGGCGGGCAGGTTTTGGCCAGCTGGTTTAGGATGGTGGTGGAT
GGCGCCATGTTTAATCAGAGGTTTATATGGTACCGGGAGGTGGTGAATTACAACATGCCAAAAGAGGTAATGTTT
ATGTCCAGCGTGTTTATGAGGGGTCGCCACTTAATCTACCTGCGCTTGTGGTATGATGGCCACGTGGGTTCTGTG
GTCCCCGCCATGAGCTTTGGATACAGCGCCTTGCACTGTGGGATTTTGAACAATATTGTGGTGCTGTGCTGCAGT
TACTGTGCTGATTTAAGTGAGATCAGGGTGCGCTGCTGTGCCCGGAGGACAAGGCGTCTCATGCTGCGGGCGGTG
CGAATCATCGCTGAGGAGACCACTGCCATGTTGTATTCCTGCAGGACGGAGCGGCGGCGGCAGCAGTTTATTCGC
GCGCTGCTGCAGCACCACCGCCCTATCCTGATGCACGATTATGACTCTACCCCCATGTAG
Ad5 VA:
Accession: AF369965.1
(SEQ ID NO: 51)
TCGATGTAGGATGTTGCCCCTCCTGACGCGGTAGGAGAAGGGGAGGGTGCCCTGCATGTCTGCCGCTGCTCTTGC
TCTTGCCGCTGCTGAGGAGGGGGGCGCATCTGCCGCAGCACCGGATGCATCTGGGAAAAGCAAAAAAGGGGCTCG
TCCCTGTTTCCGGAGGAATTTGCAAGCGGGGTCTTGCATGACGGGGAGGCAAACCCCCGTTCGCCGCAGTCCGGC
CGGCCCGAGACTCGAACCGGGGGTCCTGCGACTCAACCCTTGGAAAATAACCCTCCGGCTACAGGGAGCGAGCCA
CTTAATGCTTTCGCTTTCCAGCCTAACCGCTTACGCCGCGCGCGGCCAGTGGCCAAAAAAGCTAGCGCAGCAGCC
GCCGCGCCTGGAAGGAAGCCAAAAGGAGCGCTCCCCCGTTGTCTGACGTCGCACACCTGGGTTCGACACGCGGGC
GGTAACCGCATGGATCACGGCGGACGGCCGGATCCGGGGTTCGAACCCCGGTCGTCCGCCATGATACCCTTGCGA
ATTTATCCACCAGACCACGGAAGAGTGCCCGCTTACAGGCTCTCCTTTTGCACGGTCTAGAGCGTCAACGACTGC
GCACGCCTCACCGGCCAGAGCGTCCCGACCATGGAGCACTTTTTGCCGCTGCGCAACATCTGGAACCGCGTCCGC
GACTTTCCGCGCGCCTCCACCACCGCCGCCGGCATCACCTGGATGTCCAGGTACATCTACGGATTACG

### Example 34—Specific Binding Pair Polynucleotide Sequences

Sequences can be found in one or more of these publications, for example, in WO2022/234276A1, WO2022129547A1, WO2011/098772, WO2016/193746, WO2018/197854, WO2018/189517 or Li et al., J. Mol. Biol. 426, 309-317 (2014); or can be designed or obtained by methods known in the art, for example, as described in Zakeri et al., 2012, and in Zakeri et al., 2010.

SpyTag Polynucleotide Sequence
(SEQ ID NO: 52)
GCCCACATCGTGATGGTGGACGCCTACAAGCCGACGAAG
SpyCatcher Polynucleotide Sequence
(SEQ ID NO: 53)
GTGGATACCCTGTCCGGACTGAGCAGTGAGCAAGGCCAGTCCGGAGATATGACAATTGAAGAAGATAGCGCCACC
CATATTAAATTCTCCAAAAGAGATGAGGACGGCAAAGAGCTGGCTGGAGCAACAATGGAGCTGAGAGATTCCTCT
GGAAAGACTATTAGTACATGGATCTCTGATGGCCAAGTGAAAGATTTCTATCTGTATCCAGGAAAGTACACATTT
GTCGAAACCGCTGCACCAGACGGATATGAGGTGGCTACAGCTATTACCTTTACAGTGAATGAGCAAGGACAGGTG
ACTGTTAATGGCAAAGCTACTAAAGGAGACGCTCATATTTAA

### Example 35—SpyTag Peptide Sequence

(SEQ ID NO: 54)
AHIVMVDAYKPTK

### Example 36—Specific Binding Pair Proteins

Additional binding pair proteins are reported. See, for example, WO2022/234276A1, WO2011/098772, WO2022129547A1, WO2016/193746, WO2018/197854, WO2018/189517 or Li et al., J. Mol. Biol. 426, 309-317 (2014).

| Peptide partner | Exemplary Sequence |
|---|---|
| SpyCatcher (SEQ | VDTLSGLSSEQGQSGDMTIEEDSATHIKFSKRDEDGKELAGATMELRDSSGK |
| ID NO. 55) | TISTWISDGQVKDFYLYPGKYTFVETAAPDGYEVATAITFTVNEQGQVTVNG |
| | KATKGDAHI |
| SpyCatcher ΔN1 | DSATHIKFSKRDEDGKELAGATMELRDSSGKTISTWISDGQVKDFYLYPGKY |
| (SEQ ID NO. 56) | TFVETAAPDGYEVATAITFTVNEQGQVTVNGKATKGDAHI |
| SpyCatcher002 | VTTLSGLSGEQGPSGDMTTEEDSATHIKFSKRDEDGRELAGATMELRDSSGK |
| (SEQ ID NO. 57) | TISTWISDGHVKDFYLYPGKYTFVETAAPDGYEVATAITFTVNEQGQVTVNG |
| | EATKGDAHT |
| Spycatcher003 | VTTLSGLSGEQGPSGDMTTEEDSATHIKFSKRDEDGRELAGATMELRDSSGK |
| (SEQ ID NO. 58) | TISTWISDGHVKDFYLYPGKYTFVETAAPDGYEVATPIEFTVNEDGQVTVDG |
| | EATEGDAHT |
| SpyCatcher ΔN1 | DSATHIKFSKRDEDGKELAGATMELRDSSGKTISTWISDGQVKDFYLYPGKY |
| ΔC2 (SEQ ID | TFVETAAPDGYEVATAITFTVNEQGQVTVNG |
| NO. 59) | |
| SnoopCatcher | KPLRGAVFSLQKQHPDYPDIYGAIDQNGTYQNVRTGEDGKLTEKNLSDGKYR |
| (SEQ ID NO. 60) | LFENSEPAGYKPVQNKPIVAFQIVNGEVRDVTSIVPQDIPATYEFTNGKHYI |
| | TNEPIPPK |
| Spyligase (SEQ | DYDGQSGDGKELAGATMELRDSSGKTISTWISDGQVKDFYLYPGKYTFVETA |
| ID NO. 61) | APDGYEVATAITFTVNEQGQVTVNGKATKGGSGGSGGSGEDSATHI* |
| SdyCatcher (SEQ | LSGETGQSGNITIEEDSTTHVKFSKRDANGKELAGAMIELRNLSGQTIQSWI |
| ID NO. 62) | SDGTVKVFYLMPGTYQFVETAAPEGYELAAPITFTIDEKGQIWVDS |
| RrgACatcher (SEQ | KLGDIEFIKVNKNDKKPLRGAVESLQKQHPDYPDIYGAIDQNGTYQNVRTGE |
| ID NO. 63) | DGKLTFKNLSDGKYRLFENSEPAGYKPVQNKPIVAFQIVNGEVRDVTSIVPQ |
| Dogcatcher V1 | KLGEIEFIKVDKTDKKPLRGAVFSLQKQHPDYPDIYGAIDQNGTYQDVRTGE |
| (SEQ ID NO. 64) | DGKLTFTNLSDGKYRLIENSEPPGYKPVQNKPIVSFRIVDGEVRDVTSIVPQ |
| Dogcatcher V2 | KLGEIEFIKVDKTDKKPLRGAVFSLQKQHPDYPDIYGAIDQNGTYQDVRTGE |
| (SEQ ID NO. 65) | DGKLTFTNLSDGKYRLFENSEPPGYKPVQNKPIVAFQIVDGEVRDVTSIVPQ |
| PsCsCatcher (SEQ | EQDVVFSKVNVAGEEIAGAKIQLKDAQGQVVHSWTSKAGQSETVKLKAGTYT |
| ID NO. 66) | FHEASAPTGYLAVTDITFEVDVQGKVTVKDANGNGVKAD |
| PilinC (SEQ ID | ATTVHGETVVNGAKLTVTKNLDLVNSNALIPNIDFTFKIEPDTTVNEDGNKE |
| NO. 67) | KGVALNTPMTKVTYTNSDKGGSNTKTAEFDFSEVTFEKPGVYYYKVTEEKID |
| | KVPGVSYDTTSYTVQVHVLWNEEQQKPVATYIVGYKEGSKVPIQFKNSLDST |
| | TLTVKKKVSGTGGDRSKDFNFGLTLKANQYYKASEKVMIEKTTKGGQAPVQT |
| | EASIDQLYHFTLKDGESIKVTNLPVGVDYVVTEDDYKSEKYTTNVEVSPQDG |
| | AVKNIAGNSTEQETSTDKDMTI |
| QueenCatcher | IDTMSGLSGETGQSGNTTIEEDSTTHVKFSKRDSNGKELAGAMIELRNLSGQ |
| (SEQ ID NO. 68) | TIQSWVSDGTVKDFYLMPGTYQFVETAAPEGYELAAPITFTVNEQGQVTVNG |
| | KATKGDAHI |
| Kat I (SEQ ID | DTMSGLSGETGQSGNTTIEEDSTTHVKFSKRDSNGKELAGAMIELRNLSGQT |
| NO. 69) | IQSWVSDGTVKDFYLMPGTYQFVETAAPEGYELAAPITFTVQDNGEVQIQGK |
| | ATRGDVPI |
| Mooncake (SEQ ID | IDTMSGLSGETGQSGNTTIEEDSTTHVKFSKRDSNGKELAGAMIELRNLSGQ |
| NO. 70) | TIQSWVSDGTVKDFYLMPGTYQFVETAAPEGYELAAPITFTVQDNGEVIIQG |
| | RLTRGDVHI |
| SpyTag002 (SEQ | VPTIVMVDAYKRYK |
| ID NO. 71) | |
| SpyTag003 (SEQ | RGVPHIVMVDAYKRYK |
| ID NO. 72) | |
| SnoopTag Jr (SEQ | KLGSIEFIKVNK |
| ID NO. 73) | |
| DogTag (SEQ ID | DIPATYEFTDGKHYITNEPIPPK |
| NO. 74) | |
| PsCsTag (SEQ ID | GNKLIVIDQAAPS |
| NO. 75) | |
| RrgATag (SEQ ID | DIPATYEFTNDKHYITNEP |
| NO. 76) | |
| IsopepTag (SEQ | TDKDMTITFINKKDAE |
| ID NO. 77) | |
| Clib9 (SEQ ID | RGVPTIVMVDCYKRYK |
| NO. 78) | |
| Rum2Tag (SEQ ID | GTPIVIMVDEAKPSLP |
| NO. 79) | |
| Rum3Tag (SEQ ID | GNPLIVMIDEAEQKEI |
| NO. 80) | |
| Rum4Tag (SEQ ID | AGGIIVMKDNTTKVSI |
| NO. 81) | |
| Rum5Tag (SEQ ID | GNPIVTMIDDATLVKI |
| NO. 82) | |
| Rum6Tag (SEQ ID | GNSTITMVDDTTKVHI |
| NO. 83) | |
| Rum7Tag (SEQ ID | GTPLIVMVDDTTKVEI |
| NO. 84) | |
| KTag (SEQ ID NO. | ATHIKFSKRD |
| 85) | |

Nucleotide and amino acid sequences for specific binding partners have been published or can be designed or obtained by methods known in the art, for example, as described in Zakeri et al., 2012, and in Zakeri et al., 2010 See, for example:

| Tag/Catcher | |
|---|---|
| Peptide Sequence | Corresponding DNA Sequence |
| SpyTag (SEQ ID NO. 86) | (SEQ ID NO. 87) |
| AHIVMVDAYKPTK | GCTCACATCG TGATGGTGGA CGCTTACAAG CCCACCAAG |
| SdyTag (SEQ ID NO. 88) | (SEQ ID NO. 89) |
| DPIVMIDNDKPIT | GACCCCATCG TGATGATCGA CAACGACAAG CCCATCACC |
| SnoopTag (SEQ ID NO. 90) | (SEQ ID NO. 91) |
| KLGDIEFIKVNK | AAACTGGGTG ATATTGAATT TATTAAAGTT AATAAA |
| PhoTag (SEQ ID NO. 92) | (SEQ ID NO. 93) |
| LVTGTAHIVMVDNYKPIVETGD | CTGGTTACCG GCACCGCACA TATTGTTATG GTTGATAACT |
| | ATAAGCCGAT CGTGGAAACC GGTGAT |
| EntTag (SEQ ID NO. 94) | (SEQ ID NO. 95) |
| NTIVMVDKLKEVPPT | ACCGAAGTTA GCGGTAATAC CATTGTGATG GTGGATAAAC |
| | TGAAAGAAGT TCCGCCTACC |
| RumTag (SEQ ID NO. 96) | (SEQ ID NO. 97) |
| SENGNPLIVMVDDTTKVKIS | AGCGAAAACG GCAACCCGCT GATTGTGATG GTGGATGATA |
| | CCACCAAAGT GAAAATTAGC |
| Rum2Tag (SEQ ID NO. 98) | (SEQ ID NO. 99) |
| GTPIVIMVDEAKPSLPD | GGCACCCCGA TTGTGATTAT GGTGGATGAA GCGAAACCGA |
| | GCCTGCCGGA T |
| Rum3Tag (SEQ ID NO. 100) | (SEQ ID NO. 101) |
| GNPLIVMIDEAEQKEIP | GGCAACCCGC TGATTGTGAT GATTGATGAA GCGGAACAGA |
| | AAGAAATTCC G |
| Rum4Tag (SEQ ID NO. 102) | (SEQ ID NO. 103) |
| AGGIIVMKDNTTKVSIS | GCGGGCGGCA TTATTGTGAT GAAAGATAAC ACCACCAAAG |
| | TGAGCATTAG C |
| Rum5Tag (SEQ ID NO. 104) | (SEQ ID NO. 105) |
| GNPIVTMIDDAILVKIS | GGCAACCCGA TTGTGACCAT GATTGATGAT GCGACCCTGG |
| | TGAAAATT |
| Rum6Tag (SEQ ID NO. 106) | (SEQ ID NO. 107) |
| GNSTITMVDDTIKVHIT | GGCAACAGCA CCATTACCAT GGTGGATGAT ACCACCAAAG |
| | TGCATATTAC C |
| Rum7Tag (SEQ ID NO. 108) | (SEQ ID NO. 109) |
| GTPLIVMVDDTIKVEIS | GGCACCCCGC TGATTGTGAT GGTGGATGAT ACCACCAAAG |
| | TGGAAATTAG C |
| RumTrunkTag D9N | (SEQ ID NO. 111) |
| (SEQ ID NO. 110) | GGTAATCCGC TGATTGTGAT GGTGAATGAT ACCACCAAAG |
| GNPLIVMVNDITKVK | TGAAA |
| RumTrunkTag tag | (SEQ ID NO. 113) |
| (SEQ ID NO. 112) | GGCAACCCGC TGATTGTGAT GGTGGATGAT ACCACCAAAG |
| GNPLIVMVDDTTKVK | TGAAA |
| KTag/BacTag | (SEQ ID NO. 115) |
| (SEQ ID NO. 114) | GGTCAGTTCG AAATTGTTAA AGTTGATGCA AACGATAAAA |
| NEKVTGQFEIVKVDANDKTK | CTAAA |
| Bac2Tag (SEQ ID NO. 116) | (SEQ ID NO. 117) |
| SKSLGQFEIVKVDAQDKTK | AGCAAAAGCC TGGGCCAGTT TGAAATTGTG AAAGTGGATG |
| | CGCAGGATAA AACCAAA |
| Bac3Tag (SEQ ID NO. 118) | (SEQ ID NO. 119) |
| LGQFEIVKVDSQDKTK | CTGGGCCAGT TTGAAATTGT TAAAGTTGAT AGCCAGGATA |
| | AAACCAAA |
| Bac4Tag (SEQ ID NO. 120) | (SEQ ID NO. 121) |
| VTGQFEIVKVDAEDKTR | GTTACCGGTC AGTTTGAAAT CGTTAAAGTT GATGCCGAAG |
| | ATAAGACCCG T |
| Bac5Tag (SEQ ID NO. 122) | (SEQ ID NO. 123) |
| EKVMGQFEIMKVDANDKTK | GAAAAAGIGA TGGGCCAGTT CGAAATCATG AAAGTIGATG |
| | CCAACGACAA GACCAAA |
| Cpe0147 esterbond | (SEQ ID NO. 125) |
| forming tag (SEQ ID NO. 124) | GACACCAAGC AGGTGGTGAA GCACGAGGAC AAGAACGACA |
| DTKQVVKHEDKNDKAQTLVVEKP | AGGCCCAGAC CCTGGTGGIG GAGAAGCCC |
| SpyCatcher (SEQ ID NO. 126) | (SEQ ID NO. 127) |
| GAMVDTLSGLSSEQGQSGDMTIEEDS | GGTGCAATGG TTGATACCCT GAGCGGTCTG AGCAGCGAAC |
| ATHIKESKRDEDGKELAGATMELRDS | AGGGTCAGAG CGGTGATATG ACCATTGAAG AAGATAGCGC |
| SGKTISTWISDGQVKDFYLYPGKYTE | AACCCACATC AAATTCAGCA AACGTGATGA AGATGGTAAA |
| VETAAPDGYEVATAITFTVNEQGQVT | GAACTGGCAG GCGCAACAAT GGAACTGCGT GATAGCAGCG |
| | GTAAAACCAT TAGCACCTGG ATTAGTGATG GTCAGGTGAA |
| | AGATTTTTAT CTGTACCCTG GCAAATACAC CTTTGTTGAA |
| | ACCGCAGCAC CGGATGGTTA TGAAGTTGCA ACCGCAATTA |
| | CCTTTACCGT TAATGAACAG GGCCAGGTTA CCGTGAATGG |
| | TAAAGCAACC AAAGGTGATG CACATATT |
| SdyCatcher (SEQ ID NO. 128) | (SEQ ID NO. 129) |
| IDTMSGLSGETGQSGNITIEEDSTTH | ATGGGTATTG ATACCATGAG CGGTCTGAGC GGTGAAACCG |
| VKFSKRDSNGKELAGAMIELRNLSGQ | GTCAGAGCGG TAATACCACC ATTGAAGAGG ATAGCACCAC |
| TIQSWVSDGTVKDFYLMPGTYQFVET | ACATGTGAAA TTCAGCAAAC GCGATGCAAA CGGCAAAGAA |
| AAPEGYELAAPITFTIDEKGQIWVDS | CTGGCAGGCG CAATGATTGA ACTGCGTAAT CTGAGTGGTC |
| | AGACCATTCA GAGCTGGGTT AGTGATGGCA CCGTTAAAGA |
| | TTTTTATCTG ATGCCTGGCA CCTATCAGTT TGTTGAAACC |
| | GCAGCACCGG AAGGTTATGA GCTGGCAGCA CCGATTACCT |
| | TTACCATTGA TGAAAAAGGT CAGATTTGGG TTGATAGC |
| SnoopCatcher (SEQ ID NO. 130) | (SEQ ID NO. 131) |
| SSGLVPRGSHMKPLRGAVESLQKQHP | AGCAGCGGCC TGGTGCCGCG CGGCAGCCAT ATGAAGCCGC |
| DYPDIYGAIDQNGTYQNVRTGEDGKL | TGCGTGGTGC CGTGTTTAGC CTGCAGAAAC AGCATCCCGA |
| TEKNLSDGKYRLFENSEPAGYKPVQN | CTATCCCGAT ATCTATGGCG CGATTGATCA GAATGGGACC |
| KPIVAFQIVNGEVRDVTSIVPQDIPA | TATCAAAATG TGCGTACCGG CGAAGATGGT AAACTGACCT |
| TYEFINGKHYITNEPIPPK | TTAAGAATCT GAGCGATGGC AAATATCGCC TGTTTGAAAA |
| | TAGCGAACCC GCTGGCTATA AACCGGTGCA GAATAAGCCG |
| | ATTGTGGCGT TTCAGATTGT GAATGGCGAA GTGCGTGATG |
| | TGACCAGCAT TGTGCCGCAG GATATTCCGG CTACATATGA |
| | ATTTACCAAC GGTAAACATT ATATCACCAA TGAACCGATA |
| | CCGCCGAAA |
| FimP domain 3 (SEQ ID NO. 132) | (SEQ ID NO. 133) |
| GSLSKYGKVILTKTGIDDLADKTKYN | GGTAGCCTGA GCAAATATGG TAAAGTGATT CTGACCAAAA |
| GAQFQVYECTKTASGAILRDSDPSTQ | CCGGCACCGA TGATCTGGCA GATAAAACCA AATATAACGG |
| TVDPLTIGGEKTFTTAGQGIVEINYL | TGCACAGTTT CAGGTGTATG AATGTACCAA AACAGCAAGC |
| RANDYVNGAKKDQLTDEDYYCLVETK | GGTGCAACCC TGCGTGATAG CGATCCGAGC ACACAGACCG |
| APEGYNLQADPLPERVLAEKAEKKA | TTGATCCGCT GACCATTGGT GGTGAAAAAA CCTTTACCAC |
| | CGCAGGTCAG GGCACCGTTG AAATTAATTA TCTGCGTGCC |
| | AATGATTATG TGAACGGTGC AAAAAAAGAT CAGCTGACCG |
| | ATGAAGATTA TTACTGTCTG GTTGAAACCA AAGCACCGGA |
| | AGGTTATAAT CTGCAGGCAG ATCCGCTGCC GTTTCGTGTT |
| | CTGGCCGAAA AAGCAGAAAA AAAAGCC |
| ancillary pilin domain 2 | (SEQ ID NO. 135) |
| (SEQ ID NO. 134) | GGTAGCACCA CCAAAGTGAA ACTGATTAAA GTTGATCAGG |
| GSTTKVKLIKVDQDHNRLEGVGFKLV | ATCACAATCG TCTGGAAGGT GTTGGTTTTA AACTGGTTAG |
| SVARDVSAAAVPLIGEYRYSSSGQVG | CGTTGCACGT GATGTTAGCG CAGCAGCAGT TCCGCTGATT |
| RTLYTDKNGEIFVINLPLGNYRFKEV | GGTGAATATC GTTATAGCAG CAGCGGTCAG GTTGGTCGTA |
| EPLAGYAVTTLDIDVQLVDHQLVT | CCCTGTATAC CGATAAAAAT GGCGAAATTT TCGTTACCAA |
| | TCTGCCGCTG GGTAACTATC GTTTTAAAGA AGTTGAACCG |
| | CTGGCAGGTT ATGCAGTTAC CACACTGGAT ACCGATGTTC |
| | AGCTGGTTGA TCATCAGCTG GTGACC |
| ancillary pilin domain 3 | (SEQ ID NO. 137) |
| (SEQ ID NO. 136) | CCGCGTGGTA ATGTTGATTT TATGAAAGTT GATGGTCGCA |
| PRGNVDFMKVDGRINTSLQGAMEKVM | CCAATACCAG CCTGCAGGGT GCAATGTTTA AAGTGATGAA |
| KEESGHYTPVLQNGKEVVVTSGKDGR | AGAAGAAAGC GGTCACTATA CACCGGTGCT GCAGAATGGT |
| FRVEGLEYGTYYLWELQAPTGYVQLT | AAAGAAGTTG TTGTTACCAG CGGTAAAGAT GGTCGTTTTC |
| SPVSFTIGKDTRKELV | GTGTTGAAGG TCTGGAATAT GGCACCTATT ATCTGTGGGA |
| | ACTGCAGGCA CCGACCGGTT ATGTTCAGCT GACCAGTCCG |
| | GTTAGTTTTA CCATTGGCAA AGATACCCGT AAAGAACTGG |
| | TG |
| SpaD Domain 3 (SEQ ID NO. 138) | (SEQ ID NO. 139) |
| VVTYHGKLKVVKKDGKEAGKVLKGAE | GTTGTTACCT ATCATGGTAA ACTGAAAGTG GTGAAAAAAG |
| FELYQCTSAAVLGKGPLTVDGVKKWT | ACGGTAAAGA GGCAGGCAAA GTTCTGAAAG GTGCAGAATT |
| TGDDGTFTIDGLHVIDFEDGKEAAPA | TGAACTGTAT CAGTGTACCA GCGCAGCAGT TTTAGGTAAA |
| TKKFCLKETKAPAGYALPDPNVTEIE | GGTCCGCTGA CCGTTGATGG TGTGAAAAAA TGGACCACCG |
| FTRAKISEKDKFEGDDEVT | GTGATGATGG CACCTTTACC ATTGATGGTC TGCATGTTAC |
| | CGATTTTGAA GATGGTAAAG AAGCCGCACC GGCAACCAAA |
| | AAATTCTGTC TGAAAGAAAC CAAAGCACCG GCAGGTTATG |
| | CACTGCCTGA TCCGAATGTG ACCGAAATTG AATTTACCCG |
| | TGCAAAAATC AGCGAGAAAG ATAAATTTGA AGGCGACGAT |
| | GAAGTGACC |
| Pilin subunit (SpaA) domain 2 | (SEQ ID NO. 141) |
| (SEQ ID NO. 140) | AGCACCAATG ATACCACCAC ACAGAATGTT GTTCTGACCA |
| STNDTTTQNVVLTKYGEDKDVTAIDR | AATATGGCTT CGATAAAGAT GTTACCGCAA TTGATCGTGC |
| ATDQIWTGDGAKPLQGVDFTIYNVTA | AACCGATCAG ATTTGGACCG GTGATGGTGC AAAACCGCTG |
| NYWASPKDYKGSFDSAPVAATGTIND | CAGGGTGTTG ATTTTACCAT TTATAACGTG ACCGCCAATT |
| KGQLTQALPIQSKDASGKTRAAVYLF | ATTGGGCAAG CCCGAAAGAT TATAAAGGCA GCTTTGATAG |
| HETNPRAGYNTSADFWLTLPAKAAAD | CGCACCGGTT GCAGCCACCG GTACAACAAA TGATAAAGGC |
| GNVY | CAGCTGACCC AGGCACTGCC GATTCAGAGC AAAGATGCAA |
| | GCGGTAAAAC CCGTGCAGCA GTTTACCTGT TTCACGAAAC |
| | CAATCCGCGT GCAGGTTATA ATACCAGCGC AGATTTTTGG |
| | CTGACCCTGC CTGCAAAAGC AGCAGCAGAT GGTAATGTTT |
| | AT |
| Pilin subunit (SpaA) domain 3 | (SEQ ID NO. 143) |
| (SEQ ID NO. 142) | ACCACCTATG AACGTACCTT TGTTAAAAAA GACGCCGAAA |
| TTYERTEVKKDAETKEVLEGAGFKIS | CCAAAGAAGT TCTGGAAGGC GCAGGCTTTA AAATCAGCAA |
| NSDGKFLKLIDKDGQSVSIGEGFIDV | TAGTGATGGC AAATTCCTGA AACTGACCGA TAAAGATGGT |
| LANNYRLIWVAESDATVFTSDKSGKF | CAGAGCGTTA GCATTGGTGA AGGTTTTATT GATGTTCTGG |
| GLNGFADNITTYTAVEINVPDGYDAA | CCAATAACTA TCGTCTGACC TGGGTTGCAG AAAGTGATGC |
| ANTDEKADNS | AACCGTTTTT ACCAGCGATA AAAGCGGCAA ATTTGGTCTG |
| | AATGGTTTTG CAGATAATAC CACCACCTAT ACCGCAGTTG |
| | AAACCAATGT TCCGGATGGT TATGATGCAG CAGCAAACAC |
| | CGATTTCAAA GCCGATAATA GC |
| Surface protein Spb1 domain 3 | (SEQ ID NO. 145) |
| (SEQ ID NO. 144) | GGTCAGATTA CCATCAAAAA AATCGATGGT AGCACCAAAG |
| GQITIKKIDGSTKASLQGAIFVLKNA | CAAGCCTGCA GGGTGCAATT TTTGTTCTGA AAAATGCAAC |
| TGQFLNENDINNVEWGTEANATEYTT | CGGTCAGTTC CTGAATTTTA ACGATACCAA TAATGTTGAA |
| GADGIITITGLKEGTYYLVEKKAPLG | TGGGGCACCG AAGCAAATGC CACCGAATAT ACCACCGGTG |
| YNLLDNSQKVILGDGAIDTINSDNLL | CAGATGGTAT TATTACCATT ACCGGTCTGA AAGAAGGCAC |
| VNP | CTATTACCTG GTTGAAAAAA AAGCACCGCT GGGTTATAAT |
| | CTGCTGGATA ATTCACAGAA AGTGATTTTA GGTGATGGTG |
| | CAACCGATAC CACCAATAGC GATAACCTGC TGGTTAATCC |
| | G |
| PsCsCatcher (SEQ ID NO. 146) | (SEQ ID NO. 147) |
| EQDVVFSKVNVAGEEIAGAKIQLKDA | GAACAGGATG TTGTGTTTAG CAAAGTTAAT GTTGCCGGTG |
| QGQVVHSWISKAGQSETVKLKAGTYT | AAGAAATTGC GGGTGCAAAA ATCCAGCTGA AAGATGCACA |
| FHEASAPTGYLAVTDITFEVDVQGKV | GGGTCAAGTT GTTCATAGCT GGACCAGCAA AGCAGGTCAG |
| TVKDANGNGVKAD | AGCGAAACCG TTAAACTGAA AGCAGGCACC TATACCTTTC |
| | ATGAAGCAAG CGCACCGACC GGTTATCTGG CAGTTACCGA |
| | TATTACCTTT GAAGTTGATG TTCAGGGTAA AGTGACCGTT |
| | AAAGATGCAA ATGGTAATGG TGTGAAAGCC GAC |
| RA Catcher (SEQ ID NO. 148) | (SEQ ID NO. 149) |
| KLGDIEFIKVNKNDKKPLRGAVESLQ | AAACTGGGTG ATATTGAGTT CATCAAAGTG AACAAAAACG |
| KQHPDYPDIYGAIDQNGTYQNVRIGE | ATAAAAAACC GCTGCGTGGT GCAGTTTTTA GCCTGCAGAA |
| DGKLIFKNLSDGKYRLFENSEPAGYK | ACAGCATCCG GATTACCCGG ATATTTATGG TGCAATTGAT |
| PVQNKPIVAFQIVNGEVRDVTSIVPQ | CAGAATGGCA CCTATCAGAA TGTTCGTACC GGTGAAGATG |
| | GTAAACTGAC CTTTAAAAAC CTGAGCGACG GTAAATATCG |
| | CCTGTTTGAA AATAGCGAAC CGGCAGGTTA TAAACCGGTT |
| | CAGAATAAAC CGATTGTGGC CTTTCAGATT GTTAATGGTG |
| | AAGTTCGTGA TGTGACCAGC ATTGTTCCGC AG |
| Major Pilin SpaD Domain 1 | (SEQ ID NO. 151) |
| (SEQ ID NO. 150) | GGTAGCGAAC GTAAAGGTAG TCTGACCCTG CATAAAAAGA |
| GSERKGSLTLHKKKGAESEKRATGKE | AAGGTGCAGA AAGCGAAAAA CGTGCAACCG GTAAAGAAAT |
| MDDVAGEPLNGVIFKITKLNEDLQNG | GGATGATGTT GCCGGTGAAC CGCTGAATGG TGTTACCTTT |
| DWAKFPKTAADAKGHETSTTKEVETS | AAAATCACCA AACTGAACTT CGATCTGCAG AATGGTGATT |
| GNGTAVEDNLDIGIYLVEETKAPDGI | GGGCAAAATT TCCGAAAACC GCAGCAGATG CAAAAGGTCA |
| VTGAPFIVSIPMVNEASDAWNYNVVA | TGAAACCAGC ACCACCAAAG AAGTGGAAAC CAGCGGTAAT |
| | GGCACCGCAG TTTTTGATAA TCTGGATCTG GGTATTTACC |
| | TGGTGGAAGA AACCAAAGCA CCGGATGGTA TTGTTACAGG |
| | TGCACCGTTT ATTGTTAGCA TTCCGATGGT TAATGAAGCA |
| | AGTGATGCCT GGAATTATAA CGTTGTTGCA |
| Cpe0147 esther-forming split- | (SEQ ID NO. 153) |
| protein pair (SEQ ID NO. 152) | AACCTGCCCG AGGTGAAGGA CGGCACCCTG AGGACCACCG |
| NLPEVKDGTLRITVIADGVNGSSEKE | TGATCGCCGA CGGCGTGAAC GGCAGCAGCG AGAAGGAGGC |
| ALVSFENSKDGVDVKDTINYEGLVAN | CCTGGTGAGC TTCGAGAACA GCAAGGACGG CGTGGACGTG |
| QNYTLTGTLMHVKADGSLEEIATKTT | AAGGACACCA TCAACTACGA GGGCCTGGTG GCCAACCAGA |
| NVTAGENGNGTWGLDFGNQKLQVGEK | ACTACACCCT GACCGGCACC CTGATGCACG TGAAGGCCGA |
| YVVFENAESVENLIDTDKDYNLDTKQ | CGGCAGCCTG GAGGAGATCG CCACCAAGAC CACCAACGTG |
| VVKHEDKNDKAQTLVVEKP | ACCGCCGGCG AGAACGGCAA CGGCACCTGG GGCCTGGACT |
| | TCGGCAACCA GAAGCTGCAG GTGGGCGAGA AGTACGTGGT |
| | GTTCGAGAAC GCCGAGAGCG TGGAGAACCT GATCGACACC |
| | GACAAGGACT ACAACCTGGA CACCAAGCAG GTGGTGAAGC |
| | ACGAGGACAA GAACGACAAG GCCCAGACCC TGGTGGTGGA |
| | GAAGCCC |

### Example 37—Herpes Simplex Virus (HSV) Polynucleotide Sequences

HSV polynucleotides can be selected from any serotype, and representative polynucleotides are exemplified below. Weindler and Heilbronn 1991 (Weindler, Friedrich W., and R.E.G I.N.E. Heilbronn. “A subset of herpes simplex virus replication genes provides helper functions for productive adeno-associated virus replication.” Journal of virology 65.5 (1991): 2476-2483); Ward et al. 2001 (Ward et al. Rep-dependent initiation of adeno-associated virus type 2 DNA replication by a herpes simplex virus type 1 replication complex in a reconstituted system. J. Virol. 2001; 75:10250-10258); Herpes Clément et al. 2009 (Clément, Nathalie, David R. Knop, and Barry J. Byrne. “Large-scale adeno-associated viral vector production using a herpesvirus-based system enables manufacturing for clinical studies.” Human gene therapy 20.8 (2009): 796-806); and Meier et al. 2020 (Meier, Anita F., Cornel Fraefel, and Michael Seyffert. “The Interplay between Adeno-Associated Virus and Its Helper Viruses.” Viruses 12.6 (2020)) disclose seven HSV replication genes (UL5, UL8, UL9, UL29, UL30, UL42, and UL52) that led to productive AAV replication, of which HSV-1 helicase-primase complex (HP; UL5/UL8/UL52) and the single-strand DNA binding protein ICP8 (gene UL29) is sufficient to restore AAV progeny production. HSV replication gene (UL5, UL8, UL52, UL29, UL9, UL30, and UL42) sequences as available at the GenBank are listed below:

### UL5 Helicase-Primase Helicase Subunit [Human Alphaherpesvirus 1 (Herpes Simplex Virus Type 1)] Gene ID: 2703420. NCBI Reference Sequence: NC_001806.2.

| (SEQ ID NO. 154) | |
|---|---|
| 1 | atggcggcgg ccggcgggga gcgccagcta gacggacaga aacccggccc gccgcacctt |
| 61 | cagcaacccg gggaccgacc agccgttcca gggagggccg aggccttttt aaattttacg |
| 121 | tctatgcacg gggtgcagcc aatccttaag cgcatccgag agctctcgca acaacagctc |
| 181 | gacggagcgc aagtgcccca tctgcagtgg ttccgggacg tggcggcctt agagtccccc |
| 241 | gcaggcctgc ccctcaggga gtttccgttc gcggtgtatc ttatcaccgg caacgctggc |
| 301 | tccggaaaga gcacgtgcgt gcagacaatc aacgaggtct tggactgtgt ggtgacgggc |
| 361 | gccacgcgca ttgcggccca aaacatgtac gccaaactct cgggcgcctt tctcagccga |
| 421 | cccatcaaca ccatctttca tgaatttggg tttcgcggga atcacgtcca ggcccaactg |
| 481 | ggacagtacc cgtacaccct gaccagcaac cccgcctcgc tggaggacct gcagcgacga |
| 541 | gatctgacgt actactggga ggtgattttg gacctcacga agcgcgccct ggccgcctcc |
| 601 | gggggcgagg agttgcggaa cgagtttcgc gccctggccg ccctggaacg gaccctgggg |
| 661 | ttggccgagg gcgccctgac gcggttggcc ccggccaccc acggggcgct gccggccttt |
| 721 | acccgcagca acgtgatcgt catcgacgag gccgggctcc ttgggcgtca cctcctcacg |
| 781 | gccgtggtgt attgctggtg gatgattaac gccctgtacc acacccccca gtacgcggcc |
| 841 | cgcctgcggc ccgtgttggt gtgtgtgggc tcgccgacgc agacggcgtc cctggagtcg |
| 901 | accttcgagc accagaaact gcggtgttcc gtccgccaga gcgagaacgt gctcacgtac |
| 961 | ctcatctgca accgcacgct gcgcgagtac gcccgcctct cgtatagctg ggccattttt |
| 1021 | attaacaaca aacggtgcgt cgagcacgag ttcggtaacc tcatgaaggt gctggagtac |
| 1081 | ggcctgccca tcaccgagga gcacatgcag ttcgtggatc gcttcgtcgt cccggaaaac |
| 1141 | tacatcacca accccgccaa cctccccggc tggacgcggc tgttctcctc ccacaaagag |
| 1201 | gtgagcgcgt acatggccaa gctccacgcc tacctgaagg tgacccgtga gggggagttc |
| 1261 | gtcgtgttca ccctccccgt gcttacgttc gtgtcggtca aggagtttga cgaataccga |
| 1321 | cggctgacac accagcccgg cctgacgatt gaaaagtggc tcacggccaa cgccagccgc |
| 1381 | atcaccaact actcgcagag ccaggaccag gacgcggggc acatgcgctg cgaggtgcac |
| 1441 | agcaaacagc agctggtcgt ggcccgcaac gacgtcactt acgtcctcaa cagccagatc |
| 1501 | gcggtgaccg cgcgcctgcg aaaactggtt tttgggttta gtgggacgtt ccgggccttc |
| 1561 | gaggcagtgt tgcgtgacga cagctttgta aagactcagg gggagacttc ggtggagttt |
| 1621 | gcctacaggt tcctgtcgcg gctcatattt agcgggctta tctcctttta caactttctg |
| 1681 | cagcgcccgg gcctggatgc gacccagagg accctcgcct acgcccgcat gggagaacta |
| 1741 | acggcggaga ttctgtctct gcgccccaaa tcttcggggg tgccgacgca ggcgtcggta |
| 1801 | atggccgacg caggcgcccc cggcgagcgt gcgtttgatt ttaagcaact ggggccgcgg |
| 1861 | gacgggggcc cggacgattt tcccgacgac gacctcgacg ttattttcgc ggggctggac |
| 1921 | gaacaacagc tcgacgtgtt ttactgccac tacacccccg gggaaccgga gaccaccgcc |
| 1981 | gccgttcaca cccagtttgc gctgctgaag cgggccttcc tcgggagatt ccgaatcctc |
| 2041 | caagagctct tcggggaggc atttgaagtc gcccccttta gcacgtacgt ggacaacgtt |
| 2101 | atcttccggg gctgcgagat gctgaccggc tcgccgcgcg gggggctgat gtccgtcgcc |
| 2161 | ctgcagacgg acaattatac gctcatggga tacacgtacg cacgggtgtt tgcctttgcg |
| 2221 | gacgagctgc ggaggcggca cgcgacggcc aacgtggccg agttactgga agaggccccc |
| 2281 | ctgccttacg tggtcttgcg ggaccaacac ggcttcatgt ccgtcgtcaa caccaacatc |
| 2341 | agcgagtttg tcgagtccat tgactctacg gagctggcca tggccataaa cgccgactac |
| 2401 | ggcatcagct ccaagcttgc catgaccatc acgcgctccc agggccttag cctggacaag |
| 2461 | gtcgccatct gctttacgcc cggcaacctg cgcctcaaca gcgcgtacgt ggccatgtcc |
| 2521 | cgcaccacct cctccgaatt ccttcgcatg aacttaaatc cgctccggga gcgccacgag |
| 2581 | cgcgatgacg tcattagtga gcacatacta tcggctctgc gcgatccgaa cgtggtcatt |
| 2641 | gtctattaac ccgccgtccc cttacagttc caccgaaccc ggcccggggg actcactacc |
| 2701 | caccgcgaga tgtccaatcc acagacgacc atcgcgtata gcctatgcca cgccagggcc |
| 2761 | tcgctgacca gcgcactgcc cgacgccgcg caggtggtgc atgtttttga gtacggcacc |
| 2821 | cgcgcgatca tggtacgggg ccgggagcgc caggaccgcc tgccgcgcgg aggcgttgtt |
| 2881 | atccagcaca cccccattgg gctgttggtg attatcgact gtcgcgccga attttgtgcc |
| 2941 | taccgcttta taggccggga cagcaaccag aagctcgaac gcgggtggga cgcccatatg |
| 3001 | tacgcgtatc cgttcgactc ctgggtcagc tcctcgcgcg gcgaaagcgc ccggagcgcc |
| 3061 | acggccggca ttttgaccgt ggtctggacc gcggacacca tttacatcac tgcaaccatt |
| 3121 | tacgggtcgc ccccagagga gacgccaggc gcggcacacg gggtgggcgc cgcgcctcca |
| 3181 | cccccgacaa ccgcctgccc cgggacggcc gagtttctcc agcccaccgc ggacctgctg |
| 3241 | gtagaggtgc tgcgggagat tcaactgagc cccgccctgg aatacgcaga caaacttttg |
| 3301 | gggtcctagg atcccggccg gatcgcgctc gtcacccgac actgaaatgc cccccccccc |
| 3361 | ttgcgggcgg tccattaaa |

### UL8 Helicase-Primase Subunit [Human Alphaherpesvirus 1 (Herpes Simplex Virus Type 1)] Gene ID: 2703432. NCBI Reference Sequence: NC_001806.2.

| (SEQ ID NO. 155) | |
|---|---|
| 1 | atggacaccg cagatatcgt gtgggtggag gagagcgtca gcgccattac cctttacgcg |
| 61 | gtatggctgc ccccccgcgc tcgcgagtac ttccacgccc tggtgtattt tgtatgtcgc |
| 121 | aacgccgcag gggagggtcg cgcgcgcttt gcggaggtct ccgtcaccgc gacggagctg |
| 181 | cgggatttct acggctccgc ggacgtctcc gtccaggccg tcgtggcggc cgcccgcgcc |
| 241 | gcgacgacgc cggccgcctc cccgctggag cccctggaga acccgactct gtggcgggcg |
| 301 | ctgtacgcgt gcgtcctggc ggccctggag cgccagaccg ggccggtggc cctgttcgcc |
| 361 | ccgctgcgta tcggctcgga cccacgcacg ggactggtgg tgaaagttga gagagcgtcg |
| 421 | tggggcccgc ccgccgcccc tcgcgccgct ctcctggtcg cggaggccaa cattgacatc |
| 481 | gaccctatgg ccctggcggc gcgcgttgcc gagcatcccg acgcgcggct ggcgtgggcg |
| 541 | cgcctggcgg ccattcgcga caccccccag tgcgcgtccg ccgcttcgct gaccgttaac |
| 601 | atcaccaccg gaaccgcgct atttgcgcgc gaataccaga ctcttgcgtt tccgccgatc |
| 661 | aagaaggagg gcgcgttcgg ggacctggtc gaggtgtgcg aggtgggcct gcggccacgc |
| 721 | gggcacccgc aacgagtcac ggcacgggtg ctgctgcccc gcgattacga ctactttgta |
| 781 | agcgccggcg agaagttctc cgcgccggcg ctcgtcgccc ttttccggca gtggcatacc |
| 841 | acggtccacg ccgcccccgg ggccctggcc cccgtctttg cctttctggg gcccgagttt |
| 901 | gaggtccggg ggggacccgt cccgtacttt gccgtcctgg ggtttccggg ttggcccacg |
| 961 | ttcaccgtgc cggccacggc cgagtcggca cgggacctgg tgcgcggggc cgcggccgct |
| 1021 | tacgccgcgc tcctgggggc ctggcccgcg gtgggggcca gggtcgtcct ccccccgcga |
| 1081 | gcctggcccg gcgtggcctc ggcggcagcc ggatgcctcc tgcccgcggt gcgggaggcg |
| 1141 | gtggcgcggt ggcatcccgc cactaaaatc atccaactgt tagacccgcc cgcggccgtc |
| 1201 | gggcccgtct ggacggcgcg gttttgcttc cccggacttc gcgcccagct cctggcggcc |
| 1261 | ctggccgacc tcggggggag cgggctggcg gacccccacg gccggacggg cctagcaaga |
| 1321 | ctggacgcgc tggtggtggc cgctccctca gagccctggg ccggggccgt cttggagcgc |
| 1381 | ctggtcccgg acacgtgcaa cgcctgccct gcgctgcggc agctcctggg tggggtaatg |
| 1441 | gccgccgtct gcctgcagat cgaggagacg gccagctcgg tgaagttcgc ggtctgcggg |
| 1501 | ggcgatgggg gtgcgttctg gggtgtcttt aacgtggacc cccaagacgc ggatgcggct |
| 1561 | tccggggtga tcgaggacgc ccggcgggcc atcgagacgg ccgtgggagc cgtgcttagg |
| 1621 | gccaacggcc tccggctgcg gcacccactg tgcctggccc tcgagggcgt ctacacccac |
| 1681 | gcagtcgcct ggagccaggc gggagtgtgg ttctggaact cccgcgacaa cactgaccat |
| 1741 | cttgggggat ttcctctccg cgggcccgcg tacaccacgg cggcaggggt cgtacgcgac |
| 1801 | acgctgcgac gggtcctggg cctgacaacg gcatgcgtgc cggaggagga cgcactcacg |
| 1861 | gcccggggcc ttatggagga cgcctgcgac cgccttatct tggacgcgtt taataaacgg |
| 1921 | ttggacgcgg agtactggag cgttcgggtg tccccctttg aggccagcga ccccttgccc |
| 1981 | cccactgcct tccgcggcgg cgccttgctg gacgcagagc actactggcg gcgcgtcgtg |
| 2041 | cgtgtctgtc ccggaggcgg ggagtcggtc ggcgtccccg tcgatctata cccgcggccc |
| 2101 | cttgtgctcc cccccgtgga ctgcgctcat cacctgcgcg aaatcctgcg cgagattgag |
| 2161 | ttggtgttta ccggggtgct ggcgggagta tggggcgagg gggggaagtt tgtgtatccc |
| 2221 | tttgacgaca agatgtcgtt tctgtttgcc tgagtttgac caataaa |

### UL52 Helicase-Primase Primase Subunit [Human Alphaherpesvirus 1 (Herpes Simplex Virus Type 1)]. Gene ID: 2703423. NCBI Reference Sequence: NC_001806.2.

| (SEQ ID NO. 156) | |
|---|---|
| 1 | atggggcagg aagacgggaa ccgcggggag aggcggggag ccgggactcc cgtggaggtg |
| 61 | accgcgcttt atgcgaccga cgggtgcgtt attacctctt cgatcgccct cctcacaaac |
| 121 | tctctactgg gggccgagcc ggtttatata ttcagctacg acgcatacac gcacgatggc |
| 181 | cgtgccgacg ggcccacgga gcaagacagg ttcgaagaga gtcgggcgct ctaccaagcg |
| 241 | tcgggcgggc taaatggcga ctccttccga gtaacctttt gtttattggg gacggaagtg |
| 301 | ggtgggaccc accaggcccg cgggcgaacc cgacccatgt tcgtctgtcg cttcgagcga |
| 361 | gcggacgacg tcgccgcgct acaggacgcc ctggcgcacg ggaccccgct acaaccggac |
| 421 | cacatcgccg ccaccctgga cgcggaggcc acgttcgcgc tgcatgcgaa catgatcctg |
| 481 | gctctcaccg tggccatcaa caacgccagc ccccgcaccg gacgcgacgc cgccgcggcg |
| 541 | cagtatgatc agggcgcgtc cctacgctcg ctcgtggggc gcacgtccct gggacaacgc |
| 601 | ggccttacca cgctatacgt ccaccacgag gtgcgcgtgc ttgccgcgta ccgcagggcg |
| 661 | tattatggaa gcgcgcagag tcccttctgg tttcttagca aattcgggcc ggacgaaaaa |
| 721 | agcctggtgc tcaccactcg gtactacctg cttcaggccc agcgtctggg gggcgcgggg |
| 781 | gccacgtacg acctgcaggc catcaaggac atctgcgcca cctacgcgat tccccacgcc |
| 841 | ccccgccccg acaccgtcag cgctgcgtcc ctgacctcgt ttgccgccat cacgcggttc |
| 901 | tgttgcacga gccagtacgc ccgcggggcc gcggcggccg ggtttccgct ttacgtggag |
| 961 | cgccgtattg cggccgacgt ccgcgagacc agtgcgctgg agaagttcat aacccacgat |
| 1021 | cgcagttgcc tgcgcgtgtc cgaccgtgaa ttcattacgt acatctacct ggcccatttt |
| 1081 | gagtgtttca gccccccgcg cctagccacg catcttcggg ccgtgacgac ccacgacccc |
| 1141 | aaccccgcgg ccagcacgga gcagccctcg cccctgggca gggaggccgt ggaacaattt |
| 1201 | ttttgtcacg tgcgcgccca actgaatatc ggggagtacg tcaaacacaa cgtgaccccc |
| 1261 | cgggagaccg tcctggatgg cgatacggcc aaggcctacc tgcgcgctcg cacgtacgcg |
| 1321 | cccggggccc tgacgcccgc ccccgcgtat tgcggggccg tggactccgc caccaaaatg |
| 1381 | atggggcgtt tggcggacgc cgaaaagctc ctggtccccc gcgggtggcc cgcgtttgcg |
| 1441 | cccgccagtc ccggggagga cacggcgggc ggcacgccgc ccccacagac ctgcggaatt |
| 1501 | gtcaagcgcc tcctgagact ggccgccacg gaacagcagg gccccacacc cccggcgatc |
| 1561 | gcggcgctta tccgtaatgc ggcggtgcag actcccctgc ccgtctaccg gatatccatg |
| 1621 | gtccccacgg gacaggcatt tgccgcgctg gcctgggacg actgggcccg cataacgcgg |
| 1681 | gacgctcgcc tggccgaagc ggtcgtgtcc gccgaagcgg cggcgcaccc cgaccacggc |
| 1741 | gcgctgggca ggcggctcac ggatcgcatc cgcgcccagg gccccgtgat gccccctggc |
| 1801 | ggcctggatg ccggggggca gatgtacgtg aatcgcaacg agatattcaa cggcgcgctg |
| 1861 | gcaatcacaa acatcatcct ggatctcgac atcgccctga aggagcccgt cccctttcgc |
| 1921 | cggctccacg aggccctggg ccactttagg cgcggggctc tggctgcggt tcagctcctg |
| 1981 | tttcccgcgg cccgcgtgga ccccgacgca tatccctgtt attttttcaa aagcgcatgt |
| 2041 | cggcccggcc cggcgtccgt gggttccggc agcggactcg gcaacgacga cgacggggac |
| 2101 | tggtttccct gctacgacga cgccggtgat gaggagtggg cggaggaccc gggcgccatg |
| 2161 | gacacatccc acgatccccc ggacgacgag gttgcctact ttgacctgtg ccacgaagtc |
| 2221 | ggccccacgg cggaacctcg cgaaacggat tcgcccgtgt gttcctgcac cgacaagatc |
| 2281 | ggactgcggg tgtgcatgcc cgtccccgcc ccgtacgtcg tccacggttc tctaacgatg |
| 2341 | cggggggtgg cacgggtcat ccagcaggcg gtgctgttgg accgagattt tgtggaggcc |
| 2401 | atcgggagct acgtaaaaaa cttcctgttg atcgatacgg gggtgtacgc ccacggccac |
| 2461 | agcctgcgct tgccgtattt tgccaaaatc gcccccgacg ggcctgcgtg cggaaggctg |
| 2521 | ctgccagtgt ttgtgatccc ccccgcctgc aaagacgttc cggcgtttgt cgccgcgcac |
| 2581 | gccgacccgc ggcgcttcca ttttcacgcc ccgcccacct atctcgcttc cccccgggag |
| 2641 | atccgtgtcc tgcacagcct gggtggggac tatgtgagct tctttgaaag gaaggcgtcc |
| 2701 | cgcaacgcgc tggaacactt tgggcgacgc gagaccctga cggaggtcct gggtcggtac |
| 2761 | aacgtacagc cggatgcggg ggggaccgtc gaggggttcg catcggaact gctggggcgg |
| 2821 | atagtcgcgt gcatcgaaac ccactttccc gaacacgccg gcgaatatca ggccgtatcc |
| 2881 | gtccggcggg ccgtcagtaa ggacgactgg gtcctcctac agctagtccc cgttcgcggt |
| 2941 | accctgcagc aaagcctgtc gtgtctgcgc tttaagcacg gccgggcgag tcgcgccacg |
| 3001 | gcgcggacat tcgtcgcgct gagcgtcggg gccaacaacc gcctgtgcgt gtccttgtgt |
| 3061 | cagcagtgct ttgccgccaa atgcgacagc aaccgcctgc acacgctgtt taccattgac |
| 3121 | gccggcacgc catgctcgcc gtccgttccc tgcagcacct ctcaaccgtc gtcttgataa |
| 3181 | cggcgtacgg cctcgtgctc gtgtggtaca ccgtcttcgg tgccagtccg ctgcaccgat |
| 3241 | gtatttacgc ggtacgcccc accggcacca acaacgacac cgccctcgtg tggatgaaaa |
| 3301 | tgaaccagac cctattgttt ctgggggccc cgacgcaccc ccccaacggg ggctggcgca |
| 3361 | accacgccca tatctgctac gccaatctta tcgcgggtag ggtcgtgccc ttccaggtcc |
| 3421 | cacctgacgc catgaatcgt cggatcatga acgtccacga ggcagttaac tgtctggaga |
| 3481 | ccctatggta cacacgggtg cgtctggtgg tcgtagggtg gttcctgtat ctggcgttcg |
| 3541 | tcgccctcca ccaacgccga tgtatgtttg gcgtcgtgag tcccgcccac aagatggtgg |
| 3601 | ccccggccac ctacctcttg aactacgcag gccgcatcgt atcgagcgtg ttcctgcagt |
| 3661 | acccctacac gaaaattacc cgcctgctct gcgagctgtc ggtccagcgg caaaacctgg |
| 3721 | ttcagttgtt tgagacggac ccggtcacct tcttgtacca ccgccccgcc atcggggtca |
| 3781 | tcgtaggctg cgagttgatg ctacgctttg tggccgtggg tctcatcgtc ggcaccgctt |
| 3841 | tcatatcccg gggggcatgt gcgatcacat accccctgtt tctgaccatc accacctggt |
| 3901 | gttttgtctc caccatcggc ctgacagagc tgtattgtat tctgcggcgg ggcccggccc |
| 3961 | ccaagaacgc agacaaggcc gccgccccgg ggcgatccaa ggggctgtcg ggcgtctgcg |
| 4021 | ggcgctgctg ttccatcatc ctctcgggca tcgcagtgcg attgtgttat atcgccgtgg |
| 4081 | tggccggggt ggtgctcgtg gcgcttcact acgagcagga gatccagagg cgcctgtttg |
| 4141 | atgtatgacg tcacatccag gccggcggaa accgtaacgg catatgcaaa ttggaaactg |
| 4201 | tcctgtcttg gggcccaccc acccgacgcg tcatatgcaa atgaaaatcg gtcccccgag |
| 4261 | gccacgtgta gcctggatcc caacgacccc gcccatgggt cccaattggc cgtcccgtta |
| 4321 | ccaagaccaa cccagccagc gtatccaccc ccgcccgggt ccccgcggaa gcggaacggg |
| 4381 | gtatgtgata tgctaattaa a |

### UL29 (ICP8) Single-Stranded DNA-Binding Protein [Human Alphaherpesvirus 1 (Herpes Simplex Virus Type 1)] Gene ID: 2703458. NCBI Reference Sequence: NC_001806.2.

| (SEQ ID NO. 157) | |
|---|---|
| 1 | atggagacaa agcccaagac ggcaaccacc atcaaggtcc cccccgggcc cctgggatac |
| 61 | gtgtacgctc gcgcgtgtcc gtccgaaggc atcgagcttc tggcgttact gtcggcacgc |
| 121 | agcggcgatt ccgacgtcgc cgtggcgccc ctggtcgtgg gcctgaccgt ggagagcggc |
| 181 | tttgaggcca acgtggccgt ggtcgtgggt tctcgcacga cggggctcgg gggtaccgcg |
| 241 | gtgtccctga aactgacgcc ctcgcactac agctcgtccg tgtacgtctt tcacggcggc |
| 301 | cggcacctgg accccagcac ccaggccccg aacctgacgc gactttgcga gcgggcacgc |
| 361 | cgccattttg gcttttcgga ctacaccccc cggcccggcg acctcaaaca cgagacgacg |
| 421 | ggggaggcgc tgtgtgagcg cctcggcctg gacccggacc gcgccctcct gtatctggtc |
| 481 | gttaccgagg gcttcaagga ggccgtgtgc atcaacaaca cctttctgca cctgggaggc |
| 541 | tcggacaagg taaccatagg cggggcggag gtgcaccgca tacccgtgta cccgttgcag |
| 601 | ctgttcatgc cggattttag ccgtgtcatc gcagagccgt tcaacgccaa ccaccgatcg |
| 661 | atcggggaga attttaccta cccgcttccg ttttttaacc gccccctcaa ccgcctcctg |
| 721 | ttcgaggcgg tcgtgggacc cgccgccgtg gcactgcgat gccgaaacgt ggacgccgtg |
| 781 | gcccgcgcgg ccgcccacct ggcgtttgac gaaaaccacg agggcgccgc cctccccgcc |
| 841 | gacattacgt tcacggcctt cgaagccagc cagggtaaga ccccgcgggg cgggcgcgac |
| 901 | ggcggcggca agggcccggc gggcgggttc gaacagcgcc tggcctccgt catggccgga |
| 961 | gacgccgccc tggccctcga gtctatcgtg tcgatggccg tctttgacga gccgcccacc |
| 1021 | gacatctccg cgtggccgct gttcgagggc caggacacgg ccgcggcccg cgccaacgcc |
| 1081 | gtcggggcgt acctggcgcg cgccgcggga ctcgtggggg ccatggtatt tagcaccaac |
| 1141 | tcggccctcc atctcaccga ggtggacgac gccggcccgg cggacccaaa ggaccacagc |
| 1201 | aaaccctcct tttaccgctt cttcctcgtg cccgggaccc acgtggcggc caacccacag |
| 1261 | gtggaccgcg agggacacgt ggtgcccggg ttcgagggtc ggcccaccgc gcccctcgtc |
| 1321 | ggcggaaccc aggaatttgc cggcgagcac ctggccatgc tgtgtgggtt ttccccggcg |
| 1381 | ctgctggcca agatgctgtt ttacctggag cgctgcgacg gcggcgtgat cgtcgggcgc |
| 1441 | caggagatgg acgtgtttcg atacgtcgcg gactccaacc agaccgacgt gccctgtaac |
| 1501 | ctatgcacct tcgacacgcg ccacgcctgc gtacacacga cgctcatgcg cctccgggcg |
| 1561 | cgccatccaa agttcgccag cgccgcccgc ggagccatcg gcgtcttcgg gaccatgaac |
| 1621 | agcatgtaca gcgactgcga cgtgctggga aactacgccg ccttctcggc cctgaagcgc |
| 1681 | gcggacggat ccgagaccgc ccggaccatc atgcaggaga cgtaccgcgc ggcgaccgag |
| 1741 | cgcgtcatgg ccgaactcga gaccctgcag tacgtggacc aggcggtccc cacggccatg |
| 1801 | gggcggctgg agaccatcat caccaaccgc gaggccctgc atacggtggt gaacaacgtc |
| 1861 | aggcaggtcg tggaccgcga ggtggagcag ctgatgcgca acctggtgga ggggaggaac |
| 1921 | ttcaagtttc gcgacggtct gggcgaggcc aaccacgcca tgtccctgac gctggacccg |
| 1981 | tacgcgtgcg ggccgtgccc cctgcttcag cttctcgggc ggcgatccaa cctcgccgtg |
| 2041 | taccaggacc tggccctgag tcagtgccac ggggtgttcg ccgggcagtc ggtcgagggg |
| 2101 | cgcaactttc gcaatcaatt ccaaccggtg ctgcggcggc gcgtgatgga catgtttaac |
| 2161 | aacgggtttc tgtcggccaa aacgctgacg gtcgcgctct cggagggggc ggctatctgc |
| 2221 | gcccccagcc taacggccgg ccagacggcc cccgccgaga gcagcttcga gggcgacgtt |
| 2281 | gcccgcgtga ccctggggtt tcccaaggag ctgcgcgtca agagccgcgt gttgttcgcg |
| 2341 | ggcgcgagcg ccaacgcgtc cgaggccgcc aaggcgcggg tcgccagcct ccagagcgcc |
| 2401 | taccagaagc ccgacaagcg cgtggacatc ctcctcggac cgctgggctt tctgctgaag |
| 2461 | cagttccacg cggccatctt ccccaacggc aagcccccgg ggtccaacca gccgaacccg |
| 2521 | cagtggttct ggacggccct ccaacgcaac cagcttcccg cccggctcct gtcgcgcgag |
| 2581 | gacatcgaga ccatcgcgtt cattaaaaag ttttccctgg actacggcgc gataaacttt |
| 2641 | attaacctgg cccccaacaa cgtgagcgag ctggcgatgt actacatggc aaaccagatt |
| 2701 | ctgcggtact gcgatcactc gacatacttc atcaacaccc ttacggccat catcgcgggg |
| 2761 | tcccgccgtc cccccagcgt gcaggctgcg gccgcgtggt ccgcgcaggg cggggcgggc |
| 2821 | ctggaggccg gggcccgcgc gctgatggac gccgtggacg cgcatccggg cgcgtggacg |
| 2881 | tccatgttcg ccagctgcaa cctgctgcgg cccgtcatgg cggcgcgccc catggtcgtg |
| 2941 | ttggggttga gcatcagcaa gtactacggc atggccggca acgaccgtgt gtttcaggcc |
| 3001 | gggaactggg ccagcctgat gggcggcaaa aacgcgtgcc cgctccttat ttttgaccgc |
| 3061 | acccgcaagt tcgtcctggc ctgtccccgg gccgggtttg tgtgcgcggc ctcaagcctc |
| 3121 | ggcggcggag cgcacgaaag ctcgctgtgc gagcagctcc ggggcattat ctccgagggc |
| 3181 | ggggcggccg tcgccagtag cgtgttcgtg gcgaccgtga aaagcctggg gccccgcacc |
| 3241 | cagcagctgc agatcgagga ctggctggcg ctcctggagg acgagtacct aagcgaggag |
| 3301 | atgatggagc tgaccgcgcg tgccctggag cgcggcaacg gcgagtggtc gacggacgcg |
| 3361 | gccctggagg tggcgcacga ggccgaggcc ctagtcagcc aactcggcaa cgccggggag |
| 3421 | gtgtttaact ttggggattt tggctgcgag gacgacaacg cgacgccgtt cggcggcccg |
| 3481 | ggggccccgg gaccggcatt tgccggccgc aaacgggcgt tccacgggga tgacccgttt |
| 3541 | ggggaggggc cccccgacaa aaagggagac ctgacgttgg atatgctgtg aggggttggg |
| 3601 | gggtggggga acctagggcg gggcggggaa tgtgtgtaaa ataaa |

### UL9 DNA Replication Origin-Binding Helicase [Human Alphaherpesvirus 1 (Herpes Simplex Virus Type 1)] Gene ID: 2703434. NCBI Reference Sequence: NC_001806.2.

| (SEQ ID NO. 158) | |
|---|---|
| 1 | atgcctttcg tggggggcgc ggagtcggga gatcctctgg gggccgggcg tcccattggg |
| 61 | gacgacgagt gcgaacagta cacgtcgagc gtatcgctag cgcggatgtt gtacgggggg |
| 121 | gatttggccg aatgggtgcc ccgggttcac ccgaaaacaa cgatcgagcg gcagcagcac |
| 181 | ggacccgtca ccttccccaa cgcgagcgcc ccgacggcca ggtgcgtgac tgtggtccgc |
| 241 | gcgccaatgg ggtcgggaaa aactaccgcg ctgatccgct ggctgcggga agcgatccac |
| 301 | tctccggaca cgagtgtgct cgtcgtctcc tgtcgtcgga gttttaccca gaccctagcg |
| 361 | acgcggttcg ctgagtcagg cctggtcgac tttgtcacct acttctcatc caccaattac |
| 421 | attatgaacg accgcccctt ccaccgactt atcgtccagg tggaaagcct tcatcgcgtg |
| 481 | ggccccaacc ttctgaacaa ctacgacgtc ctcgttctgg acgaggttat gtcgacgctg |
| 541 | ggccagctct attcgccaac gatgcagcaa ctgggccgcg tggatgcgtt aatgctacgc |
| 601 | ctgctgcgca cctgtcctcg gatcatcgcc atggacgcaa ccgccaacgc gcagttggtg |
| 661 | gacttcctgt gcggtctccg gggcgaaaaa aacgtgcatg tggtggtcgg cgagtacgcc |
| 721 | atgcccgggt tttcggcgcg ccggtgcctg tttctcccgc gtctggggac cgagctcctg |
| 781 | caggctgccc tgcgcccgcc cgggccgccg agcggcccgt ctccggacgc ctctccggac |
| 841 | gcccgggggg ccacgttctt tggggagctg gaagcgcgcc ttggcggggg cgataacatc |
| 901 | tgcatttttt cgtcgacggt ctccttcgcg gagatcgtgg cccggttctg ccgtcagttt |
| 961 | acggaccgcg tgctgttgct tcactcgctc acccccctcg gggacgtgac cacgtggggc |
| 1021 | caataccgcg tggttatata cacgacggtc gtaaccgtgg gcctcagctt cgatcccctg |
| 1081 | cactttgatg gcatgttcgc ctacgtgaaa cccatgaact acggaccgga catggtgtcc |
| 1141 | gtgtaccagt ccctgggacg ggtgcgcacc ctccgcaagg gggagctact gatttacatg |
| 1201 | gacggctccg gggcgcgctc ggagcccgtc tttacgccca tgctccttaa tcacgtggtc |
| 1261 | agttcctgcg gccagtggcc cgcgcagttc tcccaggtca caaacctgct gtgtcgccgg |
| 1321 | ttcaaggggc gctgtgacgc gtcggcatgc gacacgtcgc tggggcgggg gtcgcgcatc |
| 1381 | tacaacaaat tccgttacaa acactacttt gagagatgca cgctggcgtg tctctcggac |
| 1441 | agccttaaca tccttcacat gctgctgacc ctaaactgca tacgcgtgcg cttctgggga |
| 1501 | cacgacgata ccctgacccc aaaggacttc tgtctgtttt tgcggggcgt acatttcgac |
| 1561 | gccctcaggg cccagcgcga tctacgggag ctgcggtgcc gggatcccga ggcgtcgctg |
| 1621 | ccggcccagg ccgccgagac ggaggaggtg ggtcttttcg tcgaaaaata cctccggtcc |
| 1681 | gatgtcgcgc cggcggaaat tgtcgcgctc atgcgcaacc tcaacagcct gatgggacgc |
| 1741 | acgcggttta tttacctggc gttgctggag gcctgtctcc gcgttcccat ggccacccgc |
| 1801 | agcagcgcca tatttcggcg gatctatgac cactacgcca cgggcgtcat ccccacgatc |
| 1861 | aacgtcaccg gagagctgga gctcgtggcc ctgcccccca ccctgaacgt aacccccgtc |
| 1921 | tgggagctgt tgtgcctgtg cagcaccatg gccgcgcgcc tgcattggga ctcggcggcc |
| 1981 | gggggatctg ggaggacctt cggccccgat gacgtgctgg acctactgac cccccactac |
| 2041 | gaccgctaca tgcagctggt gttcgaactg ggccactgta acgtaaccga cggacttctg |
| 2101 | ctctcggagg aagccgtcaa gcgcgtcgcc gacgccctaa gcggctgtcc cccgcgcggg |
| 2161 | tccgttagcg agacggacca cgcggtggcg ctgttcaaga taatctgggg cgaactgttt |
| 2221 | ggcgtgcaga tggccaaaag cacgcagacg tttcccgggg cggggcgcgt taaaaacctc |
| 2281 | accaaacaga caatcgtggg gttgttggac gcccaccaca tcgaccacag cgcctgccgg |
| 2341 | acccacaggc agctgtacgc cctgcttatg gcccacaagc gggagtttgc gggcgcgcgc |
| 2401 | ttcaagctac gcgtgcccgc gtgggggcgc tgtttgcgca cgcactcatc cagcgccaac |
| 2461 | cccaacgctg acatcatcct ggaggcggcg ctgtcggagc tccccaccga ggcctggccc |
| 2521 | atgatgcagg gggcggtgaa ctttagcacc ctataagtct cgggaccgca ctcgttcggt |
| 2581 | acgtggtcgt ccgcggaccg gcggcgctgt tgccggaacg caccgagggg ccaagttggc |
| 2641 | ccccggaccc gggccgtttc ccacccccac cccaacccca aaaaccgccc cccccccgtc |
| 2701 | accggtttcc gcgacccacc gggcccggcc aggcacggca gcatgggacc cacagaccgc |
| 2761 | ccgtgatcct taggggccgt gcgatggaca ccgcagatat cgtgtgggtg gaggagagcg |
| 2821 | tcagcgccat taccctttac gcggtatggc tgcccccccg cgctcgcgag tacttccacg |
| 2881 | ccctggtgta ttttgtatgt cgcaacgccg caggggaggg tcgcgcgcgc tttgcggagg |
| 2941 | tctccgtcac cgcgacggag ctgcgggatt tctacggctc cgcggacgtc tccgtccagg |
| 3001 | ccgtcgtggc ggccgcccgc gccgcgacga cgccggccgc ctccccgctg gagcccctgg |
| 3061 | agaacccgac tctgtggcgg gcgctgtacg cgtgcgtcct ggcggccctg gagcgccaga |
| 3121 | ccgggccggt ggccctgttc gccccgctgc gtatcggctc ggacccacgc acgggactgg |
| 3181 | tggtgaaagt tgagagagcg tcgtggggcc cgcccgccgc ccctcgcgcc gctctcctgg |
| 3241 | tcgcggaggc caacattgac atcgacccta tggccctggc ggcgcgcgtt gccgagcatc |
| 3301 | ccgacgcgcg gctggcgtgg gcgcgcctgg cggccattcg cgacaccccc cagtgcgcgt |
| 3361 | ccgccgcttc gctgaccgtt aacatcacca ccggaaccgc gctatttgcg cgcgaatacc |
| 3421 | agactcttgc gtttccgccg atcaagaagg agggcgcgtt cggggacctg gtcgaggtgt |
| 3481 | gcgaggtggg cctgcggcca cgcgggcacc cgcaacgagt cacggcacgg gtgctgctgc |
| 3541 | cccgcgatta cgactacttt gtaagcgccg gcgagaagtt ctccgcgccg gcgctcgtcg |
| 3601 | cccttttccg gcagtggcat accacggtcc acgccgcccc cggggccctg gcccccgtct |
| 3661 | ttgcctttct ggggcccgag tttgaggtcc gggggggacc cgtcccgtac tttgccgtcc |
| 3721 | tggggtttcc gggttggccc acgttcaccg tgccggccac ggccgagtcg gcacgggacc |
| 3781 | tggtgcgcgg ggccgcggcc gcttacgccg cgctcctggg ggcctggccc gcggtggggg |
| 3841 | ccagggtcgt cctccccccg cgagcctggc ccggcgtggc ctcggcggca gccggatgcc |
| 3901 | tcctgcccgc ggtgcgggag gcggtggcgc ggtggcatcc cgccactaaa atcatccaac |
| 3961 | tgttagaccc gcccgcggcc gtcgggcccg tctggacggc gcggttttgc ttccccggac |
| 4021 | ttcgcgccca gctcctggcg gccctggccg acctcggggg gagcgggctg gcggaccccc |
| 4081 | acggccggac gggcctagca agactggacg cgctggtggt ggccgctccc tcagagccct |
| 4141 | gggccggggc cgtcttggag cgcctggtcc cggacacgtg caacgcctgc cctgcgctgc |
| 4201 | ggcagctcct gggtggggta atggccgccg tctgcctgca gatcgaggag acggccagct |
| 4261 | cggtgaagtt cgcggtctgc gggggcgatg ggggtgcgtt ctggggtgtc tttaacgtgg |
| 4321 | acccccaaga cgcggatgcg gcttccgggg tgatcgagga cgcccggcgg gccatcgaga |
| 4381 | cggccgtggg agccgtgctt agggccaacg gcctccggct gcggcaccca ctgtgcctgg |
| 4441 | ccctcgaggg cgtctacacc cacgcagtcg cctggagcca ggcgggagtg tggttctgga |
| 4501 | actcccgcga caacactgac catcttgggg gatttcctct ccgcgggccc gcgtacacca |
| 4561 | cggcggcagg ggtcgtacgc gacacgctgc gacgggtcct gggcctgaca acggcatgcg |
| 4621 | tgccggagga ggacgcactc acggcccggg gccttatgga ggacgcctgc gaccgcctta |
| 4681 | tcttggacgc gtttaataaa cggttggacg cggagtactg gagcgttcgg gtgtccccct |
| 4741 | ttgaggccag cgaccccttg ccccccactg ccttccgcgg cggcgccttg ctggacgcag |
| 4801 | agcactactg gcggcgcgtc gtgcgtgtct gtcccggagg cggggagtcg gtcggcgtcc |
| 4861 | ccgtcgatct atacccgcgg ccccttgtgc tcccccccgt ggactgcgct catcacctgc |
| 4921 | gcgaaatcct gcgcgagatt gagttggtgt ttaccggggt gctggcggga gtatggggcg |
| 4981 | agggggggaa gtttgtgtat ccctttgacg acaagatgtc gtttctgttt gcctgagttt |
| 5041 | gaccaataaa |

### UL30 DNA Polymerase Catalytic Subunit [Human Alphaherpesvirus 1 (Herpes Simplex Virus Type 1)] Gene ID: 2703462. NCBI Reference Sequence: NC_001806.2.

| (SEQ ID NO. 159) | |
|---|---|
| 1 | atgttttccg gtggcggcgg cccgctgtcc cccggaggaa agtcggcggc cagggcggcg |
| 61 | tccgggtttt ttgcgcccgc cggccctcgc ggagccagcc ggggaccccc gccttgtttg |
| 121 | aggcaaaact tttacaaccc ctacctcgcc ccagtcggga cgcaacagaa gccgaccggg |
| 181 | ccaacccagc gccatacgta ctatagcgaa tgcgatgaat ttcgattcat cgccccgcgg |
| 241 | gtgctggacg aggatgcccc cccggagaag cgcgccgggg tgcacgacgg tcacctcaag |
| 301 | cgcgccccca aggtgtactg cgggggggac gagcgcgacg tcctccgcgt cgggtcgggc |
| 361 | ggcttctggc cgcggcgctc gcgcctgtgg ggcggcgtgg accacgcccc ggcggggttc |
| 421 | aaccccaccg tcaccgtctt tcacgtgtac gacatcctgg agaacgtgga gcacgcgtac |
| 481 | ggcatgcgcg cggcccagtt ccacgcgcgg tttatggacg ccatcacacc gacggggacc |
| 541 | gtcatcacgc tcctgggcct gactccggaa ggccaccggg tggccgttca cgtttacggc |
| 601 | acgcggcagt acttttacat gaacaaggag gaggtcgaca ggcacctaca atgccgcgcc |
| 661 | ccacgagatc tctgcgagcg catggccgcg gccctgcgcg agtccccggg cgcgtcgttc |
| 721 | cgcggcatct ccgcggacca cttcgaggcg gaggtggtgg agcgcaccga cgtgtactac |
| 781 | tacgagacgc gccccgctct gttttaccgc gtctacgtcc gaagcgggcg cgtgctgtcg |
| 841 | tacctgtgcg acaacttctg cccggccatc aagaagtacg agggtggggt cgacgccacc |
| 901 | acccggttca tcctggacaa ccccgggttc gtcaccttcg gctggtaccg tctcaaaccg |
| 961 | ggccggaaca acacgctagc ccagccgcgg gccccgatgg ccttcgggac atccagcgac |
| 1021 | gtcgagttta actgtacggc ggacaacctg gccatcgagg ggggcatgag cgacctaccg |
| 1081 | gcatacaagc tcatgtgctt cgatatcgaa tgcaaggcgg ggggggagga cgagctggcc |
| 1141 | tttccggtgg ccgggcaccc ggaggacctg gtcatccaga tatcctgtct gctctacgac |
| 1201 | ctgtccacca ccgccctgga gcacgtcctc ctgttttcgc tcggttcctg cgacctcccc |
| 1261 | gaatcccacc tgaacgagct ggcggccagg ggcctgccca cgcccgtggt tctggaattc |
| 1321 | gacagcgaat tcgagatgct gttggccttc atgacccttg tgaaacagta cggccccgag |
| 1381 | ttcgtgaccg ggtacaacat catcaacttc gactggccct tcttgctggc caagctgacg |
| 1441 | gacatttaca aggtccccct ggacgggtac ggccgcatga acggccgggg cgtgtttcgc |
| 1501 | gtgtgggaca taggccagag ccacttccag aagcgcagca agataaaggt gaacggcatg |
| 1561 | gtgaacatcg acatgtacgg gattataacc gacaagatca agctctcgag ctacaagctc |
| 1621 | aacgccgtgg ccgaagccgt cctgaaggac aagaagaagg acctgagcta tcgcgacatc |
| 1681 | cccgcctact acgccgccgg gcccgcgcaa cgcggggtga tcggcgagta ctgcatacag |
| 1741 | gattccctgc tggtgggcca gctgtttttt aagtttttgc cccatctgga gctctcggcc |
| 1801 | gtcgcgcgct tggcgggtat taacatcacc cgcaccatct acgacggcca gcagatccgc |
| 1861 | gtctttacgt gcctgctgcg cctggccgac cagaagggct ttattctgcc ggacacccag |
| 1921 | gggcgattta ggggcgccgg gggggaggcg cccaagcgtc cggccgcagc ccgggaggac |
| 1981 | gaggagcggc cagaggagga gggggaggac gaggacgaac gcgaggaggg cgggggcgag |
| 2041 | cgggagccgg agggcgcgcg ggagaccgcc ggcaggcacg tggggtacca gggggccagg |
| 2101 | gtccttgacc ccacttccgg gtttcacgtg aaccccgtgg tggtgttcga ctttgccagc |
| 2161 | ctgtacccca gcatcatcca ggcccacaac ctgtgcttca gcacgctctc cctgagggcc |
| 2221 | gacgcagtgg cgcacctgga ggcgggcaag gactacctgg agatcgaggt gggggggcga |
| 2281 | cggctgttct tcgtcaaggc tcacgtgcga gagagcctcc tcagcatcct cctgcgggac |
| 2341 | tggctcgcca tgcgaaagca gatccgctcg cggattcccc agagcagccc cgaggaggcc |
| 2401 | gtgctcctgg acaagcagca ggccgccatc aaggtcgtgt gtaactcggt gtacgggttc |
| 2461 | acgggagtgc agcacggact cctgccgtgc ctgcacgttg ccgcgacggt gacgaccatc |
| 2521 | ggccgcgaga tgctgctcgc gacccgcgag tacgtccacg cgcgctgggc ggccttcgaa |
| 2581 | cagctcctgg ccgatttccc ggaggcggcc gacatgcgcg cccccgggcc ctattccatg |
| 2641 | cgcatcatct acggggacac ggactccatc tttgtgctgt gccgcggcct cacggccgcc |
| 2701 | gggctgacgg ccgtgggcga caagatggcg agccacatct cgcgcgcgct gtttctgccc |
| 2761 | cccatcaaac tcgagtgcga aaagacgttc accaagctgc tgctgatcgc caagaaaaag |
| 2821 | tacatcggcg tcatctacgg gggtaagatg ctcatcaagg gcgtggatct ggtgcgcaaa |
| 2881 | aacaactgcg cgtttatcaa ccgcacctcc agggccctgg tcgacctgct gttttacgac |
| 2941 | gataccgtct ccggagcggc cgccgcgtta gccgagcgcc ccgcggagga gtggctggcg |
| 3001 | cgacccctgc ccgagggact gcaggcgttc ggggccgtcc tcgtagacgc ccatcggcgc |
| 3061 | atcaccgacc cggagaggga catccaggac tttgtcctca ccgccgaact gagcagacac |
| 3121 | ccgcgcgcgt acaccaacaa gcgcctggcc cacctgacgg tgtattacaa gctcatggcc |
| 3181 | cgccgcgcgc aggtcccgtc catcaaggac cggatcccgt acgtgatcgt ggcccagacc |
| 3241 | cgcgaggtag aggagacggt cgcgcggctg gccgccctcc gcgagctaga cgccgccgcc |
| 3301 | ccaggggacg agcccgcccc ccccgcggcc ctgccctccc cggccaagcg cccccgggag |
| 3361 | acgccgtcgc ctgccgaccc cccgggaggc gcgtccaagc cccgcaagct gctggtgtcc |
| 3421 | gagctggccg aggatcccgc atacgccatt gcccacggcg tcgccctgaa cacggactat |
| 3481 | tacttctccc acctgttggg ggcggcgtgc gtgacattca aggccctgtt tgggaataac |
| 3541 | gccaagatca ccgagagtct gttaaaaagg tttattcccg aagtgtggca ccccccggac |
| 3601 | gacgtggccg cgcggctccg gaccgcaggg ttcggggcgg tgggtgccgg cgctacggcg |
| 3661 | gaggaaactc gtcgaatgtt gcatagagcc tttgatactc tagcatgagc cccccgtcga |
| 3721 | agctgatgtc cctcatttta caataaa |

### UL42 DNA Polymerase Processivity Subunit [Human Alphaherpesvirus 1 (Herpes Simplex Virus Type 1)] Gene ID: 24271471. NCBI Reference Sequence: NC_001806.2.

| (SEQ ID NO. 160) | |
|---|---|
| 1 | atgacggatt cccctggcgg tgtggccccc gcctcccccg tggaggacgc gtcggacgcg |
| 61 | tccctcgggc agccggagga gggggcgccc tgccaggtgg tcctgcaggg cgccgaactt |
| 121 | aatggaatcc tacaggcgtt tgccccgctg cgcacgagcc ttctggactc gcttctggtt |
| 181 | atgggcgacc ggggcatcct tatccataac acgatctttg gggagcaggt gttcctgccc |
| 241 | ctggaacact cgcaattcag tcggtatcgc tggcgcggac ccacggcggc gttcctgtct |
| 301 | ctcgtggacc agaagcgctc cctcctgagc gtgtttcgcg ccaaccagta cccggaccta |
| 361 | cgtcgggtgg agttggcgat cacgggccag gccccgtttc gcacgctggt tcagcgcata |
| 421 | tggacgacga cgtccgacgg cgaggccgtt gagctagcca gcgagacgct gatgaagcgc |
| 481 | gaactgacga gctttgtggt gctggttccc cagggaaccc ccgacgttca gttgcgcctg |
| 541 | acgaggccgc agctcaccaa ggtccttaac gcgaccgggg ccgatagtgc cacgcccacc |
| 601 | acgttcgagc tcggggttaa cggcaaattt tccgtgttca ccacgagtac ctgcgtcacc |
| 661 | tttgctgccc gcgaggaggg cgtgtcgtcc agcaccagca cccaggtcca gatcctgtcc |
| 721 | aacgcgctca ccaaggcggg ccaggcggcc gccaacgcca agacggtgta cggggaaaat |
| 781 | acccatcgca ccttctctgt ggtcgtcgac gattgcagca tgcgggcggt gctccggcga |
| 841 | ctgcaggtcg gcgggggcac cctcaagttc ttcctcacga cccccgtccc cagtctgtgc |
| 901 | gtcaccgcca ccggtcccaa cgcggtatcg gcggtatttc tcctgaaacc ccagaagatt |
| 961 | tgcctggact ggctgggtca tagccagggg tctccttcag ccgggagctc ggcctcccgg |
| 1021 | gcctctggga gcgagccaac agacagccag gactccgcgt cggacgcggt cagccacggc |
| 1081 | gatccggaag acctcgatgg cgctgcccgg gcgggagagg cgggggcctt gcatgcctgt |
| 1141 | ccgatgccgt cgtcgaccac gcgggtcact cccacgacca agcgggggcg ctcggggggc |
| 1201 | gaggatgcgc gcgcggacac ggccctaaag aaacctaaga cggggtcgcc caccgcaccc |
| 1261 | ccgcccgcag atccagtccc cctggacacg gaggacgact ccgatgcggc ggacgggacg |
| 1321 | gcggcccgtc ccgccgctcc agacgcccgg agcggaagcc gttacgcgtg ttactttcgc |
| 1381 | gacctcccga ccggagaagc aagccccggc gccttctccg ccttccgggg gggcccccaa |
| 1441 | accccgtatg gttttggatt cccctgacgg ggcggggcct tggcggccgc ccaactctcg |
| 1501 | caccatcccg ggttaatgta aataaa |

### Example 38—Human Papilloma Virus (HPV) Helper Polynucleotide Sequences

HPV polynucleotides can be selected from any serotype, and representative polynucleotides are exemplified below. Meier et al. 2020; Cao et al. 2012 (Cao, M., et al. “HPV-16 E1, E2 and E6 each complement the Ad5 helper gene set, increasing rAAV2 and wt AAV2 production.” Gene therapy 19.4 (2012): 418-424); You et al. 2006 (You, Hong, et al. “Multiple human papillomavirus genes affect the adeno-associated virus life cycle.” Virology 344.2 (2006): 532-540); and Ogston et al. 2000 (Ogston, P.; Raj, K.; Beard, P. Productive Replication of Adeno-Associated Virus Can Occur in Human Papillomavirus Type 16 (HPV-16) Episome-Containing Keratinocytes and Is Augmented by the HPV-16 E2 Protein. J. Virol. 2000, 74, 3494-3504) disclose four HPV early genes E1, E2, E6 and E7, of which E1 shows the highest helping activity, E2 and E6 with intermediate helper activity and E7 with little effect or possibly a slight decrease in cap expression. The three HPV genes (E1, E2, and E6) are unable to stimulate significant rAAV replication in HEK293 cells when used alone. However, when used in conjunction (complementation) with the standard Ad5 helper gene set, E1, E2 and E6 are each capable of significantly boosting rAAV DNA replication and virus particle yield. HPV early gene (E1, E2, E6 and E7) sequences as disclosed at the GenBank are listed below:

### E1 Replication Protein E1 [Human Papillomavirus Type 16] Gene ID: 1489075. NCBI Reference Sequence: NC_001526.4.

| (SEQ ID NO. 161) | |
|---|---|
| 1 | atggctgatc ctgcaggtac caatggggaa gagggtacgg gatgtaatgg atggttttat |
| 61 | gtagaggctg tagtggaaaa aaaaacaggg gatgctatat cagatgacga gaacgaaaat |
| 121 | gacagtgata caggtgaaga tttggtagat tttatagtaa atgataatga ttatttaaca |
| 181 | caggcagaaa cagagacagc acatgcgttg tttactgcac aggaagcaaa acaacataga |
| 241 | gatgcagtac aggttctaaa acgaaagtat ttgggtagtc cacttagtga tattagtgga |
| 301 | tgtgtagaca ataatattag tcctagatta aaagctatat gtatagaaaa acaaagtaga |
| 361 | gctgcaaaaa ggagattatt tgaaagcgaa gacagcgggt atggcaatac tgaagtggaa |
| 421 | actcagcaga tgttacaggt agaagggcgc catgagactg aaacaccatg tagtcagtat |
| 481 | agtggtggaa gtgggggtgg ttgcagtcag tacagtagtg gaagtggggg agagggtgtt |
| 541 | agtgaaagac acactatatg ccaaacacca cttacaaata ttttaaatgt actaaaaact |
| 601 | agtaatgcaa aggcagcaat gttagcaaaa tttaaagagt tatacggggt gagtttttca |
| 661 | gaattagtaa gaccatttaa aagtaataaa tcaacgtgtt gcgattggtg tattgctgca |
| 721 | tttggactta cacccagtat agctgacagt ataaaaacac tattacaaca atattgttta |
| 781 | tatttacaca ttcaaagttt agcatgttca tggggaatgg ttgtgttact attagtaaga |
| 841 | tataaatgtg gaaaaaatag agaaacaatt gaaaaattgc tgtctaaact attatgtgtg |
| 901 | tctccaatgt gtatgatgat agagcctcca aaattgcgta gtacagcagc agcattatat |
| 961 | tggtataaaa caggtatatc aaatattagt gaagtgtatg gagacacgcc agaatggata |
| 1021 | caaagacaaa cagtattaca acatagtttt aatgattgta catttgaatt atcacagatg |
| 1081 | gtacaatggg cctacgataa tgacatagta gacgatagtg aaattgcata taaatatgca |
| 1141 | caattggcag acactaatag taatgcaagt gcctttctaa aaagtaattc acaggcaaaa |
| 1201 | attgtaaagg attgtgcaac aatgtgtaga cattataaac gagcagaaaa aaaacaaatg |
| 1261 | agtatgagtc aatggataaa atatagatgt gatagggtag atgatggagg tgattggaag |
| 1321 | caaattgtta tgtttttaag gtatcaaggt gtagagttta tgtcattttt aactgcatta |
| 1381 | aaaagatttt tgcaaggcat acctaaaaaa aattgcatat tactatatgg tgcagctaac |
| 1441 | acaggtaaat cattatttgg tatgagttta atgaaatttc tgcaagggtc tgtaatatgt |
| 1501 | tttgtaaatt ctaaaagcca tttttggtta caaccattag cagatgccaa aataggtatg |
| 1561 | ttagatgatg ctacagtgcc ctgttggaac tacatagatg acaatttaag aaatgcattg |
| 1621 | gatggaaatt tagtttctat ggatgtaaag catagaccat tggtacaact aaaatgccct |
| 1681 | ccattattaa ttacatctaa cattaatgct ggtacagatt ctaggtggcc ttatttacat |
| 1741 | aatagattgg tggtgtttac atttcctaat gagtttccat ttgacgaaaa cggaaatcca |
| 1801 | gtgtatgagc ttaatgataa gaactggaaa tcctttttct caaggacgtg gtccagatta |
| 1861 | agtttgcacg aggacgagga caaggaaaac gatggagact ctttgccaac gtttaaatgt |
| 1921 | gtgtcaggac aaaatactaa cacattatga |

### E2 Regulatory Protein E2 [Human Papillomavirus Type 16] Gene ID: 1489080. NCBI Reference Sequence: NC_001526.4.

| (SEQ ID NO. 162) | |
|---|---|
| 1 | atggagactc tttgccaacg tttaaatgtg tgtcaggaca aaatactaac acattatgaa |
| 61 | aatgatagta cagacctacg tgaccatata gactattgga aacacatgcg cctagaatgt |
| 121 | gctatttatt acaaggccag agaaatggga tttaaacata ttaaccacca ggtggtgcca |
| 181 | acactggctg tatcaaagaa taaagcatta caagcaattg aactgcaact aacgttagaa |
| 241 | acaatatata actcacaata tagtaatgaa aagtggacat tacaagacgt tagccttgaa |
| 301 | gtgtatttaa ctgcaccaac aggatgtata aaaaaacatg gatatacagt ggaagtgcag |
| 361 | tttgatggag acatatgcaa tacaatgcat tatacaaact ggacacatat atatatttgt |
| 421 | gaagaagcat cagtaactgt ggtagagggt caagttgact attatggttt atattatgtt |
| 481 | catgaaggaa tacgaacata ttttgtgcag tttaaagatg atgcagaaaa atatagtaaa |
| 541 | aataaagtat gggaagttca tgcgggtggt caggtaatat tatgtcctac atctgtgttt |
| 601 | agcagcaacg aagtatcctc tcctgaaatt attaggcagc acttggccaa ccaccccgcc |
| 661 | gcgacccata ccaaagccgt cgccttgggc accgaagaaa cacagacgac tatccagcga |
| 721 | ccaagatcag agccagacac cggaaacccc tgccacacca ctaagttgtt gcacagagac |
| 781 | tcagtggaca gtgctccaat cctcactgca tttaacagct cacacaaagg acggattaac |
| 841 | tgtaatagta acactacacc catagtacat ttaaaaggtg atgctaatac tttaaaatgt |
| 901 | ttaagatata gatttaaaaa gcattgtaca ttgtatactg cagtgtcgtc tacatggcat |
| 961 | tggacaggac ataatgtaaa acataaaagt gcaattgtta cacttacata tgatagtgaa |
| 1021 | tggcaacgtg accaattttt gtctcaagtt aaaataccaa aaactattac agtgtctact |
| 1081 | ggatttatgt ctatatga |

### E6 Protein E6*; Transforming Protein E6 [Human Papillomavirus Type 16] Gene ID: 1489078. NCBI Reference Sequence: NC_001526.4.

| (SEQ ID NO. 163) | |
|---|---|
| 1 | atgcaccaaa agagaactgc aatgtttcag gacccacagg agcgacccag aaagttacca |
| 61 | cagttatgca cagagctgca aacaactata catgatataa tattagaatg tgtgtactgc |
| 121 | aagcaacagt tactgcgacg tgaggtatat gactttgctt ttcgggattt atgcatagta |
| 181 | tatagagatg ggaatccata tgctgtatgt gataaatgtt taaagtttta ttctaaaatt |
| 241 | agtgagtata gacattattg ttatagtttg tatggaacaa cattagaaca gcaatacaac |
| 301 | aaaccgttgt gtgatttgtt aattaggtgt attaactgtc aaaagccact gtgtcctgaa |
| 361 | gaaaagcaaa gacatctgga caaaaagcaa agattccata atataagggg tcggtggacc |
| 421 | ggtcgatgta tgtcttgttg cagatcatca agaacacgta gagaaaccca gctgtaa |

### E7 Transforming Protein E7 [Human Papillomavirus Type 16] Gene ID: 1489079. NCBI Reference Sequence: NC_001526.4.

| (SEQ ID NO. 164) | |
|---|---|
| 1 | atgcatggag atacacctac attgcatgaa tatatgttag atttgcaacc agagacaact |
| 61 | gatctctact gttatgagca attaaatgac agctcagagg aggaggatga aatagatggt |
| 121 | ccagctggac aagcagaacc ggacagagcc cattacaata ttgtaacctt ttgttgcaag |
| 181 | tgtgactcta cgcttcggtt gtgcgtacaa agcacacacg tagacattcg tactttggaa |
| 241 | gacctgttaa tgggcacact aggaattgtg tgccccatct gttctcagaa accataa |

### Example 39—Bocavirus 1 and Baculovirus Helper Polynucleotide Sequences

Bocavirus polynucleotides can be selected from any serotype, and representative polynucleotides as disclosed at the GenBank are exemplified below. Meier et al 2020; Wang, Zekun, et al. 2017 (Wang, Zekun, et al. “Human bocavirus 1 is a novel helper for adeno-associated virus replication.” Journal of virology 91.18 (2017): 10-1128); Guido, Marcello, et al. 2016 (Guido, Marcello, et al. “Human bocavirus: current knowledge and future challenges.” World journal of gastroenterology 22.39 (2016): 8684); and Ning, Kang, et al. 2022 (Ning, Kang, et al. “The small nonstructural protein NP1 of human bocavirus 1 directly interacts with Ku70 and RPA70 and facilitates viral DNA replication.” PLoS pathogens 18.6 (2022): el 010578) disclose that human bocavirus 1 (HBoV1) NS2 (but not NS4), NP1, and BocaSR were required for AAV2 DNA replication and progeny virion formation. Novel small NS proteins (NS2, NS3 and NS4) have been identified in HBoV1, which contain the predictive domains of NS1 activities. HBoV1 expresses one large nonstructural protein (NS1), four small nonstructural proteins (NS2, NS3, NS4, and NP1), one small noncoding RNA (bocavirus-encoded small RNA, BocaSR), and three viral capsid proteins (VP1, VP2, and VP3) from a single precursor mRNA (pre-mRNA) via alternative splicing. NS1, NP1, and BocaSR are essential for DNA replication of HBoV1.

| HBOV1 NP1 | |
|---|---|
| (SEQ ID NO. 165) | |
| 1 | atgacgaaga tgagctcagg gaatatgaaa gacaagcatc gctictacaa aagaaaaggg |
| 61 | agtccagaaa gaggggagag gaagagacac tggcagacaa ctcatcacag gagcaggagc |
| 121 | cgcagcccga tccgacacag tggggagaga ggctcgggct catatcatca ggaacaccca |
| 181 | atcagccacc tattgtcttg cactgcttcg aagacctcag accaagtgat gaagacgagg |
| 241 | gagagtacat cggggaaaaa agacaataga acaaatccat acactgtatt cagtcaacac |
| 301 | agagcttcca atcctgaagc tccagggtgg tgtgggttct actggcactc tactcgcatt |
| 361 | gctagagatg gtactaattc aatctttaat gaaatgaaac aacagtttca acagctacaa |
| 421 | attgataata aaataggatg ggataacact agagaactat tgtttaatca aaagaaaaca |
| 481 | ctagatcaaa aatacagaaa tatgttctgg cactttagaa ataactctga ttgtgaaaga |
| 541 | tgtaattact gggatgatgt gtaccgtaga cacttagcta atgtttcctc acagacagaa |
| 601 | gcagacgaga taactgacga ggaaatgctt tctgctgctg aaagcatgga agcagatgcc |
| 661 | tccaattaag agacagccta gagggtgggt gctgcctgga tacagatatc ttgggc |
| HBOV1 NS1 | |
| (SEQ ID NO. 166) | |
| 1 | gccggcagac atattggatt ccaagatggc gtctgtacaa ccacgtcaca tataaaataa |
| 61 | taaatattca caaggaggag tggttatatg atgtaatcca taaccactcc caggaaatga |
| 121 | cgtatgatag ccaatcagaa ttaagtatta aacctatata agctgctgca cttcctgatt |
| 181 | caatcagact gcatccggtc tccggcgagt gaacatctct ggaaaaagct ccacgcttgt |
| 241 | ggtgagtcta ctatggcttt caatcctcct gtgattagag ctttttctca acctgctttt |
| 301 | acttatgtct tcaaatttcc atatccacaa tggaaagaaa aagaatggct gcttcatgca |
| 361 | cttttagctc atggaactga acaatctatg atacaattaa gaaactgcgc ttctcatccg |
| 421 | gatgaagaca taatccgtga tgacttgctt atttctttag aagatcgcca ttttggggct |
| 481 | gttctctgca aggctgttta catggcaaca actactctca tgtcacacaa acaaaggaat |
| 541 | atgtttcctc gttgtgacat catagttcag tctgagctag gagagaaaaa cttacactgc |
| 601 | catattatag ttgggggaga aggactaagc aagaggaatg ctaaatcatc ctgtgctcag |
| 661 | ttctatggtt taatactagc tgagataatt caacgctgca aatctcttct ggctacacgt |
| 721 | ccttttgaac ctgaggaggc tgacatattt cacactctaa aaaaggctga gcgagaggca |
| 781 | tggggtggag ttactggcgg caacatgcag atccttcaat atagagatcg cagaggagac |
| 841 | cttcatgcac aaacagtgga tcctcttcgc ttcttcaaaa actacctttt acctaaaaat |
| 901 | agatgtattt catcttacag caaacctgat gtttgtactt ctcctgacaa ctggttcatt |
| 961 | ttagctgaaa aaacttactc tcacactctt attaacgggc tgccgcttcc agaacattac |
| 1021 | agaaaaaact accacgcaac cctagataac gaagtcattc cagggcctca agcaatggcc |
| 1081 | tatggaggac gtggtccgtg ggaacatctt cctgaggtag gagatcagcg cctagctgcg |
| 1141 | tcttctgtta gcactactta taaacctaac aaaaaagaaa aacttatgct aaacttgcta |
| 1201 | gacaaatgta aagagctaaa tctattagtt tatgaagact tagtagctaa ttgtcctgaa |
| 1261 | ctactcctta tgcttgaagg tcaaccagga ggggcacgcc ttatagaaca agtcttgggc |
| 1321 | atgcaccata ttaatgtttg ttctaacttt acagctctca catatctttt tcatctacat |
| 1381 | cctgttactt cgcttgactc agacaataaa gctttacagc ttttgttgat tcaaggctat |
| 1441 | aatcctctag ccgttggtca cgccctgtgc tgtgtcctga acaaacaatt cgggaaacaa |
| 1501 | aacactgttt gcttttacgg gcctgcctca acaggtaaaa caaatatggc caaggcaatc |
| 1561 | gtccaaggga ttagacttta tgggtgtgtt aatcatttga acaaaggatt tgtatttaat |
| 1621 | gactgcagac aacgcctagt tgtttggtgg gaggagtgct taatgcacca ggattgggtg |
| 1681 | gaacctgcaa agtgtatctt gggcgggaca gaatgcagaa ttgacgtcaa gcatagagac |
| 1741 | agtgtacttt taactcaaac acctgtaatt atatccacta accacgatat ctacgcggtt |
| 1801 | gttggtggca attctgtttc tcatgttcac gcggctccat taaaagaaag agtgattcag |
| 1861 | ctaaatttta tgaaacaact tcctcaaaca tttggagaaa tcactgctac tgagattgca |
| 1921 | gctcttctac agtggtgttt caatgagtac gactgtactc tgacaggatt taaacaaaaa |
| 1981 | tggaatttag acaaaattcc aaactcattt cctcttgggg tcctttgtcc tactcattca |
| 2041 | caggacttta cacttcacga aaacggatac tgcactgatt gcggtggtta ccttcctcat |
| 2101 | agtgctgaca attctatgta cactgatcgc gcaagcgaaa ctagcacagg agacatcaca |
| 2161 | ccaagtaagt aaatacgcat gcgcaagtaa ttcttttact ttcacttcgc tatttttacc |
| 2221 | aatttttact tttaggtgac ttgggggatt cggacggaga agacaccgag cctgagacat |
| 2281 | cgcaagtgga ctattgtcca cccaagaaac gtcgtctaac tgctccagca agtcctccaa |
| 2341 | actcacctgc gagctctgta agtactatta ctttctttaa cacttggcac gcacagccac |
| 2401 | gtgacgaaga tgagctcagg gaatatgaaa gacaagcatc gctcctacaa aagaaaaggg |
| 2461 | agtccagaaa gaggggagag gaagagacac tggcagacaa ctcatcacag gagcaggagc |
| 2521 | cgcagcccga tccgacacag tggggagaga ggctcgggct catatcatca ggaacaccca |
| 2581 | atcagccacc tatcgtcttg cactgcttcg aagacctcag accaagtgat gaagacgagg |
| 2641 | gaaagtacat cggggaaaaa agacaataga acaaatccat acactgtatt cagtcaacac |
| 2701 | agagcttcca atcctgaagc tccagggtgg tgtgggttct actggcactc tactcgcatt |
| 2761 | gctagagatg gtactaattc aatctttaat gaaatgaaac aacagtttca acagctacaa |
| 2821 | attgataata aaataggatg ggataacact agagaactat tgtttaatca aaagaaaaca |
| 2881 | ctagatcaaa aatacagaaa tatgttctgg cactttagaa ataactctga ttgtgaaaga |
| 2941 | tgtaattact gggatgatgt gtaccgtaga cacttagcta atgtttcctc acagacagaa |
| 3001 | gcagacgaga taactgacga ggaaatgctt tctgctgctg aaagcatgga agcagatgcc |
| 3061 | tccaattaag agacagccta gagggtgggt gctgcctgga tacagatatc ttgggccatt |
| 3121 | taatccactt gataacggtg aacctgtaaa taacgctgat cgcgctgctc aattacatga |
| 3181 | tcacgcctac tctgaactaa taaagagtgg taaaaatcca tacctgtatt tcaataaagc |
| 3241 | tgatgaaaaa ttcattgatg atctaaaaga cgattggtca attggtggaa ttattggatc |
| 3301 | cagttttttt aaaataaagc gcgccgtggc tcctgctttg ggaaataaag agagagccca |
| 3361 | aaaaagacac ttttactttg ctaactcaaa taaaggtgca aaaaaaacaa aaaaaagtga |
| 3421 | acctaaacca ggaacctcaa aaatgtctga cactgacatt caagaccaac aacctgatac |
| 3481 | tgtggacgca ccacaaaaca cctcaggggg aggaacagga agtattggag gaggaaaagg |
| 3541 | atctggtgtg gggatttcca ctggagggtg ggtcggaggt tctcactttt cagacaaata |
| 3601 | tgtggttact aaaaacacaa gacaatttat aaccacaatt caaaatggtc acctctacaa |
| 3661 | aacagaggcc attgaaacaa caaaccaaag tggaaaatca cagcgctgcg tcacaactcc |
| 3721 | atggacatac tttaacttta atcaatacag ctgtcacttc tcaccacagg attggcagcg |
| 3781 | ccttacaaat gaatataagc gcttcagacc taaagcaatg caagtaaaaa tttacaactt |
| 3841 | gcaaataaaa caaatacttt caaatggtgc tgacacaaca tacaacaatg acctcacagc |
| 3901 | tggcgttcac atcttttgtg atggagagca tgcttaccca aatgcatctc atccatggga |
| 3961 | tgaggacgtc atgcctgatc ttccatacaa gacctggaaa ctttttcaat atggatatat |
| 4021 | tcctattgaa aatgaactcg cagatcttga tggaaatgca gctggaggca atgctacaga |
| 4081 | aaaagcactt ctgtatcaga tgcctttttt tctacttgaa aacagtgacc accaagtact |
| 4141 | tagaactggt gagagcactg aatttacttt taactttgac tgtgaatggg ttaacaatga |
| 4201 | aagagcatac attcctcctg gactaatgtt taatccaaaa gtcccaacaa gaagagttca |
| 4261 | gtacataaga caaaacggaa gcacagcagc cagcacaggc agaattcagc catactcaaa |
| 4321 | accaacaagc tggatgacag gacctggcct gctcagtgca caaagagtag gaccacagtc |
| 4381 | atcagacact gctccattca tggtttgcac taacccagaa ggaacacaca taaacacagg |
| 4441 | tgctgcagga tttggatctg gctttgatcc tccaagcgga tgtctggcac caactaacct |
| 4501 | agaatacaaa cttcagtggt accagacacc agaaggaaca ggaaataatg gaaacataat |
| 4561 | tgcaaaccca tcactctcaa tgcttagaga ccaactccta tacaaaggaa accagaccac |
| 4621 | atacaatcta gtgggggaca tatggatgtt tccaaatcaa gtctgggaca gatttcctat |
| 4681 | caccagagaa aatccaatct ggtgcaaaaa accaagggct gacaaacaca caatcatgga |
| 4741 | tccatttgat ggatcaattg caatggatca tcctccaggc actattttta taaaaatggc |
| 4801 | aaaaattcca gttccaactg cctcaaatgc agactcatac ctaaacatat actgtactgg |
| 4861 | acaagtcagc tgtgaaattg tatgggaggt agaaagatac gcaacaaaga actggcgtcc |
| 4921 | agaaagaaga catactgcac tcgggatgtc actgggagga gaaagcaact acacgcctac |
| 4981 | ataccacgtg gatccaacag gagcatacat ccagcccacg tcatatgatc agtgtatgcc |
| 5041 | agtaaaaaca aacatcaata aagtgttgta atcttataag cctctttttt gcttctgctt |
| 5101 | acaagttcct cctcaatgga caagcggaaa gtgaagggtg actgtagtcc tgagctcatg |
| 5161 | ggttcaagac cacagcccga tggtagtggt gttaccgtct cgaacctagc cgacagccct |
| 5221 | tgtacattgt ggggggagct gttttgtttg cttatgcaat cgcgaaactc tatatctttt |
| 5281 | aatgtgttgt tgttgtaca |

It is to be understood that the description, specific examples and data, while indicating exemplary aspects, are given by way of illustration and are not intended to limit the present inventions. Various changes and modifications within the present inventions, including combining aspects in whole and in part, will become apparent to the skilled artisan from the discussion, disclosure and data contained herein, and thus are considered part of the inventions.

1. A method of producing covalently surface modified adeno-associated virus using in vitro conjugation, wherein the method comprises the steps of
(I) combining recombinant AAV (rAAV) with retargeting molecules in a ratio selected to achieve a desired level of conjugation;
(II) incubating the AAV and retargeting molecules for a period of time and under conditions to achieve the desired level of conjugation.

2. A method of producing a covalently surface modified adeno-associated virus according to claim 1, wherein the rAAV is obtained by
(A) transfecting a cell with:
(1) a plasmid comprising a gene of interest flanked by adeno-associated virus (AAV) inverted terminal repeats;
(2) a plasmid comprising an AAV rep gene and an AAV cap gene;
(3) a plasmid comprising AAV rep and cap genes and a polynucleotide sequence encoding a first member of a specific binding pair; and
(4) a plasmid comprising one or more helper polynucleotide sequences;
(B) culturing the transfected cell from step (A) to allow expression of plasmids (1) to (4) and assembly of proteins formed by the expression of plasmids (1) to (4); and
(C) harvesting the recombinant AAV.

3. The method according to claim 2, wherein the cell is a mammalian cell.

4. The method according to claim 3, wherein the mammalian cell is a human cell.

5. The method according claim 1, wherein the recombinant AAV comprises a gene of interest.

6. The method according to claim 1, wherein the recombinant AAV comprises a recombinant capsid protein.

7. The method according to claim 6, wherein the recombinant capsid protein comprises an amino acid sequence of a first member of a specific binding pair.

8. The method according to claim 7, wherein the first member of the specific binding pair is SpyTag.

9. The method according to claim 1, wherein the retargeting molecule is bound to a second cognate member of a specific binding pair.

10. The method according to claim 9, wherein the second cognate member of the specific binding pair is SpyCatcher

11. The method according to claim 1, wherein the retargeting molecule is an Fc-containing protein.

12. The method according to claim 11, wherein the Fc-containing protein is a monoclonal antibody or a monoclonal antibody fragment.

13.-14. (canceled)

15. The method according to claim 1, wherein the ratio of AAV to retargeting molecule is 1:20 to 1:1000, 1:30 to 1:300, or about 1:300.

16.-17. (canceled)

18. The method according to claim 1, wherein the incubating step is about 4 to 72 hours long.

19. The method according to claim 1, wherein the (I) combining step further comprises an additive.

20. The method of claim 19, wherein the additive is isopropyl alcohol.

21. (canceled)

22. The method according to claim 20, wherein isopropyl alcohol is at a concentration of about 5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, or 25% v/v.

23. (canceled)

24. The method according to claim 1, wherein the method achieves optimum conjugation of at least 40%, at least 50%, at least 60%, at least 70%, at least 80% or at least 90%.

25. (canceled)

26. The method according to claim 2, wherein the helper polynucleotide sequences encode adenovirus E4, adenovirus E2, and VA RNA.

27. The method of claim 2, wherein the ratio of the plasmids comprising AAV rep and cap genes and a polynucleotide sequence encoding a first member (3) of a specific binding pair to total plasmids comprising AAV rep and cap genes (2 and 3) is selected from the group consisting of 1/1 to 1/30; 1/4 to 1/13; 1/5 to 1/12; 1/6 to 1/11, 1/7 to 1/10.5 or 1/7.5 to 1/10.

28. A method of separating a covalently surface modified adeno-associated virus (AAV) species from unconjugated retargeting molecule species, wherein the method comprises the steps of
(a) providing a sample comprising the covalently surface modified adeno-associated virus species produced using in vitro conjugation;
(b) subjecting the covalently surface modified adeno-associated virus to anion exchange media using a buffering agent and a salt to separate the covalently surface modified AAV species from unconjugated retargeting molecule species to provide at least one fraction that is enriched in covalently surface modified AAV species; and
(c) harvesting the enriched fraction comprising covalently surface modified AAV species with a defined level of covalent surface modifications.

29. The method according to claim 28, wherein the buffering agent is selected from the group consisting of Bis-Tris-Propane, Bis-Tris, Tris, Glycine, Bicine, Tricine, Acetate, Borate, Citrate, Carbonate, Phosphate, Formate, Sulfate, Succinic acid, Sulfonic acid and variants thereof (for example, MES, PIPES, HEPES, CHES, CAPS, MMS, PBMS), Diethanolamine, and Imidazole, and the salt is selected from the group consisting of NaCl, KCl, MgCl₂, CaCl₂), NH₄Cl, Na₂SO₄, CaSO4, K₂SO₄, MgSO₄, (NH₄)₂SO₄, sodium citrate, and tetramethylammonium chloride (TMAC).

30. The method according to claim 28, wherein the anion exchange media is selected from the group consisting of CIM QA, CIM DEAE, PRIMA T, Capto Q, Capto Q ImpRes, CAPTO DEAE, POROS HQ, POROS XQ, POROS PI, POROS D, Fractogel EMD TMAE, Fractogel EMD DEAE, Nuvia Q, Nuvia HP-Q, Sartobind STIC PA, Sartobind Q, Natrix Q.

31. A method of separating a covalently surface modified adeno-associated virus (AAV) species from unconjugated retargeting molecule species, wherein the method comprises the steps of
(a) providing a sample comprising the covalently surface modified adeno-associated virus species produced using an in vitro conjugation;
(b) subjecting the covalently surface modified adeno-associated virus to cation exchange media using a buffering agent and a salt to separate the covalently surface modified AAV species from unconjugated retargeting molecule species to provide at least one fraction that is enriched in covalently surface modified AAV species; and
(c) harvesting the enriched fraction comprising covalently surface modified AAV species.

32. The method according to claim 31, wherein the cation exchange media is selected from the group consisting of CIM SO₃, Capto S, Capto S ImpRes, Capto S ImpAct, Capto SP, POROS HS, POROS XS, CM Sepharose, Nuvia S, Nuvia HR-Sartobind S, Natrix CH.

33. A method of separating a covalently surface modified adeno-associated virus (AAV) species from unconjugated retargeting molecule species, wherein the method comprises the steps of
(a) providing a sample comprising the covalently surface modified adeno-associated virus species produced using an in vitro conjugation;
(b) subjecting the covalently surface modified adeno-associated virus to multimodal ion exchange using a buffering agent and a salt and an inorganic salt for loading and a low salt and low pH buffer for elution in order to separate the covalently surface modified AAV species from unconjugated retargeting molecule species to provide a fraction that is enriched in covalently surface modified AAV species; and
(c) harvesting at least one enriched fraction comprising covalently surface modified AAV species.

34. A method of separating a covalently surface modified adeno-associated virus (AAV) species from unconjugated retargeting molecule species, wherein the method comprises the steps of
(a) providing a sample comprising the covalently surface modified adeno-associated virus species produced using an in vitro conjugation;
(b) subjecting the covalently surface modified adeno-associated virus to multimodal ion exchange using a buffering agent is selected from the group consisting of Bis-Tris-Propane, Bis-Tris, Tris, Glycine, Bicine, Tricine, Acetate, Borate, Citrate, Carbonate, Phosphate, Formate, Sulfate, Succinic acid, Sulfonic acid and variants thereof (for example, MES, PIPES, HEPES, CHES, CAPS, MMS, PBMS), Diethanolamine, and Imidazole, and the salt is selected from the group consisting of NaCl, KCl, MgCl₂, CaCl₂), NH₄Cl, Na₂SO₄, CaSO4, K₂SO₄, MgSO₄, (NH₄)₂SO₄, sodium citrate, and tetramethylammonium chloride (TMAC) in order to separate the covalently surface modified AAV species from unconjugated retargeting molecule species to provide a fraction that is enriched in covalently surface modified AAV species; and
(c) harvesting the enriched fraction comprising covalently surface modified AAV species.

35. The method according to claim 34, wherein beads of the multimodal binding and size separation bead chromatography are functionalized with an octyl amine that is hydrophobic and positively charged.

36. The method according to claim 35, wherein the beads comprise a matrix that provides size exclusion.

37. The method according to claim 34, where the beads are Capto™ Core 400 or Capto™ Core 700.

38. A method according to claim 28, wherein the method further comprises the use of single-pass tangential flow filtration.

39.-44. (canceled)

45. The method according to claim 2, wherein the cell is a rodent cell.

46. The method according to claim 45, wherein the rodent cell is a CHO (Chinese hamster ovary) or BHK (baby hamster kidney) cell.
